(19) United States
(12) Patent Application Publication
(10) Pub. No.: US 2023/0081195 A1
(43) Pub. Date: Mar. 16, 2023

(54) METHODS OF CONTROLLING GRAIN SIZE AND WEIGHT

(71) Applicant: INSTITUTE OF GENETICS AND DEVELOPMENT BIOLOGY CHINESE ACADEMY OF SCIENCES, Chaoyang District, Beijing (CN)

(72) Inventors: Yunhai LI, Chaoyang District, Beijing (CN); Jia LYU, Chaoyang District, Beijing (CN); Penggen DUAN, Chaoyang District, Beijing (CN); Yapei LIU, Chaoyang District, Beijing (CN); Limin ZHANG, Chaoyang District, Beijing (CN); Baolan ZHANG, Chaoyang District, Beijing (CN)

(21) Appl. No.: 17/760,160

(22) PCT Filed: Feb. 8, 2021
(86) PCT No.: PCT/EP2021/052951

§ 371 Date: Aug. 4, 2022

(30) Foreign Application Priority Data
Feb. 7, 2020 (CN) PCT/CN2020/074530

(51) Int. Cl. C12N 15/82; C07K 14/415; C12N 9/12

(52) U.S. Cl. CPC C12N 15/8262; C07K 14/415; C12Y 207/11001; C12N 9/12

(57) ABSTRACT

The invention relates to methods of increasing grain size and/or weight in a plant, as well as plants with increased grain size and/or weight.

### FIELD OF THE INVENTION

The invention relates to methods of increasing grain size and/or weight in a plant, as well as plants with increased grain size and/or weight by reducing the expression and/or activity of OML4. Alternatively, the invention relates to methods of increasing grain number by increasing the expression and/or activity of OML4.

### BACKGROUND OF THE INVENTION

The world population continues to increase rapidly, and this increase has led to a growing demand for staple crops, such as rice, wheat and maize. Grain yield is determined by tiller number, grain number and grain weight. As grain size is a key component of grain weight, regulation of grain size is a crucial strategy to increase grain production. Grain growth is restricted by spikelet hulls, which influence final grain size in rice. In turn, the growth of the spikelet hull is determined by cell proliferation and cell expansion processes. Several genes that regulate grain size by influencing cell proliferation in the spikelet hull have been described in rice, such as GW2, GW5/GSE5, GW8/OsSPL16, GS3, GS9, OsMKKK10-OsMKK4-OsMPK6 and MKP1. In addition, several genes that control grain size by influencing cell expansion in the spikelet hulls have been reported in rice, such as GS2/OsGRF4, OsGSK5, GLW7 (SPL13), GL7, PGL1/2 and APG. However, the genetic and molecular relationships between these factors remain largely unknown. There therefore exists a need to increase grain size and/or grain weight in staple crops. There also exists a need to increase grain number in staple crops. The present invention addresses this need.

### SUMMARY OF THE INVENTION

We have identified genes whose loss and gain of functions lead to opposite effects on grain size. Here we report that the Mei2-Like protein 4 (OML4) encoded by the LARGE1 gene is phosphorylated by the glycogen synthase kinase 2 (GSK2) and negatively controls grain size and weight in rice. Loss of function of OML4 leads to large and heavy grains, while overexpression of OML4 causes small and light grains. OML4 regulates grain size by restricting cell expansion in the spikelet hull. OML4 is expressed in developing inflorescences (e.g. panicles of rice) and grains, and expression (indicated by GFP-OML4 fusion protein) is localized in the nuclei. Biochemical analyses show that GSK2 physically interacts with OML4 and phosphorylates it, therefore possibly influencing the stability of OML4. Genetic analyses support that GSK2 and OML4 act, at least in part, in a common pathway to control grain size in rice. Therefore, our findings reveal a significant genetic and molecular mechanism to control both grain size and weight in crops.

In a first aspect of the invention, there is provided a method of increasing grain size and/or weight, the method comprising reducing or abolishing the expression and/or activity of Mei2-Like protein 4 (OML4).

Preferably, the method comprises introducing at least one mutation into at least one nucleic acid sequence encoding OML4 and/or at least one mutation into the promoter of OML4.

In a further embodiment, the method further comprises additionally reducing or abolishing the expression and/or activity of a SHAGGY-like kinase (GSK2). Preferably, the method comprises introducing at least one mutation into at least one nucleic acid sequence encoding GSK2 and/or at least one mutation into the promoter of GSK2.

In one embodiment, the mutation is a loss of function or partial loss of function mutation. Preferably, the mutation is introduced using targeted genome modification, preferably ZFNs, TALENs or CRISPR/Cas9 or mutagenesis, preferably TILLING or T-DNA insertion. Alternatively, the method comprises using RNA interference to reduce or abolish the expression of a OML4 nucleic acid sequence or a GSK2 nucleic acid sequence.

In another aspect of the invention, there is provided a genetically modified plant, plant cell or part thereof characterised by reduced or abolished expression of OML4. Preferably, the plant comprises at least one mutation in at least one nucleic acid sequence encoding a OML4 gene and/or at least one mutation into the promoter of OML4. Most preferably the plant part is a seed or grain (such terms can be used interchangeably). Also provided, are progeny plants obtained or obtainable from the seeds, as well as seeds obtained from said progeny plants.

In another embodiment, the plant further comprises at least one mutation in at least one nucleic acid sequence encoding GSK2 and/or at least one mutation into the promoter of GSK2.

Preferably, the mutation is a loss of function or partial loss of function mutation.

In an alternative embodiment, the plant comprises an RNA interference construct that reduces or abolishes the expression of OML4.

In another aspect of the invention, there is provided a method of producing a plant with increased grain size and/or weight, the method comprising introducing at least one mutation into at least one nucleic acid sequence encoding a OML4 polypeptide and/or at least one mutation into the promoter of OML4. In one embodiment, the method further comprises introducing at least one mutation into at least one nucleic acid sequence encoding a GSK2 polypeptide and/or at least one mutation into the promoter of GSK2. Preferably, the mutation is a loss of function or partial loss of function mutation.

According to any aspect of the invention, in one embodiment, the OML4 nucleic acid sequence encodes a polypeptide comprising SEQ ID NO: 1 or a functional variant or homolog thereof, and preferably the nucleic acid sequence encoding OML4 comprises a nucleic sequence as defined in SEQ ID NO: 2. In another embodiment, the promoter of OML4 comprises a sequence as defined in SEQ ID NO: 3 or a functional variant or homolog thereof.

In a further embodiment, the GSK2 nucleic acid sequence encodes a polypeptide as defined in SEQ ID NO: 4 or a functional variant or homolog thereof, and preferably, the GSK2 nucleic acid sequence comprises a nucleic acid sequence as defined in SEQ ID NO: 5 or a functional variant or homolog thereof. In another embodiment, the GSK2 promoter comprises a nucleic acid sequence as defined in SEQ ID NO: 6 or a functional variant or homolog thereof.

In one embodiment of any of the above described methods, the mutation is introduced using targeted genome modification, preferably ZFNs, TALENs or CRISP/Cas9, or the mutation is introduced using mutagenesis, preferably TILLING or T-DNA insertion.

According to any aspect of the invention, in one embodiment, the plant is a crop plant. Preferably, the plant is selected from rice, wheat, maize, soybean and brassicas.

### DESCRIPTION OF THE FIGURES

The invention is further described in the following non-limiting figures:

FIG. 1 shows that LARGE1 influences grain size and plant morphology. (A, B) ZHJ and large1-1 grains. (C, D) ZHJ and large1-1 plants. (E) ZHJ (left) and large1-1 (right) panicles. (F, G) Grain length and width of ZHJ and large1-1. (H) 1000-grain weight of ZHJ and large1-1. (I) Plant height of ZHJ and large1-1. (J) Panicle length of ZHJ and large1-1. (K) The number of ZHJ and large1-1 primary panicle branches. (L) The number of ZHJ and large1-1 secondary panicle branches. Values in F-H are given as mean+SD (n≥50). Values in I-L are given as means+SD (n=20). Asterisks indicate significant differences between ZHJ and large1-1. **P<0.01 compared with the wild type (ZHJ) by Student's t-test. Bars: 2 mm in A and B; 10 cm in C-E.

FIG. 2 shows that the large1 forms large grains due to increased cell expansion in the spikelet hull. (A, B) SEM analysis of the outer surface of ZHJ (A) and large1-1 (B) lemmas. (C, D) SEM analysis of the inner surface of ZHJ (C) and large1-1 (D) lemmas. (E, F) The average length (E) and width (F) of outer epidermal cells in ZHJ and large1-1 lemmas. (G) Outer epidermal cell number in the longitudinal direction in ZHJ and large1-1 lemmas. (H) Outer epidermal cell number in the transverse direction in ZHJ and large1-1 lemmas. (I, J) The average length (I) and width (J) of inner epidermal cells in the longitudinal direction in ZHJ and large1-1 lemmas. Values in E-J are given as the means+SD (n≥50). **P<0.01 compared with the wild type by Student's t-test. Bars: 50 μm in A-D.

FIG. 3 shows that LARGE1 encodes the mei2-like protein OML4. (A) The LARGE1/OML4 gene structure. The coding sequence was shown using the black box, and introns were indicated using black lines. ATG and TGA represent the start codon and the stop codon, respectively. (B) OML4 and mutated protein encodes by large1. The OML4 protein contains three RNA recognition motif (RRM) domains. The mutation results in a premature termination codon in OML4, causing a truncated protein. (C) The dCAPS1 marker was developed according to the large1-1 mutation. The PCR products were digested by the restriction enzyme Hph I. (D, E) Mature paddy (D) and brown (E) rice grains of ZHJ, large1-1, gLARGE1; large1-1 #1 and gLARGE1; large1-1 #2. (F, G) Grain length (F) and width (G) of ZHJ, large1-1, gLARGE1; large1-1 #1 and gLARGE1; large1-1 #2. Asterisks indicate significant differences between ZHJ and large1-1. **P<0.01 compared with the wild type by Student's t-test. (H) The relative OML4 gene expression level in young panicle of 1 cm (YP1) to 15 cm (YP15) in ZHJ. Values are given as mean±SD. Three biological replicates were used (n=3). (I) OML4 expression activity was monitored by proOML4::GUS transgene expression. Histochemical analysis of GUS activity in panicles at different developmental stages. (J, K) Mature paddy (J) and brown (K) rice grains of ZHJ, large1-1, gLARGE1-GFP; large1-1 #1. (L-O) Subcellular location of OML4-GFP in gLARGE1-GFP; large1-1 #1 root cells. GFP fluorescence of GFP-OML4 (L), DAPI staining (M), DIC (N) and merged (O) images are shown. Bars: 2 mm in D, E, J and K; 1 cm in I; 10 μm in L-O.

FIG. 4 shows that Overexpression of OML4 results in smaller grains. (A, B) ZHJ and proActin:OML4 grains. (C, D) Grain length and width of ZHJ and proActin:OML4 transgenic lines. (E) 1000-grain weight of ZHJ and proActin:OML4 transgenic lines. (F) Expression level of OML4 in ZHJ and proActin:OML4 transgenic lines. Three biological replicates were used (n=3). ACTIN1 was used to normalize expression. (G) ZHJ and proActin:OML4 plants. (H) Plant height of ZHJ and proActin:OML4 transgenic lines. (I) ZHJ and proActin:OML4 panicles. (J) Panicle length of ZHJ and proActin:OML4 transgenic lines. (K, L) The primary and secondary panicle branch number of ZHJ and proActin:OML4 transgenic lines. (M) Total grain number per panicle of ZHJ and proActin:OML4 transgenic lines. (N, O) SEM analysis of the outer surface of ZHJ (N) and proActin:OML4 #1 (0) lemmas. (P, Q) The average length and width of outer epidermal cells in the longitudinal direction in ZHJ and proActin:OML4 #1 lemmas. (R, S) The number of outer epidermal cells in the longitudinal and transverse direction in ZHJ and proActin:OML4 #1 lemmas. Values in C-E, and P-S are given as the means±SD (n≥50). Value F is given as the mean±SD. Values H, and J-M are given as the means±SD (n=20). Asterisks indicate significant differences between ZHJ and proActin:OML4 transgenic lines. *P<0.05; **P<0.01 compared with the wild type by Student's t-test. Bars: 2 mm in A and B; 10 cm in G and I; 50 μm in N and 0.

FIG. 5 shows that OML4 physically interacts with GSK2 in Vitro and in Vivo. (A) OML4 interacts with GSK2 in yeast cells. Yeast cells were cultured on SD/-Trp-Leu or SD/-Trp-Leu-His-Ade media. (B) OML4 associates with GSK2 in N. benthamiana. OML4-nLUC and GSK2-cLUC were co-expressed in N. benthamiana leaves. Luciferase activity was observed 48 hours after infiltration. The range of luminescence intensity was scaled by the pseudocolor bar. (C) Bimolecular fluorescence complementation (BiFC) assays shown that OML4 interacts with GSK2 in N. benthamiana. OML4-cYFP was coexpressed with GSK2-nYFP in leaves of N. benthamiana. (D) OML4 binds GSK2 in vitro. GSK2-GST was incubated with OML4-MBP and pulled down by OML4-MBP and detected by immunoblot with anti-GST antibody. IB: immunoblot. (E) Interaction between OML4 and GSK2 in the Co-IP assays. Anti-MYC beads were used to immunoprecipitate GSK2-GFP proteins. Gel blots were probed with anti-MYC or anti-GFP antibody. Bars: 50 μm in C.

FIG. 6 shows that GSK2 is required for the phosphorylation of OML4. (A) GSK2 phosphorylates OML4 in vitro. The phosphorylated OML4-FLAG, nOML4-FLAG (the N-terminal of OML4) and cOML4-FLAG (the C-terminal of OML4) were separated by phos-tag SDS-PAGE. The phosphorylated protein was marked with the red vertical line. (B) Detection of phosphorylation sites of OML4 by LC-MS/MS after in vitro phosphorylation reaction. OML4 contains 1001 residues. The phosphorylate residues detected by LC-MS/MS were shown in red. Two important residues shown by underline, were substituted into phosphor-dead residues. (C) S(105) and S(607) partially influence the phosphorylation of OML4. The phosphorylated nOML4-FLAG, nOML4(S105A)-FLAG, cOML4-FLAG and cOML4(S607A)-FLAG were separated by phos-tag SDS-PAGE. The phosphorylated protein was marked with the red vertical line. (D) S(105) and S(607) partially influence the phosphorylation of OML4. The phosphorylated OML4-MBP, OML4S105A, S607A-MBP and GSK2-GST were separated by phos-tag SDS-PAGE. The phosphorylated protein was marked with red vertical line. (E) GSK2 influences the abundance of OML4. GSK2-GFP and OML4-MYC were co-expressed in tobacco leaves and protein levels were detected by western blotting. This result was repeated for three times. (F) S(105) and S(607) partially influence the abundance of OML4. GSK2-GFP and OML4-MYC or OML4S105A, S607A-MYC were co-expressed in tobacco leaves and protein levels were detected by western blotting. This result was repeated for three times.

FIG. 7 shows that GSK2 acts genetically with OML4 to regulate seed size. (A, B) ZHJ and GSK2-RNAi grains. (C) Expression level of GSK2 in ZHJ and GSK2-RNAi transgenic lines. Three biological replicates were used (n=3). ACTIN1 was used to normalize expression. (D, E) Grain length (D) and width (E) of ZHJ and GSK2-RNAi transgenic lines. (F) 1000-grain weight of ZHJ and GSK2-RNAi transgenic lines. (G, H) SEM analysis of the outer surface of ZHJ (G) and GSK2-RNAi #1 (H) lemmas. (I, J) The average length and width of outer epidermal cells in the longitudinal direction in ZHJ and GSK2-RNAi #1 lemmas. (K) Grains of ZHJ, large1-1, GSK2-RNAi #1 and large1-1; GSK2-RNAi #1. (L) Grain length of ZHJ, large1-1, GSK2-RNAi #1 and large1-1; GSK2-RNAi #1. Values in D-F, I-J, and L are given as the means+SD (n50). *P<0.05; **P<0.01 compared with the wild type by Student's t-test. Bars: 2 mm in A, B and K; 50 μm in G and H.

FIG. 8 shows the expression level of the indicated genes in ZHJ and large1-1 panicles. ACTIN1 was used to normalize expression. Values are means+SD relative to the ZHJ value set at 1. Three biological replicates were used (n=3). *P<0.05; **P<0.01 compared with the wild type by Student's t-test.

FIG. 9 shows the CDS and protein sequence of OML4. (A) The full-length cDNA sequence of OML4. The deletion sequence in large1-1 in the OML4 gene is show in red. (B) The amino acid sequence of OML4. (C) The amino acid sequence of large1-1.

FIG. 10 shows the plant height, panicle size and grain number per panicle of gLARGE1;large1-1. (A) Plants of ZHJ, large1-1, gLARGE1;large1-1 #1 and gLARGE1;large1-1 #2. (B) Phenotypes of ZHJ (left), large1-1 (middle) and gLARGE1;large1-1 #1 (right) panicles. (C) Plant height of ZHJ, large1-1 and gLARGE1;large1-1 #1. (D) Panicle length of ZHJ, large1-1 and gLARGE1;large1-1 #1. (E) The number of ZHJ, large1-1 and gLARGE1;large1-1 #1 primary panicle branches. (F) 1000-grain weight of ZHJ, large1-1 and gLARGE1;large1-1 #1. Values in C-E are given as the means+SD (n=20). Value F is given as the mean+SD (n=100). Asterisks indicate significant differences between ZHJ and large1-1 or ZHJ and gLARGE1;large1-1 #1. **P<0.01 compared with the wild type by Student's t-test. Bars: 10 cm in A and B.

FIG. 11 shows the structural features and phylogenetic tree of OML4. (A) Amino acid sequence alignment of MEI2-LIKE proteins in rice. The three conserved RNA Recognition Motif (RRM) are marked. (B) Phylogenetic tree of MEI2-LIKE proteins in rice and Arabidopsis. OML1, OML2, OML3, OML4, and OML5 are from O. sativa, TE1 and LOC103653544 (MEI2-LIKE protein 1) are from Z. mays, AML1, AML2, AML3, AML4, and AML5 are from Arabidopsis. The multiple sequence alignment and construction of phylogenetic tree were performed with MEGA7 using neighbor-joining method with 100 bootstrap replicates.

FIG. 12 shows the identification of the large1-1 mutation. CHR, chromosome; POS, position in chromosome. The whole genome sequencing reveals the one deletion in the LOC_Os02g31290 gene, which has a SNP/INDEL-index=1.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of botany, microbiology, tissue culture, molecular biology, chemistry, biochemistry and recombinant DNA technology, bioinformatics, which are within the skill of the art. Such techniques are explained fully in the literature.

As used herein, the words “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences.

The terms “polypeptide” and “protein” are used interchangeably herein and refer to amino acids in a polymeric form of any length, linked together by peptide bonds.

The aspects of the invention involve recombinant DNA technology and exclude embodiments that are solely based on generating plants by traditional breeding methods.

Methods of Increasing Grain Size and/or Weight

In a first aspect of the invention, there is provided a method of increasing grain size and/or weight in a plant, wherein the method comprises reducing or abolishing the expression and/or activity of Mei2-Like protein 4 (OML4).

In one embodiment, an “increase” in grain size and/or weight may comprise an increase of at least 5%, 10%, 15%, 20%, 25%, 30%, 35%, 40%, 45% or 50% compared to the grain size and/or weight in a wild-type or control plant. In one embodiment, the increase may be between 5 and 30% and even more preferably between 10 and 25% compared to the grain size and/or weight in a wild-type or control plant. In one embodiment grain size may comprise one of grain length and/or grain width. In a further embodiment, the grain weight may comprise thousand-grain weight. Any of the above can be measured using standard techniques in the art.

In a further aspect of the invention, there is provided a method of increasing yield the method comprising reducing or abolishing the expression or activity of the OML4 gene. The term “yield” in general means a measurable produce of economic value, typically related to a specified crop, to an area, and to a period of time. Individual plant parts directly contribute to yield based on their number, size and/or weight. The actual yield is the yield per square meter for a crop and year, which is determined by dividing total production (includes both harvested and appraised production) by planted square metres.

In one example, yield is increased by at least 5%, 10%, 15%, 20%, 25%, 30%, 35%, 40%, 45%, 50% 55%, 60%, 65%, 70%, 75%, 80%, 85%, 90%, or 95% compared to a control or wild-type plant. In a preferred embodiment, yield is increased by at least 10%, and even more preferably between 10 and 60% compared to a control or wild-type plant.

In a further aspect of the invention, the method further comprises reducing or abolishing the expression or activity of SHAGGY-like kinase (GSK2).

In one embodiment the method comprises introducing at least one mutation into OML4. In a further embodiment, the method comprises introducing at least one mutation into OML4 and at least one mutation into GSK2.

“By at least one mutation” is meant that where the OML4 or GSK2 gene is present as more than one copy or homoeologue (with the same or slightly different sequence) there is at least one mutation in at least one gene. Preferably all genes are mutated in OML4 and/or GSK2.

The terms “reducing” means a decrease in the levels of OML4 or GSK2 expression and/or activity by up to 10%, 20%, 30%, 40%, 50%, 60%, 70%, 80% or 90% when compared to the level in a wild-type or control plant. The term “abolish” expression means that no expression of OML4 or GSK2 polypeptide is detectable or that no functional OML4 or GSK2 polypeptide is produced. Methods for determining the level of OML4 or GSK2 polypeptide expression and/or activity would be well known to the skilled person. These reductions can be measured by any standard technique known to the skilled person. For example, a reduction in the expression and/or content levels of at least OML4 or GSK2 expression may be a measure of protein and/or nucleic acid levels and can be measured by any technique known to the skilled person, such as, but not limited to, any form of gel electrophoresis or chromatography (e.g. HPLC).

In one embodiment, the method comprises introducing at least one mutation into the, preferably endogenous, gene encoding OML4 and/or the OML4 promoter. In another embodiment, the method comprises introducing a further mutation into the, preferably endogenous, gene encoding GSK2 and/or the GSK2 promoter. Preferably, said mutation is in the coding region of the OML4 or the GSK2 gene. In a further embodiment, at least one mutation or structural alteration may be introduced into the OML4 or GSK2 promoter such that the OML4 or GSK2 gene is either not expressed (i.e. expression is abolished) or expression is reduced, as defined herein. In an alternative embodiment, at least one mutation may be introduced into the OML4 or GSK2 gene such that the altered gene does not express a full-length (i.e. expresses a truncated) OML4 or GSK2 protein or does not express a fully functional OML4 or GSK2 protein. In this manner, the activity of the OML4 or GSK2 polypeptide can be considered to be reduced or abolished as described herein. In any case, the mutation may result in the expression of OML4 or GSK2 with no, significantly reduced or altered biological activity in vivo. Alternatively, OML4 or GSK2 may not be expressed at all.

In one embodiment, the sequence of the OML4 gene comprises or consists of a nucleic acid sequence as defined in SEQ ID NO: 2 (genomic) or a functional variant or homologue thereof and encodes a polypeptide as defined in SEQ ID NO: 1 or a functional variant or homologue thereof.

By “OML4 promoter” is meant a region extending for at least 2000-2500 bp, preferably 2049 bp upstream of the ATG codon of the OML4 ORF (open reading frame). In one embodiment, the sequence of the OML4 promoter comprises or consists of a nucleic acid sequence as defined in SEQ ID NO: 3 or a functional variant or homologue thereof. Similarly, by “GSK2 promoter” is meant a region extending at least 200-300 bp, preferably 247 bp upstream of the ATG codon of the GSK2 ORF (open reading frame). In one embodiment, the sequence of the GSK2 promoter comprises or consists of a nucleic acid sequence as defined in SEQ ID NO: 6 or a functional variant or homologue thereof.

In the above embodiments an ‘endogenous’ nucleic acid may refer to the native or natural sequence in the plant genome. In one embodiment, the endogenous sequence of the OML4 gene comprises SEQ ID NO: 2 and encodes an amino acid sequence as defined in SEQ ID NO: 1 or homologs thereof. Also included in the scope of this invention are functional variants (as defined herein) and homologs of the above identified sequences. Examples of OML4 homologs are shown in SEQ ID NOs: 7-9, 13-15, 19-21 and 25-27. Accordingly, in one embodiment, the homolog encodes a polypeptide selected from SEQ ID NOs: 7, 13, 19 or 25 or the homolog comprises or consists of a nucleic acid sequence selected from SEQ ID NOs: 8, 14, 20, 26. In a further embodiment, the endogenous sequence of the GSK2 gene comprises SEQ ID NO: 5 and encodes an amino acid sequence as defined in SEQ ID NO: 4 or homologs thereof. Also included in the scope of this invention are functional variants (as defined herein) and homologs of the above identified sequences. Examples of GSK2 homologs are shown in SEQ ID NOs: 10-12, 16-18, 22-24 and 28-30. Accordingly, in one embodiment, the homolog encodes a polypeptide selected from SEQ ID NOs: 10, 16, 22 or 28 or the homolog comprises or consists of a nucleic acid sequence selected from SEQ ID NOs: 11, 17, 23 or 29.

The term “functional variant of a nucleic acid sequence” as used herein with reference to any SEQ ID describes herein refers to a variant gene sequence or part of the gene sequence which retains the biological function of the full non-variant sequence. A functional variant also comprises a variant of the gene of interest which has sequence alterations that do not affect function, for example in non-conserved residues. Also encompassed is a variant that is substantially identical, i.e. has only some sequence variations, for example in non-conserved residues, compared to the wild type sequences as shown herein and is biologically active. Alterations in a nucleic acid sequence which result in the production of a different amino acid at a given site that do not affect the functional properties of the encoded polypeptide are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

In one embodiment, a functional variant has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic acid or amino acid sequence.

The term homolog, as used herein, also designates a OML4 or GSK2 promoter or OML4 or GSK2 gene orthologue from other plant species. A homolog may have, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the amino acid represented by any of SEQ ID NO: 1 or 4 or to the nucleic acid sequences as shown in SEQ ID NO: 2 or 5. Functional variants of OML4 homologs as defined above are also within the scope of the invention.

The “OML4” or “LARGE1” gene (such terms are used interchangeably herein) encodes a Mei-2 like protein, OML4. This protein is characterised by three RNA recognition motifs or RRMs.

In one embodiment, the sequence of the RRMs is selected from:

SEQ ID NO: 37
SRTLFVRNINSNVEDSELKLLFEHFGDIRALYTACKHRGFVM
ISYYDIRSALNAKMELQNKALRRRKLDIHYSIPKD:
SEQ ID NO: 38
QGTIVLFNVDLSLTNDDLHKIFGDYGEIKEIRDTPQKGHHKI
IEFYDVRAAEAALRALNRNDIAGKKIKLE;
and
SEQ ID NO: 39
LMIKNIPNKYTSKMLLAAIDENHKGTYDFIYLPIDFKNKCNV
GYAFINMTNPQHIIPFYQTFNGKKWEKFNSEKVASLAYARIQ
GK:

Accordingly, in one embodiment, the OML4 nucleic acid (coding) sequence encodes a OML4 protein comprising at least one RRM motif, preferably all three motifs as defined above, or a variant thereof, wherein the variant has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to at least one of SEQ ID No 37 to 39 as defined herein.

The “GSK2” gene (SHAGGY-like kinase) encodes a serine/threonine kinase, which is an ortholog of BIN2, and is involved in BR signalling.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognised that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Non-limiting examples of algorithms that are suitable for determining percent sequence identity and sequence similarity are the BLAST and BLAST 2.0 algorithms.

Suitable homologues can be identified by sequence comparisons and identifications of conserved domains. There are predictors in the art that can be used to identify such sequences. The function of the homologue can be identified as described herein and a skilled person would thus be able to confirm the function, for example when overexpressed in a plant.

Thus, the nucleotide sequences of the invention and described herein can also be used to isolate corresponding sequences from other organisms, particularly other plants, for example crop plants. In this manner, methods such as PCR, hybridization, and the like can be used to identify such sequences based on their sequence homology to the sequences described herein. Topology of the sequences and the characteristic domains structure can also be considered when identifying and isolating homologs. Sequences may be isolated based on their sequence identity to the entire sequence or to fragments thereof. In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen plant. The hybridization probes may be genomic DNA fragments, cDNA fragments, RNA fragments, or other oligonucleotides, and may be labelled with a detectable group, or any other detectable marker. Methods for preparation of probes for hybridization and for construction of cDNA and genomic libraries are generally known in the art and are disclosed in Sambrook, et al., (1989) Molecular Cloning: A Library Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length. Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Duration of hybridization is generally less than about 24 hours, usually about 4 to 12. Stringent conditions may also be achieved with the addition of destabilizing agents such as formamide.

In a further embodiment, a variant as used herein can comprise a nucleic acid sequence encoding a OML4 or a GSK2 polypeptide as defined herein that is capable of hybridising under stringent conditions as defined herein to a nucleic acid sequence as defined in SEQ ID NO: 2 or 5 respectively.

In one embodiment, there is provided a method of increasing grain size and/or weight in a plant, as described herein, the method comprising reducing or abolishing the expression of at least one nucleic acid encoding a OML4 polypeptide, as described herein, wherein the method comprises introducing at least one mutation into at least OML4 gene and/or promoter, wherein the OML4 gene comprises or consists of
a. a nucleic acid sequence encoding a polypeptide as defined in one of SEQ ID NO:1; or
b. a nucleic acid sequence as defined in one of SEQ ID NO: 2; or
c. a nucleic acid sequence with at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to either (a) or (b); or
d. a nucleic acid sequence encoding a OML4 polypeptide as defined herein that is capable of hybridising under stringent conditions as defined herein to the nucleic acid sequence of any of (a) to (c).
   and wherein the OML4 promoter comprises or consists of
e. a nucleic acid sequence as defined in SEQ ID NO: 3;
f. a nucleic acid sequence with at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to (e); or
g. a nucleic acid sequence capable of hybridising under stringent conditions as defined herein to the nucleic acid sequence of any of (e) to (f).

In a preferred embodiment, the mutation that is introduced into the endogenous OML4 gene or promoter or the GSK2 gene or promoter thereof to silence, reduce, or inhibit the biological activity and/or expression levels of the OML4 or GSK2 gene or protein can be selected from the following mutation types
1. a “missense mutation”, which is a change in the nucleic acid sequence that results in the substitution of an amino acid for another amino acid;

2. a “nonsense mutation” or “STOP codon mutation”, which is a change in the nucleic acid sequence that results in the introduction of a premature STOP codon and, thus, the termination of translation (resulting in a truncated protein); plant genes contain the translation stop codons “TGA” (UGA in RNA), “TAA” (UAA in RNA) and “TAG” (UAG in RNA); thus any nucleotide substitution, insertion, deletion which results in one of these codons to be in the mature mRNA being translated (in the reading frame) will terminate translation.
3. an “insertion mutation” of one or more amino acids, due to one or more codons having been added in the coding sequence of the nucleic acid;
4. a “deletion mutation” of one or more amino acids, due to one or more codons having been deleted in the coding sequence of the nucleic acid;
5. a “frameshift mutation”, resulting in the nucleic acid sequence being translated in a different frame downstream of the mutation. A frameshift mutation can have various causes, such as the insertion, deletion or duplication of one or more nucleotides.
6. a “splice site” mutation, which is a mutation that results in the insertion, deletion or substitution of a nucleotide at the site of splicing.

As used herein, a “deletion” may refer to the deletion of at least one nucleotide. In one embodiment, said deletion may be between 1 and 20 base pairs. In a preferred embodiment, the at least one mutation is a deletion of at least one nucleotide.

In general, the skilled person will understand that at least one mutation as defined above and which leads to the insertion, deletion or substitution of at least one nucleic acid or amino acid compared to the wild-type OML4 or GSK 2 promoter or OML4 or GSK2 nucleic acid or protein sequence can affect the biological activity of the OML4 protein or GSK2 protein respectively.

In one embodiment, the mutation is a loss of function mutation such as a premature stop codon, or an amino acid change in a highly conserved region that is predicted to be important for protein structure.

In one embodiment, the mutation may be introduced into at least one RRM as defined herein of the OML4 gene. In an alternative or further embodiment, the mutation may be a substitution or deletion of a phosphorylation site in OML4. In one embodiment, the mutation may be at position S105, S146 and/or S607 of SEQ ID NO: 1 or a homologous position in a homologous sequence. Preferably, the mutation prevents the phosphorylation of OML4 at one or more of these sites. As described in the examples, preventing phosphorylation (by GSK2) of OML4 at one or more of these sites reduces the protein levels of OML4.

In another embodiment, the mutation is introduced into the OML4 or GSK2 promoter and is at least the deletion and/or insertion of at least one nucleic acid. Other major changes such as deletions that remove functional regions of the promoter are also included as these will reduce the expression of OML4 and GSK2.

In one embodiment at least one mutation may be introduced into the OML4 promoter and at least one mutation is introduced into the OML4 gene. In a further embodiment, at least one mutation may also be introduced into the GSK2 gene and at least one mutation is introduced into the GSK2 promoter.

In one embodiment, the mutation is introduced using mutagenesis or targeted genome editing. That is, in one embodiment, the invention relates to a method and plant that has been generated by genetic engineering methods as described above, and does not encompass naturally occurring varieties.

Targeted genome modification or targeted genome editing is a genome engineering technique that uses targeted DNA double-strand breaks (DSBs) to stimulate genome editing through homologous recombination (HR)-mediated recombination events. To achieve effective genome editing via introduction of site-specific DNA DSBs, four major classes of customisable DNA binding proteins can be used: meganucleases derived from microbial mobile genetic elements, ZF nucleases based on eukaryotic transcription factors, transcription activator-like effectors (TALEs) from Xanthomonas bacteria, and the RNA-guided DNA endonuclease Cas9 from the type II bacterial adaptive immune system CRISPR (clustered regularly interspaced short palindromic repeats). Meganuclease, ZF, and TALE proteins all recognize specific DNA sequences through protein-DNA interactions. Although meganucleases integrate nuclease and DNA-binding domains, ZF and TALE proteins consist of individual modules targeting 3 or 1 nucleotides (nt) of DNA, respectively. ZFs and TALEs can be assembled in desired combinations and attached to the nuclease domain of FokI to direct nucleolytic activity toward specific genomic loci.

In a preferred embodiment, the genome editing method that can be used according to the various aspects of the invention is CRISPR. The use of this technology in genome editing is well described in the art, for example in U.S. Pat. No. 8,697,359 and references cited herein. In short, CRISPR is a microbial nuclease system involved in defense against invading phages and plasmids. CRISPR loci in microbial hosts contain a combination of CRISPR-associated (Cas) genes as well as non-coding RNA elements capable of programming the specificity of the CRISPR-mediated nucleic acid cleavage (sgRNA). Three types (I-III) of CRISPR systems have been identified across a wide range of bacterial hosts. One key feature of each CRISPR locus is the presence of an array of repetitive sequences (direct repeats) interspaced by short stretches of non-repetitive sequences (spacers). The non-coding CRISPR array is transcribed and cleaved within direct repeats into short crRNAs containing individual spacer sequences, which direct Cas nucleases to the target site (protospacer). The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer.

One major advantage of the CRISPR-Cas9 system, as compared to conventional gene targeting and other programmable endonucleases is the ease of multiplexing, where multiple genes can be mutated simultaneously simply by using multiple sgRNAs each targeting a different gene. In addition, where two sgRNAs are used flanking a genomic region, the intervening section can be deleted or inverted (Wiles et al., 2015).

Cas9 is thus the hallmark protein of the type II CRISPR-Cas system, and is a large monomeric DNA nuclease guided to a DNA target sequence adjacent to the PAM (protospacer adjacent motif) sequence motif by a complex of two noncoding RNAs: CRISPR RNA (crRNA) and trans-activating crRNA (tracrRNA). The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with an sgRNA can introduce site-specific double strand breaks (DSBs) into genomic DNA of live cells from various organisms. For applications in eukaryotic organisms, codon optimized versions of Cas9, which is originally from the bacterium Streptococcus pyogenes, have been used. Alternatively, Cpf1, which is another Cas protein, can be used as the endonuclease. Cpf1 differs from Cas9 in several ways: Cpf1 requires a T-rich PAM sequence (TTTV) for target recognition, Cpf1 does not require a tracrRNA, (i.e. only a crRNA is required) and the Cpf1-cleavage site is located distal and downstream to the PAM sequence in the protospacer sequence (Li et al., 2017). Furthermore, after identification of the PAM motif, Cpf1 introduces a sticky-end-like DNA double-stranded break with several nucleotides of overhang. As such, the CRISPR/CPf1 system consists of a Cpf1 enzyme and a crRNA. In a further alternative embodiment, the nuclease may be MAD7.

The single guide RNA (sgRNA) is the second component of the CRISPR/Cas(Cpf or MAD7) system that forms a complex with the Cas9/Cpf1/MAD7 nuclease. sgRNA is a synthetic RNA chimera created by fusing crRNA with tracrRNA. The sgRNA guide sequence located at its 5′end confers DNA target specificity. Therefore, by modifying the guide sequence, it is possible to create sgRNAs with different target specificities. The canonical length of the guide sequence is 20 bp.

Cas9 (or Cpf1/MAD7) expression plasmids for use in the methods of the invention can be constructed as described in the art. Cas9 or Cpf1 or MAD7 and the one or more sgRNA molecules may be delivered as separate or as single constructs. Where separate constructs are used for the delivery of the CRISPR enzyme (i.e. Cas9 or Cpf1 or MAD7) and the sgRNA molecule (s), the promoters used to drive expression of the CRISPR enzyme/sgRNA molecule may be the same or different. In one embodiment, RNA polymerase (Pol) II-dependent promoters or the CaMV35S promoter can be used to drive expression of the CRISPR enzyme. In another embodiment, Pol III-dependent promoters, such as U6 or U3, can be used to drive expression of the sgRNA.

Accordingly, using techniques known in the art it is possible to design sgRNA molecules (such as https://chopchop.cbu.uib.no/) it is possible to find target sites and design sgRNA molecules that target a OML4 or GSK2 gene or promoter sequence as described herein. In one embodiment, the sgRNA molecules target a sequence selected from SEQ ID No: 33 (OML4 target sequence) or SEQ ID NO: 34 (GSK2 target sequence) or a variant thereof as defined herein. In a further embodiment, the sgRNA molecules comprises a protospacer sequence selected from SEQ ID No: 35 (OML4 target sequence) or SEQ ID NO: 36 (GSK2 target sequence) or a variant thereof, as defined herein.

In one embodiment, the method uses the sgRNA constructs defined in detail below to introduce a targeted mutation into a OML4 gene and/or promoter, and in a further embodiment, to additionally introduce a mutation into a GSK2 gene and/or promoter.

Thus, aspects of the invention involve targeted mutagenesis methods, specifically genome editing, and in a preferred embodiment exclude embodiments that are solely based on generating plants by traditional breeding methods.

The genome editing constructs may be introduced into a plant cell using any suitable method known to the skilled person (the term “introduced” can be used interchangeably with “transformation”, which is described below). In an alternative embodiment, any of the nucleic acid constructs described herein may be first transcribed to form a preassembled Cas9(or other CRISP nuclease)-sgRNA ribonucleoprotein and then delivered to at least one plant cell using any of the above described methods, such as lipofection, electroporation, bolistic bombardment or microinjection.

Specific protocols for using the above described CRISPR constructs would be well known to the skilled person. As one example, a suitable protocol is described in Ma & Liu (“CRISPR/Cas-based multiplex genome editing in monocot and dicot plants”) incorporated herein by reference.

The invention also extends to a plant obtained or obtainable by any method described herein.

Alternatively, more conventional mutagenesis methods can be used to introduce at least one mutation into a OML4 gene or OML4 promoter sequence, or into a GSK2 gene or GSK2 promoter sequence. These methods include both physical and chemical mutagenesis. A skilled person will know further approaches can be used to generate such mutants, and methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein.

In one embodiment, insertional mutagenesis is used, for example using T-DNA mutagenesis (which inserts pieces of the T-DNA from the Agrobacterium tumefaciens T-Plasmid into DNA causing either loss of gene function or gain of gene function mutations), site-directed nucleases (SDNs) or transposons as a mutagen. Insertional mutagenesis is an alternative means of disrupting gene function and is based on the insertion of foreign DNA into the gene of interest (see Krysan et al, The Plant Cell, Vol. 11, 2283-2290, December 1999). Accordingly, in one embodiment, T-DNA is used as an insertional mutagen to disrupt the OML4 or GSK2 gene or OML4 or GSK2 promoter expression. T-DNA not only disrupts the expression of the gene into which it is inserted, but also acts as a marker for subsequent identification of the mutation. Since the sequence of the inserted element is known, the gene in which the insertion has occurred can be recovered, using various cloning or PCR-based strategies. The insertion of a piece of T-DNA in the order of 5 to 25 kb in length generally produces a disruption of gene function. If a large enough population of T-DNA transformed lines is generated, there are reasonably good chances of finding a transgenic plant carrying a T-DNA insert within any gene of interest. Transformation of spores with T-DNA is achieved by an Agrobacterium-mediated method which involves exposing plant cells and tissues to a suspension of Agrobacterium cells.

The details of this method are well known to a skilled person. In short, plant transformation by Agrobacterium results in the integration into the nuclear genome of a sequence called T-DNA, which is carried on a bacterial plasmid. The use of T-DNA transformation leads to stable single insertions. Further mutant analysis of the resultant transformed lines is straightforward and each individual insertion line can be rapidly characterized by direct sequencing and analysis of DNA flanking the insertion. Gene expression in the mutant is compared to expression of the OML4 or GSK2 nucleic acid sequence in a wild type plant and phenotypic analysis is also carried out.

In another embodiment, mutagenesis is physical mutagenesis, such as application of ultraviolet radiation, X-rays, gamma rays, fast or thermal neutrons or protons. The targeted population can then be screened to identify a OML4 or GSK2 loss of function mutant.

In another embodiment of the various aspects of the invention, the method comprises mutagenizing a plant population with a mutagen. The mutagen may be a fast neutron irradiation or a chemical mutagen, for example selected from the following non-limiting list: ethyl methanesulfonate (EMS), methylmethane sulfonate (MMS), N-ethyl-N-nitrosurea (ENU), triethylmelamine (1′EM), N-methyl-N-nitrosourea (MNU), procarbazine, chlorambucil, cyclophosphamide, diethyl sulfate, acrylamide monomer, melphalan, nitrogen mustard, vincristine, dimethylnitosamine, N-methyl-N′-nitro-Nitrosoguanidine (MNNG), nitrosoguanidine, 2-aminopurine, 7,12 dimethyl-benz(a)anthracene (DMBA), ethylene oxide, hexamethylphosphoramide, bisulfan, diepoxyalkanes (diepoxyoctane (DEO), diepoxybutane (BEB), and the like), 2-methoxy-6-chloro-9 [3-(ethyl-2-chloroethyl)aminopropylamino]acridine dihydrochloride (ICR-170) or formaldehyde. Again, the targeted population can then be screened to identify a OML4 or GSK2 gene or promoter mutant.

In another embodiment, the method used to create and analyse mutations is targeting induced local lesions in genomes (TILLING), reviewed in Henikoff et al, 2004. In this method, seeds are mutagenised with a chemical mutagen, for example EMS. The resulting M1 plants are self-fertilised and the M2 generation of individuals is used to prepare DNA samples for mutational screening. DNA samples are pooled and arrayed on microtiter plates and subjected to gene specific PCR. The PCR amplification products may be screened for mutations in the target gene using any method that identifies heteroduplexes between wild type and mutant genes. For example, but not limited to, denaturing high pressure liquid chromatography (dHPLC), constant denaturant capillary electrophoresis (CDCE), temperature gradient capillary electrophoresis (TGCE), or by fragmentation using chemical cleavage. Preferably the PCR amplification products are incubated with an endonuclease that preferentially cleaves mismatches in heteroduplexes between wild type and mutant sequences. Cleavage products are electrophoresed using an automated sequencing gel apparatus, and gel images are analyzed with the aid of a standard commercial image-processing program. Any primer specific to the OML4 or GSK2 nucleic acid sequence may be utilized to amplify the OML4 or GSK2 nucleic acid sequence within the pooled DNA sample. Preferably, the primer is designed to amplify the regions of the OML4 or GSK2 gene where useful mutations are most likely to arise, specifically in the areas of the genes that are highly conserved and/or confer activity as explained elsewhere. To facilitate detection of PCR products on a gel, the PCR primer may be labelled using any conventional labelling method. In an alternative embodiment, the method used to create and analyse mutations is EcoTILLING. EcoTILLING is molecular technique that is similar to TILLING, except that its objective is to uncover natural variation in a given population as opposed to induced mutations. The first publication of the EcoTILLING method was described in Comai et al. 2004.

Rapid high-throughput screening procedures thus allow the analysis of amplification products for identifying a mutation conferring the reduction or inactivation of the expression of the OML4 or GSK2 gene as compared to a corresponding non-mutagenised wild type plant. Once a mutation is identified in a gene of interest, the seeds of the M2 plant carrying that mutation are grown into adult M3 plants and screened for the phenotypic characteristics associated with the target gene. Loss of and reduced function mutants with increased grain weight and/or grain size compared to a control can thus be identified.

Plants obtained or obtainable by such method which carry a partial or complete loss of function mutation in the endogenous OML4 gene or promoter locus are also within the scope of the invention

In an alternative embodiment, the expression of the OML4 or GSK2 gene may be reduced at either the level of transcription or translation. For example, expression of a OML4 or GSK2 nucleic acid as defined herein, can be reduced or silenced using a number of gene silencing methods known to the skilled person, such as, but not limited to, the use of small interfering nucleic acids (siNA) against OML4 or GSK2.

“Gene silencing” is a term generally used to refer to suppression of expression of a gene via sequence-specific interactions that are mediated by RNA molecules. The degree of reduction may be so as to totally abolish production of the encoded gene product, but more usually the abolition of expression is partial, with some degree of expression remaining. The term should not therefore be taken to require complete “silencing” of expression.

In one embodiment, the siNA may include, short interfering RNA (siRNA), double-stranded RNA (dsRNA), micro-RNA (miRNA), antagomirs and short hairpin RNA (shRNA) capable of mediating RNA interference.

The inhibition of expression and/or activity can be measured by determining the presence and/or amount of OML4 or GSK2 transcript using techniques well known to the skilled person (such as Northern Blotting, RT-PCR and so on).

Transgenes may be used to suppress endogenous plant genes. This was discovered originally when chalcone synthase transgenes in petunia caused suppression of the endogenous chalcone synthase genes and indicated by easily visible pigmentation changes. Subsequently it has been described how many, if not all plant genes can be “silenced” by transgenes. Gene silencing requires sequence similarity between the transgene and the gene that becomes silenced. This sequence homology may involve promoter regions or coding regions of the silenced target gene. When coding regions are involved, the transgene able to cause gene silencing may have been constructed with a promoter that would transcribe either the sense or the antisense orientation of the coding sequence RNA. It is likely that the various examples of gene silencing involve different mechanisms that are not well understood. In different examples there may be transcriptional or post-transcriptional gene silencing and both may be used according to the methods of the invention.

The mechanisms of gene silencing and their application in genetic engineering, which were first discovered in plants in the early 1990s and then shown in Caenorhabditis elegans are extensively described in the literature.

RNA interference (RNAi) is another post-transcriptional gene-silencing phenomenon which may be used according to the methods of the invention. This is induced by double-stranded RNA in which mRNA that is homologous to the dsRNA is specifically degraded. It refers to the process of sequence-specific post-transcriptional gene silencing mediated by short interfering RNAs (siRNA). The process of RNAi begins when the enzyme, DICER, encounters dsRNA and chops it into pieces called small-interfering RNAs (siRNA). This enzyme belongs to the RNase III nuclease family. A complex of proteins gathers up these RNA remains and uses their code as a guide to search out and destroy any RNAs in the cell with a matching sequence, such as target mRNA.

Artificial and/or natural microRNAs (miRNAs) may be used to knock out gene expression and/or mRNA translation. MicroRNAs (miRNAs) miRNAs are typically single stranded small RNAs typically 19-24 nucleotides long. Most plant miRNAs have perfect or near-perfect complementarity with their target sequences. However, there are natural targets with up to five mismatches. They are processed from longer non-coding RNAs with characteristic fold-back structures by double-strand specific RNases of the Dicer family. Upon processing, they are incorporated in the RNA-induced silencing complex (RISC) by binding to its main component, an Argonaute protein. miRNAs serve as the specificity components of RISC, since they base-pair to target nucleic acids, mostly mRNAs, in the cytoplasm. Subsequent regulatory events include target mRNA cleavage and destruction and/or translational inhibition. Effects of miRNA overexpression are thus often reflected in decreased mRNA levels of target genes. Artificial microRNA (amiRNA) technology has been applied in Arabidopsis thaliana and other plants to efficiently silence target genes of interest. The design principles for amiRNAs have been generalized and integrated into a Web-based tool (http://wmd.weigelworld.org).

Thus, according to the various aspects of the invention a plant may be transformed to introduce a RNAi, shRNA, snRNA, dsRNA, siRNA, miRNA, ta-siRNA, amiRNA or cosuppression molecule that has been designed to target the expression of an OML4 or GSK2 nucleic acid sequence and selectively decreases or inhibits the expression of the gene or stability of its transcript. Preferably, the RNAi, snRNA, dsRNA, shRNA siRNA, miRNA, amiRNA, ta-siRNA or cosuppression molecule used according to the various aspects of the invention comprises a fragment of at least 17 nt, preferably 22 to 26 nt and can be designed on the basis of the information shown in any of SEQ ID NOs:2, 5, 8, 11, 14, 17, 20, 23, 26 and 29. Guidelines for designing effective siRNAs are known to the skilled person. Briefly, a short fragment of the target gene sequence (e.g., 19-40 nucleotides in length) is chosen as the target sequence of the siRNA of the invention. The short fragment of target gene sequence is a fragment of the target gene mRNA. In preferred embodiments, the criteria for choosing a sequence fragment from the target gene mRNA to be a candidate siRNA molecule include 1) a sequence from the target gene mRNA that is at least 50-100 nucleotides from the 5′ or 3′ end of the native mRNA molecule, 2) a sequence from the target gene mRNA that has a G/C content of between 30% and 70%, most preferably around 50%, 3) a sequence from the target gene mRNA that does not contain repetitive sequences (e.g., AAA, CCC, GGG, TTT, AAAA, CCCC, GGGG, TTTT), 4) a sequence from the target gene mRNA that is accessible in the mRNA, 5) a sequence from the target gene mRNA that is unique to the target gene, 6) avoids regions within 75 bases of a start codon. The sequence fragment from the target gene mRNA may meet one or more of the criteria identified above. The selected gene is introduced as a nucleotide sequence in a prediction program that takes into account all the variables described above for the design of optimal oligonucleotides. This program scans any mRNA nucleotide sequence for regions susceptible to be targeted by siRNAs. The output of this analysis is a score of possible siRNA oligonucleotides. The highest scores are used to design double stranded RNA oligonucleotides that are typically made by chemical synthesis. In addition to siRNA which is complementary to the mRNA target region, degenerate siRNA sequences may be used to target homologous regions. siRNAs according to the invention can be synthesized by any method known in the art. RNAs are preferably chemically synthesized using appropriately protected ribonucleoside phosphoramidites and a conventional DNA/RNA synthesizer. Additionally, siRNAs can be obtained from commercial RNA oligonucleotide synthesis suppliers.

The silencing RNA molecule is introduced into the plant using conventional methods, for example a vector and Agrobacterium-mediated transformation. Stably transformed plants are generated and expression of the OML4 or GSK2 gene compared to a wild type control plant is analysed.

Silencing of the OML4 or GSK2 nucleic acid sequence may also be achieved using virus-induced gene silencing.

Thus, in one embodiment of the invention, the plant expresses a nucleic acid construct comprising a RNAi, shRNA snRNA, dsRNA, siRNA, miRNA, ta-siRNA, amiRNA or cosuppression molecule that targets the OML4 nucleic acid sequence as described herein and reduces expression of the endogenous OML4 nucleic acid sequence. A gene is targeted when, for example, the RNAi, snRNA, dsRNA, siRNA, shRNA miRNA, ta-siRNA, amiRNA or cosuppression molecule selectively decreases or inhibits the expression of the gene compared to a control plant. Alternatively, a RNAi, snRNA, dsRNA, siRNA, miRNA, ta-siRNA, amiRNA or cosuppression molecule targets a OML4 or GSK2 nucleic acid sequence when the RNAi, shRNA snRNA, dsRNA, siRNA, miRNA, ta-siRNA, amiRNA or cosuppression molecule hybridises under stringent conditions to the gene transcript.

A further approach to gene silencing is by targeting nucleic acid sequences complementary to the regulatory region of the gene (e.g., the promoter and/or enhancers) of OML4 or GSK2 to form triple helical structures that prevent transcription of the gene in target cells. Other methods, such as the use of antibodies directed to an endogenous polypeptide for inhibiting its function in planta, or interference in the signalling pathway in which a polypeptide is involved, will be well known to the skilled man. In particular, it can be envisaged that man-made molecules may be useful for inhibiting the biological function of a target polypeptide, or for interfering with the signalling pathway in which the target polypeptide is involved.

In another aspect, the invention relates to a silencing construct obtainable or obtained by a method as described herein and to a plant cell comprising such construct. In one example an RNAi construct to silence GSK2 comprises or consists of the sequence defined in SEQ ID NO: 31 or a functional variant thereof.

In another aspect, the invention extends to a plant obtained or obtainable by a method as described herein.

Methods of Increasing Grain Number

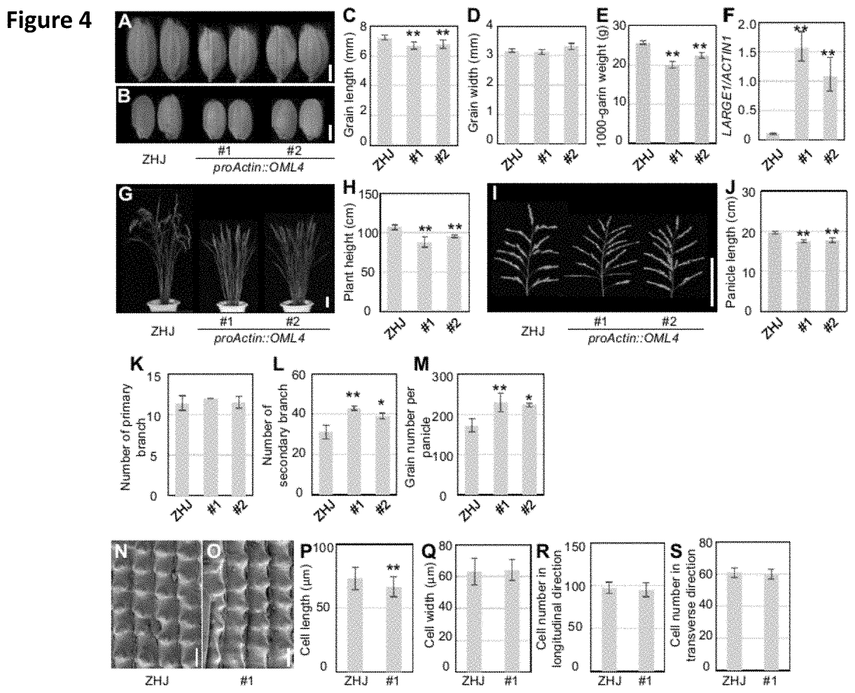

In another aspect of the invention, there is provided a method of increasing the grain number in a plant. As shown in FIG. 4(m) overexpressing OML4 results in a significant increase in grain number. Accordingly, in a further aspect of the invention, there is provided a method of increasing grain number in a plant, the method comprising increasing the expression and/or activity of OML4. Preferably said increase is relative to a wild-type or control plant.

In one embodiment, an “increase” in grain number may comprise an increase of at least 5%, 10%, 15%, 20%, 25%, 30%, 35%, 40%, 45% or 50% compared to the grain number in a wild-type or control plant. In one embodiment, an increase in grain number may be an increase in grain number per panicle. Any of the above can be measured using standard techniques in the art.

In a further aspect of the invention, the method further comprises increasing the expression or activity of SHAGGY-like kinase (GSK2).

In one embodiment, the method may comprise introducing and expressing in a plant or plant cell a nucleic acid construct comprising a nucleic acid sequence encoding an OML4 polypeptide as defined in SEQ ID NO: 1 or a homolog or functional variant thereof, as defined herein. Preferably, the nucleic acid sequence is operably linked to a regulatory sequence, preferably a promoter. In another embodiment, the nucleic acid construct may comprise a first nucleic acid sequence encoding an OML4 polypeptide as defined above and a second nucleic acid sequence encoding a GSK2 polypeptide as defined in SEQ ID NO: 4 or a homolog or functional variant thereof. Preferably, the first and second nucleic acid sequences are operably linked to a regulatory sequence, preferably a promoter. The first and second nucleic acid sequences may be operably linked to the same or a different regulatory sequence.

In an alternative embodiment, the method may comprise introducing and expressing a first nucleic acid construct comprising a nucleic acid sequence encoding an OML4 polypeptide as defined above and a second nucleic acid construct comprising a nucleic acid sequence encoding a GSK2 polypeptide as defined above. Again, the nucleic acid sequences are preferably operably linked to a regulatory sequence. The second nucleic acid construct may be introduced and expressed in the plant before, after or concurrently with the first nucleic acid construct.

Methods for the introduction of a nucleic acid construct as described above into a plant or plant cell (also called “transformation” (such terms may be used interchangeably)) are described herein. In one embodiment, the progeny plant is stably transformed with the nucleic acid construct described herein and comprises the exogenous polypeptide or polypeptides that are heritably maintained in the plant cell. The method may also comprise the additional step of collecting seeds from the selected progeny plant.

The method may further comprise the step of regenerating a transgenic plant from the plant cell wherein the transgenic plant comprises in its genome a nucleic acid sequence selected from SEQ ID NO: 2 and a nucleic acid sequence selected from SEQ ID NO: 5 or a homolog or functional variant thereof, and obtaining progeny derived from the transgenic plant, where the progeny exhibits an increase in grain number.

In a further embodiment, the method may comprise introducing a mutation into the plant genome, where said mutation is the insertion of at least one or more additional copy(ies) of a nucleic acid encoding a OML4 polypeptide or a homolog or variant thereof such that said sequence is operably linked to a regulatory sequence and wherein said mutation is introduced using targeted genome editing. Preferably, said mutation results in an increase in the expression of a OML4 nucleic acid compared to a control or wild-type plant. In an additional embodiment, the method may further comprise introducing one or more further mutations into the plant genome, where the one or more further mutations is the insertion of at least one or more additional copy(ies) of a nucleic acid encoding a GSK2 polypeptide or a homologue or functional variant thereof such that said sequence is operably linked to a regulatory sequence. Again, preferably the mutation is introduced using targeted genome editing. Preferably the mutation also results in an increase in the expression of a GSK2 polypeptide compared to a control or wild-type plant. The genomic and amino acid sequence of rice OML4 and GSK2 and its homologs are defined below.

In one embodiment, the mutation is introduced using CRISPR as described herein.

The invention also extends to plants obtained or obtainable by any method described herein.

Genetically Altered or Modified Plants and Methods of Producing Such Plants

In another aspect of the invention there is provided a genetically altered plant, part thereof or plant cell characterised in that the plant does not express OML4, has reduced levels of OML4 expression, does not express a functional OML4 protein or expresses a OML4 protein with reduced function and/or activity. For example, the plant is a reduction (knock down) or loss of function (knock out) mutant wherein the function of the OML4 nucleic acid sequence is reduced or lost compared to a wild type control plant. To this end, a mutation is introduced into either the OML4 gene sequence or the corresponding promoter sequence, which disrupts the transcription of the gene.

Therefore, preferably said plant comprises at least one mutation in the promoter and/or gene for OML4. In one embodiment the plant may comprise a mutation in both the promoter and gene for OML4.

In a further embodiment, the genetically altered plant, part thereof or plant cell is further characterised in that the plant also does not express GSK2 has reduced levels of GSK2 expression, does not express a functional GSK2 protein or expresses a GSK2 protein with reduced function and/or activity.

In a further aspect of the invention, there is provided a plant, part thereof or plant cell characterised by an increase in grain weight and/or size compared to a wild-type or control pant, wherein preferably, the plant comprises at least one mutation in the OML4 gene and/or its promoter.

The plant may be produced by introducing a mutation, preferably a deletion, insertion or substitution into the OML4 gene and/or promoter sequence by any of the above described methods. Preferably said mutation is introduced into a least one plant cell and a plant regenerated from the at least one mutated plant cell.

Alternatively, the plant or plant cell may comprise a nucleic acid construct expressing an RNAi molecule targeting the OML4 or GSK2 gene as described herein. In one embodiment, said construct is stably incorporated into the plant genome. These techniques also include gene targeting using vectors that target the gene of interest and which allow integration of a transgene at a specific site. The targeting construct is engineered to recombine with the target gene, which is accomplished by incorporating sequences from the gene itself into the construct. Recombination then occurs in the region of that sequence within the gene, resulting in the insertion of a foreign sequence to disrupt the gene. With its sequence interrupted, the altered gene will be translated into a nonfunctional protein, if it is translated at all.

In another aspect of the invention there is provided a method for producing a genetically altered plant as described herein. In one embodiment, the method comprises introducing at least one mutation into the OML4 gene and/or OML4 promoter of preferably at least one plant cell using any mutagenesis technique described herein. In a further embodiment, the method comprises further introducing at least one mutation into the GSK2 gene and/or GSK2 promoter Preferably, said method further comprising regenerating a plant from the mutated plant cell.

The method may further comprise selecting one or more mutated plants, preferably for further propagation. Preferably said selected plants comprise at least one mutation in the target gene(s) and/or promoter sequence (s). Preferably said plants or said seeds of said plant are characterised by abolished or a reduced level of OML4 expression and/or a reduced level of OML4 polypeptide activity. Expression and/or activity levels of OML4 can be measured by any standard technique known to the skilled person. A reduction is as described herein.

The selected plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

In a further aspect of the invention there is provided a plant obtained or obtainable by the above-described methods.

In another aspect of the invention, there is provided a genetically altered plant, part thereof or plant cell characterised in that the expression of OML4 is increased compared to the level of expression in a control or wild-type plant. Preferably, the plant expresses a polynucleotide that is either exogenous or endogenous to that plant. That is, a polynucleotide that is introduced into the plant by any means other than a sexual cross. In one embodiment of the method, an exogenous nucleic acid is expressed in the transgenic plant, which is a nucleic acid construct comprising a nucleic acid construct as described above. Alternatively, the plant carries a mutation in its genome where the mutation is the insertion of at least one or more additional copy of a nucleic acid sequence encoding an OML4 polypeptide, as defined herein, or a homolog or variant thereof such that said sequence is operably linked to a regulatory sequence.

The plant may further comprise a second mutation in the plant genome, wherein the mutation is the insertion of at least one or more additional copy of a nucleic acid sequence encoding a GSK2 polypeptide, as defined herein, or a homolog or variant thereof such that said sequence is operably linked to a regulatory sequence. Preferably the mutation is introduced using targeted genome editing.

For the purposes of the invention, a “genetically altered plant” or “mutant plant” is a plant that has been genetically altered compared to the naturally occurring wild type (WT) plant. In one embodiment, a mutant plant is a plant that has been altered compared to the naturally occurring wild type (WT) plant using a mutagenesis method, such as any of the mutagenesis methods described herein. In one embodiment, the mutagenesis method is targeted genome modification or genome editing. In one embodiment, the plant genome has been altered compared to wild type sequences using a mutagenesis method. Such plants have an altered phenotype as described herein, such as an increased disease resistance. Therefore, in one example, increased grain weight and/or size is conferred by the presence of an altered plant genome, for example, a mutated endogenous OML4 gene or OML4 promoter sequence. In one embodiment, the endogenous promoter or gene sequence is specifically targeted using targeted genome modification and the presence of a mutated gene or promoter sequence is not conferred by the presence of transgenes expressed in the plant. In other words, the genetically altered plant can be described as transgene-free.

A plant according to the various aspects of the invention, including the transgenic plants, methods and uses described herein may be a monocot or a dicot plant. Preferably, the plant is a crop plant. By crop plant is meant any plant which is grown on a commercial scale for human or animal consumption or use.

Preferably, the crop plant is selected from rice, wheat, maize, soybean and brassicas, such as for example, B. napus. More preferably, the crop plant is rice and even more preferably the japonica or indica variety.

The term “plant” as used herein encompasses whole plants and progeny of the plants and plant parts, including seeds, fruit, shoots, stems, leaves, roots (including tubers), flowers, tissues and organs, wherein each of the aforementioned comprise at least one of the mutations described herein or a sgRNA or an RNAi construct as described herein. The term “plant” also encompasses plant cells, suspension cultures, callus tissue, embryos, meristematic regions, gametophytes, sporophytes, pollen and microspores, again wherein each of the aforementioned comprises at least one of the mutations described herein or nucleic acid construct, a sgRNA or an RNAi construct as described herein. Accordingly, in one embodiment, the plat part is a grain or seed.

The invention also extends to harvestable parts of a plant of the invention as described herein, but not limited to seeds, leaves, fruits, flowers, stems, roots, rhizomes, tubers and bulbs. The aspects of the invention also extend to products derived, preferably directly derived, from a harvestable part of such a plant, such as dry pellets or powders, oil, fat and fatty acids, starch or proteins. Another product that may derived from the harvestable parts of the plant of the invention is biodiesel. The invention also relates to food products and food supplements comprising the plant of the invention or parts thereof. In one embodiment, the food products may be animal feed. In another aspect of the invention, there is provided a product derived from a plant as described herein or from a part thereof.

In a most preferred embodiment, the plant part or harvestable product is a seed or grain. Therefore, in a further aspect of the invention, there is provided a seed produced from a genetically altered plant as described herein.

In an alternative embodiment, the plant part is pollen, a propagule or progeny of the genetically altered plant described herein. Accordingly, in a further aspect of the invention there is provided pollen, a propagule or progeny of the genetically altered plant as described herein.

A control plant as used herein according to all of the aspects of the invention is a plant which has not been modified according to the methods of the invention. Accordingly, in one embodiment, the control plant does not have reduced expression of a OML4 nucleic acid and/or reduced activity of a OML4 polypeptide. In an alternative embodiment, the plant been genetically modified, as described above. In one embodiment, the control plant is a wild type plant. The control plant is typically of the same plant species, preferably having the same genetic background as the modified plant.

Genome Editing Constructs for Use with the Methods for Targeted Genome Modification Described Herein

By “crRNA” or CRISPR RNA is meant the sequence of RNA that contains the protospacer element and additional nucleotides that are complementary to the tracrRNA.

By “tracrRNA” (transactivating RNA) is meant the sequence of RNA that hybridises to the crRNA and binds a CRISPR enzyme, such as Cas9 thereby activating the nuclease complex to introduce double-stranded breaks at specific sites within the genomic sequence of at least one OML4 or GSK2 nucleic acid or promoter sequence.

By “protospacer element” is meant the portion of crRNA (or sgRNA) that is complementary to the genomic DNA target sequence, usually around 20 nucleotides in length. This may also be known as a spacer or targeting sequence.

By “sgRNA” (single-guide RNA) is meant the combination of tracrRNA and crRNA in a single RNA molecule, preferably also including a linker loop (that links the tracrRNA and crRNA into a single molecule). “sgRNA” may also be referred to as “gRNA” and in the present context, the terms are interchangeable. The sgRNA or gRNA provide both targeting specificity and scaffolding/binding ability for a Cas nuclease. A gRNA may refer to a dual RNA molecule comprising a crRNA molecule and a tracrRNA molecule.

By “TAL effector” (transcription activator-like (TAL) effector) or TALE is meant a protein sequence that can bind the genomic DNA target sequence (e.g. a sequence within the OML4 gene or promoter sequence) and that can be fused to the cleavage domain of an endonuclease such as FokI to create TAL effector nucleases or TALENS or meganucleases to create megaTALs. A TALE protein is composed of a central domain that is responsible for DNA binding, a nuclear-localisation signal and a domain that activates target gene transcription. The DNA-binding domain consists of monomers and each monomer can bind one nucleotide in the target nucleotide sequence. Monomers are tandem repeats of 33-35 amino acids, of which the two amino acids located at positions 12 and 13 are highly variable (repeat variable diresidue, RVD). It is the RVDs that are responsible for the recognition of a single specific nucleotide. HD targets cytosine; NI targets adenine, NG targets thymine and NN targets guanine (although NN can also bind to adenine with lower specificity).

In another aspect of the invention there is provided a nucleic acid construct wherein the nucleic acid construct encodes at least one DNA-binding domain, wherein the DNA-binding domain can bind to a sequence in the OML4 gene, wherein said sequence is comprises or consists of SEQ ID NO: 33 or a variant thereof. In an alternative embodiment, the DNA-binding domain can bind to a sequence in the GSK2 gene, wherein said sequence comprises or consists of SEQ ID NO: 34 or a variant thereof. In one embodiment, said construct further comprises a nucleic acid encoding a SSN, such as FokI or a Cas protein.

In one embodiment, the nucleic acid construct encodes at least one protospacer element wherein the sequence of the protospacer element is selected from SEQ ID No: 35 (to target OML4) or SEQ ID NO: 36 (to target GSK2) or a variant thereof.

In a further embodiment, the nucleic acid construct comprises a crRNA-encoding sequence. As defined above, a crRNA sequence may comprise the protospacer elements as defined above and preferably additional nucleotides that are complementary to the tracrRNA. An appropriate sequence for the additional nucleotides will be known to the skilled person as these are defined by the choice of Cas protein.

In another embodiment, the nucleic acid construct further comprises a tracrRNA sequence. Again, an appropriate tracrRNA sequence would be known to the skilled person as this sequence is defined by the choice of Cas protein.

In a further embodiment, the nucleic acid construct comprises at least one nucleic acid sequence that encodes a sgRNA (or gRNA). Again, as already discussed, sgRNA typically comprises a crRNA sequence, a tracrRNA sequence and preferably a sequence for a linker loop.

In a further embodiment, the nucleic acid construct may further comprise at least one nucleic acid sequence encoding an endoribonuclease cleavage site. Preferably the endoribonuclease is Csy4 (also known as Cas6f). Where the nucleic acid construct comprises multiple sgRNA nucleic acid sequences the construct may comprise the same number of endoribonuclease cleavage sites. In another embodiment, the cleavage site is 5′ of the sgRNA nucleic acid sequence. Accordingly, each sgRNA nucleic acid sequence is flanked by a endoribonuclease cleavage site.

The term ‘variant’ refers to a nucleotide sequence where the nucleotides are substantially identical to one of the above sequences. The variant may be achieved by modifications such as an insertion, substitution or deletion of one or more nucleotides. In a preferred embodiment, the variant has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to any one of the above sequences. In one embodiment, sequence identity is at least 90%. In another embodiment, sequence identity is 100%. Sequence identity can be determined by any one known sequence alignment program in the art.

The invention also relates to a nucleic acid construct comprising a nucleic acid sequence operably linked to a suitable plant promoter. A suitable plant promoter may be a constitutive or strong promoter or may be a tissue-specific promoter. In one embodiment, suitable plant promoters are selected from, but not limited to U3 and U6.

The nucleic acid construct of the present invention may also further comprise a nucleic acid sequence that encodes a CRISPR enzyme. By “CRISPR enzyme” is meant an RNA-guided DNA endonuclease that can associate with the CRISPR system. Specifically, such an enzyme binds to the tracrRNA sequence. In one embodiment, the CRIPSR enzyme is a Cas protein (“CRISPR associated protein), preferably Cas 9 or Cpf1, more preferably Cas9. In a specific embodiment Cas9 is a codon-optimised Cas9 (specific for the plant in question). In one embodiment, Cas9 has the sequence described in SEQ ID NO: 32 or a functional variant or homolog thereof. In another embodiment, the CRISPR enzyme is a protein from the family of Class 2 candidate x proteins, such as C2c1, C2C2 and/or C2c3. In one embodiment, the Cas protein is from Streptococcus pyogenes. In an alternative embodiment, the Cas protein may be from any one of Staphylococcus aureus, Neisseria meningitides, Streptococcus thermophiles or Treponema denticola. Alternatively, the CRISPR enzyme is MAD7.

The term “functional variant” as used herein with reference to Cas9 refers to a variant Cas9 gene sequence or part of the gene sequence which retains the biological function of the full non-variant sequence, for example, acts as a DNA endonuclease, or recognition or/and binding to DNA. A functional variant also comprises a variant of the gene of interest, which has sequence alterations that do not affect function, for example non-conserved residues. Also encompassed is a variant that is substantially identical, i.e. has only some sequence variations, for example in non-conserved residues, compared to the wild type sequences as shown herein and is biologically active. In one embodiment, a functional variant of SEQ ID NO: 32 has at least 90%, 91%, 92%, 93%, 94%, 95%, 96%, 97%, 98%, or 99% overall sequence identity to the nucleic acid acid represented by SEQ ID NO: 32. In a further embodiment, the Cas9 protein has been modified to improve activity.

Suitable homologs or orthologs can be identified by sequence comparisons and identifications of conserved domains. The function of the homolog or ortholog can be identified as described herein and a skilled person would thus be able to confirm the function when expressed in a plant.

In an alternative aspect of the invention, the nucleic acid construct comprises at least one nucleic acid sequence that encodes a TAL effector, wherein said effector targets a OML4 sequence, such as SEQ ID NO: 33 or a GSK2 sequence such as SEQ ID NO: 34. Methods for designing a TAL effector would be well known to the skilled person, given the target sequence. Examples of suitable methods are given in Sanjana et al., and Cermak T et al, both incorporated herein by reference. Preferably, said nucleic acid construct comprises two nucleic acid sequences encoding a TAL effector, to produce a TALEN pair. In a further embodiment, the nucleic acid construct further comprises a sequence-specific nuclease (SSN). Preferably such SSN is a endonuclease such as FokI. In a further embodiment, the TALENs are assembled by the Golden Gate cloning method in a single plasmid or nucleic acid construct.

In another aspect of the invention, there is provided a sgRNA molecule, wherein the sgRNA molecule comprises a crRNA sequence and a tracrRNA sequence and wherein the crRNA sequence can bind to at least one sequence such as SEQ ID NO: 33 (for OML4) or SEQ ID NO: 34 (for GSK2) or a variant thereof.

A “variant” is as defined herein. In one embodiment, the sgRNA molecule may comprise at least one chemical modification, for example that enhances its stability and/or binding affinity to the target sequence or the crRNA sequence to the tracrRNA sequence. Such modifications would be well known to the skilled person, and include for example, but not limited to, the modifications described in Randar et al., 2015, incorporated herein by reference. In this example the crRNA may comprise a phosphorothioate backbone modification, such as 2′-fluoro (2′-F), 2′-O-methyl (2′-O-Me) and S-constrained ethyl (cET) substitutions.

In another aspect of the invention, there is provided an isolated nucleic acid sequence that encodes for a protospacer element (as defined in any of SEQ ID NO: 35 or 36.)

In another aspect of the invention, there is provided a plant or part thereof or at least one isolated plant cell transfected with at least one nucleic acid construct as described herein. Cas9 and sgRNA may be combined or in separate expression vectors (or nucleic acid constructs, such terms are used interchangeably). In other words, in one embodiment, an isolated plant cell is transfected with a single nucleic acid construct comprising both sgRNA and Cas9 as described in detail above. In an alternative embodiment, an isolated plant cell is transfected with two nucleic acid constructs, a first nucleic acid construct comprising at least one sgRNA as defined above and a second nucleic acid construct comprising Cas9 or a functional variant or homolog thereof. The second nucleic acid construct may be transfected below, after or concurrently with the first nucleic acid construct. The advantage of a separate, second construct comprising a cas protein is that the nucleic acid construct encoding at least one sgRNA can be paired with any type of cas protein, as described herein, and therefore is not limited to a single cas function (as would be the case when both cas and sgRNA are encoded on the same nucleic acid construct).

In one embodiment, the nucleic acid construct comprising a cas protein is transfected first and is stably incorporated into the genome, before the second transfection with a nucleic acid construct comprising at least one sgRNA nucleic acid. In an alternative embodiment, a plant or part thereof or at least one isolated plant cell is transfected with mRNA encoding a cas protein and co-transfected with at least one nucleic acid construct as defined herein.

Cas9 expression vectors for use in the present invention can be constructed as described in the art. In one example, the expression vector comprises a nucleic acid sequence as defined herein or a functional variant or homolog thereof, wherein said nucleic acid sequence is operably linked to a suitable promoter. Examples of suitable promoters include, but are not limited to Cas9, 35S and Actin.

In an alternative aspect of the present invention, there is provided an isolated plant cell transfected with at least one sgRNA molecule as described herein.

In a further aspect of the invention, there is provided a genetically modified or edited plant comprising the transfected cell described herein. In one embodiment, the nucleic acid construct or constructs may be integrated in a stable form. In an alternative embodiment, the nucleic acid construct or constructs are not integrated (i.e. are transiently expressed). Accordingly, in a preferred embodiment, the genetically modified plant is free of any sgRNA and/or Cas protein nucleic acid. In other words, the plant is transgene free.

The term “introduction”, “transfection” or “transformation” as referred to throughout the application encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The resulting transformed plant cell may then be used to regenerate a transformed plant in a manner known to persons skilled in the art. The transfer of foreign genes into the genome of a plant is called transformation. Transformation of plants is now a routine technique in many species. Any of several transformation methods known to the skilled person may be used to introduce any of the nucleic acid constructs described herein or, a sgRNA molecule of interest into a suitable ancestor cell. The methods described for the transformation and regeneration of plants from plant tissues or plant cells may be utilized for transient or for stable transformation.

Transformation methods include the use of liposomes, electroporation, chemicals that increase free DNA uptake, injection of the DNA directly into the plant (microinjection), gene guns (or biolistic particle delivery systems (bioloistics)) as described in the examples, lipofection, transformation using viruses or pollen and microprojection. Methods may be selected from the calcium/polyethylene glycol method for protoplasts, ultrasound-mediated gene transfection, optical or laser transfection, transfection using silicon carbide fibers, electroporation of protoplasts, microinjection into plant material, DNA or RNA-coated particle bombardment, infection with (non-integrative) viruses and the like. Transgenic plants, can also be produced via Agrobacterium tumefaciens mediated transformation, including but not limited to using the floral dip/Agrobacterium vacuum infiltration method as described in Clough & Bent (1998) and incorporated herein by reference.

Accordingly, in one embodiment, at least one nucleic acid construct or sgRNA molecule as described herein can be introduced to at least one plant cell using any of the above described methods. In an alternative embodiment, any of the nucleic acid constructs described herein may be first transcribed to form a preassembled Cas9-sgRNA ribonucleoprotein and then delivered to at least one plant cell using any of the above described methods, such as lipofection, electroporation or microinjection.

Optionally, to select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility is growing the seeds, if appropriate after sterilization, on agar plates using a suitable selection agent so that only the transformed seeds can grow into plants. As described in the examples, a suitable marker can be bar-phosphinothricin or PPT. Alternatively, the transformed plants are screened for the presence of a selectable marker, such as, but not limited to, GFP, GUS (β-glucuronidase). Other examples would be readily known to the skilled person. Alternatively, no selection is performed, and the seeds obtained in the above-described manner are planted and grown and OML4 expression or protein levels measured at an appropriate time using standard techniques in the art. This alternative, which avoids the introduction of transgenes, is preferable to produce transgene-free plants.

Following DNA transfer and regeneration, putatively transformed plants may also be evaluated, for instance using PCR to detect the presence of the gene of interest, copy number and/or genomic organisation. Alternatively or additionally, integration and expression levels of the newly introduced DNA may be monitored using Southern, Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques.

In a further related aspect of the invention, there is also provided, a method of obtaining a genetically modified plant as described herein, the method comprising
a. selecting a part of the plant;
b. transfecting at least one cell of the part of the plant of paragraph (a) with at least one nucleic acid construct as described herein or at least one sgRNA molecule as described herein, using the transfection or transformation techniques described above;
c. regenerating at least one plant derived from the transfected cell or cells;
d. selecting one or more plants obtained according to paragraph (c) that show silencing or reduced expression of OML4.

In a further embodiment, the method also comprises the step of screening the genetically modified plant for SSN (preferably CRISPR)-induced mutations in the OML4 gene or promoter sequence. In one embodiment, the method comprises obtaining a DNA sample from a transformed plant and carrying out DNA amplification to detect a mutation in at least one OML4 gene or promoter sequence.

In a further embodiment, the methods comprise generating stable T2 plants preferably homozygous for the mutation (that is a mutation in at least one OML4 gene or promoter sequence).

Plants that have a mutation in at least one OML4 gene and/or promoter sequence can also be crossed with another plant also containing at least one mutation in at least one OML4 gene and/or promoter sequence to obtain plants with additional mutations in the OML4 gene promoter sequence. The combinations will be apparent to the skilled person. Accordingly, this method can be used to generate a T2 plants with mutations on all or an increased number of homoeologs, when compared to the number of homoeolog mutations in a single T1 plant transformed as described above.

A plant obtained or obtainable by the methods described above is also within the scope of the invention.

A genetically altered plant of the present invention may also be obtained by transference of any of the sequences of the invention by crossing, e.g., using pollen of the genetically altered plant described herein to pollinate a wild-type or control plant, or pollinating the gynoecia of plants described herein with other pollen that does not contain a mutation in at least one of the OML4 gene or promoter sequence. The methods for obtaining the plant of the invention are not exclusively limited to those described in this paragraph; for example, genetic transformation of germ cells from the ear of wheat could be carried out as mentioned, but without having to regenerate a plant afterward.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure. “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

The foregoing application, and all documents and sequence accession numbers cited therein or during their prosecution (“appin cited documents”) and all documents cited or referenced in the appin cited documents, and all documents cited or referenced herein (“herein cited documents”), and all documents cited or referenced in herein cited documents, together with any manufacturers instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

The invention is now described in the following non-limiting example.

### Example

The Large1 Forms Large and Heavy Grains

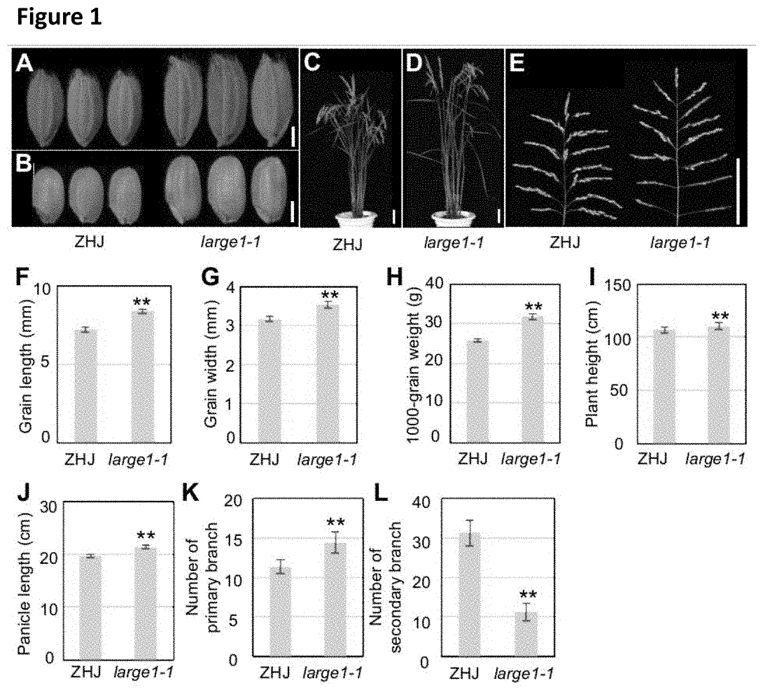

We have identified a number of grain size mutants in rice. The large1-1 mutant was isolated from γ-ray-treated M2 populations of the japonica variety Zhonghuajing (ZHJ). The large1-1 mutant displayed large grains and high plants (FIG. 1A-1E). The length of large1-1 grains was increased by 16.24% compared with that of ZHJ grains (FIG. 1F). Similarly, the width of large1-1 grains was increased by 11.54% compared with that of ZHJ grains (FIG. 1G). The large1-1 grains were also significantly heavier than ZHJ grains (FIG. 1H). The weight of large1-1 grains was increased by 23.11% compared with that of ZHJ grains. These results indicate that LARGE1 negatively regulates grain size and weight in rice.

Mature large1-1 plants were significantly higher than ZHJ plants (FIG. 1I). The large1-1 panicles were long and loose in comparison to the wild-type panicles (FIG. 1J), indicating that LARGE1 also negatively influences panicle length. As panicle structure and shape are determined by panicle branches, we investigated ZHJ and large1-1 panicle branches. The primary branches of large1-1 panicles were more than those of ZHJ (FIG. 1K), and the large1-1 had fewer secondary branches than ZHJ (FIG. 1L).

LARGE1 Regulates Cell Expansion in Spikelet Hulls

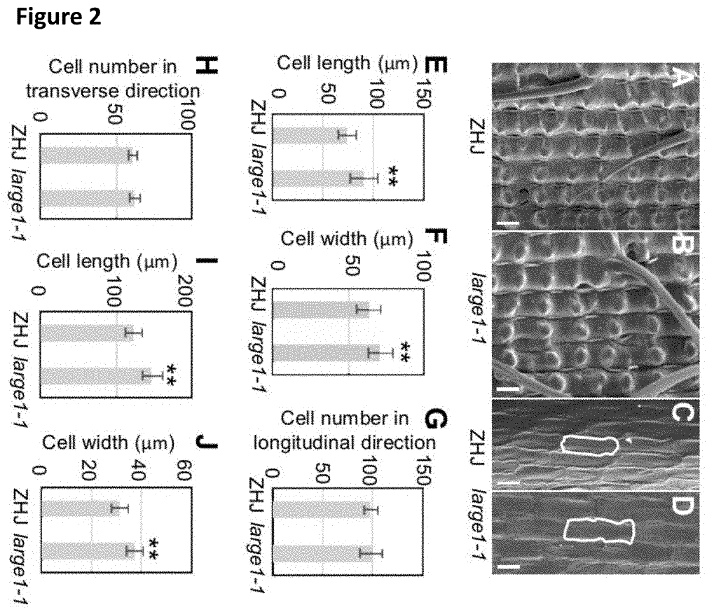

Grain growth is limited by spikelet hulls, and spikelet hull growth is determined by cell proliferation and cell expansion processes. To uncover cellular basis for LARGE1 in grain growth, we investigated cells in ZHJ and large1-1 spikelet hulls. As shown in FIG. 2, the outer epidermal cells in large1-1 lemmas were longer and wider cells than those of ZHJ lemmas, while cell number in large1-1 lemmas were similar to that in wild-type lemmas in both longitudinal and transverse directions (FIGS. 2A, 2B, 2E-2H). Similarly, the average length and width of inner epidermal cells of large1-1 was longer and wider than that of ZHJ (FIG. 2C, 2D, 2I, 2J). These results indicate that the long and wide grain phenotypes of large1-1 results from the long and wide cells in spikelet hulls. Thus, LARGE1 regulates grain size by limiting cell expansion in spikelet hulls.

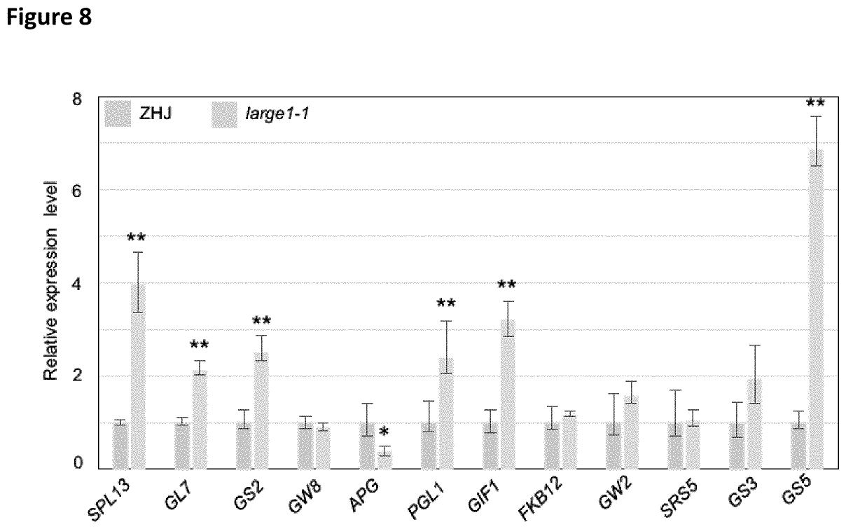

As several genes were reported to regulate grain size by influencing cell expansion in spikelet hulls, we investigated their expression levels in wild-type and large1-1 panicles (FIG. 8). SPL13/GWL7, a transcription factor, positively influences grain length by increasing cell expansion (Si, et al. 2016). Higher expression level of SPL13 in large1-1 panicles was observed. GL7/GW7/SLG7 promotes cell elongation in spikelet hulls, resulting in long grains (Wang, et al. 2015; Wang, et al. 2015; Zhou, et al. 2015), although GL7/GW7/SLG7 is also proposed to increase grain length by influencing cell proliferation (Wang, et al. 2015). Expression of GL7 was obviously increased in large1-1 compared with that in ZHJ (FIG. 8). The putative serine carboxypeptidase GS5 and the transcription factor GS2 affect grain growth by increasing both cell expansion and cell proliferation (Li, et al. 2011; Duan, et al. 2015; Hu, et al. 2015). Expression levels of GS5 and GS2 in large1-1 were significantly higher than those in ZHJ (FIG. 8). The bHLH transcription factor PGL1 controls grain length by increasing cell expansion (Heang and Sassa 2012a, b). APG, another bHLH transcription factor, regulates grain length by restricting cell expansion in spikelet hulls (Heang and Sassa 2012a, b). Expression levels of APG and PGL1 in large1-1 were lower and higher than those in ZHJ, respectively (FIG. 8). These data indicate that LARGE1 influences expression of several grain size genes that regulate cell expansion.

LARGE1 Encodes the Mei-2 Like Protein OML4

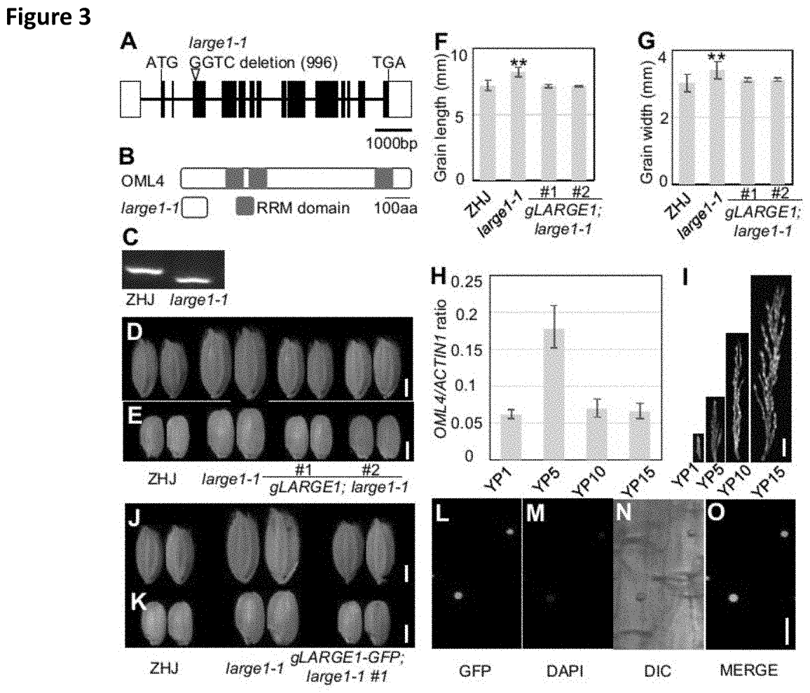

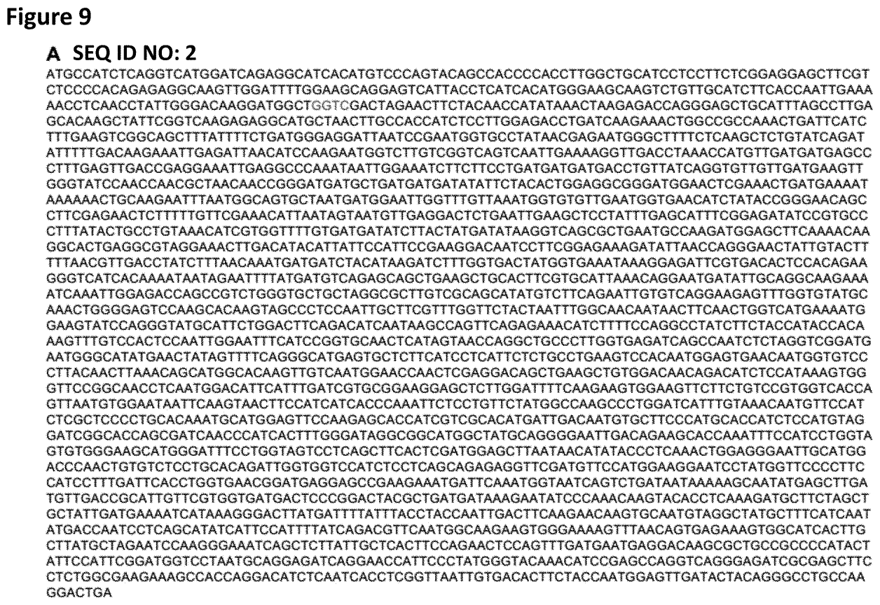

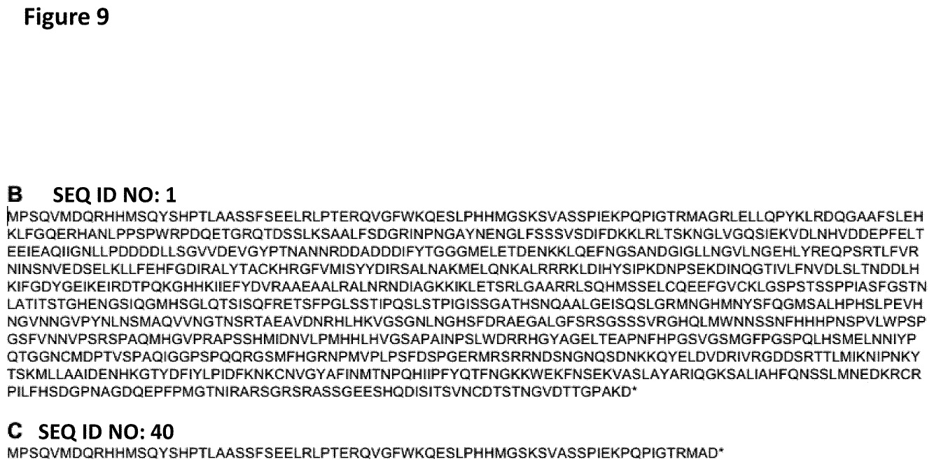

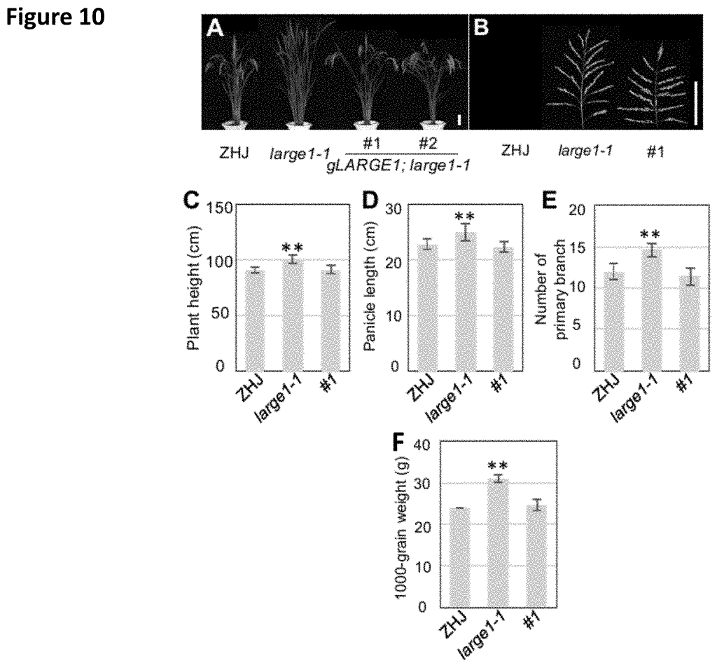

The MutMap approach was used to identify the large1-1 mutation. We crossed ZHJ with large1-1 and generated an F2 population. In the F2 population, the progeny segregation showed that the single recessive mutation determines the large grain phenotype of large1-1. The genomic DNAs from F2 plants with large-grain phenotype were pooled and applied for whole-genome resequencing. The wild-type ZHJ was also sequenced as a control. SNP analyses were performed as described previously (Fang, et al. 2016; Huang, et al. 2017). We detected 3913 SNPs and 1280 INDELs between ZHJ and the pooled F2 plants with large1-1 phenotypes. The SNP/INDEL ratio in the pooled F2 plants was calculated in the whole genome. Among them, only one INDEL in the coding region had a SNP/INDEL−ratio=1. This INDEL contains a 4-bp deletion in large1-1 in the gene (LOC_Os02g31290) (FIG. 3A; FIG. 9; Table 13), which leads to a premature stop codon (FIG. 3B). We further confirmed this deletion in LOC_Os02g31290 by developing dCAPS1 marker (FIG. 3C). These results indicate that LOC_Os02g31290 is the candidate gene for LARGE1.

The genetic complementation test was conducted to confirm whether the deletion in LOC_Os02g31290 was responsible for the large1-1 phenotypes. The genomic fragment of LOC_Os02g31290 (gLARGE1) was transformed into the large1-1 mutant and generated eleven transgenic lines. The gLARGE1 construct complemented the large grain phenotypes of the large1-1 mutant (FIGS. 3D and 3E). The grain length and width of gLARGE1;large1-1 transgenic plants were similar to those of ZHJ (FIGS. 3F and 3G). Genomic complementary plants also recovered to the wild type in plant height and morphology (FIG. 10). Therefore, the complementation test supported that the LARGE1 gene is LOC_Os02g31290.

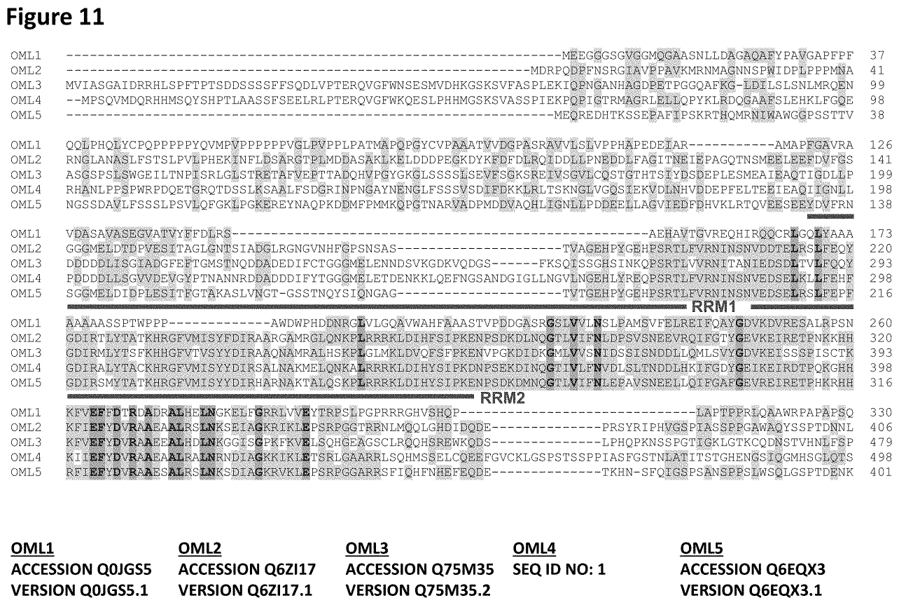

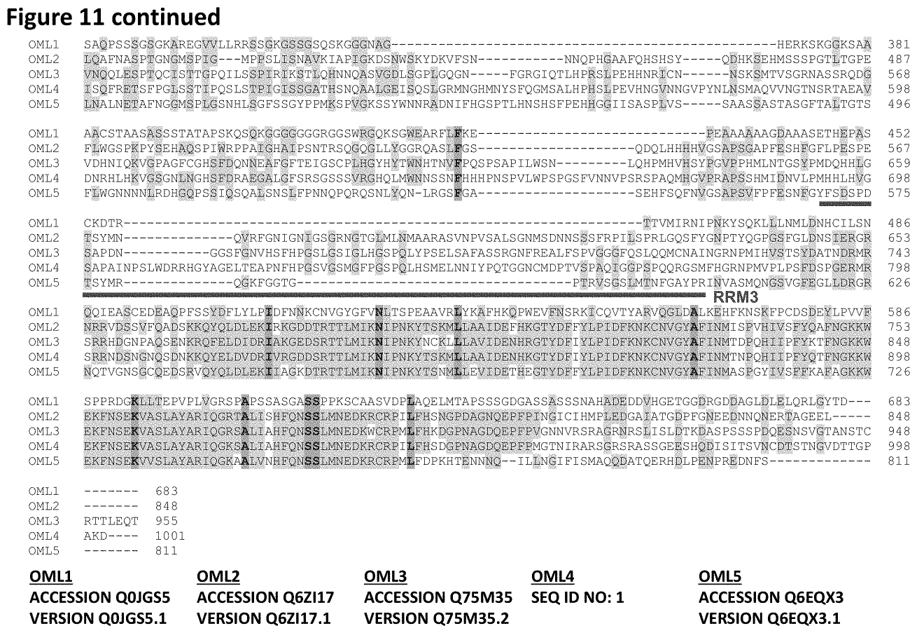

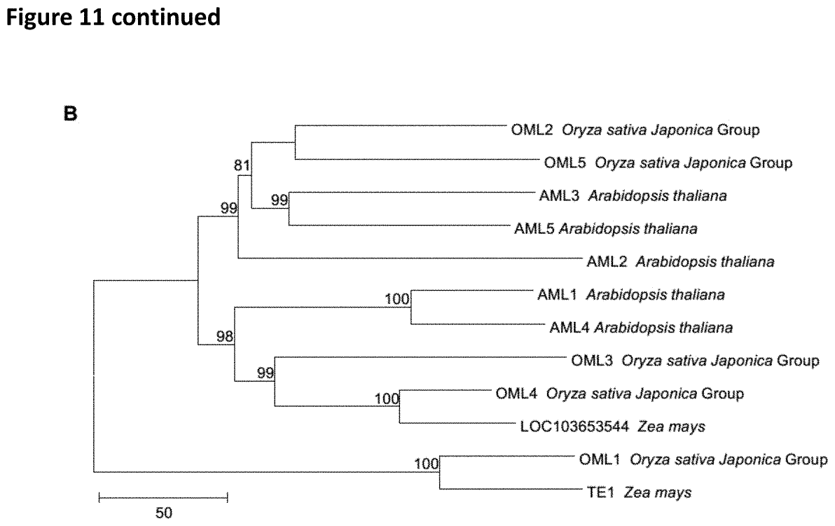

LARGE1/LOC_Os02g31290 encodes the Mei-2 like protein OML4 with three RNA Recognition Motifs (RRMs) (FIG. 3B and FIG. 11). Homologs of OML4 were found in crops (FIG. 11) but the role of OML4 and its homologs in grain size control are totally unknown so far. The mutation in large1-1 resulted in a premature stop codon. The proteins encoded by large1-1 (OML4^{large1-1}) lacked RRM motifs (FIG. 3B), which indicated that large1-1 is a loss of function allele.

Expression and Subcellular Localization of OML4

We investigated the expression of OML4 in developing panicles using quantitative RT-PCR analysis. The OML4 gene expression was detected and was also variable during panicle development (FIG. 3H). We further generated the OML4 promoter:GUS transgenic plants (proOML4:GUS) and examined the expression patterns of OML4 in developing panicles. During panicle development, GUS activity was detected in the panicles with about 1 cm of length. The strongest GUS activity was observed in the panicles with about 5 cm of length. The GUS activity was then gradually decreased during panicle development (FIG. 3I). Similarly, quantitative RT-PCR analysis indicate that expression of OML4 was relatively high in the panicles with about 5 cm of length (FIG. 3H).

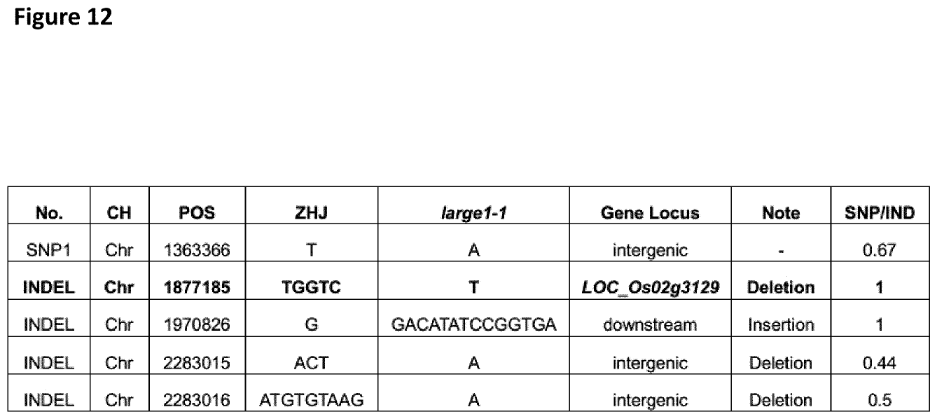

To investigate the subcellular localization of OML4 in rice, we generated gLARGE1-GFP transgenic plants. As shown in FIGS. 3J and 3K, the gLARGE1-GFP construct rescued the phenotypes of the large1-1 mutant (FIGS. 3J and 3K), indicating that the LARGE1-GFP fusion protein is functional. GFP signal in gLARGE1-GFP;large1-1 roots was predominantly detected in nuclei (FIG. 3L-30). Thus, this finding indicated that OML4 is localized in nuclei in rice

Overexpression of OML4 Results in Short Grains Due to Short Cells in Spikelet Hulls

To further reveal functions of OML4 in grain growth, we conducted the proActin:OML4 construct, transformed it into ZHJ and generated fourteen transgenic lines. The proActin:OML4 transgenic plants had short grains compared with ZHJ (FIG. 4A-4C), while the width of proActin:OML4 grains was similar to that of ZHJ (FIG. 4D). The grains were also significantly lighter than ZHJ (FIG. 4E). Grain length of proActin:OML4 transgenic lines was associated with the expression levels of OML4 (FIG. 4F). These data reveals that OML4 functions to restrict grain growth in rice.

Mature proActin:OML4 transgenic plants were shorter than ZHJ (FIGS. 4G and 4H). The average length of proActin:OML4 panicles was significantly decreased compared with that of ZHJ panicles (FIGS. 41 and 4J). The primary panicle branches of proActin:OML4 were comparable to those of ZHJ, while the secondary panicle branches of proActin:OML4 were obviously increased in comparison to those of ZHJ (FIGS. 4K and 4L), resulting in the increased grain number per panicle (FIG. 4M).

As proActin:OML4 transgenic lines produced short grains, we tested whether overexpression of OML4 could decrease cell length in spikelet hulls. We examined the size of outer epidermal cells in wild-type and proActin:OML4 spikelet hulls (FIGS. 4N and 4O). Outer epidermal cells in proActin:OML4 spikelet hulls were shorter than those of ZHJ spikelet hulls (FIGS. 4P and 4Q). By contrast, the number of epidermal cells in the longitudinal and transverse direction in proActin:OML4 spikelet hulls was similar to that in ZHJ spikelet hulls (FIGS. 4R and 4S). These results further revealed that OML4 affects grain growth by limiting cell expansion in spikelet hulls. X

OML4 Interacts with GSK2

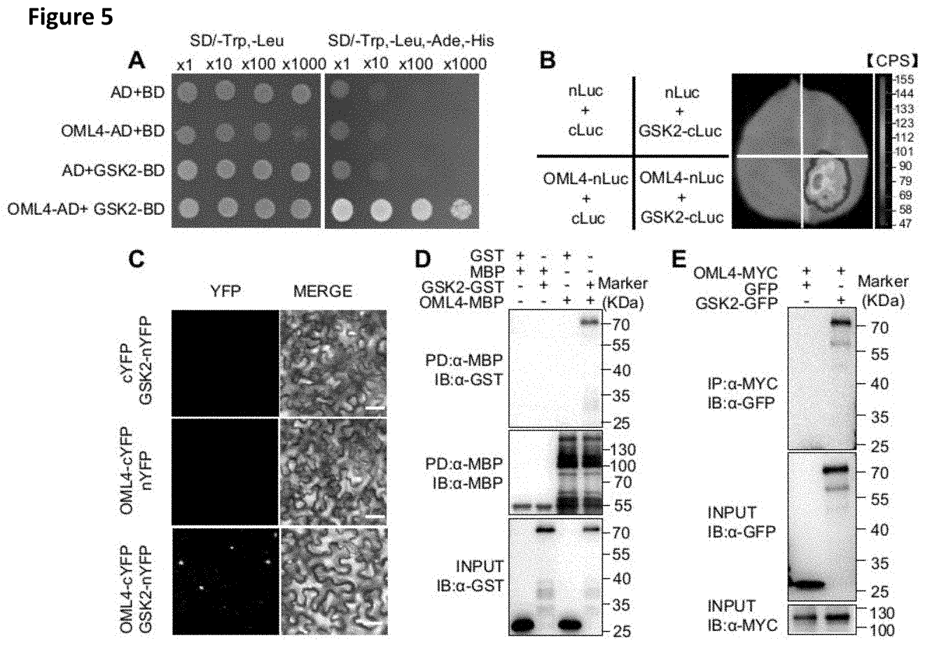

To further understand the molecular role of OML4 in grain growth control, we identified its interacting partners through a yeast two-hybrid (Y2H) assay. The OML4 full-length protein was used as the bait. Among several interacting proteins, six different clones corresponding to GSK2 were found in this screen. As GSK2 has been reported to restrict grain growth in rice, suggesting that GSK2 is a candidate OML4-interacting partner. We further confirmed the interaction of OML4 with the full length GSK2 in yeast cells (FIG. 5A).

We next verified the interaction between OML4 and GSK2 in plant cells using the firefly luciferase (LUC) complementation imaging assay (FIG. 5B). The OML4-nLUC and GSK2-cLUC were transformed and co-expressed in N. benthamiana leaves. The LUC activity was detected when we co-expressed OML4-nLUC and GSK2-cLUC, while no signal was observed in both combinations of OML4-nLUC/cLUC and nLUC/GSK2-cLUC. We then performed bimolecular fluorescence complementation (BiFC) assay to test the interaction between OML4 and GSK2 in plant cells (FIG. 5C). OML4 was fused with the C-terminus of the yellow fluorescent protein (OML4-cYFP), and GSK2 was fused with the N-terminus of the yellow fluorescent protein (GSK2-nYFP). Confocal laser scanning microscopy observation showed that a strong YFP fluorescence was observed in nuclei when we co-expressed OML4-cYFP and GSK2-nYFP in N. benthamiana leaves. These results indicate that OML4 associates with GSK2 in plant cells.

To investigate whether OML4 could directly interact with GSK2, we performed an in vitro pull-down assay (FIG. 5D). We expressed maltose binding protein (MBP)-fused OML4 (OML4-MBP) and GST tag-fused GSK2 (GSK2-GST) proteins in E. coli cells. As shown in FIG. 5D, OML4-MBP physically interacted with GSK2-GST but not the negative control (GST) in vitro. The co-immunoprecipitation (Co-IP) analyses were used to examine the association of GSK2 and OML4 in N. benthamiana. We co-expressed GSK2-GFP and OML4-MYC in N. benthamiana leaves (FIG. 5E). Total proteins were isolated and incubated with MYC beads to immunoprecipitate OML4-MYC. The anti-MYC and anti-GFP antibodies were used to detect immunoprecipitated proteins, respectively. GSK2-GFP proteins were detected in the immunoprecipitated OML4-MYC complexes (FIG. 5E), indicating that GSK2 associated with OML4 in vivo. These results reveal that OML4 can directly interact with GSK2 in vitro and in vivo.

GSK2 Phosphorylates OML4 and Modulates its Protein Level

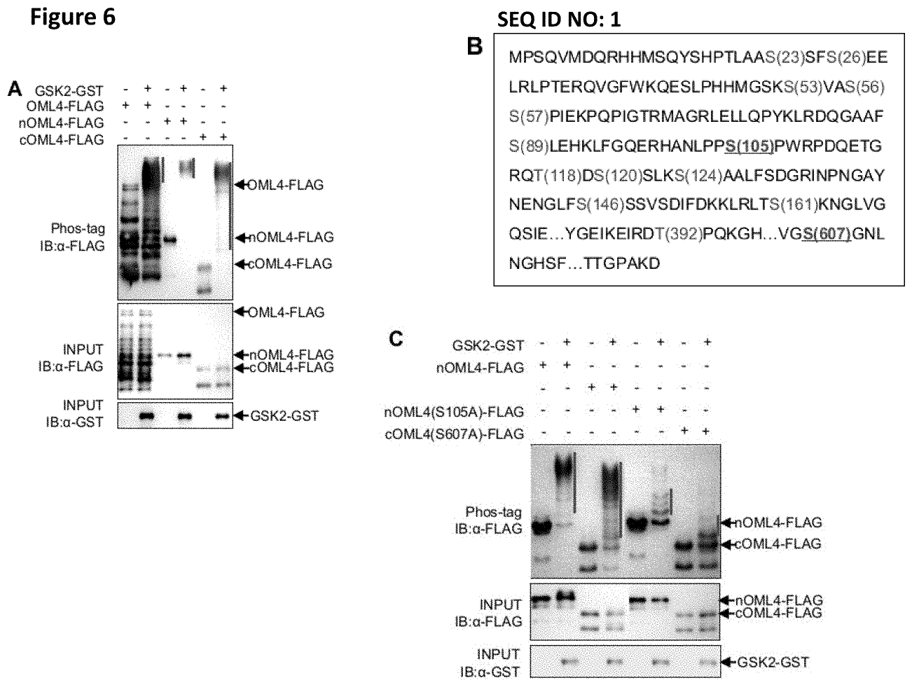

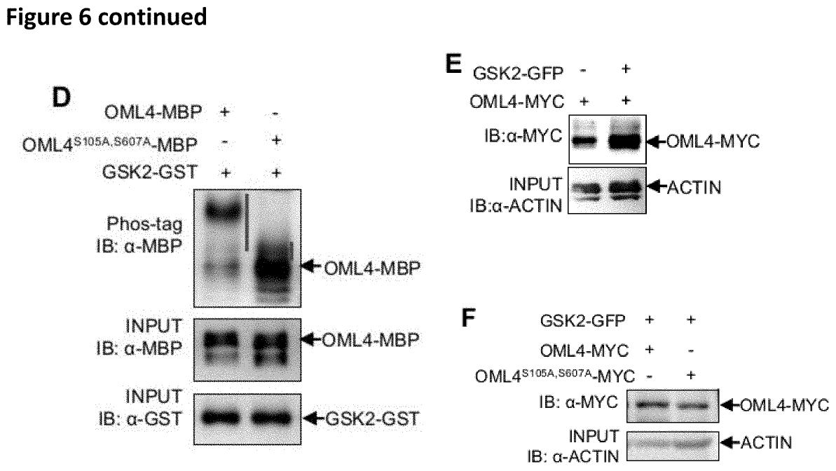

As GSK2 possesses kinase activity and interacts with OML4, we examined whether GSK2 could phosphorylate OML4. To test this, we performed an in vitro kinase assay. GST-fused GSK2 (GSK2-GST) proteins were incubated with OML4-Flag, the N-terminal region of OML4-fused Flag (nOML4-Flag), and the C-terminal region of OML4-fused Flag (cOML4-Flag) in an in vitro kinase assay buffer, respectively. The phosphorylated OML4-Flag, nOML4-Flag and cOML4-Flag were detected in the presence of GSK2-GST, while the phosphorylated OML4-Flag, nOML4-Flag and cOML4-Flag were not found in the absence of GSK2-GST (FIG. 6A). These results show that GSK2 can phosphorylate OML4 in vitro.

To further verify that GSK2 can phosphorylate OML4, we investigated phosphorylation sites of OML4. To identify the phosphorylation sites in OML4, the recombinant OML4 was incubated with the recombinant GSK2 in an in vitro kinase assay buffer, separated by SDS-PAGE electrophoresis, and then subjected to LC-MS/MS analysis for phosphopeptides. We identified 18 phosphopeptides of OML4, which correspond to 14 phosphosites (FIG. 6B). Among 14 phosphorylation sites of OML4, we observed that S105, S146 and S607 are Ser/Thr, Ser and Ser in its closest homologs in different plant species, respectively, suggesting that these three amino acids are possible conserved phosphorylation sites. We then mutated two amino acids into phosphor-dead alanine (OML4^{S105A,S607A}) and detected their phosphorylation levels by GSK2. Mutations of the two aforementioned Ser residues to Ala reduced the phosphorylation level of OML4, although OML4^{S105A,S607A} was still phosphorylated by GSK2 (FIGS. 6C and 6D), indicating that S105 and S607 partially contribute to its phosphorylation by GSK2. This result further supports that GSK2 can phosphorylate OML4 in vitro.

Considering that GSK2 can interact with and phosphorylate OML4 in vitro, we asked if the protein level of OML4 could be affected by GSK2. As shown in FIG. 6E, we found that the level of OML4-MYC was increased when GSK2-GFP was coexpressed in leaves of N. benthamiana. Considering that the phosphorylation level of OML4^{S105A,S607A} was lower than that of OML4 in vitro, we asked whether mutations in S105 and S607 could influence the protein level of OML4. As shown in FIG. 6F, the level of OML4^{S105A,S607A} was obviously lower than that of OML4 when we transiently overexpressed GSK2-GFP with OML4-MYC or OML4^{S105A,S607A}-MYC in leaves of N. benthamiana. These results indicate that GSK2 affects the level of OML4 possibly by influencing its phosphorylation.

GSK2 Acts Genetically with OML4 to Regulate Grain Size

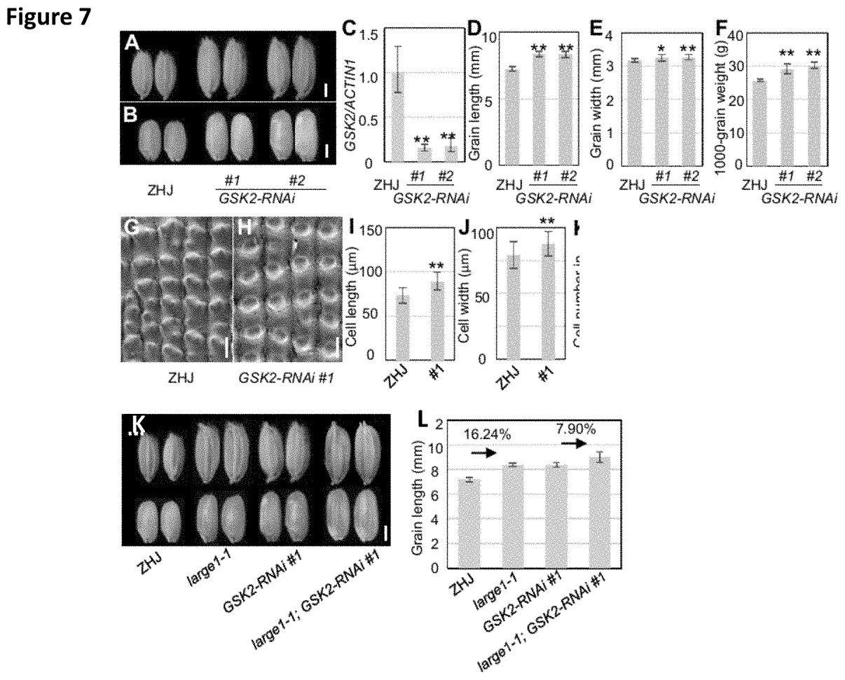

Although GSK2 has been described to affect grain size, the function of GSK2 in grain size control has not been characterized in detail. To carefully investigate the role of GSK2 in grain size control, we downregulated the expression of GSK2 using RNA interference (RNAi) approach (GSK2-RNAi), as described previously (Tong, et al. 2012). GSK2-RNAi lines showed longer and slightly wider grains than ZHJ (FIG. 7A-7E), indicating that GSK2 predominantly regulates grain length in rice. The grain weight of GSK2-RNAi transgenic lines was also significantly increased in comparison to that of ZHJ (FIG. 7F). We then observed epidermal cells in ZHJ and GSK2-RNAi spikelet hulls. GSK2-RNAi spikelet hulls contained longer and slightly wider epidermal cells than ZHJ spikelet hulls (FIG. 7G-7J). These results demonstrate that GSK2 controls grain growth by limiting cell elongation in spikelet hulls.

GSK2-RNAi produced long grains, like that observed in large1-1 mutant, and GSK2 and OML4 restrict cell elongation in spikelet hulls (FIG. 2 and FIG. 7). In addition, GSK2 can phosphorylate OML4 in vitro. We therefore speculated that GSK2 and OML4 could function in a common pathway to regulate grain length in rice. To test this, we crossed large1-1 with GSK2-RNAi and isolated large1-1;GSK2-RNAi plants (FIG. 7K). As shown in FIG. 7L, the length of large1-1 grains was increased by 16.24% in comparison to that of ZHJ, while the length of large1-1;GSK2-RNAi grains was increased by 7.90% compared with GSK2-RNAi. The results suggest that GSK2 acts, at least in part, in a common genetic pathway with OML4 to control grain length.

In addition, we also used the CRISPR constructs described herein to introduce at least one mutation into GSK2. In these CRISPR lines the grain length of gsk2-cri(7.99±0.30) was increased compared with ZHJ(7.20±0.17).

Discussion

Grain size and weight are critical determinants of grain yield, but the genetic and molecular mechanisms of grain size control in rice are still limited. In this study, we identify OML4 as a novel regulator of grain size and weight. GSK2 interacts with and phosphorylates OML4. GSK2 and OML4 function, at least in part, in a common pathway to control grain length in rice. These findings reveal an important genetic and molecular mechanism of the GSK2-OML4 regulatory module in grain size control.

The large1-1 mutant produced long, wide and heavy grains in comparison to the wild type. By contrast, overexpression of LARGE1 caused short and light grains. Thus, LARGE1 is a negative regulator of grain size and weight. Cellular analyses support that LARGE1 controls grain size by restricting cell expansion. Consistent with this, expression of several genes (e.g. SPL13, GS2, GS5 and GL7) (Li, et al. 2011; Che, et al. 2015; Duan, et al. 2015; Hu, et al. 2015; Zhou, et al. 2015; Si, et al. 2016), which control grain size by regulating cell expansion, was altered in large1-1 (FIG. 8).

LARGE1 encodes the Mei2-like protein (OML4) in rice. There are many Mei2-like proteins in plants, which have the conserved RRMs, but appear to have taken on distinct functions in plant development (Jeffares, et al. 2004). The Arabidopsis-Mei2-Like (AML) genes contain a five-member gene family, which play a role in meiosis and vegetative growth (Kaur, et al. 2006). In maize, TERMINAL EAR 1 (TE1), encoding a Mei2-like protein, plays a role in regulating leaf initiation (Veit, et al. 1998). In rice, PLASTOCHRON2(PLA2)/LEAFY HEAD2 (LHD2) encodes a Mei2-like protein (OML1) (Kawakatsu, et al. 2006). The pla2 mutant exhibited precocious maturation of leaves, shortened plastochron, and ectopic shoot formation during the reproductive phase (Kawakatsu, et al. 2006). However, the function of Mei2-like proteins in seed/grain size control has not been reported in plants. In this study, we identify OML4 as a negative regulator of grain size in rice.

We further identified the OML4-interacting proteins. Interestingly, one of them is the GSK2, a homologue of Arabidopsis BIN2 (BRASSINOSTEROID INSENSITIVE2) kinase, which has been reported to influence grain size and multiple growth processes in rice (Tong, et al. 2012). Previous studies showed that GSK2 interacts with several grain size regulators. However, the effect of GSK2 on cell proliferation and/or cell expansion in spikelet hulls has not been characterized in detail. In this study, we found that downregulation of GSK2 formed large grains as a result of large cells in spikelet hulls (FIGS. 7D and 7I). These results indicate that GSK2 restricts cell expansion rather than cell proliferation in spikelet hulls. Consistent with this, it has been proposed that GSK2 regulates grain size by interacting with GS2 that predominately promotes cell expansion in spikelet hulls (Che et al., 2015). GSK5, a homolog of GSK2, has been reported to control grain size by restricting cell expansion in spikelet hulls (Hu, et al. 2018). Considering that GSK2 is a functional protein kinase, we presumed that GSK2 could phosphorylate OML4. Consistent with this idea, we found that GSK2 can interact and phosphorylate OML4. We further observed that GSK2 influences the level of OML4 (FIG. 6E). It is possible that GSK2 might phosphorylate OML4 and prevent the degradation of OML4. Supporting this, we observed that mutations in S105 and S607 partially influence the abundance of OML4 (FIG. 6F). In addition, our genetic analyses suggest that GSK2 and OML4 function, at least in part, in a common pathway to control grain length in rice. Therefore, our findings reveal an important genetic and molecular mechanism of grain size control involving the GSK2-OML4 regulatory module in rice, suggesting this module is a promising target for grain size improvement in crops.

Materials and Methods

Plant Materials and Growth Conditions

The γ-rays was used to irradiate the grains of the wild type Zhonghuajing (ZHJ), and the large1-1 mutant was isolated from the M2 population. Rice plants were grown in the field according to a previous report (Huang, et al. 2017). Rice plants were cultivated in Lingshui from December 2016 to April 2017, December 2017 to April 2018 and Zhejiang Academy of Agricultural Sciences (Hangzhou) from July 2017 to November 2017, July 2018 to November 2018, respectively.

Phenotypic Evaluation and Cellular Analysis

The ZHJ and large1-1 plants grown in the paddy fields were taken photographs after completing grouting. MICROTEK Scan Marker i560 (MICROTEK, Shanghai, China) was used to scan mature seeds. We use the WSEEN Rice Test System (WSeen, Zhejiang, China) to measure the grain length and width. We also measured the 1000-grain weight with three replicates (Huang, et al. 2017).

We use a scanning electron microscope (SEM) to observe the cell size and cell number. SEM observation was performed as described previously (Duan, et al. 2015). Image J software was explored to measure cell length and width.

RNA Extraction and Real-Time RT-PCR Analysis

Total RNA of seedlings or young panicles were extracted using a RNA Pre Pure Plant Kit (Tiangen, Beijing). cDNAs was synthesized according to the previous study (Duan, et al. 2015). Real-time RT-PCR was conducted on an AB17500 real-time PCR system using a SYBR Green Mix Kit (Bio-Rad, Hercules, Calif.). Rice Actin1 gene was used as an internal control.

Identification of the LARGE1 Gene

We crossed large1-1 with the wild type ZHJ to produce F2 populations. We clone the LARGE1 gene using the F2 population. The whole genome of wild-type ZHJ and mixed-pool of 50 individual plants with mutant phenotypes were resequenced using NextSeq 500 (Illumine, American). The MutMap was used to isolate LARGE1 gene as described previously (Abe, et al. 2012), and the SNP/INDEL-ratio was analysed as described previously (Fang, et al. 2016).

Constructs and Plant Transformation

The genomic sequence of OML4, which contained a 2049-bp 5′ flanking region, the whole gene region and a 1259-bp 3′ flanking region, was amplified using the primers gOML4-99-F and gOML4-99-R. We used the GBclonart Seamless Cloe Kit to fuse the OML4 genomic sequence to the pMDC99 vector and generated the gOML4 recombinant construct. The latter series of the recombinant vectors were constructed using the same kit and similar methods. The related vectors we used in this study were pIPKB003 (containing the ACTIN promoter and fused with the CDS of the OML4 gene), pMDC107 (constructing the gOML4-GFP plasmid), and pMDC164 (constructing the proOML4:GUS vector). The plasmids gOML4, proACTIN:OML4, gOML4-GFP and proOML4:GUS were introduced into the Agrobacterium strain GV3101, respectively. The gOML4 and gOML4-GFP were transferred into large1-1, and other plasmids were transferred into the wild type according to a previous report (Hiei, et al. 1994).

GUS Staining and Subcellular Localization of OML4

GUS staining of panicles in different developmental stages was performed as described previously (Fang, et al. 2016). The GFP fluorescence of gOML4-GFP transgenic seedlings was observed using the Zeiss LSM 710 confocal microscopy. The 4′, 6-diamidino-2-phenylindole (DAPI) (1 μg/mL) was used to stain cell nuclei.

Yeast Two-Hybrid Assays

The cDNA sequences of GSK2 and OML4 were amplified using gene-specific primers (Table S4), and products were fused into the linearized pGADT7 and pGBKT7 vectors, respectively. Yeast two-hybrid analysis was conducted according to the manufacturer's instruction (Clontech, USA).

BiFC Assay

Full-length cDNA fragments of OML4 and GSK2 were recombined into the pGBW414-cYFP and pGBW414-nYFP vectors. The constructs were transformed into Nicotiana benthamiana mesophyll cells by acetosyringone (AS) for transient expression. Confocal imaging analysis was performed using a Zeiss LSM 710 confocal microscopy.

Pull Down Assay

Recombinant proteins (OML4-MBP and MBP) and the prey proteins (GSK2-GST and GST) were incubated in TGH buffer (50 mM HEPES, PH 7.5, 10% glycerol, 150 mM NaCl, Triton X-100, 1.5 mM MgCl₂, 1 mM EGTA, and protease inhibitor cocktail tablet) for 0.5 hr at 4° C. with 20 μl MBP-beads per tube. Centrifuge 500 rpm for 2 mins and discard supernatant to stop the reaction. Wash beads with ice-cold TGH buffer for 5 times and then add 50 μl SDS-loading buffer. Denatured the samples at 98° C. for 5 mins and finally subjected to the SDS-PAGE analysis. We used anti-MBP (Beyotime) and Anti-GST (Beyotime) to detect the input and the pull-down samples, respectively.

Phosphorylation Analysis

The coding sequences of OML4, nOML4 and cOML4 were amplified using the specific primers (OML4-FLAG-F/R, nOML4-FLAG-F/R and cOML4-FLAG-F/R) in Table S4. The products were cloned to the vector pETnT to construct OML4-FLAG, nOML4-FLAG and cOML4-FLAG plasmids. The GSK2 coding sequence was amplified using the primers GSK2-GST-F/R and subcloned to the vector pGEX4T-1 to construct GSK2-GST plasmid.

All these plasmids were transformed into Escherichia coli (host strain BL21). Induction, isolation and purification of OML4-FLAG, nOML4-FLAG, cOML4-FLAG and GSK2-GST proteins were done as described previously (Xia, et al. 2013). 10 μL of GSK2-GST was incubated with 5 μL of OML4-FLAG, nOML4-FLAG and cOML4-FLAG in 20 μL reaction buffer (25 mM Tris-HCl, PH 7.5, 10 mM MgCl₂, 1 mM DTT, 50 mM ATP) for 2 hours, respectively. Phosphorylated products were analyzed by phos-tag SDS-PAGE. Anti-GST and anti-FLAG and anti-GST antibodies were utilized to detect the phosphorylated products and the input.

SEQUENCE LISTING
Rice
SEQ ID NO: 1: OML4 amino acid sequence
MPSQVMDQRHHMSQYSHPTLAASSFSEELRLPTERQVGFWKQESLPHHMGSKSVASSPIEKP
QPIGTRMAGRLELLQPYKLRDQGAAFSLEHKLFGQERHANLPPSPWRPDQETGRQTDSSLKS
AALFSDGRINPNGAYNENGLFSSSVSDIFDKKLRLTSKNGLVGQSIEKVDLNHVDDEPFELTEEI
EAQIIGNLLPDDDDLLSGVVDEVGYPTNANNRDDADDDIFYTGGGMELETDENKKLQEFNGSA
NDGIGLLNGVLNGEHLYREQPSRTLFVRNINSNVEDSELKLLFEHFGDIRALYTACKHRGFVMIS
YYDIRSALNAKMELQNKALRRRKLDIHYSIPKDNPSEKDINQGTIVLFNVDLSLTNDDLHKIFGDY
GEIKEIRDTPQKGHHKIIEFYDVRAAEAALRALNRNDIAGKKIKLETSRLGAARRLSQHMSSELC
QEEFGVCKLGSPSTSSPPIASFGSTNLATITSTGHENGSIQGMHSGLQTSISQFRETSFPGLSST
IPQSLSTPIGISSGATHSNQAALGEISQSLGRMNGHMNYSFQGMSALHPHSLPEVHNGVNNGV
PYNLNSMAQVVNGTNSRTAEAVDNRHLHKVGSGNLNGHSFDRAEGALGFSRSGSSSVRGHQ
LMWNNSSNFHHHPNSPVLWPSPGSFVNNVPSRSPAQMHGVPRAPSSHMIDNVLPMHHLHVG
SAPAINPSLWDRRHGYAGELTEAPNFHPGSVGSMGFPGSPQLHSMELNNIYPQTGGNCMDPT
VSPAQIGGPSPQQRGSMFHGRNPMVPLPSFDSPGERMRSRRNDSNGNQSDNKKQYELDVD
RIVRGDDSRTTLMIKNIPNKYTSKMLLAAIDENHKGTYDFIYLPIDFKNKCNVGYAFINMTNPQHII
PFYQTFNGKKWEKFNSEKVASLAYARIQGKSALIAHFQNSSLMNEDKRCRPILFHSDGPNAGD
QEPFPMGTNIRARSGRSRASSGEESHQDISITSVNCDTSTNGVDTTGPAKD
(RRM domains are underlined)
SEQ ID NO: 2: OML4 nucleic acid sequence (genomic) [symbol]
CCATCTCAGGTCATGGATCAGAGGCATCACATGTCCCAGTACAGCCACCCCACCTTG
GCTGCATCCTCCTTCTCGGAGGAGCTTCGTCTCCCCACAGAGGTACTCCATAATTGCGATA
ATTTTGGTCCAAATCTTCCTTCTGGAAGTCTTTTCTATGTGATGGCTAATGGTGATCTGTCT
GGAAATTTTATTTGTTTAGCCTTTCCTGGTGACCTGGTTATGATTCATATCTACAAATCTTTA
CCAATTATTCTCACCATGTTTATATATTCATTATGATGAATATCTATAATTTGTACTAATTTTT
CTCTCACCATGTTCATCTCTTCTTCTATCTTTGCAGAGGCAAGTTGGATTTTGGAAGCAGGA
GTCATTACCTCATCACATGGGTTAGTGCTGAGTTTGATTTAACTTATACTGGGTTTTGTTCTA
CATTTGTCTATTAGTATGCCTTGCGGTTGCAGCTTTAAATTTTCACGCTGTTGGGGGCATGT
ACTTAGTCGTTTCTTTATGCATGGATAGCAAAACTTTGGGGACATCTATTGGCTCTTTTTTCT
GCATGAATTACAAACCATCTATAGGAGGGCTTTCTTTGAAAGGTTTACCTGGCCTTGACAG
CCATCTAGCCTGCCTAAATTGAGTTAACACTAGGTGCTGGCCTTGCCACCTGATTAGTGCC
TTGGTGAACATTGGTTTTAAGTATTTTCCCCTCTATTTATGTTAGATTAATTTGCAATAAATAA
ATAAATAAATAAATAAACATGCATGTTCTTCTTATATATGCAATTGGTTGTTGTGTTTTTTCTT
GTTATGGTTACTTTCTTTGTTCTATTGTACTACTCTTTGAGTCTTTGATAATGTGATGGTTCA
TAAATATGTGGGTTTCCCATGATATTTTCTCATAACTAGGTGGGTTTCCAATATTGACAGGA
AGCAAGTCTGTTGCATCTTCACCAATTGAAAAACCTCAACCTATTGGGACAAGGATGGCTG
GTCGACTAGAACTTCTACAACCATATAAACTAAGAGACCAGGGAGCTGCATTTAGCCTTGA
GCACAAGCTATTCGGTCAAGAGAGGCATGCTAACTTGCCACCATCTCCTTGGAGACCTGAT
CAAGAAACTGGCCGCCAAACTGATTCATCTTTGAAGTCGGCAGCTTTATTTTCTGATGGGA
GGATTAATCCGAATGGTGCCTATAACGAGAATGGGCTTTTCTCAAGCTCTGTATCAGATATT
TTTGACAAGAAATGTGAGTGGTTTTTCTTTATCATTTGCATTTGCTTCATCAAAATGCTTGAT
TCTATGAAACACAGACTCGAGAAATTTCCATTCCATTGATAGTAAATGTGCTGAAATATACC
ATCACATGACATATGTATTGGCAACTACAACGCTTCCTTACGATCTTACATTCTATACTTAAT
GCTTCTCATGAATGAATAGAAATGTACAAAAGTAAAACAAAAAATACAACTGAAATGAAAGG
GTAGTAAAATGAAATGACTTTCATTCCCTTCCCCTTTTTCCATAAGAATCTTGCCTCCTTTAT
CTCCTGTTTCTTTCTAGTGGCTAAAAGAATCAATCCACTTTAGTTTGGTATCGTAGTCCGTC
TGTTATTCTTGTACATTCTTTTGCCAAAAAAAAGTCTGCACTCTGGTTCAACCTTTATTCTAT
TGTAATATGTTATCTCCAATTTCCAATCATTGACCACTGTCTGATTTTATTTGTAACCTGTGC
AGTGAGATTAACATCCAAGAATGGTCTTGTCGGTCAGTCAATTGAAAAGGTTGACCTAAAC
CATGTTGATGATGAGCCCTTTGAGTTGACCGAGGAAATTGAGGCCCAAATAATTGGAAATC
TTCTTCCTGATGATGATGACCTGTTATCAGGTGTTGTTGATGAAGTTGGGTATCCAACCAAC
GCTAACAACCGGGATGATGCTGATGATGATATATTCTACACTGGAGGCGGGATGGAACTC
GAAACTGATGAAAATAAAAAACTGCAAGAATTTAATGGCAGTGCTAATGATGGAATTGGTTT
GTTAAATGGTGTGTTGAATGGTGAACATCTATACCGGGAACAGCCTTCGAGAACTCTTTTT
GTTCGAAACATTAATAGTAATGTTGAGGACTCTGAATTGAAGCTCCTATTTGAGGTTAGTTA
CTTATTTCTTCTTCTTTGAATCACTCTTCTGTTACAACAGATTTGACATCTGAGAAGCCATCT
GTTCTTCTATGCAGCATTTCGGAGATATCCGTGCCCTTTATACTGCCTGTAAACATCGTGGT
TTTGTGATGATATCTTACTATGATATAAGGTCAGCGCTGAATGCCAAGATGGAGCTTCAAAA
CAAGGCACTGAGGCGTAGGAAACTTGACATACATTATTCCATTCCGAAGGTAACCATCAAA
TCATCAATTGCCACTTAACTGAAAATGCTTATCTGCATTTTCTGTTGCCTGTTCTTGTGCTTA
GAATGTTATTATTCTAGATATTCACTAAAATTGAGCACATTTGCTTTTCTTTCCCCACAGGAC
AATCCTTCGGAGAAAGATATTAACCAGGGAACTATTGTACTTTTTAACGTTGACCTATCTTTA
ACAAATGATGATCTACATAAGATCTTTGGTGACTATGGTGAAATAAAGGAGGTACGATATTT
CATTTGCTGACTACTATTATAGCTAGAAAGTATGACTCACTAGTTCTATTTGCAGATTCGTG
ACACTCCACAGAAGGGTCATCACAAAATAATAGAATTTTATGATGTCAGAGCAGCTGAAGC
TGCACTTCGTGCATTAAACAGGAATGATATTGCAGGCAAGAAAATCAAATTGGAGACCAGC
CGTCTGGGTGCTGCTAGGCGGTAAGTCATTTGGGTCTTGTCAACAGTGATAATACTCTGTT
TGCTGTTTTCTTTTTAGTTCTTACTACTACTTTCTTCATCACTTTTATAACATACATATTCACC
ATTTTAACATTTTTGACATACTAGCTGAATGCCCATACATTGCAATGGGAATTAATTATTAGA
GAACCACACTGCACACTCTAAAGCCTCAAAAATTAATATAAAACTATCCTCAATGTAAATCTT
AGGGTCATATTTTTTGTCGTCATTTTCACCTCCAATTTGTTTTCCCTGTTAGACGGCTTGAG
GTTAGGAAAGGGACAAAAGTCCACCTACCTCACTGTTTGGGGGACTCACATAGCAGTGGT
GGTGGGTGGTGGGTGGTGGCAGTGGTAGAGTATAGAGTATATATTTTGAATGCATAGTGTA
TCTTCTTTTATGTTTGAGTTTCTTATCCACATAATGTTCATGCTGAGCTGTGCAGGAATAGTT
TAGTTGAATGCAGCATATTGAATAAACGAAAAAAATGTCAAACATGTTGGTAGAATGGCATT
TCTCTGAGTATTTTAATTGTAGCTATTGCTTTGACTGATTTCAATGCTCTCTATCACAGCTTG
TCGCAGCATATGTCTTCAGAATTGTGTCAGGAAGAGTTTGGTGTATGCAAACTGGGGAGTC
CAAGCACAAGTAGCCCTCCAATTGCTTCGTTTGGTATGCTGTTTTCCTTTTTCATCTCAATG
TATGTTTTGCTGATAGGTGCATTTTCTGACACGGATGGTTATATTGCAAGGTTCTACTAATTT
GGCAACAATAACTTCAACTGGTCATGAAAATGGAAGTATCCAGGGTATGCATTCTGGACTT
CAGACATCAATAAGCCAGTTCAGAGAAACATCTTTTCCAGGCCTATCTTCTACCATACCACA
AAGTTTGTCCACTCCAATTGGAATTTCATCCGGTGCAACTCATAGTAACCAGGCTGCCCTT
GGTGAGATCAGCCAATCTCTAGGTCGGATGAATGGGCATATGAACTATAGTTTTCAGGGCA
TGAGTGCTCTTCATCCTCATTCTCTGCCTGAAGTCCACAATGGAGTGAACAATGGTGTCCC
TTACAACTTAAACAGCATGGCACAAGTTGTCAATGGAACCAACTCGAGGACAGCTGAAGCT
GTGGACAACAGACATCTCCATAAAGTGGGTTCCGGCAACCTCAATGGACATTCATTTGATC
GTGCGGAAGGAGGTAATTTGTATATCCTAATCTCCTTTGTTTGAAAAATCTGTTATGTTAAG
AGGAACTGAACTATCCTAGGATATGTTGGTTCCATCATGGGTCATGCCATGATTTTGGTGG
GATGAATTCCTCGTTTTCTATAATTACATGCTTTTGTGGGATGAGGTGGTGATCGACCAAAC
ACATTTCGTTTCTCAAACCAATGAAAGTTGTGTAATGTTTGGATGAAAGAAATTACATCTGG
ATCAATCTACAAGCCTTATATGTTATCTAATCATTCCTTGAATGTGTATTTTTTTTTTCACTTG
CAGCTCTTGGATTTTCAAGAAGTGGAAGTTCTTCTGTCCGTGGTCACCAGTTAATGTGGAA
TAATTCAAGTAACTTCCATCATCACCCAAATTCTCCTGTTCTATGGCCAAGCCCTGGATCAT
TTGTAAACAATGTTCCATCTCGCTCCCCTGCACAAATGCATGGAGTTCCAAGAGCACCATC
GTCGCACATGATTGACAATGTGCTTCCCATGCACCATCTCCATGTAGGATCGGCACCAGCG
ATCAACCCATCACTTTGGGATAGGCGGCATGGCTATGCAGGGGAATTGACAGAAGCACCA
AATTTCCATCCTGGTAGTGTGGGAAGCATGGGATTTCCTGGTAGTCCTCAGCTTCACTCGA
TGGAGCTTAATAACATATACCCTCAAACTGGAGGGAATTGCATGGACCCAACTGTGTCTCC
TGCACAGATTGGTGGTCCATCTCCTCAGCAGAGAGGTTCGATGTTCCATGGAAGGAATCCT
ATGGTTCCCCTTCCATCCTTTGATTCACCTGGTGAACGGATGAGGAGCCGAAGAAATGATT
CAAATGGTAATCAGTCTGATAATAAAAAGCAATATGAGCTTGATGTTGACCGCATTGTTCGT
GGTGATGACTCCCGGACTACGCTGATGATAAAGAATATCCCAAACAAGTATGTGTAACAAC
AATTGACTTGGGATTCCAGGTACACCTCAAAGATGCTTCTAGCTGCTATTGATGAAAATCAT
AAAGGGACTTATGATTTTATTTACCTACCAATTGACTTCAAGGTGATCTAGATTTATTTAGTA
TGCAACTAATACATCATATTTGTTCAGATAGTCTTGCCTAATCGAATTACTGAATGGGATGT
GTCCTACTTTTCAGAACAAGTGCAATGTAGGCTATGCTTTCATCAATATGACCAATCCTCAG
CATATCATTCCATTTTATCAGGTGAGAGATACTATCTATAGGGCCTGCCCAGCTGAGCTGG
CTGCAACTGCATCACAGCCAGCTGCTGCCCGAAGCAGCAATGCCAGTGGCTTGCTCCTGC
AGCCAGCTCAGCCAAGAGAAACCATTATCAAGTGCTAGTCGCATGAAGGCAATAGCTTACG
TTCTGCATGCGGCTTGTCAACTTTGGACATTGTACATTATCCAATTTGAAATAAATCAATATT
GTGCCCTCATCCCTTTTTTGCAGACGTTCAATGGCAAGAAGTGGGAAAAGTTTAACAGTGA
GAAAGTGGCATCACTTGCTTATGCTAGAATCCAAGGGAAATCAGCTCTTATTGCTCACTTCC
AGAACTCCAGTTTGATGAATGAGGACAAGCGCTGCCGCCCCATACTATTCCATTCGGATGG
TCCTAATGCAGGAGATCAGGTATGATCTTTCTCTCTCTCTCTCTCTCTCTCTCTCTCTCTCT
CTCTCGTTGATAAATGGAGTTAAAGCAGCAGATGACACTTGGACACAGTTTGCTGTTTTATG
GCAAGTTCTTTTTTGTTAGCAGGCCTTTTCTGCTGTATTTGAATGTATTTTATCACAAATAGA
CCTATATTTTGTGGTTGTTTCTGTTCTGCAGTTCCAAATTTCATGCCACATTGTGGGTTCCTT
CTCACTCTCTTTTTTCTTTTGCATGCCATGTCATGGTCTCTTTCCTATATATTACAGTTGCAA
GCACCATTCCTTCTCATTTCTTTGGGAACTAGAAGATAATAGTATCTGTTACTTATTATTCTC
TCCTAATGGCACTGAGTTTGCTCCATAATCACTAGTCATTCTTGTTTGGTCTTTCAGAACCT
TTTATGTTAGCTCTGAAAGGTTTATTGTTCCATGCAGATTGCTATTCCTTTAACTATATGATT
AACACCTTTTGTCCTTTTGTTGTCCATTAGGAACCATTCCCTATGGGTACAAACATCCGAGC
CAGGTCAGGGAGATCGCGAGCTTCCTCTGGCGAAGAAAGCCACCAGGACATCTCAATCAC
CTCGGTTAATTGTGACACTTCTACCAATGGAGTTGATACTACAGGGCCTGCCAAGGAC [symbol]
GTAACACAACTGCTCTGGATCACTAACCCCCAAATCCCAAATCATAACTTTTGCGACGCGG
TTTCCATTTCCCAGTTTTCCGCCCTTTTTCCCCCAACTTTGGTTTTTTTGGTATGACCCCCAA
TCTGTATTTATTAACTTCCATGAATGCGGGTTACCGAAGACTTGGCTAGATTGCTGCAACAT
TTTGTCCCTGATGGAAACATGGATAGAGAGACAGAGAGGGTGCTTCCAGTTTCCCCTGAAC
CTACCATTATCATATTAACCTGAAGGCCGAGAAAGGTGAAAGGCGCAGCGAGAGCTTCCA
GATTTTGGTCACTTTTTAAGAATGTATTAACCCCATGTTGTATAGCAGTTTCCAGTAACTGTG
CTGAGGGGAGAGAGAGAAAGAGAGGAGAGCAAGGAGACAATTTACATGAGTTTTTAGTGG
TGGTGTGGAGAGGAAGTCTTTCCCTGCATTTTCTTTTGGAACCTTTTCTGGCGTCTTCATCT
ATGTTCCATTTTGAGTTGAGGTCTCCTCTTTTAAGTTGTGTGCAGAGGAGTTCCGATTTTGT
CTTCAGGGAACTTTGACCGTATCTATCGACCTTCATATGTAAATCAACATCTCTATATAGTTT
GTGTGCCCTCTGTTGTATGCCTGCGGCCCCTTGCACCAAACGAATTGTCTCTCTAACTCGT
GAGATTGCTGTCCTCGTTTGGTCGTATTACATCTGAATCTAAGCATTTGATGTTACGCAAAT
ACATGCCAATGGCTGCATTGCGACATGTAGCAGACGGCCAATGTTCAAACAAAAATCTTAA
CTTATGAAGTATACTAGTACCTCCATCCTAAAATATAACAATTTGGGACTGATGTGTATATCC
TAGTCCAATGAATCTGGCCCTTGTCTAAATTCATTGGACTGGATATGTCTCATCCACTTTCA
AATAGCTATATTTTGGGACGACACTTTCAAATAACTATATTTTGGAACGGAGGGAGTAAATA
ATTATAAATACTAGTACTATCAATTTGGCACATGGTGTCAAGTCCACTTGGTGCATGGTCAT
CTAGGCTTCCCTTTGGTGCCTCTCTTAAGAACCTTCTAAGCGTTTAACACAAATTAAAATCG
AAGTAAGAATCTGACACGAATTGAATTCGAAATTTGCTCTCACAATGAGACAAAAACAAAAG
AATTTGGCGAATACAGCAGTAACGCCGTGGACGAAGACAATAATAATAGTCTCGGACTCGG
GAGTTGTTCAGTCAGTGTCCGTCA
SEQ ID NO: 3 OML4 promoter sequence
cttactgtcagatggactactttgagaaaaaaagggggcaaaataactatatcaataaattaacctctgtcaaaacaggcaacaatta
aaattaagagcagcttagaccattctttctaattttctagttataagatgcacattctacttcagttttcgttagcgcgtttttcaaactgctaaa
cgatatgttccgtgcgaaaactttctatataagtagcttaaagatatcaaataaatccattattcaattttgtaataatcaaaaactcaatta
atcatacgctaatgactttatattcccttactcaatcttcatctatcttaaattgggccatgtctctttttttaattaagatgcagattttacttcggtt
ttcattagcacgatttttaaatcgctaaatggtgtgtttcatacggaggatctacttttgaaaatttttaatgatttaaactcaattatttatatatta
atagctctctcattctgcgtgcccttacttaatcctcatcctcattacttacaaacactgcataacggagtaatagtattattattaatgttatgtt
aatcctgatcctaatccctaatccaaagagaaccatctaaatacccggcgcaagcaaccccctctgctctgtcgtaaccaaaaatttcc
ctctcccctgcgaactcccaccacccaaatttaactcccccaacctcccgcccgtcgcgccagctgacccgtcactgacagggtggg
ccccacgccccggcgcggtgggtcccacgcgtcagcgaccgtgggtagggtgggcgcgggtgcgccccccccccacccggtccc
gtgctccgcggtggcggtcaccgggtgcggggggtgggccgcgtatataggcgggccgccgcgccgcgcgctGCTGGCTAG
GTGTAGGAGCTTCAGCTTTGGCCCACATCGCCCCCCTCTCGCCCTCTTCCTTCGCTTTCGT
CTCACCGCCCCCACCGCCTCGCCTGGGGGAGGGGAGGGGAGGGGAGCCCTTCGCCGGA
GCGGCCAGGTTCCGGCGAGCATCTAGAGGAGGAGGAGGGGGAGGGCGGGGAATGGGGA
GCGGCGGCCGGAAGAGGGGGCACGTCGTCGCTGCTGCTGCTGCTGTTGTTGTTGCGTCC
CTCTAGGGTTAGGTAGGGGCGTTGCTGGAGTAGCTTTCTCCCACCCCCAATTTTTTTTGTT
CGTTCTCTTTCGCTCTCGAGGTCTCTCTCTCTCTCTCTCTCTCCCCACCTCCGCCCCGCCG
CGTCGGGGGGTTGGTCCTCCTTGCCGGCGGCGTTCGTCGTCGTCGTCGTCGCATTGAGG
GGGGAGAGGTGATCCGGCCGTAGTCCATTCCAGCTCGGGGAAGGGGGGGGGCATGGGG
GCAGCTGGTCCGCGTGGTGGTGCCGCCGCTCTCGAATTCGTGCGGGGATTTTGGTTTTGA
AGAGGGAGGTGACCCGCACGCGCCGATCTGGTGAGGCCTTGCTCGTTTTGTGCTGTTTTT
TGTGCCTAGCTTTGGTCGGAGGTGTTTGAATTGTTGGGGAATTTTGAGCTTTTGCTGTGAT
CTGAGCTTCAAATTTCGGTGGGGGTTAACTTGGCCTGGGCACCTCGGAATTTCTGTTTAAT
TTTTGGTGGGGTTTCTTTGATCACAAGATACTTGCTTGCTTGGAGCTTTGGGAGCCCGAGG
CGCATTAAATTCCACATCTTCTGCGCTGTTTTATCGGGAAATTAAACATTTCGTGCTCAAGT
CTGTGGGGGGGTTTTTCCCTCGGATTTGTCAAATCTGGCGGCTCTTGTTCGAAAATTTTCA
TCTTGGGAGCTTACGAACGCAAAATTCTTCACATTTCTTTTGCTTCCTGGCTTGGAAGCTGT
GGAATCCAAATTTTTATGTGCTGAATTGACATGGTTAGCCATGTTTTTTTTCCACAGAACCA
CATGATTTTAGCAAAATTTCGCCATTTCTACTTTGATCCGGTGGAATCTAGTTGCCAGATGT
GTCGACTGGTACCTTGTCTAACTAGCTCCATGGCTATGCGCTTGCAGG
SEQ ID NO: 4: GSK2 amino acid sequence
MDQPAPAPEPMLLDAQPPAAVACDKKQQEGEAPYAEGNDAVTGHIISTTIGGKNGEPKRTISY
MAERVVGTGSFGIVFQAKCLETGETVAIKKVLQDRRYKNRELQLMRAMDHPNVISLKHCFFSTT
SRDELFLNLVMEYVPETLYRVLKHYSNANHRMPLIYVKLYMYQLFRGLAYIHTVPGVCHRDVKP
QNVLVDPLTHQVKLCDFGSAKTLVPGEPNISYICSRYYRAPELIFGATEYTTSIDIWSAGCVLAEL
LLGQPLFPGESAVDQLVEIIKVLGTPTREEIRCMNPNYTEFRFPQIKAHPWHKVFHKRMPPEAID
LASRLLQYSPSLRCTALDACAHPFFDELREPNARLPNGRPFPPLFNFKHELANSSQELISRLIPE
HVRRQATHNFFNTGS
SEQ ID NO: 5: GSK2 nucleic acid sequence [symbol]
GACCAGCCGGCGCCGGCGCCGGAGCCGATGCTGCTCGACGCGCAGCCGCCCGCCG
CCGTCGCCTGCGACAAGGTATGTGAGTAACCGGATCTTGGCGTGCTGATCCGTGGTTTTG
CGGTTCTTTGCTGTGTGCTGATTTAGTGTGCTGTTCTTGGTGGAGCAGAAGCAGCAGGAG
GGGGAGGCGCCGTACGCGGAGGGGAATGACGCGGTGACCGGGCACATCATCTCCACCA
CCATCGGGGGCAAGAACGGCGAGCCCAAGAGGGTGAGACACGAGCCTTCCCCCCCCCCC
CTTTGTTGTTTTGGTCTTGGTTCCATTTCTTGAGTTGCAGTGAAATGCTGCCGGTTCTTGGT
TTAGGAAGGTGTTCTTGTGTGTTCTGCAGCTAGTTTCTTAGCTCCGTGTAGTGATTTTTGGT
GATGGGAAAGCCATTGGCTCTAAGAGAGGCATGTGGATTAGTGGTCAGATTTTGCAAAAGA
AGTAAACTGTTGGTAGATATCAGCCAATTTATTTAGTGTTAGTTGTTCATGTTCTTGTATTAC
TGCAAGATCTGTTGTAAATAACTAAATATGGCTTGTTTGGTGCTCATTTTTGGTGGTTTGTA
GGGGAAAAAGTTGGGTGTGTTGGATTACATTGTTGTGAACACTAGTGCTCATAATTAAATTT
TGGTCTTAAGATGGTAATTTTGTACTTGATTTTCAGACAATTAGCTACATGGCGGAGCGCGT
TGTGGGCACTGGTTCTTTCGGTATCGTCTTTCAGGTGATTCATCTTTCAGAAAGTTGTTATT
TGTTTCTTTCTTTTCGTGCTGTCGACTTGTTGGTCTGATGTTTAGCTTGCTGGTTTCATGTGT
AGGCTAAATGCTTGGAGACAGGAGAGACTGTTGCCATTAAGAAGGTATTGCAGGACCGAC
GGTACAAGAACCGTGAGCTTCAGCTTATGCGCGCCATGGACCACCCCAATGTCATCTCCC
TGAAGCATTGCTTCTTCTCAACCACAAGTAGGGATGAGCTGTTCCTCAATCTTGTCATGGAA
TATGTTCCAGAAACACTCTACCGTGTGCTTAAGCACTACAGCAATGCCAACCACCGGATGC
CACTTATCTACGTCAAGCTTTACATGTATCAGGTGTGTGGATTGCTAATCAATCATAAATTTT
GAAATGCCTGCCTTCCTGTGTGTCTCTTCTAAGTCTATTCTACATTGGCTGCAGTTATTTAG
GGGGCTTGCGTACATTCATACTGTTCCAGGGGTCTGTCATAGGGATGTGAAGCCACAAAAT
GTTTTGGTAGGTATTCATGATCAGATTATTATTTTGCTATGCGATGGCCTTTGATTATTGGCT
CTGAACTCCTTTCTTGCAATACAGGTGGATCCTCTAACTCATCAAGTCAAGCTCTGTGACTT
TGGGAGCGCAAAAACACTGGTATTGGCCTTTTCCACCCTAAAGTTTTGTAATACGCACACA
TTACTTTAGACTTTCTTTTTTTTAATTGGACTTTAGACGATTCTTGCTGTAGACTAGTCAGTTT
TGAATCTTACCATTTGTTAAGTTGGAGCTAGCCCTGTGTTACTGAATCGTTCAAAGAACTCT
TATATACTTGGTGAATCTTACCCCTTTTTTTCTTCCTTTTTATTATGCTTGATGGAAGTTTCAT
GGAAATTCCTTAGTTTTACACCTTTTTCCACCTTATTCCAGATGTTTGCTACAATTGTACTTT
TGATAATTTTGATCTTACTGTCCTAATATCCATTAATTTACTATTCCATCAGGTCCCAGGTGA
ACCCAATATATCATATATATGCTCACGCTACTACCGAGCACCGGAGCTCATATTTGGTGCAA
CTGAATATACTACATCAATAGATATATGGTCAGCTGGGTGTGTTCTTGCAGAGCTACTCCTT
GGTCAGGTTGGTTTCTTTTTTCTATGGTTGACAGATCTGCAAACTTTTGGTTTAGTTATTTAA
GCATGATGTCATCACTGTTGCTGTGATTTTGATTATCTTGTATTTGTTTTTGCTAGCCATTGT
TTCCAGGGGAGAGTGCAGTCGATCAGCTTGTAGAGATAATTAAGGTACTGCAAGACATGCC
ATGCAGTTCTAATTTTGCTCCTACTATTGAGTATGGGCATCTTCTCTAACCTTGTATGATATT
CTTGCAGGTTCTTGGTACACCAACCCGTGAGGAAATACGTTGCATGAACCCGAACTATACA
GAGTTTAGGTTTCCACAGATAAAAGCTCACCCTTGGCACAAGGTAAGCATACAATCTTATCC
ATGTTGAGTCATATATCACGTCATCTTTTATAGTTTCCTGGACAACTATGAAAATGTAGCTG
GGCTCATTTCCAATAATAGATTCTGGACACCAGATAGCTTTACAATGCAATGTATAAATAAG
GAGGTGCATACAGGTACTGATTTTTCTAACTTCTGCGTAGGTTTTCCACAAGAGGATGCCT
CCTGAAGCAATAGACCTCGCTTCACGCCTTCTTCAATATTCACCGAGTCTCCGCTGCACTG
CTGTGAGTATATTCTTGCTGCAATTTTAAGTAGCAGAACAGTAGAAAAGTGATTTTTCACTA
CTGCTCACAGCAGGGGTACTGTAAAACGCCCCTTTTCTTATTGCTGTTATGCAAGTTTGCCT
ACTGTAGCTGGTCATATGAGCTGTTACTTTTCACCCTTTAAGAGTTGCACAAATTTGAGCGT
AACCAAGGAATTTTCTTAATCACTTTGCCCTCCAAGTGCTCTTTGATTTGTGCAACTCCTGA
AATGGGGTGGAGTGGAGAAACACTCCTTGTTTCTTTCTCTTTTTTCTTTTTTCCTAAAGTAGA
TTGAAGAATGCTAGTCTTCACTAACTTTGGTTTTAGTGGGGCATGGCCATTATGGTTATGAT
CTTTAGTGGTCCATTACCAAATCAATGTTGGGGTGGATGAATGATAGTTGTCTCATGTTTAG
TCGTTATTCAGTGTAATTGCAATAGCCAGATGACAACTTAATATTGATTTTTTTTCCGATGTG
CTTATTCATTTGAATATCTTTATGCAGCTTGATGCATGTGCACATCCTTTCTTTGATGAGCTG
CGAGAGCCGAATGCACGCTTGCCAAACGGACGTCCATTTCCACCACTATTCAACTTCAAAC
ACGAAGTAAGTGAATCAGATGAAACATAATCTGCTACACAACTTCAGATCTTGGTATCCATG
AGAAAATGTGTACTCTCCTTGGTGCTCATTGGTGCTGCCTTTTGGTCTCTACAGCTAGCAAA
TTCTTCTCAAGAGCTCATCAGCAGGCTCATACCAGAACATGTTCGACGGCAAGCTACCCAC
AACTTCTTCAATACTGGGAGC [symbol] AAATGCTAAATGCACACCACCAGACCTTTTGTTGGATC
GTTTTCGCGGAACCGGTGAAGTTCACATGAAGGCTGAGTCAGATGATTCTTCGAATCCCCG
CAAAACAAGAAGAATAGAAAATATGATTCCTCAGATGATGATATGCAAATGCTTCGTTGGAA
GTTCAATTCAATCATCGAAGAAGAACAACATTGTAAATCGAGAAGTTTTTGCATCGCGAGTT
TGGTAGTGAAACCGGGATCAGCTGGTATGACGGAGGAAACCGAAATGTTTAGATCCATGA
CTGAGTTTTCTTTCATTTTTTTTGCCCAATTGTAACAGAAGAATATAGTTCCCTAATGTAGGC
GTAGTTGTAACCTGTAAACTGCCACTGTTTTGTTCACATTCCATGATGTAAATGCCACCATG
CCTCTGATGAATAACTCTCCTTGTAACCTTGTTCCTTCCATCCTTGACTGTTTACCTTAAAGC
CGTGGACAGTGTACACTGTACATGTACCGTGCTACACGGAAGGACATATTTGAATTTTTTTT
CTCTCTCTCGAAAGACTACATCAAGCATTGCTGGATTTTTTTTTAAAAAAATGGCACAACTTT
CGATGGTCAACCATAAGCAATTAGTGTCGTTTTAAAACCCCTTACTCCCATATGCACAATAC
TTATCTTTCATTTTTCTAGATTGTTTAGCATAAAATAAGATTTAAAAAGAAAGAAAACTATATT
GCTATTTAATTGTTGGGTGTAGAATGGGAAAACTTTTTAAATGAAAGATGATTATTTCTTAAT
GTAACAAGTAGTACTGTAGTGTGGATTGAATTGGGGCAAACTTTAAACTCTAAAACGAGAA
CTATTTTTGAATAGAGCGAGCATTTAAAAGATGAATTACATACCACCTATAGAGATGAAAAA
AAAGGCATGGGACAGTTAGGGCTTGGGCCCCATAGCTTGTTAGGTTTGTAAGATTAAAATT
TAGAGTAAATTTCACAAAACTACACATACTATGACCAAACTATCACAAAACTATATATTTAAC
TCGATGTATCATAAAACTACACATTTAAGATGAAATGTTACAAAACTACATGTTTAGTTACTA
CATTATTACAGAACTATAGGTTTAGAACCAATTTAGTTACAAAACAATAATGTTTATTGCTCT
AGCATAATAATGGTGCTAGGGATTTAAACTCTAAATTGTGATAACTTCAATATTAAATATGCA
GTTTTGTGATACTTAACTTTAAATCTATAGTTTTATGATACAAC
SEQ ID NO: 6 GSK2 promoter sequence
CCGTACTGATTTCGGCAGCATCAAGGACTAGAGGAGGAGGAGGAGCAATAAACAAGTGCC
GCCATGTCGCTTGCCCGGCTTTCAGGGGCGCTTTTGGAATTCTCGTTTGACCGCCTACAAA
CCCACAGCCTGTCCCTGCAACCAATTTGGCCCTGCGCCACGCCACCCCCAAAGCTATTAG
TACTAACCACACCCCCTCTCTCTCTCTCTACACTTAGCAGTAGTACTAAGACCCTCTTTTAT
AAAATTTTTAAGTCACTATCACATTCGTTAGCAGTAGTACTAAGACCCTCTTTGTAAAACTTT
TAAGTCCCTATCACATCGAATGTTTGGACACTAATTATAAATATTAAACGTAGACTATTAATA
AAACCCATCCATAATCTTAGACTAATTCGCGAGACGAATCTATTGAGCCTAATTAAGTCATG
ATTAGCCTATGTGATGATACAGTAAACATTCTCTAATTATGGATTAATTAGACTTAAAAAATT
TGTCTCGCAAATTAGCTTTCATTTATATAATTAGTTTTGTAAATAGTCTATATTTAATACTCTA
AATTAGTGTTTAAAACAGAGACTAAAGTTAAGTCCATGATCCAAACACCACCTAACATGGAC
AATTAGGCTGTACTACAACCTTTTGCCAAGCTACGTGTACAGGTAAATCGCACACATGTTGT
CATCTTTGGAGGCTGAAACATGGGGAAATATCATGTGAAAACCGTTAAATAAGTGAAAACT
CATGAAAATTATATTTAAAAGTTCTCTAAATTTATATAAAAATTAATAGAGATAAATATAGACA
TGAAATACATTTACATCAAATCTCAAGTCGAAACTCAACTTTTATTTGAGAGAATATAAAAGA
CAAATTTTAGGTGAATAGTGTTCTATTATTTTTCATCCGAAATTTGGCATTTTTGTTACTCCC
AAATAAAGTTGAGTTTAACTTGATATTTGGTGAATATATTTCATGCCTACACTTCTCTCCAGT
AATTTTTCATGAATTTATTAAACTTTTAGTTCCGATTTTCACGTGTTTTCACTTATTTGATGGT
TTCCATCGGATATGTCCCTGAAATATGGTATTGAAGTACAACATAGTTCATACTTGGGTCGT
GTTTGAAGTCATGTAAGGGCGTTATAAGCTCATAGGTTTTGCTTACATACAATTGGTGGGAT
AAAAAGGCACCGGTAATTTCTTCAAGATTGATAAAATAAATGTCTAGCGCTATAAGGCCATG
GCACACATCAAATGTTGTTTAGAACAGGTTATTTCTAGCTCCATAAATTGTTGGATTTGAATT
TGTGATTACATTGATAATAATTGATTGAATCAGTTTGTTCTTATTTTAGAGAAAATAAAAAAAA
TAAACCACTATAATTTAACTTACAAACTCCAAAACTAGTCTGGATCTGTAATTTAGGTTGTGC
TAAACAAGGCCTAAAGAAAAGAAGTAATGTTTGGAGAACATGTTTTTAAATCAATATGGACC
ATCTTCTAAAATGGGCACACTGTGCAACCGAAATGGTTATTAGCAACTTAATATCCAATCCT
TAAAAAAACTACATTGAAAATATCCTAAATCCCAAAATTAAATTTTAAAATCTAATTTTGGTAG
CGATTGATTATTTGTAGGGGCAAATGATGAGGCCCTAAATCAACCATGTTTAGCTACTTCCT
CGCTTTCTTTAAGTATGTTTCATACGCTACAAACTGATATTTTTTGCAAATACTTTTTATTAAA
AAAATTATTTTAAGTCTGCAAAAGCTAATGTTTAATTAGTCCTACACTAATAATCCTCCTTGT
TTGGCTTGCCGCTGATAAGCTTAGTCAAAACCCTGATCCGAACTGCACGTAAGAACGGTCA
AGAAACCATTTCGGTTACATCACACAACACAGCCTCATCTCTCATGCTGTCATGCTTGTGGT
GCACCTAGCAATTCCTCCCTCCCCATCTGTCTTCCTCCTCTAATCTAATCCACCTCCCCACT
AATCCACCAGCTGTGTACACTGCAGCAGCAGCAGCAGCTAACCACTCTCACTAAAAACTAT
AGCAGCTGCAGTAACAGCAGCAGCATCACCCACCTTCTTCTTGGTCAAAGCCATCCATCCC
ACCACTCACCCATCCCTCCCAGTATAAGCCAAACCAATCCATAGAGGAGGAAGAGGAGGA
CCAGGTGGTGGCACACCTAAGCTTTGTGCAGTGCCATTCACGCACCTGCAGCTTCCAGCT
TTGCCAC
Wheat
SEQ ID NO: 7: OML4 amino acid sequence
MEPYKLMDQKTPFGERKLLGHQRHVNLPPTPWRADQDPLQQHDSFSKPLALFPNARKGHLN
MTQYENGLFSSSLPDIFDNKLRLTPKNGLVGQPAEKEVNHADDEPFELTQEIEAQVIGNLLPDD
DDLLSGVLYNVGHPARANNMDDIDDDIFSTGGGMELEADENNKLLKLNGGANTGQTGFNGLLY
GENPSRTLSIRNINTNVEDTELKLLFEQYGDIRTLYTAYKHHGLVMISYYDIRSAERAMKALQSK
PFRQWKLEIHYSIPKENPLENDNNQGTLAVINLDQSVTNDDLRHIFGGYGEIKAIHGTSQNGHH
KYVDFFDTRAAEAALYALNMRDIAGKKIRLERCCAGDGKRLTTLHRPPELEQEEYGACKLGNA
NSLPSTYYGSVNMASMTSAGPEHGISRVLRPRVQPPIHQFREGAFLDVPSSTMQSISSPVRIAT
AVTHNNRSTVGENGHSLGKMGGQINGHLNYGFHGVGAFNPHSLPDFRNGQSNGISCNLGTIS
PIGVKSNSRTAEGMESRHLYKVGSANLGGHSSGHTEAPGFSRTGSCPLHGHQVAWNNSNNS
HHHTSSPMLWPNSGSFINNIPSRPPTQAHGISRTSRMLENVLPVNHHVGSAPAVNPSILDRRT
GYAGELMEAPSFHPGSAGSMGFSGSPHLHQLELTSMFPQSGGNQAMSPAHIGARSPQQRGH
MFHGRGHIGPPPSSFDSPGERARSRRNESCANQSDNKRQYELDIERIVCGEDSRTTLMIKNIP
NKYTSKMLLTAIDENHKGTYDFIYLPIDFFQNKCNVGYAFINMISPEHIVPFYKIFHGKRWEKFNS
EKVASLAYARIQGKSSLIAHFQNSSLMNEDKRCRPILFHSDGPNAGDQEPFPMGTHVRSRPGR
SRVLSCEESHRDTLSSSANNWTPSNGGGHASGYSKEADPTTA
SEQ ID NO: 8: OML4 nucleic acid sequence [symbol]
GACCCATACAAGTTGATGGACCAGAAAACTCCCTTTGGTGAGCACAAGTTGTTGGGCC
ATCAAAGGCATGTTAACCTGCCGCCAACCCCCTGGAGGGCTGATCAAGATCCTCTACAACA
ACATGATTCGTTTTCGAAGCCGTTGGCTTTATTTCCTAATGCTAGAAAAGGACATTTAAATAT
GACCCAATATGAGAATGGACTTTTCTCAAGCTCCCTTCCAGACATTTTTGACAACAAATGTA
AGCCCTTGATCCTTGTCTCTTGCAGTTTTTATTTCATTTATTGTAGCACTTCATAACACTGAA
CTATGAACTGCGTCCATCCGATATGGTACTCCTCCCTTCAGTTCATATAAATAATACTCCCT
CCGTCCCAAAATGTAAGACGCTTTTTGACACTATACTAATGTTAAAAAGCGTCTTATATTATG
GGACGGAGGGAGTAGTATGCAGATAACGGAAAGGGTAAACAAAAGAAGATAAGGAAAATA
TTTTTATTTGCTTATTAATAAAAAGCTTGTTTGCTTTTATTGACTGTTTCACTTCAGTGAAATC
TGAGCTTTTCTTGCTACATCCAAGTGAGAAACGAGACAAACTGGCCTGAGCTTTTCATGCT
ACATCCAATTGAGAAATGAGAGTCTGTCCTGTGCTTTTCATGCTAAGTCCAAGTGAGAAAA
GAGACAATCTGCAGTAATATTAGTGCTTAATACTAAACCACTTTTAATTTGCTGATGTGCAG
TGAGACTAACACCTAAGAATGGCCTTGTTGGCCAGCCAGCTGAAAAGGAACTCAACCATGC
AGATGACGAGCCTTTTGAATTAACTCAGGAAATTGAGGCACAAGTAATTGGCAATCTCCTC
CCTGATGATGACGACTTATTGTCAGGTGTTCTTTATAATGTGGGTCACCCTGCCCGTGCTA
ATAACATGGATGACATTGATGACGATATATTCTCTACTGGAGGTGGAATGGAATTGGAAGC
TGATGAAAATAACAAATTGCTAAAACTTAATGGAGGTGCCAACACCGGTCAGACTGGGTTC
AACGGCCTACTGTATGGCGAAAACCCCTCGAGAACCCTTTCCATTAGAAACATTAATACCA
ATGTTGAGGATACTGAATTGAAACTCCTATTTGAGGTAAGTTCCATCTTCCAGCTTGACTTT
CTCCCAACTCTGAAGGCAATATATTTCACCTGATAGCATTTATTTTCTTTGTAGCAATATGGA
GACATCCGAACACTTTACACTGCCTACAAACATCATGGTTTAGTGATGATATCTTACTATGA
TATAAGATCGGCAGAACGTGCCATGAAAGCGCTTCAAAGCAAGCCATTCAGGCAGTGGAA
ACTTGAGATACATTACTCCATCCCAACGGTATTTCCTTGATATAATGCCATTCTGACTTGATA
TGATGTGGTGCTTTGACATTACTTAATGTGATATTACTACGATGTTTGCTTGCCATTATTTGT
TGCATTGGTACTTAATTGGCACTGGAAATGTATTTATACTTGCAAGAATGTTCACATTCTAAT
GCTGACTTTGTTCCAATAGGAGAACCCTTTGGAGAATGACAATAACCAGGGCACACTTGCA
GTGATTAACCTAGACCAGTCTGTAACTAATGATGATCTTCGTCATATATTTGGTGGCTATGG
TGAAATCAAGGCGGTATGGCCTGCGCACTAACCAACTCTTATGTCAGCTAGTACACTACAG
ATACTAACTTCCTTGTTTATCAGATTCACGGGACATCACAAAATGGCCATCACAAATACGTT
GAGTTTTTTGATACCAGAGCAGCAGAAGCTGCACTTTATGCTTTGAACATGAGAGATATTGC
AGGAAAGAAAATCAGATTAGAGCGCTGCTGCCTGGGCGACGGTAAACGGTATTACTGTGA
CCATAATTTTGCGCATCTGTCCATTTTTAGTGCTTCTAGTGCCTTCGCTTTTCGAAGAGTCT
GCTGTTATTTTTTATTAGAGGAAGGTACTGAAATGGCACAAGGTATGACCAATGGAAGCCA
AAAATGAACAGAAAAACTAAAAAACCAGCAAACAAAAAACCAAAAAGGCTAAGAAGACTAAC
AAAATCTAACCAAAACTAGCATAATGACCTATATATACTATACCAATTAGGAAGCAAGAGAC
CTGAAGCCCCAGTAAGCAGCAGAATATGGCGGTCCAGTCGGTAGCAGCAACCTTCGCAGA
TCAACTTTGTATAAAGTTATCTGGGTATCTGCGAATTGAGGAAATATATAATTCAACGGTGT
TTATGGTCTTGCATATACTGTGAAGTTGGTAAACATATCGTCAATGGACATATACAGTATAC
ACGGGGCTGATGCAATTCCTGTCTCTTCAATAAAATATGTTTTAGTTATTAAACACGCAAAC
TTGTGATTGACGTTTAATATGATTTTTTAGAGTCGTCATTTGCACTTGAATTCAAAGTTGGTT
GTATACTTGTATATTTCTTGTTTTAGGGAAGTGTGCTTTGGAGTTTGGAGGAAATTGGTAGG
TGTAAAAAAATCTTTTCATATGATGTGCAGGAAGATGTGTTTTAGAACTTGATGCAGAACGT
CCCCCCTATGGATTATTATGCTTGTCTAAACTTTATTTTTGGAGGAAGAAACAAGAGCATCT
GACTTTCCTTATGCCTATTCTTACAACTGTATTAGTAATGCTAGTTTTTGCACAACAGTTTGA
CGCGGCACAGGCCTCCTGAGTTGGAGCACGAAGAGTATGGTGCATGCAAGCTAGGAAATG
CAAACAGTCTGCCGTCAACTTACTACGGTATGCAGTTTGATTTCAAATCACGAGACATGTTT
CTGCTGCTAATCGCATTTACTAACCTATGTATGGCATAATACAAGGTTCTGTCAACATGGCT
TCCATGACTTCCGCTGGTCCTGAACATGGGATCTCTCGGGTTCTGCGTCCCAGAGTTCAG
CCACCAATACACCAATTCAGGGAGGGAGCTTTCCTGGATGTTCCCTCAAATACTATGCAAA
GTATATCCTCTCCTGTTAGAATTGCAACTGCAGTAACGCATAACAACCGGTCGACTGTCGG
TGAGAATGGTCATTCACTTGGAAAGATGGGTGGACAGATTAATGGACACTTGAATTATGGA
TTTCATGGGGTTGGAGCTTTCAATCCACATTCCCTTCCTGACTTTCGCAACGGCCAAAGTA
ATGGTATTTCTTGCAACTTAGGCACAATATCACCCATTGGAGTTAAGAGCAACTCTAGAACT
GCTGAAGGAATGGAGAGCAGACATCTTTACAAAGTTGGTTCTGCTAACCTTGGTGGTCATT
CTTCTGGTCATACCGAAGGTACTAATTTGGGTGCCTTATTTACTGATGTAGCCATATGTTTA
TGGAGACGCACTGTTTCCATTAGGTTCATTTGCCATCTCTTTCCCTTCCAGTCATTTTCTTG
AAAATGTCAATTTTGAAAGAACATATGCTTTGATATCAATAATACAGAAGCTTTTATAGCTTA
ATGGTAATTGGTGTAGCCTAAATTATACTATTTTTGAGGTTGCAACTATTCTGTTTAGACAAT
GCAATTAGGCTTACATGGGCATGCCTTGTGTTCTTGTAGCACCCGGGTTTTCAAGAACTGG
AAGCTGCCCCCTTCATGGCCACCAAGTAGCGTGGAATAATTCAAATAACTCCCATCACCAT
ACCTCCTCTCCCATGCTATGGACGAACTCAGGATCATTTATCAATAATATACCATCTCGACC
TCCCATGCAAGCGCATGGAATTTCAAGAACATCTCGCATGCTTGAAAATGTCCTTCCAGTG
AATCATCATGTTGGATCTGCACCAGCTGTCAATCCATCAATTTTGGATAGGAGAACTGGTTA
TGTAGGGGAGCTGATGGAAGCGCCAAGTTTCCACCCTGGGAGTGCTGGAAGCATGGGTTT
CTCTGGTAGTCCGCATCTGCATCAGTTGGAGCTCACTAGCATGTTTCCTCAGAGTGGAGG
GAACCAAGCCATGTCCCCTGCACACATTGGTGCTCGATCTCCTCAGCAGAGGGGGCATAT
GTTTAATGGAAGGGGTCATATAGGTCCCCCTCCATCTTCATTTGATTCACCAGGTGAACGT
GCAAGGAGCCGAAGAAACGAGTCATGTGCTAATCAATCGGATAATAAAAGGCAGTATGAG
CTAGACATTGAGCGTATAGTCTGCGGCGAGGATTCCCGGACTACTTTAATGATAAAGAACA
TCCCAAACAAGTATACATCTGGGACTTTCTGATTTTGTTCTAGTTTATGTGCAAGTGTCACT
CTATTTGAAGTCACGCCATGTTTTGATGTTTCTATTGCCTTAATGGTATTTCAGGTACACCTC
TAAGATGCTTTTGACCGCTATTGATGAAAATCACAAGGGAACTTATGATTTTATCTATCTTCC
AATTGACTTTAAGGTGAATGGAGCTTTTGTAAACAGCTGTTGCATGTTTATCCTTGGTTCGA
CATTACTTGCATACAACGAACTAATGGTGCTCATGTGCATTTTCAGAATAAATGCAACGTGG
GCTACGCATTCATCAATATGATAAGTCCTGAACATATTGTTCCATTCTATAAGGTGAGAGTG
AGATGTTACAAGTTATGAAATGGCGGCAGTGTATTAGATAAAGCTTCATGTTGACATTTTTA
TATGATTTTTCACCCTCTGCTTTCCGTCGTCATTTCTTTTTCCATAACTACCTGTATTACACT
ATCATGCTACAATTGCATGGATTTTGGATATCGCATGTCAGGTAGTCAGTAGTACCTTTACC
ATTTCTGGTTTCACGCTCTAAGCATTTTTTACCTAATGCCAGTCGATAAATGAACAACATACA
TGCCTGTCTCTTTCAGATATTTCATGGGAAAAGGTGGGAGAAATTCAATAGCGAGAAGGTA
GCATCACTTGCATATGCTAGAATCCAAGGAAAATCATCTCTAATTGCGCACTTCCAAAATTC
AAGTTTGATGAATGAGGATAAACGCTGCCGCCCTATACTTTTTCACTCAGATGGTCCAAATG
CAGGGGATCAAGTATGTTCTCTGATTGTCCATATCCTTTGCTGTATTACTGTTTCGATAGGG
CACCTGACTTGGTGCCACTAACTAGATGACCTGTATATCTTATTGTGTGCCCATCCAATACA
TGATCGGTGAAGTCCACACACATACCTAATTTTATATCATTATATTTTTATTATCTTGCATCT
GAAATTAAGCAGTAGACCTTACACAGTTTAGTATGTTTTTTTCTTATGCTACGTCAGAACTTT
TCCTGAGTATTTCTTTCCTTTAGAATTGTATTGACGCGGAAAGAAATACTGAGGAAAAATTC
TTACTCCCTCCGTTCCAAATTACTCGTCGTGGTTTTAGTTCAAGAGTACTAATTAAAATCCTA
CAAATCATGGAATATGATCCTCAATTTATTCTAAATCCTTTGAAACAAGGAGGTCCTTGAGG
ATTCAAGCATAGGCTCATCGTTCTTGTTTCCTAGTGTTGCTATGTTCTTTTTAATGATACATA
TAGCTGTATAGGCTCATCATTTTCTTCACATATGGTGGTGTTTGATGCGCAATTGGGATACA
TTGTGGGTTAGGGAGCGGAAAATAAAACCATCTTGTTAACATTAGGCAGGGCTATGAGTTT
GGAGTAGAGAATATAGTGCATACATGACAAGATTGCCCCTCGATAATGGCTTTACTTAATTT
TGTGTGTATATATTTTTTGTATTTTTAACATTATTACTCAACCTGTCTACAAAAAACCATCGTT
CTGATTGGACTTCAAACTGTGGTATATGAAACTACATATCCCATGCCAAACACCCCAATAGA
TTGAACTCCTCCCACCCACTATTTCCATTCTTACCTCCCATGATGAGTCTGAACTGACCATG
TTTTTGTTGTAAACTTTTCTCAGGAACCTTTTCCAATGGGAACACACGTCCGTTCTAGGCCT
GGGCGATCCAGGGTTTTGAGCTGCGAAGAAAGTCACCGGGACACTCTGTCATCTTCTGCC
AACAATTGGACTCCTTCCAACGGGGGCGGCCACGCTTCAGGCTACTCAAAGGAGGCTGAC
CCAACCACAGCT [symbol] AAGCTGAAGCACTAACCACAACATCAACATCCAACCTTTTGACATTT
GCAATCCCAGTTTTCACATTACCATCCTTTCCCACCTCTTTTTGCTTGTGGTATTTTCGGAG
TCTGTAGCTATTTAGTACTTTCTATGTCGTGGGCTACCAGAGGCTTCCTAGAGGCTGCAAA
TTTTGTCGCTGAGTAGAAGCAAGGGAACGGACGGAGGGTGCTCCCAGTTTCTCCTGAGCC
TATATGCGTGTATTAACTGAAGGCCGTGGAAGGCAAAACTCGTGGGGAGCTCTCTGAGATT
TTGGACTGTAAGGTGTAACCCAGCGTTGTACAGGGTTTCCTAGTAAGAATGCATGACGGG
GACAGCCGACACTGTATTGGTGCTGTTGTATGAAAGGCAGGCTGTGCCATGCAGCGTCTT
TTGAAACTGTTTTGATGTTAACTACTCCCTCCGTCCGCGAATAAGTGTACATCTAGCTTTTA
TTCTAACTCAAAGTTTTGAAACTTTGACCAACTTTATAGGTAAAAGTAGCATCATTTATGGCA
CTAAATTAGTATCACTAGATTCGTTCTGAAGTGTATTTTTATAATATACCAATTTGATGTCATA
GTAGAAGTACACTTATTCGAGGACGGAGGGAGTAGGAAACATGCCCGTGTGTTGCAACGG
GAGAAATAAAATCCTTGACATAATGATAATTGTT
SEQ ID NO: 9: OML4 promoter sequence
GAGGAGGGGACAACAAGAATGCCAGATGAGAAGGGGATGATGCACATGCCGGCAACATG
TGATATGTACATGTCTTGGTTAGAGACTTTTGTTTATGCAACCTATTAAAAACTATGTGCATG
TTTGCTTGATGTGTTAAACATTTAAATTTGAAGAAATCAAAATGTTTGAATGAAAAAGAATAT
GGAGACCGAGATATGTCGACTCTGAGTCTGCCGTCGCCTGTCTAGATTTCAAATGAAAAAC
GTCACATATACATTCTGACAAGCACAAATGCAGCGACATTACTCTTAGAACGCAGAAAAGTT
GCTATGAGTACAAACATGCCAACCTAACAGGAAGTGCTGTCGAGAACGAGCCCTTGCCTT
GATGGTTGGCCTTGGCGGTGGCCAACTAGCCCACCTGGGTTTGATCCCTAGGATTAACGC
GAGTGTTTCACCTGGCGCAAAAGAACCCATAACCTAGGGTTCTTTTGCCAGTTTTAAAGTAT
CTCACATGTCTATGGAGATTCACGGAGTCGAATGATGCGATTTAGGTGTCTGTCACGAAAT
TGTTTTGGTGTGAATCAAATGATTTGCCCATATTAGATGCAAAGAAGAATCATTTTAATTATT
TTCCCTATATCTGTTATCTTTAATGTATTGAAAATGTAAATAGGAACAACGTAATTTTCAAGG
CAATCAATAACAATCAATGTTGCATTTTTAGGCTTGTTTGAAAATGCATATAGCTAGTAGTAG
TATATTTTGTTTGAAAACGTGATTTCAAATATACTCCTCACTAAACTGAATTTTCCCAGTGTT
TTTGGAAGCAGAGTTCTTCCAACACGGAAGGTGGTACCAGTATAAACGTGCCAACCTAACA
GCAAGAGCCGTCGAAATCCTCGGTGGTTCGGCGGCAGCAACTCCAGCGGTCCAGTCCCG
ATCGACCCCCACACAACTCGTACTGGTGAGCGTTATCCTGTCCCACACCAGACAATCGGA
GAACGTGACCTCCGCGTCACCTCACCGCGCCAACCCCCACCCCTCCGCGAAATAATTCCG
TCCCCGTCCAAACGCCGCTTCCCACCCGGGCCGACGCGCAGCCACGCGGGTCCCGACGT
CTGACACCGGCCCCAGCTCACTGACACGTGGGGCCCCTGACCCGGGTACATGTGCCGTT
CGGCACGAACGGAATGGCGGAGGAGCTATACGGTGCCGCGGTGGGGTGCGCCGCGGGT
GAGCCTACCGCGGTGGGGACCACACCGGGCGCGGTATATAAAGGCCCCGCACCTTCTGC
ATCGCGTTTCCACTTAGTCCAATAAATAATATAGTAATACAGCATTTCGCCGCTCTTTTAATT
AGATTTTTTTGGCCTTCGTCTCCGCTTCGTCTCGTCTCGTCTCACCACCGCCAGTCCACCA
CCAGCTCGCCGGAATTCCCTCGCCGGAGACGCGCTGCGGAGGCATACCTAGGAGGAGGA
GCGAAGAATGGGGAGGAGTGGAGGGGTGCCCGCTGCCGCGCTCGCTCGCTAGGGTTAG
GTGCGCGCGTGCGTGAGGATGGAGCTCGCTCTCTGAGCTCGCCTCGACCCCCCGCTGAG
GGCATTAGCTGTGCCGTCGCTGCGCGCTGGGTGATCTGCCCTCCTGTGAGCTCCGGGGG
AGCCGTTTTGGCTTGCGCCATGGAGCCGTCTCTTCCGGGCGCGGCCACGGGTTGCGTGT
TGCGGTAAGCTCCTGGCCGCGACAAGCTGAAGGGGATCTCGACTGCCGATCTGGTGAGC
CTACAAATTCTTCCGTTTCTAACAATTTTTTTGCGGGTTTTCATGCAAATTCGGGGTGCGTTT
GTGCAGAAAAATGGTGTTTGAATTCCTGGAGGTATTTGTTTGGGGGTAATTTTGTGCGTATT
TTTCTCGCTTTGTGTGATCTGTGCTCGGGTTCGGGGGATACTTTGATGCGGTTGGGCAGTA
TTTTGGTCTCTTCTGCCTTTTTATTTTGATCACAAGTTTCTTGTGCTCTTTCGAGCTCGTCGA
GGACGAGGAGCATTAAATTTCCCGTCGTCTTTTGCGCTGTTTTATGGCTAATTAGTTTGGGA
GCACAGAGTTCTGCACGGAATTTTGCATAACCTTTTTTAATCGTTCAATTCGAGTTGTTCCG
GTCCTAAATTTTGCAGAATTTCTCCAGTGTTCCGTAGCCGGTCTCGTGAGTTCGATTTTGG
GTTCACGGTCGATCAAATCTAGGCCTCGGGACTGCATTTTCTCGCGTTTATTATTTGATGAT
CTGCTTCAGTCGAGCTACCTGAGGTGTTGAAACTTGGTATCTGTCTATCTTTCAAGGTGCTA
GCAGGATGCCAGCTAGAATCATGGAGCAGAGGCACCACATGCCCCCATTCCACCTCCCCG
TGGAGTCCGAATCGTCTTCTCCCATGTGGTAAGCCAACTGCAATAGCCATTATTGCCCGAT
ATCCTTAAATGATGTCTAATGATGGACTGCATTCTTTCTTACTTTAGGGTAGGGGGTACTAA
TTGGTTCAGTTTTGGGGTGACTTGGTCAGTATCATTAACAACTAGACCTAGGTTAATTCCTT
CATCATTATCGAATTCTTTTTGTAGAGGCCTGTTGGACACTTCAGGCAGGAATGTTTTCCTG
AGCATGTTAGTGACTACCTGAACATTCGTGTAATTTGTAGGTGCTTATTAGTTTATTCTTCG
GGTAGTTTCTCTCGGACTAAATAAAATGTGACCTAGCAGAACACTGTTACAGTTTACGGATA
TGTGGGGGCATCCGAGGTTCCACATATGGGACAATTGATCGAGCAAGATTGGAGGATGTG
TATCGTTGTTTCCAGGTAGCTTAAGGTAGCTGTCTTGCTATATATGGGTGAAGCAGCTGATT
TGGAAGGCACATGGTCTCACAGGGGTGATTGGGTATCGATTATTGACAGATGCGCATGGA
TGTTGCCTACAATGATTCTTCCATTAAATAATTCTGATGTTGCTTCATCACTTCTCTTTGCGC
TCAGTGTTTGTGTTCGTTTTTATGGCTGATTTATTTCTTGTTTTGAAAAACAGAAAAAAGATT
CATTGATTTCGGGAAGTAGGTCTGCTGCATCTTCTCCAGTCGAAAAGCCAAAGCCTATTGG
CCAAAGGTTGTGCATCAATTAGGACTT
SEQ ID NO: 10: GSK2 amino acid sequence
MEHPAPAPEPMLLDEQPPTAVACEKKQQDGEAPYAEGNDAMTGHIISTTIGGKNGEPKQTISY
MAERVVGTGSFGIVFQAKCLETGETVAIKKVLQDRRYKNRELQLMRSMIHSNVVSLKHCFFSTT
SRDELFLNLVMEYVPETLYRVLKHYSNAKQGMPLIYVKLYTYQLFRGLAYIHTVPGVCHRDVKP
QNVLVDPLTHQVKICDFGSAKVLVAGEPNISYICSRYYRAPELIFGATEYTTSIDIWSAGCVLAEL
LLGQPLFPGESAVDQLVEIIKVLGTPTREEIRCMNPNYTEFRFPQIKAHPWHKVFHKKMPPEAID
LASRLLQYSPSLRCTALDACAHPFFDELWEPNARLPNGRPFPPLFNFKHELANASQDLINRLVP
EHVRRQAGLAFVHAGS
SEQ ID NO: 11: GSK2 nucleic acid sequence [symbol]
GAGCATCCGGCGCCGGCGCCGGAGCCGATGCTGCTCGACGAGCAGCCCCCCACCG
CAGTCGCCTGCGAGAAGGTAACCGGATCTGTGCTGGGATGGTGTTGGCCGTGTGTTTCTT
GGCGTGGTGTTCCGTTGAGCTGATGTTTAGCGTGTTGTTTTCGTTGGGCGCTCTTGTTGAG
CAGAAGCAGCAGGATGGCGAGGCGCCGTATGCGGAGGGGAACGACGCCATGACCGGTC
ACATCATCTCCACCACCATCGGCGGCAAGAACGGCGAGCCCAAGCAGGTGAGCTCAGCG
TCTCTTATGTTTCGCTTGTGTCTCTTGGCCTGAGTTTGCACGGCCAGTTCTTGCCTTGGTGA
GATGTGTCTGCTCTCCTGCAGCTATTCTCTTTAGCTATGACAACTCATTGAAATATAGCTGT
GTGGATTCTTGGTTAGATTTTTCTTCGTTTACCAAATACGAAAAAAATGTTTCAAAGCGGCT
GAATTTATCAATTATCAAGGACGATGTAGCTTGTCAGCCTATTTTTGTAGTGCTCATTTGTTT
GATCCTCATGTAACTATGGTTTGCTCAAGAGATCTGTTCCAAATATGCCTGTGTGGTGTTCC
ATACTGTGGGTTTTCGGGACAAATTTGGACGGCTTCAGTTAGATTTTGGCCAACACTAGTG
CTCAAATCTGTTACTATGAGCAACAGCTGATACCTCTTTGGCGCCCAGTTGGTAATGTCCT
GCTTTGTTTTTCAGACGATTAGCTACATGGCGGAGCGCGTTGTGGGCACTGGTTCGTTTGG
CATCGTCTTTCAGGTGATTGCTCTAGCCATTGTTTGTTTCCTTGTTTGTGTTGTTGACTACC
AGCCTGATGTTTAGGGAAATGTTGCATGTGTAGGCTAAATGCCTGGAGACCGGGGAGACA
GTGGCCATTAAGAAGGTACTGCAGGACCGACGGTACAAGAATCGTGAGCTGCAGCTTATG
CGTTCGATGATCCATTCCAATGTTGTCTCCCTCAAGCACTGCTTCTTCTCAACCACAAGTAG
AGATGAGCTGTTTCTGAACCTTGTCATGGAGTATGTCCCGGAGACACTCTACCGCGTGCTT
AAGCACTACAGTAATGCCAAACAGGGGATGCCACTTATCTACGTCAAGCTTTACACCTATC
AGGTTTGTGAATTTCCAGTGAATAAATGTGAAATGTGTGTCTGTCATTGTGCAACTATTCTA
AGTCAATTTTACATTTGTGGCAGCTATTCAGGGGGCTGGCGTACATTCATACTGTTCCAGG
AGTCTGTCACAGGGATGTGAAGCCACAAAATGTTTTGGTATGTATCAGAGGCCGGGGTCTT
CCCCTTCTGAAAAAAATGTATGAGTGAACACTGAACAGATTGCTCAGTTTCATGTATGG
CTTTTCTTGCTTGATTTTGAACTTGCCTCCACTTGCTATATTATACAGGTTGATCCTTTAACA
CATCAAGTTAAGATCTGCGACTTTGGAAGCGCGAAAGTTCTGGTATGTTGGCTCTTTCCCC
AAGAGTTTAGTGATACGTACACACTGCTTCAATCCATTTGTCCTGTCGTGTAGGCTACTCAT
TCTATTCAGTATTGAACCAGAATCGGCATCATGGTCTGTGCTATTTTGATTTAGTCTTACTGT
TTTAGGCTTATAGCTGGCCAGGTGTTAAGATTAAAATTAAGTCACTTTTATATACCTTACAGT
TTGACTTCTTCAGATATTTTTGGTTTATAAACTATTATCTCTGTATTCCGCTTATTCCTTCCTA
GATTGCTGAATTCTTGCTTTAGCCGAATGCAAAGTTTCTGATCTTCACTTCATTTATTTACAT
TGGATGTCCGACACTGAATTTAAACTTTTGTTCCTTTACTACAATATCAACCTGCATAGTACT
TTGATGTTACTTACCTGCTAATCCGATATCGTTTTTCTTGTCCTGTTCTATCAGGTGGCGGG
TGAGCCCAATATATCATACATATGCTCACGCTACTACCGTGCTCCGGAGCTTATATTTGGTG
CGACTGAATATACAACATCGATAGATATATGGTCAGCTGGTTGTGTTCTTGCAGAACTGCTC
CTTGGTCAGGTTAGTTCCTTCGTTTCGTTCACATATATTGCAATCTCCTAGGTTCCAACTAA
GATGTCAATACTGTAGTTTCTGATCTTTCATTTGTTTTTGCTAGCCATTATTTCCAGGCGAGA
GTGCAGTCGATCAACTTGTAGAGATAATCAAGGTCTGCAAACATTCCATATATCTTTCTTTC
GCTTATACTATTAGATGTTGTTGACCTTTGTGATGTTCTTGTAGGTTCTTGGAACGCCAACT
CGGGAGGAAATACGTTGTATGAACCCGAATTATACGGAGTTTAGGTTTCCACAGATAAAAG
CTCATCCTTGGCACAAGGTAGGCTTGCAATCTCATTCTAATGTCCAATCATATATCACATTT
GCTGTTATTAATATATGTGGCTCACTGTTATTAATATAGGTCAGCCGTATATAAGATCTGCT
GTAATATACTTAACCATGTAATGTGATGCCTACGTGTACTGATTGCACTTTGCTGTGACCAG
GTTTTCCACAAGAAAATGCCTCCTGAAGCCATAGATCTTGCTTCACGTCTTCTTCAATATTC
ACCAAGCCTCCGTTGCACTGCTGTAAGTTTTTTCTTTTCACGTTGCTTGCTCTTCCAGGTGT
TTGTTGCGGCAAGTAGGAGAGGAACAGATGAATGTAAATGTAAATATGAATGGTCTTTTAG
AGACAATCAGATATATAGTTGTCCTTATTGATTGTTGGTAACTTATTTATGTATATGTGTGTA
GTGTACGTTTGTCAAACTAGATTGATCAGTACTAGTCTTCTTTTTTTTCTTTTCGAAAAGGGG
GGACTCCCCGGCCTCTGCATCAGAGCGATGCATACGGCCACAATTATAAATAAATAAAGTA
GTTCAACAAGGTCTTGCAATCTGCTGCAAAAAAGTAGGCTGGCTCACAAAGAGCTAGAAAA
ACAAAAAAGGCCCAAAAGCCACAACCGGCTGGCATAAGATAGATAGATAGGTAAACTAATC
GCCTATCCTATTACTAGTCTTCTATGAGCATCATAATCATAGTTATGAGGACCGCTAATCCA
GTACCATAGATCGATGCTTGGCAGATGACGAGTTGATATTTAGTCGTCTTGTAACATGTTTT
GCTATAGCGTGATGGCTATATTCACGCATTTGAATATCCGCATGCAGCTTGACGCATGTGC
GCATCCTTTCTTTGATGAGCTATGGGAGCCTAACGCGCGCCTGCCAAATGGACGCCCGTT
CCCACCTCTGTTCAACTTCAAGCATGAAGTAAGTGCATCAGAGAAAAACTAGGCTGCTCAT
TTGCAATTTGACAAAAATGTATGCAACCTGTTCGTGCTGTTGTGCTTATGGGATCTGCTTTT
TTTTTTTTCTGCAGCTGGCCAATGCTTCACAAGACCTCATCAACAGGCTTGTGCCTGAACAT
GTTCGCCGACAAGCTGGTCTTGCTTTCGTGCATGCGGGGAGC [symbol] ATATGCGCACCGGTG
CCCTCAACCTTGCACCTTATTGTTTTGCCATGGGCAGAAGGGTGGTGGTTTAAGATGGAGG
CAGGTCAGATGATCCCTGGAGCGATATATGCCAGATTCCATCATCAGGAGTACCGGTAGA
GCACCGAGGAATAACAACTGTCTAGATCATCTGCCAGGGAAGGAGACTTGCCAGGGAAAC
AGCATAGCCTTACGCCGTGGACCCGAGTTTTCTTTCAGTTTTTGCCCTATTGTAAGAGTTAT
TAATAGCTTCTTAATGTACTGTAGCTCGTAAGTTGTCAACTATTTTGTTCTCCATTCACTGAC
GTATTGTTGCAGTAAACTTCGCTGTTCAATAAGTTGTGTCATGGCAGAGCTTGCACGCCCA
CTGCCTGTCATGTAGTCAAGCTGTCTATTTTCTGTTGGGTAGTTGCGACCCGTCGTGAGAT
GGCATGGCTGAACTGGAATTAGGGTTCGTGGGATCGAGAATTGGGGAAGCTATAGGTTTA
GTATGGCCAAAGGCTCACAATATAATCCAATGCTGATTCCAGAAAAACGGGGGAGGCTTAA
ATTGCCCCGCTAGCAACAGGTAGAAAGGAAACAACTCGGCAAGTGAACTGATACAATAATA
CTCCCCTTGTCTTAAAATAACTGTCTCAATTTTGTACCAACTTTAATATAAAGTTATACTACG
GTTAAGACATCTATTTTGGAATGGCACGAGTAGTAAATAATGGTTGAATAGATGGAGTCCCA
CGAGCCGTCCGATCCTGTGACAGACGGCGAGTCCCACGAGCCGTCCGATCCTGTGACGA
GCTTCAATCTTGAGCGTCCACTAACTGAATCTTGATGAGGAGTTATATAAAGCAGTTTCGGC
CTGACAATACCTCCCCGTGAAAGACGAACTTGTCCTCAAATAGTTGATGGGCGATCGAACC
AGCCTCCTATTGTTTGCTTGAACAAGGCCGGGAAGGTGGTCTTGATGAAATCAGTGTCTTC
CCAGGATGCATCATGATGCGGAGCATCTTACAGTCATTCCCATGGACCTATCTTCGTCTCT
CGAGACACCACGTGGACCGATGACACGAGCTCGTGCAAGGGCTACCACGAACGAGGTTA
ACTCTCTCTTTGTTGAACTCTCCTTTGACCCACTTGAGACATGGCTACTACCTCAAATGGA
SEQ ID NO: 12: GSK2 promoter sequence
ACGTTCCAAAAGGATAATTCATAACCTAGCAATTTTAGATATCTATGAACTTCAGTATGTGC
CATCACGGTCTCAACAGGATATGGTACTCTGTTTTGATTTTTTATCAAAACCTAATTATCAAT
TTATATATGTGCGTACAATCTTTAACCATTATAGCTGACGATCTTTAACGTGTTTCATCATCT
GTAGGCTGTGCAATGTGAGATAATCATGTATGGTGTTACCGAATGGGCTGGAAAGTTTCTA
AATTATGAGCTCTGCATGATTAAGTGGCGCCGGGACCTGTACTTCATTTAATGCAGAGCAA
GAAAGGAACATCAAAGAAGCTGTTAAAATGGATGGGAGAATTTGCTAAAACATGTTTACCTA
TTTTTTTAAAGATTAAGTGTATTCTAGAAAGAAAATAATTGTATGTTTCATGGAGTAACAAAT
CAGGAACTGTGCAGGTATGTGTTCATCTTGATGGGAGTTTGTCCGAGATGCTGGGCGAGG
GGATTGTTTCCGCACTGCATGTATCCAAGTGTTTAGGGATGCCTTTTGCGAACAAATTTAGT
TTTTTTTTGAGCGAGGAAATTGATACTTTTTTTGTGAATGTATTCGAGTGGGTGAAATACTCC
CCATTTGTCTCGAGAGGTCCAGGTGTGGAAGCTTAGCACTGGGTTTGTCATTATTGAGGCA
AGAATAGTTTGAATATGCACACTTTCATTATGTACTTGCAGTGCGTAATGCATCATGTGTAG
AAAAAGATAGTTATATTTGTATAGAGTAAATTACAATGTTTGAGGTATTTGAATAAAAAATAC
TTTGTTGTTTCATGACATGCAACAATGCGATCTTTTGTGCTCGTTATTATAAGTTTGAGTAAA
TTTGTATTAGTTAATATAATGTACGATGACTGCCACGTTTGACTAATTTTATATTAATTTAGAC
GTGCAAACATTTACTAGTACTCAATATGGAACACAGAAACCGACTATCAAAGCATTGCTGAT
CCGATCCCGCATACTATATATAGGTCATTAACTGACATATAAAAATGTTTGGATAATTTTACT
TCTACAAATATACTCACAAAAACTGAGTACATTTTTAGCACTGGCTAAAAGGGTTATTATTCG
AACGAAAACACATAATTGTTGCGTAAAAGATGTTGTATTTTCAGTACCAAATTTACGATTTGA
TCCAAAAATAAGGAGGCATTAAAATGATGCGGATCTTTGGGTCTCGGGTGCCAATGCACTT
GAATTTGATCTTTTTAAAAGTATTGCGAAATTAGTCAAAAATTTCAGAAACTTCTTGCAAACA
AGAATGATACGGTGTTATACCCGTGTGACAAGTTTCACGAATGAATGAGTTTTATGGTATTT
TAGGTTAAACAAAACAAAATCGACACTATATAAACATATTCACACCTTTGTTTATGTCAAGG
AGTCCACGGAAGTCACTTCTTCGCTAAACTTTTTATACAAGTATAACACTACAAGATTCTCG
TCTCCGAAAATTTTCAGGAATTTTTGACTCTTTTTGTTATTTATAAATTATTATTTTTCAAACA
GGTTGCAATGGGACCCAAGATTCATTAGGTATTTCCGGGCATTAAAATGACATAGTATATAG
CACTAATAAGGTTCTCCTATATGACATGAATGCGCCATCAATTTCTCCCACGAATACCCTAG
TATATTTGTACAGCAATTAGTGTACAATTTTCACAAATTTCTCTGACGAATACCCTCGTATAT
TATCAGTTCATTTTCCGGCAGAAATTGAAAATATGCCGTAAATATATTTTAGCGGCATTGTTA
TCCTTTTGACCAAAAAATGAATCCCATTACTCGGCAATAAATGCGGCAGACTATATAAAACC
CAACCTGATGCCCGGGGTACTCCCAGCAATTTGACTCCCGAGGCTCGTCTAGTCTAATCCA
CCTCCCCACTAATCCACCAGCTGTTTACACAGGGTCAGCTAACCGCTCTCTCTATAGATCA
ACGTCACTCCCCATCTTGTTCGTCTTGGTCACCCCCACCCCCACTTTCCCTTCACTGGTCA
AAGGCACCACCACCCACATCACAGTACAAGCCAAGCCAAGCCAAGCCAAGCCAGAGAAGA
GGACCAGGCGTAGGTGGATGCAAGTGTGAGCCCACCGTGTCCGCCCCATTCACACCCTA
GCCAC
Soybean
SEQ ID NO: 13: OML4 amino acid sequence
MPSEIMEKRGVSASSRFLDDISYVSEKNTGLRKPKFIHDHFLQGKSEMAASPGIIFNTSSPHETN
AKTGLLMSQTTLSREITEDLHFGREAGNIEMLKDSTTESLNYHKRSWSNVHRQPASSSYGLVG
SKIVTNAASRESSLFSSSLSDMFSQKLRLLGNGVLSGQPITVGSLPEEEPYKSLEEIEAETIGNLL
PDEDDLFSGVNDELGCSTRTRMNDDFEDFDLFSSSGGMELEGDEHLISGKRTSCGDEDPDYF
GVSKGKIPFGEQSSRTLFVRNINSNVEDSELKALFEQYGDIRTIYTACKHRGFVMISYYDIRAAQ
NAMKALQNRSLRSRKLDIHYSIPKGNSPEKDIGHGTLMISNLDSSVLDDELKQIFGFYGEIREIYE
YPQLNHVKFIEFYDVRAAEASLRALNGICFAGKHIKLEPGLPKIATCMMHQSHKGKDEPDVGHS
LSDNISLRHKAGVSSGFIASGSSLENGYNQGFHSATQLPAFIDNSPFHVNSSIHKITRGASAGKV
SGVFEASNAFDAMKFASISRFHPHSLPEYRESLATGSPYNFSSTINTASNIGTGSTESSESRHIQ
GMSSTGNLAEFNAGGNGNHPHHGLYHMWNGSNLHQQPSSNAMLWQKTPSFVNGACSPGLP
QIPSFPRTPPHVLRASHIDHQVGSAPVVTASPWDRQHSFLGESPDASGFRLGSVGSPGFNGS
WQLHPPASHNMFPHVGGNGTELTSNAGQGSPKQLSHVFPGKLPMTLVSKFDTTNERMRNLY
SRRSEPNTNNNADKKQYELDLGRILRGDDNRTTLMIKNIPNKYTSKMLLVAIDEQCRGTYDFLY
LPIDFKNKCNVGYAFINMIDPGQIIPFHKAFHGKKWEKFNSEKVAVLAYARIQGKSALIAHFQNSS
LMNEDKRCRPILFHTDGPNAGDPEPFPLGNNIRVRPGKIRINGNEENRSQGNPSSLASGEESG
NAIESTSSSSKNSD
SEQ ID NO: 14: OML4 nucleic acid sequence [symbol]
CCTTCTGAAATAATGGAGAAGAGGGGTGTTTCTGCCTCATCTCGCTTTTTGGATGACA
TTTCCTATGTTTCTGAGGTAATTATTAATGTAACTGTCTAAGAATGGTTTGTTCTAATTTATAA
TGTGACCCTCAACAAGCTAATTGTTATTCTAACTGTCTTATAATGTTTTTTTTTATAATGATTA
TGAGTTCCAAGAACATTTTACAGCCTAAGACTTCGGTTTTCTTTGTCATTTTGTTAATCAATT
TGACCTGTATGCATGGCCTCAATGCTATTGCCTTTTCGACAATTGGTTTTCTAAACATGCGT
TAAACTTTTATGGGCAGAAGAATACAGGATTACGGAAGCCAAAATTTATTCATGACCATTTT
CTACAAGGTGAGTTCAATCAACTAATTATTATTTGTTCAAAATGGTTTGTATATCTTGTGCTG
ATTTACCTGTGTATCAATTGCATCCTTAATGCCCCAAATAGATTCACTAACAGATAGTTAAGA
TTCAGACCTTTTGAGTGAACTGTTTACACTCCAGTTTAGAAATTGGCTAGTAGCTATCATTG
AGTTTGAACGTGTGAACTTTTTGAAGAATCTTTCCATATATGTTTCTGTATACCTTATTTTTGT
ATATTTCAAAGCAATATTTCTCTCAATTTTTGTTTCAATTTTTTATCAATGTTTTGTTTGCTTTT
AGAATTAATGATTGTCAATGTTGCTAACTAGTATCCTTCATACGAGTAATTATCTTAAATTCT
AAAACTGGTATATTTATTTCACTTTATGGTGATTGGTGATAATACTTGTTGATTTGTCTTTTTT
AGCCCATACACTTCTCACTTTATGCTGAAATCAATATGTAATTTTTATTTTGCTTCTGGAATA
ATGAATATCACTAATCAACGTTGCAAATTGACATCATCTAAAATTAATGTATTTTTCTGTTTGT
GGTGACAATGTAATTGCTGCAAACCTATATAAATTGCTGATAAAAAAAAAAAAACCTATATAA
ATTAATGTTTTATAGTGAATGTATAAATTCAATACCTTGTTCTCACAACATTTTGATTGTGGTA
TAGCTGGGATAATTAATGATGATTTCATGAATTTAGATGCTGTGCTCTGCTGGACTGAAGCT
TATTTATGATTTTGGTATAAATATTATTAAGAATTTGCTTTTATTTTAATTGTGCTAATTTTGAA
TGTAGTAATAATGTAATATCTGCATGTATCCATATTTATGTTTGTTTACCTATGTTCCATTAAT
AAGCAGTTCATCTGCTGAACATGTAACTAATTTCTGGATAAAGTAATTTCTATATTCAAATTT
TCAGGGAAGAGTGAAATGGGTGCATGACCTGGCATCATTTTTAATACTTCGTCACCCCATG
AAACCAATGCAAAAACAGGCTTGTTAATGTCTCAAACTACTCTATCTCGTGAAATTACAGAA
GACCTACATTTTGGCAGAGAAGCAGGCAATATAGAGATGCTGAAGGATTCTACCACAGAAT
CATTGAATTATCACAAGAGATCATGGTCTAATGTGCATCGGCAGCCAGCATCTAGCTCATAT
GGTTTAGTTGGGAGCAAGATTGTCACCAATGCTGCCTCACGGGAAAGCAGTCTATTTTCAA
GCTCATTGTCTGACATGTTTAGCCAAAAGTGTAAGAATTTGTTTCATGGATGTTAATATAGTT
GCATGCATGTGTTATGGGTATTGTAGCATAATCAAATTCTGGTTGCTTTTACACTTCGTAAT
ATTTTAGATATGAGTTTCTGTTGCATTCATTTGCTTGTGTATTTGTCATTAGCAATTTAGCAT
AGAAGAATATATGCTTGCTATCTTTTGTAATGTAGAAGGACAATACCCTCACCAAACCCACC
ACCAAAAATATAAAACTAAGTAAAGTTATCTATTTGTTTTAGGTTTTGTGATTATACTTTAGTT
CTGCTCATGTGTCACTGTGTGTATATATGTATATCTTAATGCAGTGAGGTTATTGGGGAATG
GAGTGCTGTCTGGTCAACCCATTACTGTTGGTTCCCTTCCTGAGGAAGAACCATATAAATC
TCTCGAAGAAATTGAGGCTGAAACTATTGGAAATCTCCTTCCTGATGAAGATGACCTGTTTT
CTGGAGTCAATGATGAGTTAGGATGCAGTACTCGCACTAGAATGAATGATGATTTTGAAGA
TTTTGACTTGTTCAGCAGCAGTGGAGGCATGGAATTGGAAGGAGATGAACATCTAATTTCT
GGAAAAAGAACCAGTTGCGGGGATGAAGATCCTGATTACTTTGGAGTTTCTAAAGGAAAAA
TTCCTTTTGGTGAACAATCTTCTAGAACACTTTTTGTTAGAAACATCAATAGCAATGTAGAAG
ATTCTGAGCTAAAGGCTCTCTTTGAGGTGAACCTTTATTCTTTTATTCTGGCGGATGCTATC
TTAGAATTTTCATGAAACATTTCATACCACTAATAATGGCATGTAAATGGACTATTTTGTTTG
TTCCAGCAATATGGAGATATCCGAACCATATATACTGCCTGCAAGCATCGTGGATTTGTTAT
GATTTCTTATTATGATATAAGGGCAGCACAAAATGCAATGAAAGCACTTCAAAATAGGTCAT
TGAGATCTAGGAAACTTGATATACATTATTCAATTCCAAAGGTATCATTATTAATAACTTCTC
ATGCATGCATAATTCCTTTTTCCTTGTCATTTTGATAAAGTTGTTATTTTTATTCTTCATCATA
TCATTTATTATTACCGCCATATGTTTTGCTTGTTCAATTGCTTGCATGCCTGTTTTTATGGTT
TGCTTATAGATATATCTTGATTTGATGACATGTCAGGGCAATTCTCCAGAGAAGGATATTGG
CCATGGTACACTGATGATATCCAATCTTGATTCATCTGTTTTGGATGATGAACTAAAACAGA
TTTTTGGGTTTTATGGAGAAATTAGAGAAGTAAGTCGTTCTTGTTGGTTTTCATCCATTTTTG
GTGTTTGTGTTTTAAAATGATACAAGCATTCTTAAATATTGTCTCTGTAATTGCAGATCTATG
AATATCCACAATTGAATCATGTCAAATTTATTGAATTTTATGATGTCCGGGCTGCAGAAGCT
TCTCTTCGTGCATTAAACGGGATCTGCTTTGCTGGGAAGCACATTAAGCTTGAGCCTGGTC
TTCCCAAGATTGCAACATGGTTGCTGTTACCGCTTCTTTTTTATTTTCAATTATTTTTTTCTC
TTTTTATATCAACTTTTTCAACTGTTTTCTACTTTTTTAAATGTGCGAATCTTAAAACATTGTTT
TGTAAATGAAGTCTTTTATTTTGGATCTTCATATTTATGCTCACCACCTTTAATAGTATCCTC
ACTTTGTAGAGTTTGATAGAGTGTAAAGTTGTTATCAACCACCTTGATGGAAAAATTACATC
TTGACAATTATGCAAACCTTGCTTTTGTAGTATGATGCATCAGTCACACAAAGGAAAAGATG
AACCTGATGTTGGTCATAGTCTGAGTGACAACATATCCTTAAGACATAAAGGTATAATTTTT
GTCTGCTTTCACTTGTCTTTTTTCCCTCATAAAGCTAAAATGTTCTTGGTCTCACTAGACATA
ACTTACAGCAGGAGTGTCATCTGGATTTATTGCATCTGGTAGCAGCTTGGAAAATGGATAT
AATCAGGGATTTCATTCTGCGACACAGCTACCTGCTTTTATTGATAACTCACCGTTTCATGT
GAATTCTAGCATTCACAAGATCACAAGAGGGGCATCTGCGGGAAAAGTATCTGGTGTTTTT
GAGGCCAGTAATGCTTTTGATGCTATGAAATTTGCATCCATTTCGAGGTTCCATCCTCATTC
TTTACCTGAATATCGTGAAAGTTTAGCTACTGGCAGTCCTTACAACTTTTCAAGTACCATTAA
CACGGCTTCCAATATTGGAACTGGATCGACGGAATCATCTGAAAGCAGGCACATTCAGGG
AATGAGTTCAACTGGGAACCTAGCTGAGTTTAATGCAGGAGGTAAGTTTAATGTGCTAAGA
AAGCCTCATGTATATGCTTCCTTTATTTGCAGCAGTTTTGAAATGTTTCCTTGTCTATAGAAA
ATTCTGATAAGGAATCAATTTGTTGCAAAGGTTGAACTTATTGTTCACTTTAAATGGCATCCT
AGGAGTTTGAAACCTTATAATGAAGAGCTTGATTGTTAATTTTAATGATGATGCCAGCCTAG
GGTTTTCAATATTTTCATTCTTCTAATAACACCCAAAAATAATAATTGTTGTTTAAGAGCCAG
ACTATTGATCTATATGAATCTAGACTTGCCTGTCCGAGTATATGAGATTTAAGCATTCCAAAT
TGTAAATTGGTCGAGGTCATTTTTCCTACAAGCTTGTAAGTGGTAGAAGGTGCTGGGAAAT
TTTAGGCTGAAGCGATATCTAATATGGATTTAATAGTTCTATATTTGAATGCTGGTATGTAAC
CTTTTTGTTTGATTTTGGACCTTCAGGAAACGGAAACCACCCCCATCATGGACTTTATCATA
TGTGGAATGGGTCCAACTTGCATCAGCAACCTTCTTCAAATGCCATGCTTTGGCAAAAAAC
ACCATCCTTTGTTAATGGTGCATGTTCTCCAGGTCTTCCACAGATACCCAGCTTTCCTAGAA
CACCACCTCATGTTCTTAGAGCATCACATATAGACCACCAAGTGGGATCAGCACCAGTTGT
TACAGCCTCACCCTGGGATAGACAACATTCTTTCTTGGGAGAGTCACCTGATGCTTCTGGT
TTTAGATTGGGTTCTGTTGGAAGTCCAGGCTTTAATGGTAGCTGGCAGTTGCATCCTCCTG
CTTCTCACAATATGTTTCCTCATGTTGGTGGGAATGGTACAGAATTGACGTCAAATGCTGG
GCAGGGCTCTCCTAAGCAGTTGTCACATGTTTTCCCTGGGAAACTTCCCATGACTTTGGTT
TCTAAATTTGATACTACCAATGAACGAATGAGAAACCTCTATTCTCGTAGAAGTGAACCAAA
CACTAACAACAATGCTGATAAAAAACAATATGAACTTGACCTAGGCCGCATTTTACGTGGG
GATGACAACCGGACAACACTCATGATAAAAAATATTCCCAATAAGTATGCCAATTATCTCCA
TATCTTTTTTGTGCATTTTTGCTGCTTATGCTGTTATCTTCTCATCCTTACTTCACCAAAGAAT
GTGATTATTAGTTAAATAAGCAATTGCTTATTTGGCTTGTCCGCTTTTCATGTTGGTGCATTA
ACCATACAAGTGCCCTCTCTCTTTTGCTTGCTTACATGCCTAAATAGCATATACTTTTTACAT
AACAGATTTTACAAAATTTGAATAACAATTTTTAAAAACAAGCAAATTCTTTTGCACTGGTCT
TTCAGTTTGTCTCTTTCATTTATATTTGTTTAAATATTTTTGGCAGGTATACTTCAAAGATGCT
TCTTGTTGCCATAGATGAGCAATGTCGAGGAACTTATGATTTTCTGTATTTGCCAATTGATTT
CAAGGCAAGTATTTATTTGGACTTGCTAGTTGATTGATTCTTTACTTAAATGAAGTAATCATA
AATATGTTTCTAAGTCAATTCTACAAATGGTTGCAGAACAAATGTAATGTTGGCTATGCATTC
ATCAATATGATCGATCCTGGACAAATTATTCCATTCCACAAGGTTATTTGGAAACCCTTATG
TAATACTAATTTGATATAATTATCTGTGATTTGAATTCTGAGTTCATCTGATTCTTTTTGTTTC
TAAGAGTTGATATATCTAATAGATAAGGAATATTGTACAGGCTTTTCATGGGAAAAAATGGG
AGAAGTTCAACAGTGAAAAGGTAGCAGTACTCGCCTATGCCCGAATTCAAGGAAAATCTGC
TCTTATTGCTCATTTCCAGAATTCAAGCCTGATGAATGAAGATAAACGGTGCCGTCCTATTC
TCTTCCATACAGATGGCCCAAATGCTGGTGATCCGGTAAATCAGCTTGTTCTTTAGTTGTAA
CTATTTTCCTTTTGCTAACTACAATGTATTATGGAACTACTATATCAGCTTGTTCTTCAGTTG
TAATTGTTTTCCTTTTGCTAACTACATTACTACAATGTATTATGGAACTACTATAGGACCAGA
CTACTAATATTCCTCTCACTGATATTTTATTCCGTAGATCTGGTTTTACTGATGATACATTAA
TGTTTTGGGCTGATGCAAGTAAAGTGGGAGCTAATTATAGGCTTTCTCACTTCAAAACTTTT
TTGCCTGATGTCTAATATTAACATCCATTGGGTGTGGCAAGAAAGAGTTTCTTGAAGGAATA
TTTCCCCGATGATTATTTGACTTACACATAACAACTAAAGCATTATGTTTCTGACTTGAGTTG
GTTTTAATGGCTACAAAATGCAATCTATTAGTGTGATTTTAACTGTTTCTAGCATTATATGCA
TTTAACAAACTGGGCTTTCCATTAAAAGAATATATATTGGCTTGCAACCTAATTGTACTATTG
GTACCTGGTTCTTCCACTGATATTATATACCGAGACAAAATTAACCTAATCTGTCTCTGCAG
GAGCCTTTCCCCTTGGGTAACAATATTAGAGTGAGGCCTGGAAAAATTCGCATTAATGGTA
ATGAGGAGAATCGCAGCCAAGGGAATCCTTCATCTTTGGCAAGTGGAGAAGAGTCCGGGA
ATGCAATAGAATCTACATCGAGCTCTTCAAAAAATTCTGAC [symbol] TTTAGCATCATGATCTAA
CAGTTCAATGTTGCATGTGATATCAACTCCAAGACTGTATATTTACATTATCTTTTTGTTCGA
TCGAGCAAGAGGAGTTGGAGCTGGTAGGAAAGGGGGCTCAAAATTTTTTCCTATAGAGGA
GCCTTGCAAGAGTTTTTGGAAGTTGAGGTACATAACCCGAATGAAGTCACTGATTCTATTGT
TTTCCGTTATTTTCCTAAAATTTTGCATGGAGTACTGCTACCATCCTACAACTTTAGAGAATG
GCCTAACTGAAGCTTAAAATTTTGGCTAGCTGTGAATGGACAATGTGACACTTTGCAGTTTC
CTTGTGATAATGTGCATCATTGTGGGTTTCAAGGGTTCTTTGCTGATTTTGTTTTCATGGTC
ATCTTTGTTGTTCATATGTAATTTTGTTCCCTTATATTCCCGGGCATTGGTATCCTTATGGGT
TTTGTTGTCTATATAATGGATTTTTGAGGAAAAACATTTAAATGAAACATTTTCTTCGGTGGT
GGTAGTATTCAGATATTTCTGCTTCGCTTGTTATTTCTGTATTCTTTTATCAGTGCTTATACA
CCTGTTATGCATGTCAGGGTTCTAGTCATTGATTAGAAAAATGCTTAATTCAGCCTTAGTTA
CATGCCACTAGCAAATGCTGTTTGATAATAAAGGTTCCTGAGTCATGACTATATTCTTTGAC
AGAGAAAAAAATTGAGTATATATAGCAATTGGTTAGATAGATTTCTCTATAAACATTTAAAAG
AAAACAAGAAGGTAAAAATATTCAGTTTCTTAATAAACTAAATTCAGTTTATCCATTTAACTTT
TGGAGAAGTTAAATGAAAAGAGTTTCTATAAAAGTTAAGTGCATATGCAGTAAGTTTGATTA
CTATGAAGTTATATATATTTTCTTTGAAGTTTGATGATCATAAAGTTTTATTTTATATATGTAT
GTATATTTCAAATGACTGTTTAGACGAAGAAAACTAATGGTACAAATTTTCTGTTCACAATCA
CATACGTTGGCATTTATTTGTACGCAAGTGACTGGTGAGCTTTATGGCATGACTAGCAGTG
GTACATTTGCTGCAACCAGACCTGATGAAATGAGATTTGTTTTCTGTCCATAGATATCTGGC
TTTTCTTATCATGATAGTGACTCATCATTTTCCGAAGTTTCAAGTCTCAACATGTATTTTTTTT
TCATTTTAATTTTTTGACCATAAAGTGAAATCTGTTGAAAAAGTAGTGCAATGGTATCTACAA
TTCCAATATATGTGTCTGCGCAGTGCGCACAAACTTTACAAAACTAACCAGTGAGACATGAT
TTTGCACTTTTGCCTTTTGGTTTCAAGAGTCAAGA
SEQ ID NO: 15: OML4 promoter sequence
CACCAAACCATAAAACATATGAATGTGTGCAATTAAATTATTTCATTGGTTAGTAGATATGCA
AAAGAAAAACAGTTCCATTGTGAAAAAAACAGTTTCATCATTTGAATATAACAAATTTGATAT
ATATATATATATATATATATATATATATATATATATATATAATGATTGGAATATATTTAAAAATA
GAGTAAATACTTTCTTATTGTTCACAAAACATTATCTTAGAAGAAAGCATTTATGGAAATTTT
TTTTAGAGAAAATATTGAAAGTAGCCATTAATCAATAGAATTGTATAAAATTTTCCTAAATGA
AATTTTACAAGAATATAGAAACCCAACTTCCCATCTTAAGCTCAATACACATGCCTGCATCC
AATATAAAGAGGGGTTTCAACTTGTTGTTTTTCACTGTAAAAAAAAAAGTTTTAAGCATATTA
TTATTATAATCCACACTACTCCCTGCACCTATTTATTATCATTTTTTTAAGATAAGATTGTAAT
TAATAAAHTGHAAATAGAATTTAATGAATTAAAAGTTAGTAATTAATTATCATTTTTCTTCG
AGATAAGAATGTAATTAATAAATTTGTTAAATATAATTTAATGAATTAAAATTCAAGGTATTAA
TAAATTACAATTTTAAAAATAATTAAAATTTTCACTTAAATAGTAATTTCATTACCAAATATTTT
AAGGACACCAATAAAGTAAATGTATCTAGTAATTACTTAAAAATAAATATTTGTTTAATTTTTA
ATGATGTTATTTTTACTAAATTCCTAATATGGTAAAAAAAATTCACTTAATATTTTTCTATTTGT
TCATGGACTAATAGTTTTTCTTATGTACATTGGTAGTAATGAGGATCGAAACCATCCATCTA
CTATAAAACCCTTGCTATCAGATGACCCTAGTGAATTAGACTAATAATTTAAGCCATCTTTAG
TAGTAAATTAAATTAAATTAACTTATTATATACATGCTTATATGCTGAAAACAATAATAAATCA
CATGTGAGTAAATACATGAACAATTTATTTTAAAGACAAATTTTTTTTCTTTTGAATACGGGT
ACGTTATGAGAATCCATTTAAAACATTTATATAACCATTATTTTGTCGCATGCATATACCATT
ATGTATCCTGGGATTTTATGATGCACAATAACTATAACTACAAAATTAAATAACTATGGAATT
TTAAAAATTAAATAACAAATGTTTTTCAGTTATACCTTAAAATGCTAAATGTGTATTTAAAACT
AGGAAAATAAAATAGCAAGTACTTAGATTAATTAGTGTGGGTTATTTCAATTTATCCATTAAT
CTTGAATTAAAATCTAAAAGTATATAATTGTATTAAATATTTTTATTCAAGCAAGTGCATCTCT
TGCATGTGTTTTTTGAAATCAATATATTTTCATTGTCGGAAACTAAAAACTTTGAAACTAAAA
AGACAACTTTGACACCGACTCTCAAAGCTTAAAGATTATCAAAGTGATTTTACCTCTTTAAAC
AAAATATTTTTCATACAAATAACACATTTGGAAAACCAAAAAATTAAAAAGTATAATAAAATCA
CCAGTAATTCACAATAAAACATAGATATGTGTATAGATGAATTATTACATTAAGCTGGTATTC
AAGCAACACTAAAAAAAAAAAGAAACAGATTCATGTTGGATAAAATCAAAAAACAAAACGAA
ACGGTTTCATTATTTGAATGTAACAAACCTGACACCTGATATTATAATAATAACAACACATTA
AATTATATTTTTAAAATACAAATATTATTTTTTTGTGAATATATTAAATTTATTTGAAACCATTA
TCTTAGAGGAAACATTTATGGAAAAGAGTTTTAAAAAAAATACTGAAAAAGTAACTGTAATAA
AAGTATCCATTAGTGAATAAATTTGCATAAAAAATTCCCAAACAGAATTTTACAAGAATTTAG
AAAGCCCCACTTTCCAAGTTTCCATGTTAAGAACACACAGACTTGCATCCAATAAAGAGAGA
AGCTATCTTGTTATTTTCCACTGTAAAAAAAAAAAAAAAACAAGTTTAGTTTGAAGCTGCTT
ATTATTAAAACACACTATTTCTGCGCCTATTTATTCTCCCTCCTCATTTCTAGTTTTCATTTTT
TAATTATTATTATTTTTATTTTTTTTGTTTTTGTTTTCAAAGCCAACTAACACCCTTTTCCTTTC
ACTTTCTCTGTCAGAAGACTAAAAAAACACCTCTGTTGTTGCCTTGTCCGTTTATTTTCTCTC
CAAACCAGAGAGCGACCGCCGGCGAGTCTCACCTCGCCGGAAATTCGCTCTTCCTCGCCG
GAACCTCCATTTTTCCCTTTCCTATTGCGCTTGCTTTTTCTTCCCACCGGCTCCTTCAAGAG
GAGCCGTTTCCCTGCAATAGTTTTGTTGCTTCTTTTTTTTTTTCGTTTTCTGAGCGCCTGAG
ATTGCAATGCAGAGGGAAGGAGGTTCCTGTCGCGGCAAGAAGCGTGGGATCTCTCGTTTC
TGAAATTCCTATGATGTGGAGCGTTTGAGAGATTCGTTTTTTTCTTCTTCTTTTTTTTGTCTC
TTTCTCTTTGTTCTCGAGTTTCTGGATGAGGAGCGCGCTGAATTTTGTGGAGGAGAAGTTTT
TGTGCTAGCGAGGGCGTGTTGAAATTCAGAAGGTGTGAATTTGTTTTGTTAGCTTGAGAAA
AAAAAAGTGCTAATTATGGTTTGTGTTTATGTGTGTTTCTTTTTGCTTTTTTTTTTTTTCTGGT
TAAATGGTTTTTTCTCTTTAGTGGAAGTGGTTTTTATGAGTTTATGGAGAGATGAGAATCTCT
TTGGTGTTCATGTTTTTGATCTGCTGGTGTTTTCAGATTATGGCCTCTCTGAAATTTTGTTCT
TTTGTTTTTATTTTTTCTCTGGATCTGTTAGTGTTTGAGGCTTTCCCTGTGAAATTCCCTCTC
CATATGTGTAATTTTGTGATAGAACCAAGGTGTAGTGATATAAATTTAAGTTAAATGCTTGTT
TTTTTTTTTTTTTTTGGTTTGGGAAAAAAAGGGAGAGTTGTGGTTACAGTTGAATGGGATTTT
ATTTTTGTTTTGTTTTAATTTCCATGGATAGTTTTTGTTTTTAATTTTTTAAGTTTTTTGTTAAG
CAAATGGCCTAATAACCGCATTAGGTTGTTAGTAGGTAAGCATCGAGCTTCTTTCTTCTCCT
AATACATCCTTCCTTCTGGTAATGTATAGTTGAAGATGCAACTATTTGTGGCTTTGTTTTCCA
CTGCTCTTTTTAAAATTTGCAATTGAATATTTGAAGTGCTTTGGTAGGGTTTTCATTAGTCCA
TTTTTTTGTCACTTTTTTTTGTGGTGGTGATGTTAGTAAAGTTCCAATTGTTATGATAGATGA
TATTTTTCTGGGTCTGAATTTTTCTTTCTGCCTGTAGCAGTTATGTATGATATGAAATGCGAT
GTTCATTGTTATCAGTCCTTTCCTATGACAAGGGAATGACCTTGAATTTCTCGCAGATGTCG
CACATAGAGCTTGACGAAAACTAACAAGGAAGGAGGTTTTCAGG
SEQ ID NO: 16: GSK2 amino acid sequence
MASLPLGHHHHHHKPAAAAIHPSQPPQSQPQPEVPRRSSDMETDKDMSATVIEGNDAVTGHII
STTIGGKNGEPKETISYMAERWGTGSFGWFQAKCLETGEAVAIKKVLQDRRYKNRELQLMRL
MDHPNVISLKHCFFSTTSRDELFLNLVMEYVPESMYRVIKHYTTMNQRMPLIYVKLYTYQIFRGL
AYIHTALGVCHRDVKPQNLLVHPLTHQVKLCDFGSAKVLVKGESNISYICSRYYRAPELIFGATE
YTASIDIWSAGCVLAELLLGQPLFPGENQVDQLVEIIKVLGTPTREEIRCMNPNYTEFRFPQIKAH
PWHKVFHKRMPPEAIDLASRLLQYSPSLRCTALEACAHPFFDELREPNARLPNGRPLPPLFNFK
QELAGASPELINRLIPEHIRRQMGLSFPHSAGT
SEQ ID NO: 17: GSK2 nucleic acid sequence [symbol]
GCCTCCTTGCCCTTGGGGCACCACCACCACCACCACAAACCGGCGGCGGCGGCTAT
ACATCCGTCGCAACCGCCGCAGTCTCAGCCGCAACCCGAAGTTCCTCGCCGGAGCTCCG
ATATGGAGACAGATAAGGTACTTCCGCTCATTGTACTCTTCACGAACCCTCGGAGTGGTTT
CCGACTTTCCGGAGCTCCGATCTCCGTCGATTCGCCTCGAAGCTCCGGCGTCGCCGGAGT
TTCGACCGATCTACCGGTTTTCCGTGCTCGCCAGAGATTTTCTCCGGCGACGCCGCTGAT
CGGAATGGTTATTGTTTTCTTCGAGAGCGATGTTGATTCTCGTTGACGAACTCCAAAAATAG
AAAAGAAAATTAGGTTTTACTTTTTTGGAGTGTGTTTTGGTTGATGCTTTTTTGGTAGGGATC
TTAACACTGAAGAAAAAATTAGAATTTTCTGTTTTAGGTGTCGGAGAAAAGGAAAGGAATCA
ATGTGAAAATGTGGAATCCTGTGCTTTGATTTTTTGTTTCCTTTAATTCAAGGAGAGAGATTC
TGATTAGGTGTACTTAGCTGACCTGAGTTAACATTCTTATTTCACATTCTAACATTTTTATGT
TTCTTTCACTTATCTCTAATCTACTGTTAATTTTCTTTAGCTATGTTAATTCTGTGCTATTATA
TGGTCTATTATGGGGGTATAGTTTTTGTTTACATTTTTTGGGGTTTGTGTGTGTGATTTCCTT
TACTTCCCTTGTGGTGGATTGTTGTTCAAAAGGTCAAACGGTTATAATTTGCTTTGCTTCAG
GGAATTAGTGTCCTTAGATTCTCTCTGTATTGTGTCTTAAGTTATAGCGTTGAAGTTTTTCTT
TATGCTTTCTTGTGAGCTGCGGTTACCTGATTTAACTTTAGTTTATGTGTGTGCTCTTTGAG
CTCTTTACACTTTGCCTTTCTTCAACTTCACATTCTGAACTTTGTCTGATTTCTTCTGGTAAC
CCTCTGGTTCATATGTTTCATTGCCATGCAATTTTCTTCTCATAACACTTGTTTCAACCAGTA
AACTGTCATGAGATACCCCCTTTCCTTATATTTGCATCTTCTCAAATTAACTTCACTGTCTAT
ATGCATGTTTGTTGCCATGTCTGGCACGGCATGCGTTTGATAGTTGATAGGCACATGTTGT
TGCCATATTTTGTGGACGTTGCTAAACAAAATTATTGATGACAATATCTGTAAAGCTAAATTT
AAATATGATTGATATTGTATCAATAAAAATCTGACATTCAAGTACTGTATTAGGAGATTTGCT
TTACTGCATTAAATATCTAATTCTTGTTATAAGTTGCAGGATATGTCAGCTACTGTCATTGAG
GGGAATGATGCTGTCACTGGCCACATAATCTCCACCACAATTGGAGGCAAAAATGGGGAA
CCTAAAGAGGTGAGAATGTGTTCTAACTCCCAACCCCTTTCCTCCTGAACTTACAATTTTTA
TTAAAAAATTCATTTCATACCCTCATAAATATATGTTATTGTATATACTGATTATTGTTTTGAT
AATGGTTCACTTCCTTATGGGGATAGAGTGGAAGTAGAGTTAGTGGTTGGGGAATCTAAAT
TAATAAATTGCCTATTATATTCAAGGCCTTCCAAAGATATAATACTGGTTGTCAATCAGAGTT
TGGCTTATTTCCCCAGTGCTTACCTCATCTGATAATTTTTATTCGCCAACAATATATTAGCTC
TTACAAGATGTATAATTTTGAAGAATTTAATTATGATGGTTCAATAAATTTATGCTTAAAATTG
GTGATATTTATCTGAGTTTCCTTATGTGGGCTTGGTTGAAGGGGTGGGAAAGGGATTTCAA
TGTCCCTTTTCTCCAGTGGTCCTCAGCAATGTCCTTAGCTTTTATTTAATGCTTCTTGGAAG
GGCAGGGTTTGTGGTTTGTTCTTGAGATGTTTGTTATGTTTTTACAGAAGTATTATATTCTAT
GTATCTTTTAGTACTACTGGTACTTTTCATGCATTAATATATATTATCTTTGGAGTCCAAAAA
AAAATAAAATTTATTCTTCGTAAATAACTTATTTGTTATGATTACTTCCATGATACCACCTGCA
GACCATCAGTTACATGGCAGAACGTGTTGTTGGCACTGGATCATTTGGAGTTGTTTTTCAG
GTATGGATGAACAATCACCTAGATGACAAATATTCCTATTAAGCTTTCTCTGCTGTCACATA
TCTCATTGTTTTCCACCCCTGGATGGCATTCCTCTTTTACCTAAAATATAGGCAAAGTGCTT
GGAGACTGGAGAAGCAGTGGCTATTAAAAAGGTCTTGCAAGACAGGCGGTACAAAAATCG
TGAATTGCAGTTAATGCGCTTAATGGATCACCCTAATGTAATTTCCCTGAAGCACTGTTTCT
TCTCCACAACAAGCAGAGATGAACTTTTTCTAAACTTGGTAATGGAATATGTTCCCGAATCA
ATGTACCGAGTTATAAAGCACTACACTACTATGAACCAGAGAATGCCTCTCATCTATGTGAA
ACTGTATACATATCAAGTATGAACTTTTCTATTCTGTTTGGAATTTAGCTCATGTGTTGTTTT
ATAACATTGTAACAATCGAGTTTGGATATGATGTTTAGATCTTTAGGGGATTAGCATATATC
CATACCGCACTGGGAGTTTGCCATAGGGATGTGAAGCCTCAAAATCTTTTGGTATGCTTTC
TTTCAATGCTTTTCCTCTATGAGTTGTATTCATTTATTCTAAATCTTAACCTTTTGCATATATG
ACTACAGGTTCATCCTCTTACTCACCAAGTTAAGCTATGTGATTTTGGGAGTGCCAAAGTTC
TGGTATGTTGGTCTGCATTGTTCTTGTACACATCATTGCTTCATGTACATATGCCACCATGA
TAATGGAGGACTACTAAAATCAAATTCTTCCTACCGGACATAGCTATGCTAAAACTTGTATA
AGATCTTTCCATAAATGCAATATTGATTTAACCTGTTTATGTGATGATTTGTTATTAGTAAATA
GCAATTGAAGTGAAAATGATGCCAAGAATCTTGACTCTGACCCATTTTTTCCTATTATATGA
AAAATAAATAGAAGAAAAGTTATCGATTGGCATCATGTGGATTTTTTATTCAATTATCAATTT
CATGAAGCTTCTCATGTTCACCACTTGGTAGGATATAGTTATGATATTATTTTTCCACAAAAA
ATTTATATCAGGTCAAGGGTGAATCAAACATTTCATACATATGTTCACGTTACTATCGGGCT
CCAGAACTAATATTTGGTGCAACAGAATACACAGCTTCTATTGATATCTGGTCAGCTGGTTG
TGTTCTTGCTGAACTTCTTCTAGGACAGGTTATAAATTTCTGGAAATCTATGCATTAATGTTG
TTGATACTTAAGATTTTTTTGCTTTCTTTCTGGGATATGTTATATTGACTTCACGTAGTTTCTA
ATGTTTGTATAGCCATTATTTCCTGGAGAAAACCAAGTGGACCAACTTGTGGAAATTATCAA
GGTGATGTCCCTTCTATATGAGTGTCTCCATGGATTGCAGAATATATCTGCAGAGATAATTA
TTTAGATGTCTTCTTGTAGGTTCTTGGTACTCCAACACGCGAGGAAATCCGTTGTATGAACC
CAAATTATACAGAGTTTAGATTCCCTCAGATTAAAGCTCATCCTTGGCACAAGGTAATGACA
TTTCTCATCCATCCTCCTTTTGATATTCATCACTTGCCATTGGACTTTAAAATGGGGATTAAA
AAGATGAAAAAATAGTTGTCAAAATCAAATTCAATAGCATGCGTTACAAGTTACAACTAGGT
TTTTGAGGTTGCTTTCCATATTCTTTGTTTTGTAATTGATGAGCATGATAGCATTGATTGATG
TAACCTACTACCTCACTAATAGGATAAAGCATTGGCTAGTGAATTATGCATTTATTTTGGTG
CTCTATGTTTCAGGTTTTCCACAAGCGAATGCCTCCTGAAGCAATTGACCTTGCATCAAGG
CTTCTCCAATATTCACCTAGTCTCCGCTGCACTGCGGTGAGTAGGATGAACTATGATACCT
CCCTTCACTTTTCCCCTTTAAATAAAAGGAAAACATACACAGGAAAAAGTTTGCTTATTTTAA
CCTTCTTGCTGTGATATTATATCTATATTCCTTATGGCATGTTTTTTAATTTAGTTAACTCAAT
TGGCTTAATTTTCACTGGTGGCTTTTATTATTTCAGCTGGAAGCATGTGCACATCCTTTCTTT
GATGAGCTTCGCGAACCAAATGCCCGGCTACCTAATGGCCGTCCACTGCCCCCACTTTTC
AACTTCAAACAGGAGGTATATATCTTAGTCGTATTTTTTTTATTAAATGTGACGTGTCAAGGC
TGTTGCTTTGTCCACTGTTCATTATGTATATCTGTATGACTACTTACTTTACCATCTGTTCTG
CATGATCCAAACCAAACACAAGGGAATCCAATTAACAATTTCTCTTATATCAAAATTGTGAA
CAGTATTTAACACCAGAATATTATCTTAATCATTCTGCAATGAAAACTTAACTACAGTTAGCT
GGAGCATCACCTGAACTGATCAATAGGCTCATCCCAGAGCATATTAGGCGGCAGATGGGT
CTCAGCTTCCCGCATTCTGCCGGTACA [symbol] ATGTAAAGGGATAATGAAACGATGAGTCAAC
CTACATAGTGATCGATGTGAATCAACAGAAGGGCTGTTTGAGGCCTATGTATAACTGGGAG
TCCCAACATAATATGCAGTTTTTCCTCCCCCTTGTGAAGATGTATACATGTGTTGGTTGCTC
GGTAAAGCTTGAAAGTTGGTGATTCTGTGTAGTATTTCATTCAAGTTAAAGCATACTTATCC
CTGCATCTGTATATTGTTTTGGTCAGATTTCAGAAAGCTAGGAGTATAAAATGATAGCAATC
ATGTCTTCATAGGTAGAGGGGCCCAGCTGAATTGAGGGGCCCCTATAGTAGTTTGGCTTTG
CTTTTTATGAGATTAAATTCAGCATGTCGTTTATATTATGTTTATAACAATCTCTTGATTCAAA
ACAAGAAATTTTCTCGTTGTTTAATACTCTAGTAACCCCGTTCCTTCTACCCAAGAAGATTTT
GTTTGTCATATGTGGACAAGAAGAAAGGATTCAATCAAAAAGTTGATTACGGAAGAAAAAAA
TATGAATTCTTTATGTTGATGACAAGGGTGTGTGCACTTAGGGTGACTTGTTAACAACATAC
GTTGAGATGAGGGTTAATATACTTCGTTGCTATATATTCAATTATATTTCATTTCTATTTGTGT
TGAAGTCTAAGTCAGAATTTGAAGTCACATATGGTTAGGACTTGGGAGCAAAATATATAAGT
GAAAAAGAATCACAAACCTAACGCTTTAAGATCATCCACTATGCATATTGAATTGTTTAGAA
GCTTTTTCGGTGGTCCTACACTTCACCTCAGATTTAAAAGTTTTTTTTTCCTCCGATGATAAC
ATTAAATGAATTGTTTAAATGAACTTAAAAGATGTTTTTTTTTTTTATAAAAAAATTTGGTTAA
GCAGCAATTCTTAAATGCTATGTTATCCGCTCTAATGGTAAATTCTGTTAAACAATGTTGTTT
CTGAACGTATAATATAATGTAATCAACGAAATAAAATTACTATCAATCAAAGATACTAGGGTA
TTAACATAATTATGAGTTGATTTAGTTTGAATTTAGAAAAAAAACTGATTAAACTGGTTTGATT
TG
SEQ ID NO: 18: GSK2 promoter sequence
GAAATTATTTTAAGTAAGATATCTATTTATAAATTAGGTCCAAATTCACATTTTTTAAACATTA
TAAATAGAATTATCTACCTGAACATAAAGTGTTAAACAATTTAGAATGTCTAATTTTAAATTTG
AAAAGTAAAAAAGAAAATTTACTCAGAGTTTCACTTATCACAAAATTGATAGTAAAAAATTAG
TTTCAGTGTATAATTTTTACATTACTGAAGAAAAAAATTTGTGACTTTAAGAGCTCATTATAC
ACATTTAATATGTTTTGGATTCTAGCTAGTCTGATTTAATATAATTTAAATATAAAATATTTCA
TAATTGTTTGTTATGCATGTTTTGAACACCACTCCTTCCATAAGGGGGAGTTTACACTGTTC
AATTACTTTTACATGATCACGTCAAGGTCAAGATTATGATTCTTAATCGCATCCATAGCTAG
CAAGAAGCAAAAGCAGTTACCAGAGGTTTCTGGAATTCCCAGCTTCTCTCTCTCTCTCTCTC
TCTCTCTATATATATATATATATATATATGATTGCCAAATGTTACATTTTGGGGCTTATGTGAA
GTGATAATAAATTCAATTGAACGTCCCTTCTCTTCCTTTAGGATATTTCTTTTTCTATAACATA
AAGGATAGTTTAGAATACAATATATAACTACCTGTTTTAGGTTTTAACTATTGAATCGGGTAA
AAACTGAAAACAACTAATGCTGAAAAATAAAATAAAATCTAAAATTGGAAAATTGGCCAGGT
TAAAAATAAAAGAGGTTAATTTCTAATCTATAAATTTAATGTATGTTTAGATTGCAGTTGGAA
GAGTTTAAAATATTTTGTCTCAAAGTAGTAGTTTTTTTTTCTTCATTATTTCGTACATTAAAAT
TTTTAAAATTTATTCTCAACCTTTATTCAAATATAGTCTTATAATTAGTACTAATTAAATAGTA
GTGTCAAAAATCCTACACTCAAATATATTCCAATTAAATTTTAAAAAATATTATTTTTCATGAA
GTTACGGAGTGCTGTGCACTAATGAGATGAAACCGAGCAAATTATTAGAATATACTACATAG
TTACAATTATAATAAATGAAAATTAAAATATTTTTTACATTATTTGGATATGCATATAGAAATT
ATATACTTATATATATATATATATATATATATATATATATATATATATATATATATATATATATAT
CAGTAATATAATTTATTAAACCCAATGGAAAGTACTTATGAGAAGGAGCTGTAATTTTTTATT
TTATTTTCAAAGTATTTCCCAATAAATAAATATCTAAGTAAAAGATTAATTAATTGAAAAAAAT
AGTATGCATGAGTTTTATTAGTGAATATTTTAAAAATTTTGGTTAAAAAGTACTTAGTATATTG
TTATGAAATATTTTATTTTTCAACTAATTAAAATATTTAATATAACTGTATAATATACATATAAT
CTTATGATCACTTGTTAAAAAGACTCATTCAATTTTAAATAGATCAAACTGTACATTTGATTTA
ATGTTCATTCTTATTTTATTTCTTAAGTTGACAATTCATAACAAAGTCATAAATGCATATATGT
AGGACAGCGTTTTCATTTTGAATGAATCAATTTTCTTTAAGATGTGTTTATTTTAATTACATTT
CTTTCTTTCTTTTGTAAGAGGTTTTCAAAGATGTTCATACTATATTAACTGCGTGAACCATGC
ATCGGATGTTTCGTGTTCACAATGATTTTTAATGAATATTTAATTAATAAATAAAGAAAATATC
AAAATGTCTTTTAACGTCATCAAACGTTAAATATATATATATATATATATATATATATATATTTA
TCATAAAAAATCAAAATATTTATTAAGAAGATTAAATATAAAAATGTAAATTTATCATCAATTT
GGACTTGAGTTATGAAGCACTACCTTTCGTTTTAAATTCTCTAGATAAACTGTTTTACAAAAT
ATTGCATGCAAGTACGTAACATTATACGAACATCGAATTTGCTTCGGTTCTCCTGCCCCTTA
CGGCACCAGATCACTGCTCCCTTTCATCACGACCCTGATTCGCGCGTGCTCTCAATCTCCC
TAAACTCGCGTGAACTCACTCTTTCTCTCTTCTTGAACAAAAACAGGGCAAGAGAGAGAGA
AGAAAAACGAAGAAAGGTAATAGAGAGAGAAAGGGAAGAGGAGAGAGAAACGAAGAAGAA
GAGTGTTTCTCACATCAC
Maize
SEQ ID NO: 19: OML4 amino acid sequence
MPFQVMDPRHHLSQFTNTTVAASSFSEEQLRLPTERLVGFWKQESLHHIGSKSVASSPIEKPQ
PIGTKTMGRVDPQPYKPRGQKSAFSLEHKTFGQERHVNMPPSLWRADQDPYVQSDSSLFPD
GRSTNPYEAYNENGLFSSSLSEIFDRKLGLRSNDVLLHQPLEKVEPTHVDDEPFELTEEIEAQII
GNILPDDDDLLSGVDVGYTAHASNGDDVDDDIFYTGGGMELETVENKKSTEPNSGANDGLGSL
NGTMNGQHPYGEHPSRTLFVQNINSNVEDSELKVLFEHYGEISNLYTACKHRGFVMISYYDIRS
SWNAMRALQNKPLRHRKLDIHYSIPKDNPSGKDINQGMLVVFNVDPSVTNNDIHKIFSDYGEIK
EIRDAPQKGHHKVIEFYDVRAAEGAVRALNRSDLAGKKINLGTVGLSGVRRLTQHMSKESGQE
EFGVCKLGSLSTNSPPLPSLGSSYMVAMTSSGRENGSIHGLHSGLLTSMSPFREASFPGLSSTI
PQSLSSPIGIASATTHSNQAPLGELSHSLSRMNGHMNYGFQGLGALHPHSLPEVHDGANNGTP
YNLNTMVPIGVNSNSRTAEAVDCRHLHKVGSSNLNGHSFDRVGEGAMGFSRSGSGPVHGHQ
LMWNNSNNLQRHPNSPVLWQNPGSFVNNVPSRSPAQMHGVPRAPSHMIENVLPMHHHHVG
SAPAINPSLWDRRHGYAGELTEASSFHLGSVGSLGFPGSPQLHGLELNNIFSHTGGNRMDPTV
SSAQISAPSPQQRGPMFHGRNPMVPLPSFDSPGERIRSMRNDSGANQSDNKRQYELDVDRIM
RGVDSRTTLMIKNIPNKYTSKMLLAAIDESHKGTYDFIYLPIDFKNKCNVGYAFINMTNAQHIIPFY
QTFNGKKWEKFNSEKVASLAYARIQGKTALIAHFQNSSLMNEDKRCRPILFHSDGPNAGDQEP
FPMGTNIRARSGRSRTSSGEENHHDIQTVLTNGDTSSNGADTSGPTKDTE
SEQ ID NO: 20: OML4 nucleic acid sequence [symbol]
CCATTTCAAGTCATGGATCCGAGGCACCACCTCTCCCAGTTCACCAATACAACCGTAG
CTGCGTCCTCCTTCTCTGAGGAGCAGCTTCGCCTTCCCACAGAGGTAATAATCTGCAGTTG
CAGAATTGTTGCCCTATTTATTGTTTTCTGTTTTTGTTAGTTTATGATAAGGCTAGTGGTGTC
TTTATTGTTTTAGTTCATGTTTGATACCTACCATGTTGTCACTCGATTTTCTGGATATCTATG
ACATGCACTAATTTTTTTAATCTATCTTTGCAGAGGCTGGTGGGTTTTTGGAAGCAGGAGTC
GTTGCATCACATTGGTGAGTACTTAATTTGATTCAATACCCCTTAGCTTTTTGCTCATTTCCA
TGCAAAGAATGCTCTTTGGCTGCAAAAATCCACATGTTATTGCGGGGAAATTTTGTGCATTT
AATAACATTTTATGCGTGACTAAGGGCTAGTTTGAATCCACTAGAGCTAATAATTAGTTGTC
TAAAAAATTGCTAGTAGAATTAGCTAGCTAACAAATAACTAGCTAACTATTAGCTAATTTACT
AAAAATAGCTAATAGTTCAACTATTAGCTATATTGTTTGGATGTCTATAGAGCTAATTTTAGC
AGCTAACTATTATCTCTAGTGCATTCAAACAGGGCCTAAATAACATAAATAATTTGTTTGCTT
GTGATGAATATGATTTTAGCTTTTTACCCTAACTTTATCAGGAATAGAAGGCTTTGTTTTTGT
TGTTGTCGTGTTGGACATGTTTTATTGCACTTTTCATTTGTTGTTTATGTATTTATAGTCTCAA
GCCATTGTTTTGTGTTCACCTTGGGTTGCAATGGATTTATGACATATTTGATGTCCAGGTTA
TCCTTTGATATGCAATTGGTTGCGCTAGTTTCTACCTTATTATTCCTTTTTACTTATTTGGCA
CTCCTGTCGTACTCTCTCTTTGTTCTCACAATGGTTCATGCATTTTGTTGTTCATTATCAAGA
TGTCTTCTCAAAGGCAAGCTGTTTCTATTGTTGTCAGGGAGCAAGTCAGTTGCATCTTCTCC
AATTGAAAAGCCCCAACCCATTGGTACAAAAACAATGGGTCGGGTAGATCCACAACCATAC
AAGCCGAGAGGCCAGAAGTCTGCATTTAGCCTTGAACACAAAACTTTTGGTCAAGAGAGGC
ATGTTAACATGCCACCATCTCTGTGGAGAGCTGATCAAGACCCTTATGTTCAATCTGATTCA
TCTTTATTTCCCGATGGAAGGAGTACTAATCCATATGAGGCCTACAACGAGAATGGGCTTTT
CTCAAGCTCCCTGTCAGAAATTTTTGACAGAAAATGTGAGACAGCTTACTCTGGCACTTTCA
TCAACTTCATTAGAGCGATTGATTATACTGCAGTGAGCCTGCACCATGAGAACCATTCTCTT
CATCTTAGAAAATGCATTGAACTGTATCACACATTCCATAGTATGTATTGTGTATGTGTGTG
CCTTGAAATCAACAGAAAGGAATAAAAAGTACAATAAAGGATATTAGTGAGTATGAATGGGA
AGAAAAAATAAAAAAAATACTTAACATATTTTTTTAGCATTTTTGCATCTTATTTTCGAAGGAA
CCTTACCTGCTTTATTTTTCTTTGGCCCAAGAATCCTTTCACTTAAGTTTGGTATCGTTATCC
TTTTATTTTCAGTAACACTTTGTGCAAGATTTGGGCAGTCAGACACTCCGATTAAATCATTG
CTATTGTAGTAAGCAATACATAATTCATATTTATTGCTTTCTAACAAATTATATGCTTCAATGT
GTAGTGGGACTGAGATCAAATGATGTGCTTCTACATCAACCACTTGAAAAGGTTGAACCAA
CTCATGTAGATGATGAGCCCTTTGAGTTAACAGAGGAAATCGAGGCTCAAATAATAGGAAA
CATACTTCCTGATGATGATGATCTACTATCAGGTGTTGATGTTGGGTACACAGCCCATGCTA
GCAATGGTGATGATGTTGATGATGATATATTTTACACTGGAGGTGGGATGGAACTGGAGAC
CGTTGAAAATAAAAAAAGTACAGAACCTAACAGTGGAGCTAATGATGGTCTTGGGTCGCTA
AATGGCACAATGAATGGTCAACATCCATATGGGGAACACCCTTCAAGAACTCTTTTCGTCC
AGAACATTAATAGCAATGTTGAGGATTCTGAATTAAAGGTCCTATTTGAGGTATGTTCCTTTT
TTCTGTTTTCTGCTTAAACCTATCGTTCCTGTACAGAACATTTGTTTCTGAAAATCATTTACT
CTTTACCCACAGCATTATGGAGAAATCAGCAACCTTTACACTGCCTGCAAACATCGCGGTT
TTGTAATGATATCTTACTATGACATAAGGTCATCATGGAATGCCATGAGGGCACTTCAAAAC
AAGCCACTAAGACATAGAAAACTTGACATACATTACTCCATTCCGAAGGTATTCACGAGTCT
TACTGGCTTGATGTGTAGACATATTTTGCCCAAGGATGCCAGTATGTAGCTAGTTTACTGTT
ATCAGTTTTGTAGTTCTTGTGCTAATTTTCACCTTTTTTCCCTTAGGATAATCCTTCGGGGAA
GGATATTAACCAGGGGATGCTTGTTGTATTTAATGTTGACCCGTCTGTAACAAACAATGATA
TCCATAAGATATTTAGTGACTATGGTGAAATAAAAGAGGTATGCTATGCTCTTACATTAACTA
CCTACTACATTATAACTAGAACTATAATGTCTTAAATTAATTGCAGATTCGTGATGCACCGCA
AAAGGGCCATCACAAAGTTATAGAATTTTACGATGTCAGAGCAGCTGAAGGTGCAGTTCGT
GCTTTAAACAGGAGTGATCTTGCTGGCAAGAAAATAAATTTGGGGACTGTTGGTCTGAGTG
GTGTTAGACGGTATGCCTTTGAAATGTTATCCTGCTGTTCATTCACATATTTCAGTAACAATA
CTTATTACTTTTGGACAGTCCATATTTAACTGTTGATCATTTGATCGTGATTCTTGCTTAGGC
ATCTTTGGTATATAGTACCATCACTTATTCTATATGACGGTACCTGTCGATAGAATGCACATT
AGTTGATCTGGATTTTATTTCTTTTCTCAAGTGGAAAATCTCTTCCTGGAGCTGTAAACAT
TGCACTGTTTTTATTTTGTCATGCATAGATAGTTGATCTTTGTTTCTTTATTTCTATGTATGGG
CTCTGATGTCCTACACAAAACAGATTTTTGTTTGTTCTTTCATATTGTAGTCTTATTCTATGTA
TTGCATTTAGGTGTATGGATATATACTTAGTATGTTAGTTATCTAAGTCATCCAGAAAAAAGA
GCAATTATTATGTGACAACATTCTAATTTTGATTTTACCGTGCAAACTTTTGAAAACATTGGT
TTTAATCACTGCTCTAACATTGATTTTAATGTTGTTTTATAACAGATTAACACAGCACATGTC
CAAAGAGTCGGGGCAAGAAGAATTTGGTGTATGCAAACTGGGCAGTCTAAGCACAAATAG
CCCTCCATTGCCTTCATTGGGTATGCTGTTGGTTTTTTTCATCTTTAATGTATGTCATGTCTA
TAGCTACATTTCCTGACATGGAGGATAATTCTTCAAGGTTCATCTTATATGGTAGCCATGAC
ATCTTCTGGCCGTGAAAATGGGAGTATTCATGGTTTGCATTCTGGACTGCTCACATCAATG
AGCCCGTTCAGAGAGGCTTCTTTTCCGGGCCTATCATCTACCATACCACAGAGCCTGTCCT
CTCCCATTGGAATTGCATCTGCTACAACTCATAGTAATCAGGCTCCCCTTGGTGAGCTCAG
CCACTCACTTAGTCGGATGAATGGGCATATGAATTATGGTTTTCAAGGCTTGGGTGCTCTT
CATCCCCATTCTCTTCCTGAAGTTCACGATGGAGCAAATAATGGCACCCCGTACAATCTAA
ACACCATGGTACCAATTGGTGTGAATAGCAACTCAAGAACAGCCGAAGCAGTTGACTGCAG
ACATCTTCATAAAGTGGGTTCTAGCAACCTCAATGGACATTCATTTGATCGTGTCGGTGAAG
GAGGTAAGTTTGTAAATTTGGACATTCTAATCTCCATTTTTATGTTTGAACCCATTGTCATTT
CTATTCCTTAAACATGTGTTTTGTAATAAAGCTGTTAGGTTTATCAGGATTGTGAAAACTGAA
CTGTGAAAATTTGATCAATTAATGTATGTTATTTAACTGTTCCGTTCATGATTGCATCTGTAA
CAAATTTTGCAGCTATGGGATTTTCAAGAAGTGGAAGTGGTCCTGTCCATGGTCACCAGCT
AATGTGGAATAATTCAAATAACTTACAACGTCATCCCAATTCCCCTGTGCTGTGGCAAAATC
CAGGATCATTTGTAAATAATGTACCGTCTCGCTCCCCAGCACAAATGCATGGAGTTCCAAG
AGCACCATCACACATGATTGAGAATGTCCTTCCAATGCATCATCATCATGTGGGCTCTGCG
CCAGCAATCAATCCATCACTTTGGGACAGGCGGCATGGCTATGCAGGGGAATTGACAGAA
GCATCAAGTTTTCATCTTGGCAGTGTTGGGAGCTTGGGATTTCCTGGTAGCCCTCAGCTTC
ATGGCCTGGAGCTAAATAACATATTTTCTCACACTGGTGGGAATCGCATGGATCCAACCGT
GTCTTCGGCTCAGATCAGCGCACCATCTCCTCAACAGAGAGGTCCTATGTTCCATGGAAG
GAATCCTATGGTTCCCCTTCCATCATTTGATTCACCTGGTGAGCGGATAAGAAGCATGAGA
AATGACTCAGGTGCTAACCAGTCTGATAATAAACGGCAGTACGAGCTTGATGTTGACCGCA
TAATGCGAGGGGTAGACTCACGAACTACACTGATGATAAAGAATATCCCAAATAAGTATGTT
TTGAGATCACCAAATTTTATGCTACATTTATGTTCTGTCTCAATATATTCTTTTGTTCTGGTTG
GTTCTTTCGGGTTTCAGGTATACCTCCAAGATGCTCTTGGCTGCTATTGATGAAAGTCATAA
GGGCACTTATGACTTTATTTACTTGCCAATTGATTTTAAGGTAGTTTGAAACTTTGAATTTAA
CTCATAAGCGACCGGGGCCTTGTATTAGTTGAGACTACTTTTGTGTTCATGTTACTAAATGA
GATCAATCTCCTTTTCAGAATAAATGTAATGTTGGCTATGCTTTCATCAACATGACCAATGCT
CAGCATATCATTCCATTTTATCAGGTCAGAAAATTATTCCAATTGACGAAGTGCTACTGCAT
TGATGTAAAGTTGTAAACTAGCCTTTGGTCAACTTATATGCCTTGCCAAATTTGTACTTTGAT
AAAATATCCGGCTTGAACATCGACGTGCTATCCTGAGCCATTTTGTCATCTTTTTCAGACTT
TTAATGGTAAAAAGTGGGAGAAGTTTAACAGTGAGAAGGTGGCATCACTTGCTTATGCTAG
AATCCAAGGGAAAACAGCTCTGATTGCTCATTTCCAGAACTCTAGTTTGATGAATGAGGAC
AAACGTTGCCGCCCCATACTCTTCCACTCAGATGGTCCTAATGCAGGAGATCAGGTATGCT
TATTTCTTTTTTATTTTGTCGTTGGTACTTTCCCTGCTATCTTGTTCTCCAGTTACATTATGTT
TCGCTGCAGTGCACTGTGACGAGTCTTCTATATAATCCATATACCTTGAATCCTTGATGGG
GCTGATGGCAGATAAAAACATAGGTTTTGTGAAAATAAAATGGGGGGAGGTAAATGTCCAC
CTGCCATTTTTGCTGCATTAACTGCCCTGTGACAAGACTTCTCTATACCATCGTACAAAGGC
CCTGTTTGAATGCACTAAAGCTAATAGTTAGTTGGCTAAAAAGTTTAGAGAATTGGCTAGCT
AACAAATAGTTGGCTAACTATTAGCTGATTTGCTAGAAGTAGCTAATAGTTGAATTATTAGC
CAGACTGTTTGGATGTCTGCAGCTAATTTTAGCAGCTAACTATTAACTCTAGTGGATTCAAA
CAGGGCCAAAGTCATCAATATATACCTTGAATCCTTGATGGGCTGATGGCAGCTAAAAACA
TAGGTTTTGTGTGGCGAATCCTTCTAAATTATATGGCCCACATGCACTTGTCTTTATCCCAA
AGACCTCAGACGACTATGCATATGTACCAGATAACTTAAAAGAATTTGTCCCAGTATCTCGA
AGGACCTCGGGAAATCCACTTTACAACCAAGATCGCAAGATTAAGTACACACAAATCACAT
ACCGAAGTTTTGTAGCGGAATTCATATTACAATAAGTTTACAAATTACAATATCGAAAAGGG
CGTACCCAATGCTGTAGGCTTCCCGCACTGTGCGGGGTCTGGGGGAGGGTATCTTTAAGC
GCCAAGCCTTACCCGCATAATATGTAGAGGCTGGGGCTCGAACCAGGGACCTTCCGGTTA
CAGACGGTAGGCTCTACCGCTGCACTAGGCCTGCCCTTCACAAATTACAATATCGAAATGA
GTACAAATTTGATATGAAAGTAATACAACTTTGAATGACATGAATTACAATTTTAAGTTCAAA
ATACATTGCTATCTTAAATGACAAAACTCAGGTGGAAGTACAGAAAATATACTTATATAAGAA
GACCGAGTCCACCGACACTTAGCTTCTATCTACAACAGAACAAGAACATCACTCGCAACAT
GGTGGGATAAAACCCTGAGTACACAAGTACTCCACAAGGCTTACCCGACTAAAGAAAATGA
CTCCAAGGGCATGCAAGAATTGGGGATTCAAGGTGAGGTTATAGCAAGAATAAAAAACTCC
TTTGCATAAAAGCTTACTAGAAGTGGATCCTTAAGCCATATTTGAATTTATCAACTTAGCTCT
CTCCTAAATCTAGATTAGUUTAATCTAGATCAAACACTTGCCAAACCATTGTCTTGATTTTAC
CAGATCTCATTTCTCTTCTTAACTACGATGCACTTAACCCTTGCATATGTCAACCCAATCTTC
GAGTGGTCCAAGACCAAAACGGGTTTGGGCCACCTGATAGCACAGTACTCCACCCTCCAA
CCCATGCTAGTTGGGCACACACTACTCTCCTTAATCGACTCGGACGGAAACACTGCACCGA
GACGAAAACACTGCACAAATCTCATTTTTCTCCTTAATCGACTCAGATGGAAACACTGCACC
GAGACTCCTTTCTCGATGCAAGTTACCCACCCGGTCTCATATTAATTCACCTTTTTCACATT
TCTTTAACATATCTCAATATTCAGCGGAATTGGAAACATTTTCTGAAAACCCCTAATTGGAAA
CATTACACTCTATTTGGTGCATGCAAGGAGAAAAATCTTGTTTCCCCATCTACTCGACTGGG
ACAAATAATCATGCGTCACCTTGTTTCCAGCCATATAAAGAAACGTGCATGCTCTGGTAGG
AAAATGGAGAAGGGCACATGCTTCTACAGTAGGATAGTAGTAGTACGCCTTATTTTTTAGAC
AAAATCTAAAACTTTATACGCCTTGTTTTTTAGGATGGACGAAGTATATAAGTATATATGTCC
AGAAACATATGGATGACTAAATGGACGACCAGCTCGACTAGGGTCGATTAGTCGACCTAGT
CGACGACTAATCACAACTAACAAGGTTTTAAAGTCGTTTGACTAATCGCGATTAGTCGGCCT
TATTGCTGGAGTAAGCACGATTAGGGGCTTCGACTCGACTAGGGCGACTAGGAAGCGATT
AGTCATCCTAGTCACTGACTAATCGTGATTAGTCGCCCGATTAGGGGTTATGTCCGACTAG
CTAGTCCTGTTTTGGGCCAGTAATTCGTCCTTTGTTCTATGGGCCGGCAGGCGGCCCACC
ATCTCTGCAGAAAGTAGAAAACTTGTGTTGCCCTACCTGTACCGCAGTAGCAGCACAGTAG
CCGTCGTCCCTTCTCTAGCGCGCAGTTGCGCACCCTCTGCAGCCCTTCTTCAGCGTGCGG
CTGCGTCCTCTCTGCTCCGGCTGCGATCCCTCTGCTCCTGCCAGCGCGTGGTTGTTGCAG
AGGCCTCTGTTAAGCCGATGCCCTCTAGTATGGCGCACGCCTCTGCTCCAGGACTCCACC
GAAGTCCACCCTCCAGCGCAAGAGCGTCCTCCACTGATCCACTCTGACTCCTCCATCATAC
TTCTTCAGAGTGAATTAGTTTAGAGTTTGTTCTGAACTTCAGAAATCAGAAATCAGAAATTCA
GACTTCAGACTTCGGAGTCCAGAGAACATCAGAGTTCAGACTTCTGAGTTCACAGTTCAGG
CTTTCAGAGTCTGTTGTTTTGCTATAGCTATAATATATTGTTGTCCTGCTACAGCTCTAGTGT
ACTGCTATATTGCAGTACTGCTACAGCTATATATATTGATATATATATTTATACATATAGTCC
TGTTATAGGTGGACGACTATAGGACGACTAGGAGTCTACTAGACTCGACTAATCGAGCAAA
TCGATGACTAATCGTGATTAGTCGCCTTATCGGTGCTCAGGCGACTAGAATCGACTAGCCG
ACTTTAAAACCTTGATGACTAATCGACTGGTCGGTAGCTATACGACTAGGTTCGATTAGAC
GACTTGAAAATAGTTATCTCGAGCAGACTCCCTATCCCACTTCACTCCCTATTTCAAACTAC
ACTATGCAAACAATATAATCTATAGTGCAAAACAGTACTTTGCACGCTCGTTTACATGGTAT
GCTGGAGATGACCTTAGTGCTTGTTAGACGATATTCACTTGGCGATTATCTCCCAACCTAG
CACTTGATCTGTCCATCCATCTTCAGGTTGGTCTGCCGTCATCGTCTTGTGGTTGGCTTTG
ATCCACGTTCTTTACTCCGCGTAATCAACTAACGTACCTGAATGAGATGCACGATGCATATG
TATGAGCATAAAATGAACCAATGCTACAGTGAAGAAAATCAAACACTTAATGGCAAGGCATT
GCCACAATCCTACGCAAGTACTAGATACATATTGTCACTAACCTTGATTAGGCGAGATAATA
ACCCCCTCGGGTACTGTAGCATATATATGTAGGCAGACAAGAATATATGGGCTTTATGGGC
CTTAACACCCCCTATCGAACTCAAGGCGGAAGTGGAGGATTTGAAGCATTGAGTTTGATTA
GATGAAACTGATGTTGTGCCCTAGTTTGTGCTTTTGTGAAGAAATCTGCAAGCTATAATTCT
CAGGGCACATATTGAAGATCAATGGTCTTCTGATGACAATGAGATCTAGTGAACGATACAT
CAACACCAATGTGTTTTGTGAGTTCACGCTTCACTGGATCATGACAAATTTGTATAGTTCCA
ATGTTGTCACAGTGAAGAGGCGTAGGCGAGTCACAAGAAACGCCTAGATCAGCCAAGAGC
CAACGAATCCAGATAATCTTAGCAGTAGTAGTAGCCAGGGCTCGAAGTTCTGCTTCAGTAC
TAGATCTAGATACAACAGCTTGCTTCTTGGATTTCCAAGCAACAGGGGATGATCCAAGAAG
AATACAGTAACCAGTGATGGAGCGACGATCTGTAGGATCACTGGCCCAGGTAGCATCAGA
GTAAGCACGAAGCTGAAGTGGGGAATTTGAGTCATAAAATAAACATTGTGTTTTTGTCCCTC
GTAAATATCTAAGCACACGAAGTAAGTGCCCATAATGAACTGATGTAGGAGCAGATACAAA
CTCATAATCATAGACGGTTTGACGCTGTTCACCATAGCAGCCATCACTTTACCATCATTAAG
CTGCCATGTTTTGATATCAGCAGCATTGCGACGATCATCCGCAAGAACGGGTGCCGCATCA
GTCAAATGAAAGAGTAATCCATGTCCCCTGAGTGCAGTCTCAACACAGAAAGCCCACTCCG
GATAATTTCGGCCATCAAGAGTGATATTGACCACAATAGCATTTGTCGCCATATTGAATTCA
ATGAAAATCAGGGAGAACAGGAGACCTGAAACCAAACAAACCAGAGGACGAGTTGACGGA
GGTCCTGGGCGCGGAAACCGAGTTGGACAGTCTGCTCGCAATGGCAGCCACCGGCGCAG
AAACAGGGACGACGCAGATGGCGACGAGGCAGGGCGCAGCAGACGGCAGCGCAGATCC
GGATCCGCAGACTGTTTGCGGCGTGATTATCGGATCGATGGCAGTTGCACAAATCTTCTCT
GCAGCGACTGTTTGCGGCGTGCAGCAGGCGGACGGCGACAGGGCGCAGTAGGCGGACG
ACGGCAGCCGAGCGCAGCAGGCAGGTGCAGACGGCGGACGACGGCGGCCGGGAAGCC
CAGATCCGCCCGCGGGGATGGAAGAAACCGCGGCCGGGCGCAGCAGGCAGGTGCGGAC
GGCGGCGACCGGGTAGCCCAGATCCGCCCGCGGCAGAGGCGGGGAGGGGAAAAGCCG
CGGCGGCCGGATCTGCCTCGAGGACGGCCGCGGATCTGGACGGGATCCGCGACGACGG
ACGGGCGGTGGCCGGATCTGCGCGACGGCGGACGGGATCCACGATGGCGGCCGCGGAT
CTGGACGAGGGCGGCGCAGATGAGCCCGCAACGACGGAATCCGCGACGGCGGACGGGC
GGCGGCCGGATCTGCGCGACCGCGGACGGGATCCGCGATGGCGGCCGCGGATGTGGAC
GGGGGCGACACAGATGAGCCTGCAGCCGCGACGGCGGGTGGGAGGAAGGTGGAGAGAG
GGTCGCAACGGCGGCCGGGAGGAGACGATGGTGGCTAAAAAAATCTAAGAAACCCTAATC
GTGACCTGCTCTGTTAATAGGTCACTAACCTTGATTAGGCGAGATAATAACCCCTTCGGGT
ACTGTAGCATATATATAGGCGACAAGAATATATGGGCTTTATGGGCCTTAACACATATAACT
CACTAAACACAACAATCACGTTCTTCCAGTTTAACCAGATCTAACTCAAACATCAAGAAATA
ATAAACTATGTGTAAGTCCTATATCTTCTTTAGGTAGTGCCCAACATCAGAAGACTAGCAAA
ACCTAGACTCATCATTCTTAGACACCTAAATTCAGAATGAGAATAGAAGCAATCTAACTAGC
ACTCTAAACCACCTTTTGGTGAAAGAGTAATTGTGGGAATGAGTTGATTCTATTCCACGACA
ATGTGTGCGTATACATAGGAGAGGCCGGGGTTGCTCACAAGGCAACCGCACAGGCGTACA
AGCCAATCAAGGGCAGCCTACAATCAAGGGCTGACTACCATAATTAGGCTTTCTATAATTA
CAATAGTCTAACATTTGGGACTAACTCGCATAGCACAACATCTAAATAAAACATCACACTAT
TAGATCTAGCAGGCAGAACATCATTAAAGATCACAGTCTTTCACAAAACCACAACTTAAAAC
CAAAAGACCTAAAACACTAATGTGCAATGCCCACTATGCAGTATTAAGATTTCAACTAAAGC
AGACCTAGCGATGTTATTTGCTTCGAGATACTTGGAGAAGCAATCAACATCCATCTATGACA
TTTAACCGGTCACTAAGGCCCTGTTTGGACAGCTCCAGCTCCAGAAAATTCGGTAGAGTTG
GTGGAGCAGGTCATTAGGTGCTCCATAAAATCGTGGAGTTGGAGCTGTAAGCCTTCAGAA
GACATTTTGTCTTTGATAAGTCATGCCCCCGCAGTCTAATCGGGAGCATCGCTAACGGTCA
GGCTGGACCGAAACTCCTGGAACAACGAGGTGGGTGGTCCCTTGGTGAAGACATCTGCGT
ACTGAGATGTCGTAGGAACATGAAGAACCCGAGCGTGTCCGAGGGCGACCTTCTCTCGGA
CAAAGTGGAGATCGATCTCAACATGCTTCGTCCGTTGATGCTGCACTGGATTGCTGGAGAG
ATATACAACACTGACGTTGTCACAATAGACTAGGGTGGCACGGCGAGGCGGGTGCCGAAG
CTCAATGAGTAACTAACGTAACCAAGTAGCTTCAGCAACGCCATTTGCCACAACACGGTAT
TCGGCCTCGGCACTGGACCGGGAAACCGTGTGCTGGCGCTTGGAGGACCACGACACTAG
GTTGTCTCCAAGGAACACTGCGTAGCCAGAGGTCGACCGGCGAGTGTCGGGACATCCAG
CCCAGTCGGCATCTGTGTAGACAACGAGCTTCGTAGGGGAAGATCGGCGCATAGTCAGTC
CAAGAGATATAGTGCCTTGCAGGTAGCGCAAGATGCGCTTGAGAGCCGCGAGGTGGGGC
TCTCGTGGATCATGCATATAGAGGCAAATCTGCTGAACAACGAAGGCAATGTCCGGACGG
GTGAAAGTCAAATACTGTAGAGCACCTGCCAGGCTGCGGTACTGAGTAGCGTCATCAACG
GGAGGTCCATCTGCAGATAATTTGGAGTGGAGATCAACAGGTGTGCTACACGGCTTGCAC
ACGCTCATCCCGACGCGCTCCAAAATATCCTGAGTGTACTGTCGTTGAGAGAGAAACAGAC
CATTGGCAGAACGTGTCACAGAAATGCCCAAAAAATGGTGAAGCTGACCCATGTCCGTCAT
AGCAAACTCACGCTGGAGAGCCCCAATCACATACTGAAGAAACTTTGCAGAGGAGGCAGT
GAGAACAATATCATCAACATACAGCAGCAAATAGGCAGTGTCTGGCCCTTGGTGATAGATG
AACAGTGAACTATCTGACTTGGTTTCAATAAATCCAAGGGAAAAAAGATGGGATGCGAACC
TGTGATGCCAAGCACGAGGAGCCTGCTTCAAGCCATATAGGGATTTGTTGAGCCGACAGA
CAAGATCCGGATGAGAGGAATCCACAAAACCAGAGGGTTGTACGCAGTACACTGTCTCGG
TGAGGGTGCCATGTAAGAATGCATTCTTCACATCTAGCTGATGGATGGACCAGTTCTGAGA
GAGAGCCAACGAGAGAACAACTCGAACTGTTGCAGGCTTGACAACCGGACTGAAAGTCTC
ATCATAATCCACACCGGGGCGCTGGGTAAACCCACGGAGGACCCAACGAGCCTTGTAGTG
ATCAAGAGACCCATCTGCGAGCAGTTTGTGTCGAAAAATCCACTTGCCAGTTACCACATTG
ACTCCAGGAGGCCGTGCTACTAAACTCCAGGTGTCATTGGCGAGTAGAGCATCATACTCA
GCTTGCATAGCGGAGCGCCAATTGGGGTCTGACAATGCATCACGAACGGAGCGAGGCAG
TGGCGACATAGACACAACGTGGAGGTTGAGGCGATCCACGGGCTGTGCCATGCCGGTTTT
GCCGCGAGTGTGCATGGGATGCGCATTGGCGATAGGGGTGATGGAGACCGGACGAGTGT
CTGCCGTGCGACCGGTGGCCGCGGTGCTGCTGGCAACGGCCTGTGCAGGATGCACAGG
GGCAGCATCACCCGTCGATGCGGTCGGCGAGGCGGCGGGGGCACTGCTGGCAGCAGCC
TGTGCAGGGTGCACAGGGGCAGCAGCGCCGGTCGACGTGGCGGAGGGACTGGGCGTCC
CCAATCCAGTGGGAGGAGACGTGGGTGCCTCTACACGCCCATGGGCGACGTGAGGTGTA
CCTGCATGCACAAGTCTTGCTCCAGGAATAGGAGCGGTTAGATCATGTTCATCAAGAAGAA
AATCCAAGGCGGAGGATGCCATGGGAGTGGTAGACATGGCTGCGAAAGGGAAGAAGGAC
TCGTCAAAAACGACATGTCTAGAAATAAGAATGCGGTTCGACTCAAGCTGGAGACACCAAT
AGCCTTTGTGTTCCGAGGAATAGCCGAGAAAAACGCATAAGGAGGAGCGGGGGGCAAGTT
TGTGAGGTGCTGTGGAGGACATGTTAGGATAACAGGCACTCCCAAAAACTTTAAGATGATC
ATAGGAGGGTTGGGAGGAAAAGAGGGCACTATATGGTGTGGAGAAAGCAAGGGTTTTAGT
GGGGAGCCGATTCACAAGATATGTCGCAGTGTGAAGGGCTTCAACCCAATAAGCCGGAGG
TATACTGGCCTGAAACAAAAGAGAACGCAGAATGTCATTTATGGTGCGAAGAGAACGTTCT
GCTTTCCCATTTTGCTGAGAAGTTTAGGGGCACGACATGCGTAAGACAATGCCGTGGGAG
AGAAAAAATGTGCGGGCCTGGGAATTATCAAATTCACGGCCATTGTCGCACTGGATGCTCT
TGATGACGGTGCCGAATTGGGTGCGAATATAGGTGAAAAAGTTGGCAAGGGCGGAAAAAG
TCTCGGACTTTAGACGGAGTGGAAACGTCCAAATGTAGTGGGAGCAGTCATCAAGAATTAC
CAGATAATATTTATAGCCCGACACACTAACAATTGGGGAGGTCCATAAATCACAGTGTATTA
AGTCAAAATTGTGAGAAGCTCGAGAGCTAGATGAACTGAATGGCAAACGAACATGACGACC
AAGTTGACACGCATGACAGATGTGGTTGACATCATCTTTATTACAGGAAATAACACTGGAG
GTAATAAGTTTGGACAAAGCTTGATGCCCAAGATGACCGAGACGACGATGCCACAGGGAG
GTGGGTGCAGCGAGGAATGCAGGGGTGCTGGTGGAGGGTGCATAGAACGGGTAGAGGTC
ACCGGAGCTATTGCACCTGGCGATCACGTTCCTGGTTTGCAAATCCTTCACAGAAAGGCCA
AAGGGATCAAACTCAATGGAGCAATTATTGTCGGTGGTAAAACGACGGATAGAAATTAGAT
TCTTAATAATGTTAGGAGACACGAGGACATTATTGAGAACTAAATTGTGATGCGGGAAAGA
AAAAATATGTGATCCAGTGGCTGTGACAGGAAGCAAGACACCATTTCCCACAATGATAGAT
GGAGTGAATGAAGTGGGCAAGGAAATGGTGGAAAGTTTACCAGCGTCCGAGGTCATGTGC
GATCCTGCACCGGAGTCGGCGTACCACTCTGAAGTAGCGTTCGGCGGGTTGAGGGTCAT
GGTGTTGAAAGAGTGCAAGGGCGTCCTGATGCCATGCTCCTCCGTGAGTGGGGTTCCAGG
GCGCCGCTTGGTACGCCGGTGCGGGCGCCTGGAAACCAGGGGCTCCAGTTCCCCCGTAG
TGCATACCGTACGGAGGGGATGGAGCGCCGTAGAAACTGCCATAGGCGTTGTATTGTGGT
ACTGCGTTGAATGCTGGCGGCGGTGGTGGGCGCCCGGACTGATCGTATGGCCACAACCG
TACAGTGCCAACCCAAGGATGCGCGAAGGACGGATGCATGCCGGGGGGCACCTCCCTGT
CTGGCACCAGGAGGCGTTGGTTGGCCCTGGTGATGGCCGTTGCGTCCACCGCGGCCGCG
ACGACGGCCGTTACGTTGGCCGTTTGGCACCGAGGGGCATGCTGGAGGATGTGCCCCTG
GACGTGGAGGTGCTGGAGCCCCGGGAGCCGCAGCTCGCAGCGTTGCAGCGACGAGGGC
GGATGGCGGGGACGGAGGTCGTGCGTCGATTTCCAGCTCCTCCAACAGCAGGTGCGCTC
GTGCCTCTGCGAACGTGGGGAACGGCCTGTGCATCTTGAGGATGGACACCATCTGGCGG
AACTTACCGCCGAGGCCGCGAAGGAGCGTGAGCACCATCTGCCGATCGTCGATGGGATC
GCCGAACTCGGCAAGGGAAGCCGCCATCGATTCGAGCTGGCGGCAGTAGTCGGTGATTC
TCAGGGACTCTTGGCGGAAGTTGCGGAATTTTGTTTCGAGCAGAAGCGCCCGAGACTCCC
TCTGGCCGAGGAACTCGTCCTCGAGGTAGCACCACGCCCCGCGAGCGGGGCCCTGTCGC
ATCATCAGAGATTGCTGCAGGTCACCGGAGACGGTGCTGTAGATCCATGTCAGGACGCAG
CAATTGGCTTGAACCCATGCCGGGCGCGACGGGAACGCTTCATCTTCAAGGACGTGACGA
GTCAGGGCATATTTGCCAAGGACAGTGAGGAACATGCCACGCCACTTGGTGTAGGTATTT
GTCGCCTGATCGAGAAGGACAGGGATGAGCGCCTTCACGTTGACGACGGCAGTGGCCTG
CGCCCAGAGGGCCTCATGGGCATGCTCATACGCGTCCAGAGCGGCAGCACGGAGGCGG
CCTTCCTCGGCGCGGCGGGCGTCTTTGGCACGGTGTGCAGCAGCCTCAGCCGTAGGGCG
CTGGTCCTCGGCGGCAGCGTCGTGGTCGTCCGCCATCGGGAGGGAGACGCGACGGCTG
GGCAGCCGAGCTGGAGCCGCTGCAGACTGGACGGGAGGGAGGCGCGACGGGGATTAGC
GTGGTCTGGAGGCTCGACCGCGCCCGACCAGAGGGAACGACCACGCGATCTGGACGGG
AATCAGCCGAGGGAGACGCGACGGGGATCCGCCGATCTGGTGCCGCGTCGGATCAGCGA
GCGTGGCGGGCGAGCGGATCAGCGGCCGCGCTCGCGGTCTGGAGCTGCGACCGCGCGA
CGAGGCGGGTGAGCGGATCAGCGGCCGCACCCGGCAGCAACAACGACGGGGCGGGTGA
TCGAACGGACGGCGCAGGCGATGGGATCAGCGACGCTCCAGGCGACGAGGTCTGCAGG
GGCGGCGATCGGATCGGCGACGGCGCGGTCTTGGGTTGCGGAAGTGTGGTGGATCGGA
ACCTTGATACCATGAAAGAGTAATTGTGGGAATGACTTGATTCTATTCCACGGCAATGTGTG
CGTATACATAGGAGAGGCCGGGGTTGCTCACAAGGCAACCGCACAGGCGTACAAGCCAAT
CAAGGGCAGCCTACAATCAAGGGCTGACTACCATAATTAGGCTTTCTATAATTACAATAGTC
TAACATTTGGGACTAACTCGCATCGCACAACATCTAAATAAAACATCACACTATTAGATCTA
GCAGGCAGAACATCACCAAAGATCACAGTCTTTCACAAAACCACAACTTAAAACCAAAAGA
CCTAAAACACTAATGTGCAATGCCCACTATGCAGTATTAAGATTTCAACTGAAGCAGACCTA
GCGATGTTATTTGCTTCGAGATACTTGGAGAAGCAATCAACATCCATCTATGACATTTAACC
GGTCACTAAGGCCCTGTTTGGACAGCTCCAGCTCCAGAAAATTCGGTAGAGTTGGTGGAG
CAGGTCATTAGGTGCTCCATAAAATCGTGGAGTTGGAGCTGTAAGCCTTCAGAAGACATTT
TGTCTTTGATAAGTCATTTTGATTATTATTTAGGTTAAAAATATTTTTTAAAACTATTTAAATTA
ATATTATAAACTATAGCTCCGCGCTGGAGCTGGAATTTAGAGTCATCCCAAACACCAACTAA
ATATAGAGTATAATGACCACTAGAGCAAGGCATCGACTTTATCAAATAAATAAAATCGACAC
AAACAACACTGAGAACATGTTGGCTAGCCGATTGAAATACTAAACCTATCTTTCACGTCATC
AATTGACAATACATTGCATACTTGTCTACCAAAACACTCTTCTAGGAGATGGTATCATTCTC
ACTGTTTCCAGAGCAAGTTTGGTACATAGTTTGCAAATCGCACCATACTTAAATGGTCCCAG
TGTCTGCTTAACAATTTCAGAACTTGCTGTATTTTTGTGTTTGCAGTTCTTCTAAGCACATGG
TTGTAATTTTGAGATTTTGTTGTGATCTTTCTCAGGAACCTTTCCCTATGGGTACAAACATCC
GAGCCAGGTCTGGGAGATCCCGGACTTCCTCTGGTGAAGAAAATCACCATGATATCCAGA
CAGTCTTGACCAACGGTGACACTTCTTCCAATGGAGCTGACACTTCAGGTCCCACCAAGGA
CACTGAG [symbol] CTGAACTGCAGCTTGCTGCGTTGCTGACCACAAAGGCCCAAACTATAACTT
TTTGCAAACCCATTTTCAGTTCTTTCCCCCCTTTCCCATTTTGGTTCTGTTTTGTAAAGTCTC
CCGATCTGTATTTATTGACTTCCACGATGCGGGTCACCGAAGACTTAGGTTGCTGCAAAAT
TTTGTCCCTGACGGGAAGCTATATGCAAGAGGGTGGTACTGGCTATGTGCTTGTTAACCTG
AAGGCCGAGAAAGGTGAAAAGCGCAGGGAGAGCCTCCAGATTTTGGTCGCTGTAAGAATT
AACCCCATGTTGTACAGCAGGTCCCAGTAACTTGTAGTGATGGGAGAGTGGAGTCATTTTC
ATCAGTTTTTAGTGGTGGTTGTGTGGAGAGGAAGAGTCTTGCCTGCGTTTTCTTTTGGAAC
CTTCTCTTGTGCCTTTACATTTTTTTAGTCGAGGGTTCCTCTTAAATTGTGTGCAGAGGGGG
CTCAATTUGTTAACCGGAACAAGGCGCATGTGCGTCTTGGATCAACCCCGGTCTTGTCTT
CAGGCACTGTTACCTTATTTATCAAACATATGTACACCTCCATCTATATATAGTATGAGTTTT
GATGCCTATCTATTTTGTGGCTGTCGTCTCACAAGGTTATTTATCTATATATAGTGTGAGTAA
TTCTTGTTCAAATCCTTTCTCCTTACTATAAATATTTGTCACAATACGCGATCGCTCCCAATA
ACTGCTATAAATATTTGTCTCCGCCGTGGCCTCCATCCCTAAACGGAGCACAGAGCCAGCC
CCACTCCCTTTCTCCTTACTCCGACAGGAGATGCGGATGCCGCCGAGGGCCGTTCCACAT
GGCCCCTAAAAACAGTGGGGTCCTAAGCTGCTGGACACTAGCATTTTCCCTATAGTTTATC
TGCTTTATAGTTTATCTACTTTAGACACAAATACGCAAAGAGCATCGCACTGTCATCCTGTC
TTATTTAGATTGTTATCCTAATATCTCAATTGCTTATCAAACATTATTTACTATACCCACGATG
GTTATATTGGTTGAGAGACTTTTTAAAATTGAAATTATTGGGGAACTATTTAAGGCCTGCAAT
GATTGAAGGAAGATTAAATAGTTTGGCAATTCTATGCATGGAGAAAAAGTTGGATGCTATTG
ATCTCAATGGTATAATCTTTGACTTTGTATCACAATGTTAGAAGACATTTTTAGCGTGATATG
AATGAGACGTGCGACAGCGCAGCCACACAATAGCACACACTTTTATACGG
SEQ ID NO: 21: OML4 promoter sequence
CATACTTGTTGGCAAGAGCGCCAATCACGGTGCCTCAAAACAGGTTATTGACAACGTCGAA
CATTCTCTCCTCTTCAGGAGTGAACTGTTCGGGTTTCCCCTGTGCGGCGTGATAACAGTTC
ATTGCAGCCAACCACAATATCATCTTACTACGTCATCTTTTGTAAAATGTCCTATCAAAAGGT
TCACTTGGTTTTAAAGTAGCAACAAAACCACTAACAGAAAAATGCCTAATATCAGGTTTTTG
GATTGTTAGAGAAATATGCATTTTCAGTTTTAATTTAATCCAGAAAATCACAGTGATGTATGT
GATGACATGTATGTGCATATGTGTATCACTACTCACATAAGTTGTAAACAACAGTAAATTATA
CACAAATACTAAGAACAGAGTGTACCCTGTGGAGGGACCGATGTTGCAAGGCATCAGTGG
CTCTATTCACACGAGACATCTCATGTGTATGTTCGATGTAGTCATACGCAGTCGATGTAGAC
AGATGTACGTAGTGCAGTCCCTCGAACGACGCCGGCGACGAGGAACTTGATCAGCGTTGA
TTCAGCGGACGAAGCGAGCAGTCGTGAGTACGCTCCCCAAAAACCTAATCGTCCGCACAC
CTGTGCAAGTAACAGACAGCGATTTCGGAGGCCTGCTCTCCCAAACTCTCTGTGCTCGCA
GAAGGTGGGACGAGAATGGCTGTGTGCAACGCGTCTGAGACTCTACGTGCGTACTGTGAA
TAGAAGCAGCCTCCACTCCTCCATATAAGTACACGCGCAGAGGGAGGTGAACAGACAGTA
ACAGTCACCATCAGAGCTACCGTTATAGACAGCCAGAAATTGATACCATTAGTGACGTCCG
TTACTAGCCGACAACCATTACAGCCCGTCCGTTATAGCCATAACACAGGAAACAACCAGTA
ACAGACGATAATAATGGACTGTCATTACTCTAGGCAAAATATGCAACCCTTAGGACGGAAT
ATTCGGATCAAAGTCCGATCCACCACGGCCCCGCCGGCGGCGGCGCGCGCGCATGATAG
TCCTTCATCATTTTCTCAGCTTTATCAATAGATGCACCAATGATACTTCTATTTAAGTTGATT
GAATTGTCACTTGAACTTCCGGTATGGTACTAAAGTACTAGTACACTGTAGCATTAAAATGA
GCCTTTAACATTAACTATTATTGAATATTAATTTGTGCCAGACCCACATTAATTCAACAGTCG
TTGCAACTAGCCATTTTTGGATCCAAAAAATTTAAAAAAATTGCAAAAACCACAAATTTCACC
CCAATCTCTTTAGAAATACCCTACGCGGATGGAGCTCGTTACACAAACCATTCCATTATGTT
GTGCGATTTCTGAGCGTTCAAATAAACGTGCGTGAATTACTTAATTCTGAAATAAAAAAGCT
ATAGAGGCTGTAGTCTGCTACAATCTATGTACTAGAGCATTAGAGATGAAGTGAAGTCGAG
AGCTGATATGATATGGACGAGAGGAGGATGCTGCACTAGAACGAGGCTAATCCAAGCAGT
GAGTGAGAGGAGAACAATCTGGCGCAAGCAAGCAAGCAGCAAGGCTTGCCGCCCGTCCT
AACCAACTCAGCCCAAAGCCGTCGCCTCCCCCAACTCCCACCACCCAAATTTGAACCCAC
CGCACACCAATGCACCGCTCTCTTCCGTCGATCCCACTGCAGTACTGGTCCCACCCCTGT
ATCAAGTCACTGACAAGACAGCCCGCCTAGAGTGGGCCACATCTCGTCAGTTTCAGGTGG
TATGAACAAGCCCCAGGACAGCCGCGCGCGCCGCGGTCCCGTGCTCCGCGGTGGCAGTC
ACCGGGCGTAACCGCAGGGTACGGTATAAAGGGCGTGCCGCCCGTCTCTTGCCGCCCGC
ATTTGGGTAGGTAGTTGCTGTTCCCCTGCGAGGCCCGCGTCTCCCCTTCGTCTCAACACC
CACCGCCTCCTCGCCGGAGCCAGTAAGCTTCCGGCGAAGAAATCCGGCGGGCACATCAC
AAGGGGGCCGAGCAGGGGGACCTAGGCGAGCGCCGGAATGGGGGCCAGCGCCCGGGG
GCTCGCCGTCGTCGTGGCTCTAGGGTTAGATAGGTGTCCGTAGCTTTTTCTTCGCTCGCC
CTCCCCCACGCGGCCAGGGTGTGCAGCCCCGGCGTCGCATTGGGTCCCGGGACGACCG
TAAGGCCGCGTGATCCACCCGTGCTCGAGCTCGGACAGGGTCCCGGTTGCGTGGCATGG
GGGCATCTCTTCCGCCTGCTCCCGCTGCCTGCGAGTTTGGCACCGTTTTTGAGCTCTGAA
GAGGAGGAGGTGGTGGCAGCAGGCACCGATCTGGTGAGCCCCCACTCGCTTTCCGTTCT
CTACATGGATGGTTTCTGTTTGGGATGTTTCAATTTTGGGAAATTTTGAAAGCTCTCGTATA
AGTCGTTTTGTTTCGTGGGTGTCCTTGCTTGCTGTATGTAACCTGAGCTTGAATTCGGGGT
CTGACAATTATTTTGGGTTGTGTTCTGCCGGGAATTCCTCGTTTTATTTTGATGGTTTCTTTG
ATCACTAGGGACTTGCTGGTTTGGAGCTCGTAGAGCCCGAGGCGCATTAAATTTTACATCT
TCTGTGCTGTCGTATTGGGGGAAATTAAACATTTCTCTCAAATTTGTGGGATTCGCACTCTG
GTTTGTCAAACCTACTGGTTCTGATTCAGAAGTATTGACTTTGGAAGCTCACACGAGCTAAA
ATCCGCCTTTTTCTCTGCTGCCCTGTGGCTCGGTTGTCATGGATTGACAGATTTCTGCCCG
TAAAATTGCTCCTATTCGTCATGTTAACCCCTCGACACTTCATCTTTTCCGCAAGTTTTATTA
ATTTTGCGTTGATCCTGGGCAATTGAGATACGGTGCTGTTGTCTAGGTTTGTGCCTAACAC
GTTATATGGTCTGGACGCCTGCAGG
SEQ ID NO: 22: GSK2 amino acid sequence
MEAPPVPELMDLDAPPPAAADAAAAAPVPPAVSDKKKEGEGGDTVTGHIISTTIGGKNGEPKR
TISYMAERVVGTGSFGIVFQAKCLETGETFAIKKVLQDRRYKNRELQLMRAMEHPNVICLKHCF
FSTTSRDELFLNLVMEFVPETLYRVLKHYSNANQRMPLIYVKLYMYQLFRGLAYIHNVPGVCHR
DVKPQNVLVDPLTHQVKLCDFGSAKVLIPGEPNISYICSRYYRAPELIFGATEYTTSIDIWSAGCV
LAELLLGQPLFPGESAVDQLVEIIKVLGTPTREEIRCMNPNYTEFRFPQIKAHPWHKIFHKRMPP
EAIDLASRLLQYSPSLRCSALDACAHPFFDELRAPNARLPNGRPFPPLFNFKHELANASPDLINR
LVPEQIRRQNGVNFGHTGS
SEQ ID NO: 23: GSK2 nucleic acid sequence [symbol]
GAGGCGCCGCCGGTACCGGAGCTCATGGATCTGGACGCGCCCCCTCCCGCCGCAGC
CGACGCCGCAGCCGCGGCGCCGGTTCCCCCCGCCGTCAGCGACAAGGTGAGCGAGTGC
CCCAGATCCGGAGCTGGGCTCGGATCTGCGGCCGTGGTCGCGGCTGGGCGCCTCCCGAT
CTGCTGCCTCCGCGAGCGACGTTGCTAATGGTGGTGGCCTGTCTATTTTTTCCTCTCTCAC
TTTCCGTTTGTGTTGCAGAAGAAGGAAGGGGAAGGGGGAGACACTGTTACGGGTCACATC
ATCTCCACCACCATCGGTGGGAAGAACGGCGAGCCGAAGCGGGTAAAGCTACGCTTCTCT
CGCTGTCTGTTTGTCTATCTGTCGTGCCGATGTGCGCGTGAATGCTGCTGCGGTTAGTGC
GGCTGAAGTGCCCCCGCTTGTTTCGTAGCGGCCTTGCGGTCGGAATCCGTTTTGATCTGA
CGGTTTGCGCATGGGGTCGTGTTCTGCGCCTCTTGTTTAGCGGCTACACAGCTACAGCTA
GCATGCTGGTGAAATTTGGTGGGTTTGTTCTGGTTTTGTTGATGTATTATGCTCTCCCCGCT
ACTCTGGGCCTCTGGGGATTCTGGCTGGGTTGCGCTTCCTTGGCTTAGTGTTTGCAGCTG
AATTATGTGTCTGACCGCTTCATTTCGTGCTTCGTTACTTGGTTTTTTAAGGCTAACATGCAT
TTAGGAAGCACGGTCTACCATTCTTGTGATTAGTTCTGCCGTGTGCAGAACAGAAATGGTC
TAACTGTTAGTTTAGGTCCAGGTATGAGTGAGGATTCGAATTCCTTCCTGCTCAGTTGCTCT
GACGCCTGCCTAGTTTGTTACCCTCTTCGTGTCCTCAGTTGCTCATTTGTTCTTCTTCTGGC
CTTAATTGCAGACCATCAGTTACATGGCAGAACGTGTCGTGGGTACGGGCTCATTTGGGAT
CGTCTTCCAGGTATGGTGCTTGGTCATGGGAGCTCTTCTTTGTACGTGCCTAACATTTGTT
GATGTAACATGCACTGAATTAACTTTGACATGTAGGCTAAGTGTTTGGAGACTGGAGAGAC
CTTCGCCATTAAGAAGGTGCTGCAGGATCGGCGTTACAAGAACCGGGAGCTGCAACTTAT
GCGTGCCATGGAGCACCCCAACGTCATCTGCCTGAAGCACTGCTTCTTCTCAACAACGAG
CAGGGACGAGTTGTTTCTAAACCTTGTCATGGAATTTGTCCCCGAGACCCTGTACCGTGTC
CTGAAGCACTACAGCAACGCGAACCAGAGGATGCCTCTTATCTACGTCAAGCTCTACATGT
ATCAGGTTTGTGAACCAGCATCTTAACTTATATGAAGCTGCTAATGTGTGCTTTCATTGTTTT
GCTAACTGTCTCTTTTTTTGTAATGTTCGCAGCTTTTCAGAGGCCTAGCCTATATTCATAATG
TACCAGGAGTCTGCCATAGGGATGTAAAGCCACAAAACGTTTTGGTACGTGTCATGTGGAC
AAGGTTTCGTTCTTTCTTTGATTTGGTAACTAGTTCTGAGTTGGTCTGATCCTTCTTTGATAT
ACAGGTTGATCCTCTCACCCACCAGGTCAAGCTCTGTGACTTTGGTAGCGCAAAAGTCCTG
GTATGTTGTTTTTCTTTCCTTGAGGATTTGTAGTCACATCCAGTTGTTGTATGCTTTCTCTTT
TGAAATATTCTTATCAAAGGCTTGTTTTTCTTTCCTTGAGGATATGTAGTCACATCCAGTTGT
TGTATGCTTTCTTTTTTGAAATATTCTTATCAAAGGCTATCCATACTATTGGCATGGCATTAG
TGGTTTGTGTCACTATGTAAAATGTATCATCAGTATGCTGCTATTGCCTGTTATGATTAATTG
TGATTGTAGTTGGTTGGTCCTATGGAACAAAACACATCTTGAAGGTAGCTTAAGTATAGATG
CAAGGCTCGTGGATATATTTCTCAGTGAACTATTGATACAAAAACTGTCCTGTTACATAGTT
TTGGTCTAGATATCTTGCAGATCAATGTTGGCTACATTTTAGTCAAGCTTATCAAATTTGTCT
TCATCATGTGCAGTTATATTTATCCTATTTGAGCTATGACTTATTCAATTGTTTCTGGGGCTG
TCTTTGTATGTAATAGCATTGATTCTTTTGCTTCTATCCGAAGTTCCAATCTTGGAATTGACT
GTAATTCATGTGTTATAACAATTAGATTCTTTACTTGTATGCTGTATTTTTTTATCCACATACT
AATCAGTTCCATATGTTGTTTTGTCAGATTCCTGGTGAACCGAACATATCTTACATATGCTCT
CGTTATTATCGTGCTCCAGAGCTCATATTTGGAGCGACGGAGTATACAACTTCAATAGACAT
ATGGTCAGCTGGCTGTGTTCTAGCTGAGTTGCTTCTTGGTCAGGTTGGTTGCATTCATATA
ATGATTAACCTAATATTTTTGTACCTCGATTTGACCAAATCATGTATGGTGTGATTCTAACCT
CTTGGGGTCTTGGATCTTATGTATCAGCCACTGTTTCCGGGAGAGAGTGCTGTTGATCAGT
TGGTAGAGATTATCAAGGTACTGCAAAATGTTCCAAAGTAGACATTCTATTCTTCTACCGGG
GTGTTTCTTATGGTTATGTGATGTGCCTGTAGGTTCTTGGTACTCCAACCCGTGAGGAGAT
ACGATGCATGAATCCCAACTATACTGAGTTCAGGTTTCCTCAGATAAAGGCTCATCCGTGG
CACAAGGTATTCTTATGTTAAAATCATGTTTCTGTCCACATCTATGATTCCATTTCACCAGCA
GCTACTGTAGTTATATACTTGTAGCCCACGGTCCAAAATGTTATTGAAGGGCGCTTAAAGAA
ATTGTTTGCAGATCTTAGCGAAAATTTGAGCTCAGAATGCATCAGTTACTGACTGATTGTTC
CACTTTCCGTTTTATCCTCCAGATTTTCCACAAGAGAATGCCTCCGGAAGCCATTGACCTTG
CTTCCCGTCTCCTTCAGTATTCGCCAAGTCTTCGCTGCTCTGCTGTGAGTATTTTTTTTTAC
TTTGTTTATTTAGATTAGAGTCAGCTTTGATGCTTATAGTTTGAGGGGAATAGACAATGGAA
CGCGACAGACTAAGGTCTTTGAGGTCTTTGTGTTGCATATGGTCTATTTTACTTGGCTTTGG
TTATTCGAAAGCTTCCACTGTTGTCAATTATCCGTAGTCCTGTAGACCATGACCAATTAAAA
GTGGCTAAAATCCATGTGGAATTATGTCCTCTCAAACCATAGCGTATGGTCCTGCATGTATA
TGGTAATTATGCTGCCCAGTGGTCCAGAAGGCTAGTAGAACCATCAGTTTTGATGGATGTT
AGCTGATGAAGAGTGGGTGCAACTTTATAGTCACATGTGCTTGTTAAGTGTACTAGCTAGT
GGCCCACCTAAAAAGAGCAGTCCTAGTTCTCACATGCTGTAGGGTGGCACACACCATAATC
TTTAATGCATCAGTTTGTTGGTTAGAAATGTTCTAATGTGCTTCATGTATTCTATTTCTATTCA
GCTTGATGCATGCGCTCATCCCTTCTTTGATGAGCTCCGGGCGCCGAATGCACGTCTACC
AAACGGCCGTCCATTCCCTCCGCTCTTTAACTTCAAACATGAGGTAAGCAAACTAAACACA
GTGCAAAGTTCGTTTAGCCAGACGCTTCAGTTCGGTCATTAAAGACCTGAAAGGATCCAGT
TTGCACTTGCTCTACTGTTTTGCTCATCAGTTACCCCCCCCCCTTTTTTTTTGCTATGCAGC
TAGCAAATGCCTCCCCGGACCTCATCAACAGGCTTGTACCGGAGCAGATTAGACGGCAGA
ACGGTGTCAACTTTGGGCATACCGGGAGC [symbol] GAGGGCAGGCGGCTGCCATGGTCAAGT
TTTTGGTCTTGGTACCCCATGTGCAGGGCCGATTGCAGGTGACGGTGATATTGCTGCACC
ATCTGGAGAGGAGGGGCTCAGCAGTACCTGAGAGAGCTGAAACTATGTAAATTATCTGAC
CGCGAAGGAGTACGGCCAGTGTTAAGCCAGTAAACTGGCGCATGTTGGTCCAGAGTAGTT
AAGAATGTAGCAGGTGGAGACTGGTAAATGCCTAGGGTCGTTTTTAGTTGTTGTTACTAGT
ATTTTGTAATGTAATGTTCGTCGGTACTTCCCAGCAGTAGTGTAGCTGCTCATGTTTTGTTC
GCCCGTCATGATGTAAATGATCATCACCCAACTGGAACCCCTGTTATCTCGTTACATGCTTA
GGCCTCTGATCGTTCCGTAGTTGCTGTGATAGAGCTCTGACAAGTCTGAGCAGGAAAGTG
GGTAGGAATTGCTTCTGGTGAAATCTGGACAAGTTTTGTCGAATACAGATGCATCTGCTGA
TTGATCGTCTGGTTGCAAGTAGTCTGCACATTCCCAAGGCCACAGATCATTACTTTCAGATT
GTTGATAACGACCAAATGGCAAGTAACAGAAACGACCGAAATTCGCAAGCAGGCAATTACA
GACGCGGCCGCGCCAGCACATCGCCGCCGTAGTCCTTGACCTTGCTCCACAAAACCGGC
CATGCGTCCTTCCTGATGGCGGCTAGGTTCTCTCCATCCCAGTCTCTGGCTGCGAGCTCC
TCAGCCATGGCGACCGCCGCGGCCACGACGTCCTCCACGCCGCCGTCCACGGCCGCATC
CACGATGCCTCGGCGCGCGGCCTGCGCGGCCGTCATCTTCTCCCCCTTCATCACCAGGTC
CCTCCTAGCGGCCGCGTCGGGGACCTTCTGCCGAACCAGCTCGCCGACAAAATCGACGA
TCTTGATCCCGGCGTCGACCTCGCTCATGTAGAGGAACCCGCGGGAGGCGCGCATGGCG
ACGGCGTCGTGCGCCAGCGCGAGCGCGCAGCCGGCGCCCGCGGCGTGGCCCGTGACG
GCCGCGACCGTTGGCACGGGGAGCGCGAGCAGGTCGGCGACGAGGCCGCGGAACGCG
GCGCGCATCTCGGAGAGGCGGTGCCCCGGCGCCGGGGCCGGCCCCGCGCGCGCCCAC
GCGAGGTCGTAGCCGTTGCTGAAGAACTTGCCCTCGCCGGCCAGGACGAGCGCACCGGG
GGAGGCGCGGCGGGCGGCTGCGACCGCGGAGCGGAGGGCGGAGAGCAGGGCCGGGCT
GAGGCGGTGCTCCTCCGCGCCGGTGAGGGTGATGACGTGCACCCGCCCGCGCTTCTCCA
CGGCGCACAGGCTTTCCTCCATCGTTGGAGTGGATTTGGGGCTCCTCACTGCTATGCCAC
TGATAGTATGTTTACTTTTTCCCCTCTTGCATCTGGGAAGGTCCAAAATGTCCCTGGTCCAG
CTCTAGTACAGAAGTGTTCAACTAAACCTTTTCTGTTCTGGCGTCAACACCAAGGCCCTAG
AGCACAAACCAAATTTAGGAGTGAAACTAAATTATATCAGGAACATAATAATTGGAGGTGAA
TTTAATTGTATAGACTACCTTATTCAAATTATGAGCCTATTTTAATTGGATGTGACTTCAACA
TTATTAGATATGTGAAAGACAAGAACACTATGAATGGTGTTCATAAACACAAAACCATCTCA
ATTCTTTGATTCACAATTTTATGAACTTTGAGAGTTGGTAATGTGTGGTGGGCTTTTTCACTT
GGTCAAACAATCAAGAGTTCCCCATTTTGGGAAAAACTTGATCGAATTCTTGTCTCAAAAGG
TGAGAAGTCATTTTTTCCACAAGCTATGGATAAATGAATCCCTAGAGAGATTTATGAACACA
ATCCCCTGCTTCTTTCAACTGGAAACT
SEQ ID NO: 24: GSK2 promoter sequence
AAGTGAAGGATATCTTCTTTGCGAATGGGATATTCCGAGTTAACAATGGACAGGACACAAG
GTTTTGGGAGGACAAATGGCTGGGGGATTTCTCGCTCCAGCATAGATTCCCGAGCCTATAT
AACCTAGTGCAGCGGAAGAATGCTACTGTGGCCAATGTGCTAGGGTCTGTACCTCTCAATG
TATCCTACAAGAGAGGCTTACATGGTGCTAATTTGGAGAGATGGCATACCTTAGTCAGCCT
AGTAGTGGATACGACGTTGAACCAGGCAAGAGATAGTTTTCGTTGGAGCCTTCATCAAAAC
GGGTTGTTCTCCACTCAGTCTATGTATGCGGCATTGATTGGGAACGGACAAGTACGGCAG
GATGGCCTCATCTGGAAACTAAAACTCCCCTTAAAGATCAAGATCTTCTTCTGGTTCTTAAG
ACAAGGGGTAACCTTAACTAAAGATAACCTTGCCAAGAGAAATTGGTCAGGATCAAAAAAA
TGTGTTTTCTGTCCACAAGATGAAACCATTCAACATCTTTTCCTCCAGTGTCATTATGCGAG
ATTTCTATGGCGTACGGTATATTTTACATTTGGCATTAGAGAACCAACTAGTATAGAAGATA
TGTGTTCTTCTTGGCTTCAGGGGTTTCACCCTAATGTTAAAGCTAAGATATATGTGAGCGCT
ATAGCTATTTGTTGGGCGTTGTGGCTAAGTAGAAACGATGTGGTTTTTAATAAATCTCCTAC
CCAAACTTATTTACAGGTACTCTTCCGAGGAACTTACTGGTGTCGTTTCTAGGGATGCTTCA
AAGGCATAAAGAGGACACTAGAAGCATGAGGGAGGCCTGCAGACTTTTGGAGACATCGAT
GATGCAAGTCTTCTCGACGTATGGTTGGACCTTCAGTAATAGATTAACTATGTGATGTTTGT
CTATTCTCCCAACTGCGTTTGGGTTTTGTGGCCAAATGTGGCGTTGTTTCGGTACTTTATGT
TGTGGTGTGTGGACGGCCGTCATCAGCTGATGTAGGTCGGGATTTGGTTTTTTTTTCCCGT
TATCTAAAAAATATATGTGGCTAGATTTATCATCATCCAGGTAAATATAGACATAAAAATTAA
GATCTCAAATGAATAATATCTTCGACCGGATGGAGTATGACATAATTTTACATCACGATTTC
TAAACAATTGCTAAGTTCTTTCCGCTCATTCGGTCTATTGTACATATGTATCAACATCTTATA
CTCATCCGTCTCAAATTAAGATTCGTTTTACTTAATTAATGGGTTCATACAACACTTGATTTA
TATGTTATGTATGTGTCTAGGTTCATCTTCATTTATTTGAATATTGATATAAAAATCAAGAGTT
AAAACAACTATTATTTTGGGACGCGGTGAGTATTTTTTCTCCATTTCCTCGCACCTAGGGAT
TTCACGCGATGGATACACATTCTATGTAAAAAAAGATTGGGCGTTAACAGTCAGTCATTAAA
AATATTCTTTTTCTAAAAAATTAAAAAAAGAGGATCTCCATTGGAAATATGTTTTTTCGAAACT
ACTGGAGATGCTCTAGGTATTGTGAACAGTTTTTTTCTCATTAAAAAGATGCTGCAAAATCC
GTTGATGCTCCTAGATCACTCGACAACTACAGTTACCATCGTTCATGCCTTCGGTTTTAGCA
ACAAAAAACAGTGCAATCCTAAACAAAAGCATCTATTAATCACAATTGGTTGCTGCCATTGG
TACTGCACTCAGCAACTCTGTTAGCAAAGGTAATGCACTCTTGTAGTCTTTGACCGGATCTT
TTGGCTAGGGAAAACTAAGGATGCGTTTGGTTACGGGACAGGCAGGATAGAGATGTCCCC
AGGCGTACTCTCTCGTCACTCTAATTTCGAGGGGCAACTAGAGACAACATTGGAATAATCC
TGTCTCAACCCCTGATTCTGAACTAAACAACCTTATTTAAGGTACGTCCTATCTCATCCCGT
TCTGTCATTATAACCAAACGCACCCTAAAAAAATGTTCATGAAGGAGAGAATTAAAAGGTTC
CAGTTTTCAGTATGCTAGTTTAGCAACGAGTGTATTGCAATTAATTATCACTATTGTTCGGA
CCCTCCATTTTGGTAGTACAGGTAAATCCCTACTAAGCAAGAATAATATGTTTTTTTATGCTA
CACATAGGTAGCGTTTGAGTAGACTTGTATTTTAAATAAAATGCTACTGCTGATAAGACTAT
AACGGTACGGGAAAAAGAAGACAATTTAGAGCTTGCCAAATTTCTTTAGCAGCCAATTAATT
CCTACCACGGTCCTGTCCTCAGAATTTTTTTTAGTAACAAATCAGTGCACTACTGATTCCTA
AACCAGGCTGAAACCGGAAACGGCTCGCTGCGCTGCCGCTGCGTCACTGTCGCTGGCAA
AGAAAACAACTCCCGGCCAGGGGTCCGAGCAGGAGCAGCAGTATATTTTCCCGCCGCTAA
TAAAAACAGTCAGCGGCACACTTCGCCAAGCGAGGCAGGCAGCGGCTGTCCCGAGCTGT
CGAAAGCGAGGCGCGGCGGCAGTCCTCGCAGCAGGGCCGACCGGTCAAAAGCACTGCT
GCTCCACACCACCCCCACCATCCCTTTCCCCAACCCCCGAAGCCGAGCCAGCGAACCACC
CCGCCCGCAGCCGCAAGCAAGCAGCCAAGCAGTGTGAACTGACCGTCCGTTCCGTCCAG
CCCACC
B.Napus
SEQ ID NO: 25: OML4 amino acid sequence
MMPSDIMEQRGVSTPSHFREDTRISSERQFGFLKTDLIPENQGGRDRFSNLPKSSWTPESHQL
KPQSSLSGVHPSVSPNARNTTNGSQWESSLFSSSLSDTFSRKLRLQRSDMLSPMSANTVVTH
REEEPSESLEEIEAQTIGNLLPDEDDLFAEVMGDVGRKSRAGGDDLDDFDLFSSVGGMELDGD
VFPPMGPRNGERGRNNSVGEHHRAEIPSRTILAGNISSNVEDYELKVLFEQFGDIQALHTACKN
RGFIMVSYYDIRAAQNAARALHNKLLRGTKLDIRYSIPKEIPSGKDASKGALLITNIDSSISNEELN
RMVKSYGEIKEIRRTMHDNPQIYIEFFDIRASEAALGGLNGLEVAGKQLKLALTYPESQRYMSQF
VAHDAEGFLPKMPFTNTSSGHMGRHFPGIIPSTSIDGGPMGISHSSVGSPVNSFIERHRSLSIPI
GFPPLANVISASKPGIQEHVHPFDNSNMGIQSMPNLHPHSFSEYLDNFTNGSPYKSSTAFSEVV
SDGSKANDAFMLHNVRGVDGFNGGGIGSPMNQNSRRPNLNLWSNSNTQQQNPSGGMMWP
SSPSHLNSITSQRPPVTVFSRAPPVMVNMASSPVHHHIGSAPVLNSPFWDRRQAYVAESLESP
GFHIGSHGSMGFPGSSPSHPMEIGSHKSFSHVAGNRMDINSQNAVLRSPQQLSHLFPGRNPM
VSMPGSFDSPNERYRNLSHRRSESSSSHADKKLFELDVDRILRGDDVRTTLMLKNIPNKYTSK
MLLSAIDEHCKGTYDFLYLPIDFKNKCNVGYAFINLIEPEKIVPFYKAFNGKKWEKFNSEKVATLT
YARIQGKVALIAHFQNSSLMNEDKRCRPILFHTDGPNAGDQEPFPMGTNIRSRPGKPRSSSIDN
HNGFSIASVSENREEPPNGTDPFLKEN
SEQ ID NO: 26: OML4 nucleic acid sequence [symbol]
ATGCCGTCTGATATAATGGAACAGAGAGGTGTATCAACACCTTCCCACTTTCGTGAAG
ATACTCGTATTAGTTCAGAGGTAACTTTTCTTTTACTGTGTAGCACCATCTTTGTCACATTA
TCTGCCACTATTTTCTATGATGTTTAAAACTGTTTTCTTTTTGTTTCTCAAGTATACTTGTTCT
TTTGTCTGGCAGAGGCAATTTGGGTTTCTGAAAACAGACCTGATTCCTGAAAACCAAGGTG
GTCGTGATAGATTTTCAAATCTGCCAAAGAGTTCCTGGACACCTGAAAGTCACCAGCTGAA
GCCACAATCTAGCTTGTCTGGGGTGCACCCCTCTGTTAGCCCTAACGCAAGAAACACCACA
AATGGTAGCCAGTGGGAAAGTAGTTTATTTTCCAGCTCACTGTCTGATACATTTAGTAGAAA
ACGTAAGCTTCTGGTTCACTTTTATGAATTGTTACTTATTATGTTGATTTTGTTTTATCCTCTA
CGGTAAAGAAACGCCGTTTGTTAATCTAGTACATCATAGACGATCGTGAAAGTTTGTTTCTT
TCTCCTTTAACTTACTGTACTTTAACTACTTGACTGCGTCTCCAAATTCTTGGTTTTTGCAGT
ACGGTTACAGAGAAGTGATATGCTATCTCCTATGTCTGCGAACACAGTTGTTACCCACCGT
GAGGAAGAACCCTCTGAATCTTTAGAAGAAATTGAGGCGCAAACTATTGGAAATCTTCTGC
CAGATGAAGATGACCTCTTTGCAGAAGTGATGGGTGACGTTGGGCGTAAATCTCGTGCCG
GTGGAGATGATCTAGATGATTTTGACCTTTTCAGCAGTGTTGGTGGCATGGAGCTAGATGG
AGATGTTTTTCCTCCTATGGGCCCCAGAAACGGAGAGAGAGGCCGCAATAATTCTGTTGGC
GAACATCATCGAGCGGAAATTCCATCCAGAACAATTTTGGCCGGAAATATCAGTAGCAATG
TCGAAGACTATGAGCTGAAGGTCCTTTTTGAGGTACCTTATTCCAGCAGCGTTTCCCCCCA
CAGATTTGTTTATATAATCTGGAATTGATTACTTCGTACTGAGAATACTTTTACTTGTTCAGC
AATTTGGAGACATCCAGGCTCTTCATACAGCTTGCAAGAATCGTGGTTTTATCATGGTATCC
TACTATGATATAAGGGCTGCTCAAAATGCGGCGAGAGCACTCCACAATAAGCTGTTAAGAG
GAACGAAACTTGATATTCGTTATTCTATCCCTAAGGTATGATTCCTTGTTTTTATGAAATATA
TTGTCTTTGCTCTGTGGACAGTATTTGTGACTTATGTTGATTTGTATCTATCTTACAATTTTC
TTGGCTCCAGGAAATTCCTTCAGGAAAAGACGCCAGTAAAGGAGCCCTGTTGATTACTAAT
ATTGATTCGTCTATTTCAAATGAAGAACTCAATCGAATGGTCAAATCGTATGGAGAAATCAA
AGAGGTTGATATATTGAGATGCTCCGTTTAGTTACTTTTCTGAGGTAGATTCTAATGATGTTT
CTGTGGTTTGCAGATTCGTAGAACCATGCACGATAACCCACAGATATACATAGAATTCTTTG
ACATCCGAGCGTCAGAGGCTGCTCTTGGTGGCCTGAATGGACTCGAGGTTGCTGGGAAGC
AGCTTAAACTTGCGTTAACCTATCCAGAGAGTCAAAGGTGGGTGACTGGTTGTTTTTTTTTT
CTCCCTGGTTTATATTCCTTTGTGGGCTGTGAATGAATACAAAATCCTAAATCAAAATGATTT
GAACATGTGCTTTGCTGTTAAGTATTTACGAGGATGCCAGTTGTGTTGATGTATGGGGTTC
ACCCATTCTTTTTTCTTTATTTCAGGTACATGTCACAGHTGHGCACATGATGCTGAAGGG
TTTCTACCTAAAATGCCTTTTACTAATACATCATCTGGGCACATGGGTATGCTTTTGCATTCA
GCATTTGTAATTCTTTTTTTTATTGAATGATTTGTCATCTTGATACTCAAACCACTGCCGTTA
AATATCTCTGTGTCAGGGAGACATTTCCCAGGAATAATTCCTTCAACCTCCATTGATGGTGG
ACCTATGGGGATTAGTCATAGTTCTGTTGGATCGCCTGTGAACTCCTTCATTGAACGTCATA
GGAGTCTCAGCATTCCTATTGGATTTCCACCTTTGGCAAACGTCATCTCAGCCAGCAAGCC
CGGAATTCAGGAGCATGTCCACCCTTTTGACAATTCAAATATGGGGATCCAAAGCATGCCA
AACCTTCATCCTCATTCTTTTTCAGAGTACCTCGACAACTTTACAAATGGTAGTCCATATAAG
TCCTCGACAGCATTTTCTGAAGTCGTCAGTGATGGCTCGAAAGCAAATGATGCCTTTATGTT
ACATAATGTTCGTGGAGTGGATGGCTTTAACGGAGGGGGTAAGCTCTTTATCTCTAAATTG
CTACTGTTTTGATAAATTTGTCGAAGAATAATGATGATATGTAGTTGACAATTGTGAGTTTAA
GAAGAATGTCTGCCGTAGCACACTGTTAGGATGGTCCTTACAATTTTAGTGGAATCTGAAAT
GTGCTACAGCGATGAAAATTCTAGGTACTGTTTCTGTAGACAACTTTTTTTAAAAGCATTCTT
GGTGTAAAACTTGTCATCCTGGGAAAATATTATTAGTATTATGTTCTTAATTGCAGTCATATA
GACAGATAACTGTGCTGGGTTTGAAATTGAATTTGAAAGTGGCTGAAACATTCGTTGTGTAT
GTCAACAGAATTGCACAATTACTGAGTGCTAGTATTTCTTCTACTGTCATACATAATATTGTT
TTTTTCTTTCTCACTTTTAGTTGTTGTGGTCTTTTGACTGTAGGCATAGGGTCTCCCATGAAC
CAAAACTCCCGCCGCCCTAACCTTAATTTATGGAGCAATTCTAACACTCAGCAACAAAATCC
TTCAGGTGGCATGATGTGGCCTAGCTCGCCGTCTCACCTCAACGGCAAACGTCATCTCAG
CCAGCAAGCCCGGAATTCAGGAGCATGTCCACCCTTTTGACAATTCAAATATGGGGATCCA
AAGCATGCCAAACCTTCATCCTCATTCTTTTTCAGAGTACCTCGACAACTTTACAAATGGTA
GTCCATATAAGTCCTCGACAGCATTTTCTGAAGTCGTTAGTGATGGCTCGAAAGCAAATGA
TGCCTTTATGTTACATAATGTTCGTGGAGTGGATGGCTTTAACGGAGGGGGTAAGCTCTTT
ATCTCTAAATTGCTACTGTTTTGATAAATTTGTCGAAGAATAATGATGATATGTAGTTGACAA
TTGTGAGTTTAAGAAGAATGTCTGCCGTAGCACACTATTAGGATGGTCCTTACAATTTTAGT
GGAATCTGAAATGTGCTACAGCGATGAAAATTCTAGGTACTGTTTCTGTAGACAACTTTTTT
TAAAAGCATTCTTGGTGTAAAACTTGTCATCCTGGGAAAATATTATTAGTATTATGTTCTTAA
TTGCAGTCATATAGACAGATAACTGTGCTGGGTTTGAAATTGAATTTGAAAGTGGCTGAAAC
ATTCGTTGTGTATGTCAACAGAATTGCACAATTACTGAGTGCTAGTATTTCTTCTACTGTCAT
ACATAATATTGTTTTTTTCTTTCTCACTTTTAGTTGTTGTGGTCTTTTGACTGTAGGCATAGG
GTCTCCCATGAACCAAAACTCCCGCCGCCCTAACCTTAATTTATGGAGCAATTCTAACACTC
AGCAACAAAATCCTTCAGGTGGCATGATGTGGCCTAGCTCGCCGTCTCACCTCAACAGCAT
TACTAGTCAGCGCCCACCTGTTACTGTATTCTCTAGAGCACCTCCTGTTATGGTGAATATG
GCATCTTCCCCTGTGCACCACCACATTGGATCTGCGCCCGTATTAAACTCGCCTTTCTGGG
ATAGAAGACAAGCCTATGTTGCTGAATCTCTAGAATCGCCTGGCTTCCACATAGGTTCTCAT
GGTAGCATGGGGTTTCCTGGCTCTTCACCCTCACATCCAATGGAAATTGGTTCTCACAAGT
CCTTTTCCCATGTTGCTGGGAATCGCATGGATATAAATTCCCAAAATGCTGTACTGCGATCT
CCCCAACAGTTGTCTCATCTCTTCCCCGGGAGGAACCCAATGGTTTCAATGCCGGGTTCGT
TTGACTCGCCTAATGAACGATACAGGAATCTCTCACACCGTAGAAGCGAGTCTAGCTCTAG
TCATGCTGACAAGAAACTGTTTGAGCTTGATGTTGACCGTATATTACGTGGGGATGATGTC
AGGACAACACTGATGCTTAAAAACATTCCTAATAAGTAAGTGGATTCAGTGTCTTTCCTTTA
TTCCTTGTTATATATCTTTTGTTAGCTTCGTAGGTTGTTTGATGTTTTCCTTTTCAATTCTGAA
CTCTATAAAATGCTGCTATGGTTTAGGTATACTTCTAAGATGCTTCTCTCCGCCATTGACGA
GCATTGTAAAGGAACGTATGATTTCCTTTATTTGCCAATTGATTTCAAGGCAAGCAGGCGTC
CGTCCTACCTTTTTATATAATAGTCTTATGTAGAAAATGGGCTTTTGGTATTTGCAATATCAG
TATTTTTTTGCTAACCTAATTTTACCTTCTCGTTTCAGAACAAATGCAATGTGGGATACGCTT
TCATCAACCTTATTGAACCTGAAAAGATTGTACCATTTTATAAGGTACAGCCAGCCTTTTCT
GTTGCTGCTTTTTATATATTTTTTGGCTTTTTCTCTTGAAGAGCATTGGTTAAAAGTTTAAAAA
AAACTTGCAGGCTTTTAATGGAAAAAAGTGGGAAAAGTTTAACAGCGAGAAGGTGGCAACT
CTTACATATGCTCGAATTCAAGGAAAAGTAGCACTTATTGCCCATTTCCAGAACTCAAGCTT
AATGAACGAAGACAAACGTTGCCGGCCTATTCTTTTCCACACCGATGGTCCAAATGCTGGT
GATCAGGTGAATGTTACTAACACATCAGATAACATCATCTTGTTAGGGTTCTCATTTCGTAG
TAGTTGCTCAATTTCGCTCTCCCTTTGGTTGCACATATTGAAATGGGTTCTTAGTGAGATCT
CATAAGTTCAAAGATGTGGTGATGCTCAGTTACTCAATAAGAGATTGATTTGTTTCATATTTG
TCACCTTTGTTGTTATTATTTGCAGGAACCATTTCCAATGGGAACCAACATACGATCAAGAC
CAGGAAAGCCACGAAGCAGTAGCATTGATAACCACAACGGCTTTAGCATCGCTTCCGTTTC
AGAAAACAGAGAAGAACCTCCTAATGGAACCGATCCTTTCTTGAAGGAGAACTAACCAATG
AGCAAAAAAACCAAGCAGAGGTAAAAGAAAGTTAAGGAAAAATGAAGAGCTAAAGATATAA
CACAAGTTTTATATTATTATAATCATATCATCAGCACACCCTAGAGTTCTGTAAATCGGGGG
TGTTAAATTTACCCTGACAAAACTGTTTTTGCGGTGAAGATATATTTTTGGAGAGATCATTAA
ACTTTGTTGACCTCAAACCTTCACAGGTTGCTTCACCAGTTTGTTGTATATCAAATATCCC
CTGAGAAATATCTTCGAGAGTTTCTCTTTACTTTTTGTTTTTTTTTTTGTCTTGTTTGGGGTTA
TTCAAGTATTTTGTCTTCTTGCTATCGATGTAGTATGTAACAAGCCTTGGATTTACATTCAA
CGTCTTTGCTGGCTATTTGTGGCCATTTCATGTTGTAACTTTTTTGGAGATTTTAATGAATGC
TTCCTTTTTGGATAAA
SEQ ID NO: 27: OML4 promoter sequence
AGTAATTAATATTCTTTTCGTTCCACAAATATAATTTTTTTAGTATTTTCACACATATTAAGAA
AACACGCTAAACTACCATAATAAATGTATTGTTTTATGTAATTTTCAATTTTCAATAACTTTTA
ACCAATAGTAATTCAATAAAGTCAATTAATTTCTTTGAAATTTACAAATTTTTCATAGAAAACA
CAAAAATACATATTTGTGAAACAAACTTTTTCAAAAAAGTCTATCTTGATGAAACGGATGGA
GTATTATGTATAATATTTTTATTATATATTTTATTGCTAAATAAAAATTTTATGACTTTTGTTTA
CTTTTTCACCAATAAAAGACTATAATGCAAAATGTAAAATATTTAAAGTTTAATTTGAAGTTGT
TATTTCGGAAATAATCACCTTCGAAGTTTAAATTTGTAATATTGCAAACTTTATTTGGAGATG
TTTTCACGGTCGACTTGCTACATGACTCTTTTTTTTTTGTAGCATGCTACATGACCCTCTATT
CTTTTTTTTCCCCTATTTATTGTTACTTTACAATTGAAATAATAAGGCAAAATACAATAGTGGA
TGACTTTTTTCCCCATACCACCTTTTTCGGTTTTCTCTATTTGGTTGTTCGAACCTGCACATG
CTCATTTGATAGCGTGGAAGGATTGGCCATCAAACAAATAAAAATTCACAATCAAGGATATT
TATTATCAGTTTTTGTTGTTGTGCACTTCATTGTAAAATAAAAAAAATCCAAACACGGATGAT
AACAACCGTGGATCACGAGTAAACTAATTCACTCAGTCATAAAAAGAAAGAGATATAGTGA
GCAAAAAATCATTTTAAGATAGTATTGATCCAACCAACCAAACATTATCTTCAAAAATTACAA
TGTTTTTACGACAGTTGATAAAAAAAAAGCTTATTTAGTAAACATAAAAACTATGGAGTAGTT
TTTTTTGTAAACACAAACTATAGTTTCAGACTTGTTTTGTATTCTTTCAACAAAGAGTGTAA
CTATAAAAACATTCTTATCAACTTTTCGCTCAAGTTGTTACAGAAAAAAACTTAATCAAGAAT
TAAAATGACACTTATAAAATTATCAATATAATAAAATTATTAATTTATAGAGGTTATATCAATT
AAAAACTAACAACTTTTATTCGTGTTTTCATTCATATATAATAGTAAAGTGTAATTTCCTAACT
TCATTTGAACATATTCTAATAAATAGTTTGTAGATTAAAAACAAATCACACTTTGAAAAGAAA
AAAAAATCAAATAGTCCACATGTTCAATAAATAGGCTGCTCCTTGGTTACAAAACCGCGCTC
ATCGACTGCTCGCTGCCGTCGAGACTCTCGTGTGAGACCGTAATTTTTGTCAGTTTTAGTT
ATAATCTACGGTCCAGATTTAATATCGTACGAAACCACTAGATCCACGATACATCCAACACA
GAAGAGTGCTCTCCTCTCCTCAACTCTATTTTGTTTTTTTCCTCTCATTCTTTTTTTAGTCGA
AACTCTAAACCAACTAACCGAAAAAAACAAAAAACTCTTTCTCTCCTCTCCATTTCTCTCTCT
AGGAGAGACAACCGGAATCGCACGTCGACGGGAAGAGTATCGCCGGAACTATTATAATTA
CCGCCGGTCGCATAGATTATTCGTTGGAAACAACGCGTCGTGAGAGGAGAGGAAATTCGA
AAAAAAGAAGAAAAAAATTAGAAACACCGATTCACTTTTTTTTTGGGGGTTATTTTAATTGAT
TTGTGTGAATTAAATATTCTGCGATGGATGTGATTGGATAGAAGGAAACAAAAAGGAAAGG
AGGAAGATAAAAGAGAAGGCGAATTATTCTGCTCCTCTCTCTCTCTCTCTCTCTTTCTTCTC
TGTCGAACATCGCTGTTGCTGCTGTGTGTTTTCTTCGTGCATCCTTTTATTTTTCAAGGTAAT
GAATTTCACGAGATCCATTCTTCACAAGTTTCTTTCTTTTTTTAAATTTAATTTAATTTAGTGG
AAAAAATGTTTGGGAGGAAGCGTAATTGTGTTTGTTTGTAAATTAGGTAAGCTCTTTGTATTT
GTTTTTTTATTTGCTGGTGAGTAATTTAGGTTTATTTTCTTAAATTAAGTTAAACTGGGTGCC
CAAGTTTGTGAATTAGGTAGGAGTTGGTTCCCTGTTTGCATATAATGAGCTGAACAAGGAT
CATGAATTAGGCGAAATTGTAGTCTCTTATGGCTTTTTGAAATACCTAATCTTTGTCTTCCAG
GTGTTTCTACTCCGCTTTAAAGGAGAGAGGTTTAAGATGATTTTTTTCGTATTGAACTTCTTC
TTAGAGTACGTAAAGTTGCTGACTTTGTTTGGATTTAGGGTTTGATTTTGCTTAGTTCTAATT
GAATTCTTGTGTTGTTTTTTTTTGTGTCCTTTGAGTTATTTTGCTTAATCTTTTTTGTCTGGCA
AGATCCTTCTTTGCAATGAATAGTGGATTTTGTTTCTTTTGGAGACTTACTGGCTTTGAATCT
AAAACTGGTTGTTCATCTTTCAGGGGAAGTGATATGGTCCGTTGAAAAAGACTAAAAAGCTA
CAAAAGAGATTTTGTTTTATTATTCCAAATTTTGCTGTCATCTGC
SEQ ID NO: 28: GSK2 amino acid sequence
MTSLSLGPQPPATAQPPQLRDGDASRRRSDMDTDKDMSAAVIEGNDAVTGHIISTTIGGKNGE
PKQTISYMAERVVGQGSFGIVFQAKCLETGESVAIKKVLQDRRYKNRELQLMRLMDHPNVVSL
KHCFFSTTSRDELFLNLVMEYVPETLYRVLKHYTNSSQRMPIFYVKLYTYQIFRGLAYIHTVPGV
CHRDVKPQNLLVDPLTHQCKLCDFGSAKVLVKGEANISYICSRYYRAPELIFGATEYTSSIDIWS
AGCVLAELLLGQPLFPGENSVDQLVEIIKVLGTPTREEIRCMNPNYTDFRFPQIKAHPWHKVFH
KRMPPEAIDLASRLLQYSPSLRYTALEACAHPFFNELREPNARLPNGRPLPALFNFKQELAGAS
PELINRLIPEHIRRQMSGGFPSQPGH
SEQ ID NO: 29: GSK2 nucleic acid sequence [symbol]
ACATCACTATCATTGGGCCCTCAGCCTCCGGCTACTGCTCAGCCGCCGCAGCTTCGC
GACGGAGATGCTTCCAGGCGTCGTTCCGATATGGATACAGACAAGGTTGCTCTCTCCCTCT
CTCTCTCTCTCTCTCTCTCTACTTTAACGTTTGGTGAACAAATTGCATTTCGATTGCGTTTGG
TGGCTATTGTAGATCTCGGCTAGATCTAGCTTCGATTTCACTTTTTTTTTGCGGTTTCTCAG
CGAATCGATCTGTGTTTTCTCTTGCTATCGTCGTAGTTCGTAGTTCGTAGTAGCTAGCTAGT
CTTACTATTCAGCTGAATGTTTCAACCAATCATATTGAAGATCTTGAGCTATGTTTTGATTAC
TAGTATTAGGGTGAAGAACATTGGTTCTCTCTGGGTTTGAAATTCGATTTCACAGACGATGT
AGATCTTAATTACTAGATTGTTTAACTAATCACACACTTGTTCCATGACTGTAAGTGATTTGA
TGTATTGGATTTACATTTGTTTGTTATCTACGTGATTGGACTCTGAGCTAGGCCTTGACTGT
TCTTGGATTTGAAGATTTCATATGTTTAAAGAATGGTTTTGTCTATTGATTGTTTCGTAATCT
CATGTTTGTTGTTTTCAGGAGAAGAGCACTATTTTTTTTTTTAATCAGTTTTCTTTGTTCTTTC
TTGACGAGAATAGTTTGATGATATGTTGAGGTTTGGTTGCAGGATATGTCTGCTGCTGTGAT
AGAGGGAAACGATGCTGTTACAGGCCACATCATTTCTACTACAATTGGAGGCAAAAACGGT
GAACCTAAACAGGTTTGAGTTCCTTTCTTTGTTTGAAATCTTCAAATGTCATAATTAGTAACA
TTGTTAATGATTACATTTAATCATATGTTCACTTGCTTTTCCACTTACAGCTTAAAACAATAAC
TAAACAGAGACTCTTTGTGGTTCATTTATTACAACTTTAAGTAGGCTACTCACTTATGTTTTA
CTCTTTCTGTTTTTTTGCAGACCATCAGTTACATGGCCGAACGGGTTGTTGGACAAGGATC
ATTCGGAATCGTGTTCCAGGTACCTTTGTGCTTCTCAATCACTGTTACCCTTTGTAGGCGGT
AGCTTTCTTCTTTCCTTTCTGATCGAAGTATGAACTTACCATTGTAGGCCAAGTGCTTGGAA
ACTGGAGAATCAGTCGCCATTAAGAAGGTTTTGCAAGACCGGCGCTACAAGAATCGTGAG
CTGCAGTTGATGCGACTAATGGACCACCCAAATGTGGTTTCCTTGAAGCATTGTTTCTTCTC
TACAACGAGTAGAGATGAGCTCTTCCTCAATCTCGTTATGGAGTATGTACCCGAGACTTTG
TACCGGGTTTTGAAGCACTATACTAATTCAAGCCAGAGAATGCCTATTTTCTATGTCAAACT
CTACACATACCAAGTATGCATTGTTATTATGTGTTTCCCTTTCAGGCAGTATCTCTCTTTGTT
GATTCTAAAACGGGTAAGAATACTTTTTTTCTGCAGATCTTCAGAGGCTTGGCTTATATCCA
TACTGTTCCTGGTGTCTGTCACAGAGATGTGAAACCACAAAATCTTTTGGTACGTTGATTCT
ATTTTGGGTTTGTCTTTGATAATCTTGATAGATTGTTAACTAATTCTCTTGTACGTTCTGCAG
GTTGATCCTCTCACTCATCAGTGTAAGCTGTGTGATTTTGGAAGTGCAAAAGTATTGGTAAG
GAGCTTTACCTTTAATATCCTGCTTTGCTTATTTCAACTGTGTATGTGTTCTGTCTCATGAAA
TCTTTGCGACACATGATTATTCGGATTAGGTGAAAGGTGAAGCAAACATATCATACATTTGC
TCTCGGTATTACCGAGCTCCAGAGCTCATCTTTGGGGCCACAGAGTATACATCCTCCATAG
ACATATGGTCTGCTGGTTGTGTTTTGGCAGAGCTCCTTCTTGGCCAGGTTAGTGTAAACTA
TTTTATCTGTTTAACTCTAGAATGTTCCGCTATCATTTTTGATATTTATAATTTTTTATCTGTC
AGCCGTTGTTCCCGGGAGAAAATTCTGTGGACCAGCTGGTAGAGATCATCAAGGTGAAGT
TTCATTTTGATCATATGTTATCTTGCTGTCGTATTCTGTTTTGTATATAAAATTCATATAATCT
TATAGATTTGTAATGATATATGTGCTGCGTTTGTTTAGGTTCTTGGTACTCCAACTCGAGAA
GAAATCCGATGCATGAATCCAAACTACACAGACTTCAGATTCCCTCAAATCAAAGCTCACC
CGTGGCATAAGGTATTTATATGCATGTCCGATCATACAGTGGCTAAATAGTTGAATCGCTTC
TCATTATATTCGTATAAATGAAAAACTAAACAAATTCACATACTTCTCTCTGACCTTCAGGTT
TTCCATAAGAGGATGCCTCCAGAAGCCATTGACCTCGCATCTCGGCTTCTTCAATACTCAC
CGAGCCTGCGTTACACTGCGGTCAGTATCTCTAAACCACCAAGTACTCTTAATTGTTAAGA
GTGTTCTCTCTGGATTCATTGGACCTGCACTGCACTGTCCAATGTTGCTGATGTTTTCTTTT
GAACTCCGTGAGCCGAATGCTCGTCTTCCTAACGGCCGACCTCTACCAGCCTTGTTCAACT
TCAAACAAGAGGTACGTCAATCACAGCAAAAAAAAAAAAAGTAATATAGCTCCAAACCATTA
CTAGAATGTTCAGTTTTAAACAGTTGCCTAATCTGTAATCTCTCTCTCTATTCGAATGTTCAT
AACAGTTAGCTGGGGCTTCACCAGAGCTGATAAACAGGCTCATACCGGAGCACATAAGGC
GACAGATGAGTGGAGGCTTCCCATCACAGCCTGGTCAT [symbol] AAAAGGAATATGGAAACTG
GGATGCTTTTGCGGAGCAAATGCCTTATGGAAAAGAGGAGAGAAGATCTCTGATTTTTCAG
AGGGTTTAACTAAAATATCAGCTTATGAGTAGAGAGATGATTGGCCAATTAAGCTTTTTGAG
AAATCAGGAGGTGGTGATGATTGTGTCTAATATACAATTCTCTCTTTTCTCTTTTTATGTTAT
AATTCGCTTTTGACTTGTAGAGATACCTTTTCTCGTTGTATTATTTGTATATGTTTTTGTCCG
TAAGACAGCAAACCGCGATGATGGAAGAATGGAATGAATGAATGATGTCTAAAACTTAAGC
CTAATAACAAGGTCGGAGCTCATACATATATATAAAGTTAGAATGTGAGAGCTCCATGTTAA
AATAACCTTAACATTGGCACGTGAATACAATTGCATGATTGAATTTCTGGTACGTCGAGAGG
AAGTAAGTTTATAGAAAGTTGTTTGTGAACAAACAAATGGAGAAACATTTGTTTTGTTGCAAA
GAAACGTATGGTTCCATAATGTAGAAGAGGCATTTGAATGTGAGCTTTAAAACCTTTCATGA
AAGAAAAGGAAAGTTATGGGTCACTAACCGGAAAATATATCATTTGAAATGTGTATAAAACT
TAATGGGCTGAAAACTGTAGATAAGGAATTCCGGATTCTGGGAACCCTATTAACTGAGCCA
CAAGCAAAGATACGAGGACCAAACCCTAAATCTTCTCTCTTTTTTTCCCCCTCATTCAGGTG
TTTTTCATTAGTCACATTCGTTCTTTATACTTTTATTATCTTTGATTGTTAATAGATTGTCTGA
AAACGCATGTCCACTTGTTTCTGTTTTATTTGTTTTTTTCTTTTGCTGCAGGCTTTGGAAGTC
CACACTAAGGTGAAACAACTCTCCCTAATCTATACGCCTTTCACCTCTTTCCCCGCCTTTGA
TCCTTTGAGAGTTTTTTTTTCTTTTTTTTTTTTGAAAATTCAAATTTTATTCAACCTGAGAATC
GGGAAATCATATTCGGTTACAAATCCGCTTTGAACAAAAGTTCCAAAATCAAACTATTACTA
TCTTTGCCCACTCACTAAACCTGACACATTCTGCTAGCCTGTTTTTCGAAATTCTTCAGAAT
CGGTTGCCGTTCTAAACTTTTGACGAAACCCAGAGGACTCCTTGTTGCTGCAGATGCCGG
GACTTCCTATGTCGTCTCCGGAGTAGCTAGTTCCCGATGAACTCCACAAAACTCATAACCG
TCAATGTCTATGAAGCGCTTGACGGTTCAATAGGGGATGGTTTCAGGTACTTACGATTAGA
GGCTTTGCAACCACCACCAAAACCGGGCTTGTACACAAACGCCACACAGCGCTCGAAACC
GAATCCCAAACCGGGCAAGCGCCCGCTACTGCCGCCACTAAACCAACCTTCAAGAAAGCG
GGCCGATTTTGCT
SEQ ID NO: 30: GSK2 promoter sequence
ATGCGTTCTAAGTATCAAGATCCTATTACTACTACTACTACACCTTGTAATGAGAATCATAAG
GTGAAGATAAATGGATCTTCTACTCCAGAAGGGAAAGAGAGACTAGAGAACTTGAGCTCAG
CTTCACGCACTAAAACCAGCAAAAACTTTGGTGAGCTCTTGGCTAGTGATGACAATACATG
GGAACCTTATTCTGAGGCTCCTGTTGCTGAGAAAACTCTGTATGTAGACACTGTGCATTCA
GTACACAAGAAGGTACAAGAAGAGTCTTTATTAAAAGATTACCCTTCACTAGAAGTTGTTCC
TGTTAAAGAAGATGTTCAGAACTTGATTGGAGCCAGTGAAGAAGCTATCTCAGGTCTAAAA
GTTGAAGAATGTGCTGATCAAGCTATTTCTGAAGTAGTAGAGATTACAAAGGATTTTGAATG
TTCAAGGCTTCATCATCATCACATTGTTGCACCACCATCATTGCCAAAAGCTCCTTCAGATT
CTTGGTTAAAGCGTACGTTGCCAACAATCCCATCAAAGAACAACTCATTCACATGGTTGCA
GTCTCTTGGCATTGATGATAATAATAATCAAATCACCAAGAGTATTCAAGAAAATCTCAAGT
GGGAAACTATGGTCAAAACCTCCAATACACAACAAGGGTTTGTGTGCATCTCCAAGGTAAG
CTAATGTGTATTTTTCAAAGTCAATGGTTGGCCAAATGTTTTTGTTTTTTTTTTTGTTTTTGAC
AAGTTGATTAGCTTACTTTGTTGACCATTATTTTTGTCTTTCAGGACACACTCAACCCTATAC
CAGAGGCATAGCAATACCAAATTACAAGTAAATTTCAACAATAAAAAAAGGATGAGCCAATA
AAGTTTTGTTTTTGTTCATCTTCCAAATTTTCTCCTCTTTAATTATATGTAAATCTGAAATAAA
AGGTTCCTAAAAAGAGAAAAGCTATGGAGATGAAATAAAAGGTCTCAAATATTGTCTGTCAC
TTGTGGGGTTTGGGGGGGGGTCTTATTGAAGTGATGTACAGCTCATGTTAACAGAGATTTT
GTTGCAATAATACTCCATAATTCCATGTGACATTGTTTCTTTTGACCTTCTTTATATATTCTCT
GCTAGTAATAGACTTTTTGTTTTGTTCTTTTGTAATTATGTTTCTGTAATGTAGAGCACTAAA
GAGACCTGAAAACTGCAGAACTCAATTGAATGCATTGGCTAAATGGTTATGAGAGGAATTA
TTGAAACAATTTATGGTGTGAGAAGTTCAAATATTATTCTCTTTATAGTGTCATGGATAGATC
AGATATAGTTCAGGAGAAAGTAAAGAAAGAAAAAAAAACTTTATAAAGGTATCTTCATTAGTT
AAGATATACATGAAAGAAACTGCTGCTTTAGGAGATGTTTTGTTGATCTTCATGATTCTTCTA
TCTTTATCACTTGTATGATTGTATCCATGGCGGTTTTTGCTTGCTTCAAAAACAAGAAAGAG
AAGAATGGTTCCTGTAGCTGTGGCAGTTGTTGGTGGCTGCGGTTGTGGTGGTGGAGGCTG
CGTTTAGTATTACACAAATGAGATATATCTTGGTCCTTGGCGAGTTTCCTGGTAATGATTTT
GGTTTAGAGCATCTTTATCCGGGTATACCAAAGGGTTTCTTAGCCTGTGGGTCCCGTGTAG
GACCCATTTTTTTTAAGAAATCGGTTACAAAAACTACTAAATAGTAGTCGGTTATTAAGGGTT
TCTTACACTGTTCGCGGACCCCGCTAACACGTGACGGCTAACGATTGGTTCATTTTTTTTTT
TTAAATTCGAAAAAGTAAAAAAAATAAAATAAAAAAAATTAGGAAACTCTATTTGGAGTTTCA
GGGATAATGATGCTATTAGGTCTTCACCGATTGTGACTGATTTACTTAAGCAGCCATTCTAT
ATATAGTTTACATTACGTACATATAGAACAAAAATATATACATAAAATATCAGATAAATTCAG
AATCAAATATATATGCGATATGTTTTTGTAAATTATTTGTTCAAATTTTCAAGTCTACAAATAA
TGAGTCACAAAACAAAATATACCAAGAAATGGATTGCGATCGTCCATGTGATACATCCAGG
GCCCTCTAAGACTTTTAAACGTATCTCGTATTGAACCAAATGTTAAAACCCCGTTGAAAAGG
TAGCCATCTTGCTCGTATAAACGAAAATTTTCATAGATGGTAGGGGGTGATTGGTTGAACT
GTAGCAAGTGACTTTAACTTTAATTTTTATCTACAGTTTTAAAAACCATCAATCGTGCTTTAT
ATTAGTTTTTAAAGCTACCACCAAAAAATAAAAAGTACAGCCAAAAAAACAAAAAAAAAAATA
ACTGTAAAAAATTTAATTTCTAAAGCTCCATTTTTTTGGATGTAGGAAATTTTAAAGCTCTGT
TCACGCGTGGGCCATCCTTTTCAAACATACTATACTAGTTGTTATTTGTTACCCAAAATGTA
AATACATGCTATGTCCTTACTAGGCAGTATATAGAAATTAGTTTGTTTTAATGAATCTGGAAC
AATACTAACTTCAATAATTAATTGCAAGGTTATCCACCCTTGACTGATGAGGAGGTTAGTCG
CGTTCTCATTGGTGCGTTACTCTTACGCGCTCTATCGACGCGTGGACGATATCCGAAGCTC
TTTTAATAATACAAAGAGAGAGAGAGAGAGAAGGGAAAGATAGTCTTTACTCTTCAGTGGT
GGGTAGAGAGCGAAAGTTAGAGAAAGAGAGAGAAGAATAGCAC
SEQ ID NO: 31: GSK2 RNAi sequence
TCCCAGGTGAACCCAATATATCATATATATGCTCACGCTACTACCGAGCACCGGAGCTCAT
ATTTGGTGCAACTGAATATACTACATCAATAGATATATGGTCAGCTGGGTGTGTTCTTGCAG
AGCTACTCCTTGGTCAGCCATTGTTTCCAGGGGAGAGTGCAGTCGATCAGCTTGTAGAGAT
AATTAAGGTTCTTGGTACACCAACCCGTGAGGAAATACGTTGCATGAACCCGAACTATACA
GAGTTTAGGTTTCCACAGATAAAAGCTCACCCTTGGCACAAGGTTTTCCACAAGAGGATGC
CTCCTGAAGCAATAGACCTCGCTTCACGCCTTCTTCAATATTCACCGAGTCTCCGCTGCAC
TGCTCTTGATGCATGTGCACATCCTTTCTTTGATGAGCTGCGA
SEQ ID NO: 32 CAS9 nucleic acid sequence
ATGGCTCCTAAGAAGAAGCGGAAGGTTGGTATTCACGGGGTGCCTGCGGCTATGGATAAG
AAGTACAGCATTGGTCTGGACATCGGGACGAATTCCGTTGGCTGGGCCGTGATCACCGAT
GAGTACAAGGTCCCTTCCAAGAAGTTTAAGGTTCTGGGGAACACCGATCGGCACAGCATC
AAGAAGAATCTCATTGGAGCCCTCCTGTTCGACTCAGGCGAGACCGCCGAAGCAACAAGG
CTCAAGAGAACCGCAAGGAGACGGTATACAAGAAGGAAGAATAGGATCTGCTACCTGCAG
GAGATTTTCAGCAACGAAATGGCGAAGGTGGACGATTCGTTCTTTCATAGATTGGAAGAAA
GTTTCCTCGTCGAGGAAGATAAGAAGCACGAGAGGCATCCTATCTTTGGCAACATTGTCGA
CGAGGTTGCCTATCACGAAAAGTACCCCACAATCTATCATCTGCGGAAGAAGCTTGTGGAC
TCGACTGATAAGGCGGACCTTAGATTGATCTACCTCGCTCTGGCACACATGATTAAGTTCA
GGGGCCATTTTCTGATCGAGGGGGATCTTAACCCGGACAATAGCGATGTGGACAAGTTGT
TCATCCAGCTCGTCCAAACCTACAATCAGCTCTTTGAGGAAAACCCAATTAATGCTTCAGG
CGTCGACGCCAAGGCGATCCTGTCTGCACGCCTTTCAAAGTCTCGCCGGCTTGAGAACTT
GATCGCTCAACTCCCGGGCGAAAAGAAGAACGGCTTGTTCGGGAATCTCATTGCACTTTC
GTTGGGGCTCACACCAAACTTCAAGAGTAATTTTGATCTCGCTGAGGACGCAAAGCTGCAG
CTTTCCAAGGACACTTATGACGATGACCTGGATAACCTTTTGGCCCAAATCGGCGATCAGT
ACGCGGACTTGTTCCTCGCCGCGAAGAATTTGTCGGACGCGATCCTCCTGAGTGATATTCT
CCGCGTGAACACCGAGATTACAAAGGCCCCGCTCTCGGCGAGTATGATCAAGCGCTATGA
CGAGCACCATCAGGATCTGACCCTTTTGAAGGCTTTGGTCCGGCAGCAACTCCCAGAGAA
GTACAAGGAAATCTTCTTTGATCAATCCAAGAACGGCTACGCTGGTTATATTGACGGCGGG
GCATCGCAGGAGGAATTCTACAAGTTTATCAAGCCAATTCTGGAGAAGATGGATGGCACAG
AGGAACTCCTGGTGAAGCTCAATAGGGAGGACCTTTTGCGGAAGCAAAGAACTTTCGATAA
CGGCAGCATCCCTCACCAGATTCATCTCGGGGAGCTGCACGCCATCCTGAGAAGGCAGGA
AGACTTCTACCCCTTTCTTAAGGATAACCGGGAGAAGATCGAAAAGATTCTGACGTTCAGA
ATTCCGTACTATGTCGGACCACTCGCCCGGGGTAATTCCAGATTTGCGTGGATGACCAGAA
AGAGCGAGGAAACCATCACACCTTGGAACTTCGAGGAAGTGGTCGATAAGGGCGCTTCCG
CACAGAGCTTCATTGAGCGCATGACAAATTTTGACAAGAACCTGCCTAATGAGAAGGTCCT
TCCCAAGCATTCCCTCCTGTACGAGTATTTCACTGTTTATAACGAACTCACGAAGGTGAAGT
ATGTGACCGAGGGAATGCGCAAGCCCGCCTTCCTGAGCGGCGAGCAAAAGAAGGCGATC
GTGGACCTTTTGTTTAAGACCAATCGGAAGGTCACAGTTAAGCAGCTCAAGGAGGACTACT
TCAAGAAGATTGAATGCTTCGATTCCGTTGAGATCAGCGGCGTGGAAGACAGGTTTAACGC
CTCACTGGGGACTTACCACGATCTCCTGAAGATCATTAAGGATAAGGACTTCTTGGACAAC
GAGGAAAATGAGGATATCCTCGAAGACATTGTCCTGACTCTTACGTTGTTTGAGGATAGGG
AAATGATCGAGGAACGCTTGAAGACGTATGCCCATCTCTTCGATGACAAGGTTATGAAGCA
GCTCAAGAGAAGAAGATACACCGGATGGGGAAGGCTGTCCCGCAAGCTTATCAATGGCAT
TAGAGACAAGCAATCAGGGAAGACAATCCTTGACTTTTTGAAGTCTGATGGCTTCGCGAAC
AGGAATTTTATGCAGCTGATTCACGATGACTCACTTACTTTCAAGGAGGATATCCAGAAGG
CTCAAGTGTCGGGACAAGGTGACAGTCTGCACGAGCATATCGCCAACCTTGCGGGATCTC
CTGCAATCAAGAAGGGTATTCTGCAGACAGTCAAGGTTGTGGATGAGCTTGTGAAGGTCAT
GGGACGGCATAAGCCCGAGAACATCGTTATTGAGATGGCCAGAGAAAATCAGACCACACA
AAAGGGTCAGAAGAACTCGAGGGAGCGCATGAAGCGCATCGAGGAAGGCATTAAGGAGC
TGGGGAGTCAGATCCTTAAGGAGCACCCGGTGGAAAACACGCAGTTGCAAAATGAGAAGC
TCTATCTGTACTATCTGCAAAATGGCAGGGATATGTATGTGGACCAGGAGTTGGATATTAA
CCGCCTCTCGGATTACGACGTCGATCATATCGTTCCTCAGTCCTTCCTTAAGGATGACAGC
ATTGACAATAAGGTTCTCACCAGGTCCGACAAGAACCGCGGGAAGTCCGATAATGTGCCC
AGCGAGGAAGTCGTTAAGAAGATGAAGAACTACTGGAGGCAACTTTTGAATGCCAAGTTGA
TCACACAGAGGAAGTTTGATAACCTCACTAAGGCCGAGCGCGGAGGTCTCAGCGAACTGG
ACAAGGCGGGCTTCATTAAGCGGCAACTGGTTGAGACTAGACAGATCACGAAGCACGTGG
CGCAGATTCTCGATTCACGCATGAACACGAAGTACGATGAGAATGACAAGCTGATCCGGG
AAGTGAAGGTCATCACCTTGAAGTCAAAGCTCGTTTCTGACTTCAGGAAGGATTTCCAATTT
TATAAGGTGCGCGAGATCAACAATTATCACCATGCTCATGACGCATACCTCAACGCTGTGG
TCGGAACAGCATTGATTAAGAAGTACCCGAAGCTCGAGTCCGAATTCGTGTACGGTGACTA
TAAGGTTTACGATGTGCGCAAGATGATCGCCAAGTCAGAGCAGGAAATTGGCAAGGCCAC
TGCGAAGTATTTCTTTTACTCTAACATTATGAATTTCTTTAAGACTGAGATCACGCTGGCTAA
TGGCGAAATCCGGAAGAGACCACTTATTGAGACCAACGGCGAGACAGGGGAAATCGTGTG
GGACAAGGGGAGGGATTTCGCCACAGTCCGCAAGGTTCTCTCTATGCCTCAAGTGAATATT
GTCAAGAAGACTGAAGTCCAGACGGGCGGGTTCTCAAAGGAATCTATTCTGCCCAAGCGG
AACTCGGATAAGCTTATCGCCAGAAAGAAGGACTGGGATCCGAAGAAGTATGGAGGTTTC
GACTCACCAACGGTGGCTTACTCTGTCCTGGTTGTGGCAAAGGTGGAGAAGGGAAAGTCA
AAGAAGCTCAAGTCTGTCAAGGAGCTCCTGGGTATCACCATTATGGAGAGGTCCAGCTTC
GAAAAGAATCCGATCGATTTTCTCGAGGCGAAGGGATATAAGGAAGTGAAGAAGGACCTG
ATCATTAAGCTTCCAAAGTACAGTCTTTTCGAGTTGGAAAACGGCAGGAAGCGCATGTTGG
CTTCCGCAGGAGAGCTCCAGAAGGGTAACGAGCTTGCTTTGCCGTCCAAGTATGTGAACT
TCCTCTATCTGGCATCCCACTACGAGAAGCTCAAGGGCAGCCCAGAGGATAACGAACAGA
AGCAACTGTTTGTGGAGCAACACAAGCATTATCTTGACGAGATCATTGAACAGATTTCGGA
GTTCAGTAAGCGCGTCATCCTCGCCGACGCGAATTTGGATAAGGTTCTCTCAGCCTACAAC
AAGCACCGGGACAAGCCTATCAGAGAGCAGGCGGAAAATATCATTCATCTCTTCACCCTGA
CAAACCTTGGGGCTCCCGCTGCATTCAAGTATTTTGACACTACGATTGATCGGAAGAGATA
CACTTCTACGAAGGAGGTGCTGGATGCAACCCTTATCCACCAATCGATTACTGGCCTCTAC
GAGACGCGGATCGACTTGAGTCAGCTCGGTGGCGATAAGAGACCCGCAGCAACCAAGAA
GGCAGGGCAAGCAAAGAAGAAGAAGTGA
SEQ ID NO: 33 CRISPR target sequence for OML4
GTGGGTTCCGGCAACCTCAATGG
SEQ ID NO: 34 CRISPR target sequence for GSK2
AGGGGAATGACGCGGTGACCGGG
SEQ ID NO: 35: CRISPR protospacer sequence for OML4
GTGGGTTCCGGCAACCTCAA
SEQ ID NO: 36: CRISPR protospacer sequence for GSK2
AGGGGAATGACGCGGTGACC

1. A method of increasing grain size and/or weight in a plant, the method comprising reducing or abolishing the expression and/or activity of a Mei2-Like protein 4 (OML4).

2. (canceled)

3. A method of producing a plant with increased grain size and/or weight, the method comprising: introducing
at least one mutation into at least one nucleic acid sequence encoding a OML4 polypeptide, wherein the OML4 nucleic acid sequence preferably encodes a polypeptide comprising SEQ ID NO: 1 or a functional variant or homolog thereof; and/or
at least one mutation into the promoter of OML4, wherein the promoter of OML4 optionally comprises a sequence as defined in SEQ ID NO: 3 or a functional variant or homolog thereof; wherein the mutation is a loss of function or partial loss of function mutation.

4. The method of claim 1, wherein the method further comprises reducing or abolishing the expression and/or activity of a SHAGGY-like kinase (GSK2).

5. The method of claim 4, wherein the method comprises introducing
at least one mutation into at least one nucleic acid sequence encoding GSK2, wherein the nucleic acid sequence encoding GSK2 preferably encodes a polypeptide comprising SEQ ID NO: 4 or a functional variant or homolog thereof and/or introducing at least one mutation into the promoter of GSK2, wherein the GSK2 promoter optionally comprises a nucleic acid sequence as defined in SEQ ID NO: 6 or a functional variant or homolog thereof.

6. (canceled)

7. (canceled)

8. The method of claim 1, wherein the method comprises using RNA interference to reduce or abolish the expression of a OML4 nucleic acid sequence or a GSK2 nucleic acid sequence.

9. The method of claim 1, wherein the plant is a crop plant, optionally selected from rice, wheat, maize, soybean and brassicas.

10. A genetically modified plant, plant cell or part thereof characterised by reduced or abolished expression and/or activity of OML4.

11. The genetically modified plant of claim 10, wherein the plant comprises
at least one mutation in at least one nucleic acid sequence encoding a OML4 gene, wherein the OML4 nucleic acid preferably encodes a polypeptide as defined in SEQ ID NO: 1 or a functional variant or homolog thereof and/or at least one mutation into the promoter of OML4, wherein the OML4 promoter optionally comprises a nucleic acid sequence as defined in SEQ ID NO: 3 or a functional variant or homolog thereof.

12. The genetically modified plant of claim 10, wherein the plant further comprises
at least one mutation in at least one nucleic acid sequence encoding GSK2, wherein the GSK2 nucleic acid preferably encodes a polypeptide as defined in SEQ ID NO: 4 or a functional variant or homolog thereof and/or at least one mutation in the promoter of GSK2, wherein the GSK2 promoter preferably comprises a nucleic acid sequence as defined in SEQ ID NO: 6 or a functional variant or homolog thereof; wherein the mutation is a loss of function or partial loss of function mutation.

13. (canceled)

14. (canceled)

15. The genetically modified plant of claim 10, wherein the plant comprises an RNA interference construct that reduces or abolishes the expression of OML4.

16. The genetically modified plant of claim 10, wherein the plant is a crop plant, optionally selected from rice, wheat, maize, soybean and brassicas.

17. A nucleic acid construct, wherein said construct comprises a nucleic acid sequence encoding at least one single-guide RNA (sgRNA), wherein said sgRNA sequence comprises a sequence selected from SEQ ID NO: 35 and 36 or a variant thereof.

18. A method of increasing grain number in a plant, the method comprising increasing the expression and/or activity of a Mei2-Like protein 4 (OML4).

19. The method of claim 18, wherein the method comprises introducing and expressing in the plant a nucleic acid construct, wherein the construct comprises a nucleic acid sequence encoding a OML4 polypeptide as defined in SEQ ID NO: 1 or a functional variant or homolog thereof.

20. A genetically modified plant, plant cell or part thereof characterised by increased expression and/or activity of OML4, wherein the plant is preferably a crop plant.


SEQUENCE LISTING (40 sequences)

SEQ ID NO: 1 - protein, 1001 aa, Oryza sativa
  OML4 amino acid sequence (rice)
MPSQVMDQRHHMSQYSHPTLAASSFSEELRLPTERQVGFWKQESLPHHMGSKSVASSPIEKPQPIGTRMAGRLELLQPYKLRDQGAAFSLEHKLFGQERHANLPPSPWRPDQETGRQTDSSLKSAALFSDGRINPNGAYNENGLFSSSVSDIFDKKLRLTSKNGLVGQSIEKVDLNHVDDEPFELTEEIEAQIIGNLLPDDDDLLSGVVDEVGYPTNANNRDDADDDIFYTGGGMELETDENKKLQEFNGSANDGIGLLNGVLNGEHLYREQPSRTLFVRNINSNVEDSELKLLFEHFGDIRALYTACKHRGFVMISYYDIRSALNAKMELQNKALRRRKLDIHYSIPKDNPSEKDINQGTIVLFNVDLSLTNDDLHKIFGDYGEIKEIRDTPQKGHHKIIEFYDVRAAEAALRALNRNDIAGKKIKLETSRLGAARRLSQHMSSELCQEEFGVCKLGSPSTSSPPIASFGSTNLATITSTGHENGSIQGMHSGLQTSISQFRETSFPGLSSTIPQSLSTPIGISSGATHSNQAALGEISQSLGRMNGHMNYSFQGMSALHPHSLPEVHNGVNNGVPYNLNSMAQVVNGTNSRTAEAVDNRHLHKVGSGNLNGHSFDRAEGALGFSRSGSSSVRGHQLMWNNSSNFHHHPNSPVLWPSPGSFVNNVPSRSPAQMHGVPRAPSSHMIDNVLPMHHLHVGSAPAINPSLWDRRHGYAGELTEAPNFHPGSVGSMGFPGSPQLHSMELNNIYPQTGGNCMDPTVSPAQIGGPSPQQRGSMFHGRNPMVPLPSFDSPGERMRSRRNDSNGNQSDNKKQYELDVDRIVRGDDSRTTLMIKNIPNKYTSKMLLAAIDENHKGTYDFIYLPIDFKNKCNVGYAFINMTNPQHIIPFYQTFNGKKWEKFNSEKVASLAYARIQGKSALIAHFQNSSLMNEDKRCRPILFHSDGPNAGDQEPFPMGTNIRARSGRSRASSGEESHQDISITSVNCDTSTNGVDTTGPAKD

SEQ ID NO: 2 - DNA, 7759 bp, Oryza sativa
  OML4 nucleic acid sequence (genomic) (rice)
atgccatctcaggtcatggatcagaggcatcacatgtcccagtacagccaccccaccttggctgcatcctccttctcggaggagcttcgtctccccacagaggtactccataattgcgataattttggtccaaatcttccttctggaagtcttttctatgtgatggctaatggtgatctgtctggaaattttatttgtttagcctttcctggtgacctggttatgattcatatctacaaatctttaccaattattctcaccatgtttatatattcattatgatgaatatctataatttgtactaatttttctctcaccatgttcatctcttcttctatctttgcagaggcaagttggattttggaagcaggagtcattacctcatcacatgggttagtgctgagtttgatttaacttatactgggttttgttctacatttgtctattagtatgccttgcggttgcagctttaaattttcacgctgttgggggcatgtacttagtcgtttctttatgcatggatagcaaaactttggggacatctattggctcttttttctgcatgaattacaaaccatctataggagggctttctttgaaaggtttacctggccttgacagccatctagcctgcctaaattgagttaacactaggtgctggccttgccacctgattagtgccttggtgaacattggttttaagtattttcccctctatttatgttagattaatttgcaataaataaataaataaataaataaacatgcatgttcttcttatatatgcaattggttgttgtgttttttcttgttatggttactttctttgttctattgtactactctttgagtctttgataatgtgatggttcataaatatgtgggtttcccatgatattttctcataactaggtgggtttccaatattgacaggaagcaagtctgttgcatcttcaccaattgaaaaacctcaacctattgggacaaggatggctggtcgactagaacttctacaaccatataaactaagagaccagggagctgcatttagccttgagcacaagctattcggtcaagagaggcatgctaacttgccaccatctccttggagacctgatcaagaaactggccgccaaactgattcatctttgaagtcggcagctttattttctgatgggaggattaatccgaatggtgcctataacgagaatgggcttttctcaagctctgtatcagatatttttgacaagaaatgtgagtggtttttctttatcatttgcatttgcttcatcaaaatgcttgattctatgaaacacagactcgagaaatttccattccattgatagtaaatgtgctgaaatataccatcacatgacatatgtattggcaactacaacgcttccttacgatcttacattctatacttaatgcttctcatgaatgaatagaaatgtacaaaagtaaaacaaaaaatacaactgaaatgaaagggtagtaaaatgaaatgactttcattcccttcccctttttccataagaatcttgcctcctttatctcctgtttctttctagtggctaaaagaatcaatccactttagtttggtatcgtagtccgtctgttattcttgtacattcttttgccaaaaaaaagtctgcactctggttcaacctttattctattgtaatatgttatctccaatttccaatcattgaccactgtctgattttatttgtaacctgtgcagtgagattaacatccaagaatggtcttgtcggtcagtcaattgaaaaggttgacctaaaccatgttgatgatgagccctttgagttgaccgaggaaattgaggcccaaataattggaaatcttcttcctgatgatgatgacctgttatcaggtgttgttgatgaagttgggtatccaaccaacgctaacaaccgggatgatgctgatgatgatatattctacactggaggcgggatggaactcgaaactgatgaaaataaaaaactgcaagaatttaatggcagtgctaatgatggaattggtttgttaaatggtgtgttgaatggtgaacatctataccgggaacagccttcgagaactctttttgttcgaaacattaatagtaatgttgaggactctgaattgaagctcctatttgaggttagttacttatttcttcttctttgaatcactcttctgttacaacagatttgacatctgagaagccatctgttcttctatgcagcatttcggagatatccgtgccctttatactgcctgtaaacatcgtggttttgtgatgatatcttactatgatataaggtcagcgctgaatgccaagatggagcttcaaaacaaggcactgaggcgtaggaaacttgacatacattattccattccgaaggtaaccatcaaatcatcaattgccacttaactgaaaatgcttatctgcattttctgttgcctgttcttgtgcttagaatgttattattctagatattcactaaaattgagcacatttgcttttctttccccacaggacaatccttcggagaaagatattaaccagggaactattgtactttttaacgttgacctatctttaacaaatgatgatctacataagatctttggtgactatggtgaaataaaggaggtacgatatttcatttgctgactactattatagctagaaagtatgactcactagttctatttgcagattcgtgacactccacagaagggtcatcacaaaataatagaattttatgatgtcagagcagctgaagctgcacttcgtgcattaaacaggaatgatattgcaggcaagaaaatcaaattggagaccagccgtctgggtgctgctaggcggtaagtcatttgggtcttgtcaacagtgataatactctgtttgctgttttctttttagttcttactactactttcttcatcacttttataacatacatattcaccattttaacatttttgacatactagctgaatgcccatacattgcaatgggaattaattattagagaaccacactgcacactctaaagcctcaaaaattaatataaaactatcctcaatgtaaatcttagggtcatattttttgtcgtcattttcacctccaatttgttttccctgttagacggcttgaggttaggaaagggacaaaagtccacctacctcactgtttgggggactcacatagcagtggtggtgggtggtgggtggtggcagtggtagagtatagagtatatattttgaatgcatagtgtatcttcttttatgtttgagtttcttatccacataatgttcatgctgagctgtgcaggaatagtttagttgaatgcagcatattgaataaacgaaaaaaatgtcaaacatgttggtagaatggcatttctctgagtattttaattgtagctattgctttgactgatttcaatgctctctatcacagcttgtcgcagcatatgtcttcagaattgtgtcaggaagagtttggtgtatgcaaactggggagtccaagcacaagtagccctccaattgcttcgtttggtatgctgttttcctttttcatctcaatgtatgttttgctgataggtgcattttctgacacggatggttatattgcaaggttctactaatttggcaacaataacttcaactggtcatgaaaatggaagtatccagggtatgcattctggacttcagacatcaataagccagttcagagaaacatcttttccaggcctatcttctaccataccacaaagtttgtccactccaattggaatttcatccggtgcaactcatagtaaccaggctgcccttggtgagatcagccaatctctaggtcggatgaatgggcatatgaactatagttttcagggcatgagtgctcttcatcctcattctctgcctgaagtccacaatggagtgaacaatggtgtcccttacaacttaaacagcatggcacaagttgtcaatggaaccaactcgaggacagctgaagctgtggacaacagacatctccataaagtgggttccggcaacctcaatggacattcatttgatcgtgcggaaggaggtaatttgtatatcctaatctcctttgtttgaaaaatctgttatgttaagaggaactgaactatcctaggatatgttggttccatcatgggtcatgccatgattttggtgggatgaattcctcgttttctataattacatgcttttgtgggatgaggtggtgatcgaccaaacacatttcgtttctcaaaccaatgaaagttgtgtaatgtttggatgaaagaaattacatctggatcaatctacaagccttatatgttatctaatcattccttgaatgtgtattttttttttcacttgcagctcttggattttcaagaagtggaagttcttctgtccgtggtcaccagttaatgtggaataattcaagtaacttccatcatcacccaaattctcctgttctatggccaagccctggatcatttgtaaacaatgttccatctcgctcccctgcacaaatgcatggagttccaagagcaccatcgtcgcacatgattgacaatgtgcttcccatgcaccatctccatgtaggatcggcaccagcgatcaacccatcactttgggataggcggcatggctatgcaggggaattgacagaagcaccaaatttccatcctggtagtgtgggaagcatgggatttcctggtagtcctcagcttcactcgatggagcttaataacatataccctcaaactggagggaattgcatggacccaactgtgtctcctgcacagattggtggtccatctcctcagcagagaggttcgatgttccatggaaggaatcctatggttccccttccatcctttgattcacctggtgaacggatgaggagccgaagaaatgattcaaatggtaatcagtctgataataaaaagcaatatgagcttgatgttgaccgcattgttcgtggtgatgactcccggactacgctgatgataaagaatatcccaaacaagtatgtgtaacaactgttaatttaggttcatttttttttcttgcctttgccttcttttctgtcattttcatgtatttctaattgacttgggattccaggtacacctcaaagatgcttctagctgctattgatgaaaatcataaagggacttatgattttatttacctaccaattgacttcaaggtgatctagatttatttagtatgcaactaatacatcatatttgttcagatagtcttgcctaatcgaattactgaatgggatgtgtcctacttttcagaacaagtgcaatgtaggctatgctttcatcaatatgaccaatcctcagcatatcattccattttatcaggtgagagatactatctatagggcctgcccagctgagctggctgcaactgcatcacagccagctgctgcccgaagcagcaatgccagtggcttgctcctgcagccagctcagccaagagaaaccattatcaagtgctagtcgcatgaaggcaatagcttacgttctgcatgcggcttgtcaactttggacattgtacattatccaatttgaaataaatcaatattgtgccctcatcccttttttgcagacgttcaatggcaagaagtgggaaaagtttaacagtgagaaagtggcatcacttgcttatgctagaatccaagggaaatcagctcttattgctcacttccagaactccagtttgatgaatgaggacaagcgctgccgccccatactattccattcggatggtcctaatgcaggagatcaggtatgatctttctctctctctctctctctctctctctctctctctctcgttgataaatggagttaaagcagcagatgacacttggacacagtttgctgttttatggcaagttcttttttgttagcaggccttttctgctgtatttgaatgtattttatcacaaatagacctatattttgtggttgtttctgttctgcagttccaaatttcatgccacattgtgggttccttctcactctcttttttcttttgcatgccatgtcatggtctctttcctatatattacagttgcaagcaccattccttctcatttctttgggaactagaagataatagtatctgttacttattattctctcctaatggcactgagtttgctccataatcactagtcattcttgtttggtctttcagaaccttttatgttagctctgaaaggtttattgttccatgcagattgctattcctttaactatatgattaacaccttttgtccttttgttgtccattaggaaccattccctatgggtacaaacatccgagccaggtcagggagatcgcgagcttcctctggcgaagaaagccaccaggacatctcaatcacctcggttaattgtgacacttctaccaatggagttgatactacagggcctgccaaggactgagtaacacaactgctctggatcactaacccccaaatcccaaatcataacttttgcgacgcggtttccatttcccagttttccgccctttttcccccaactttggtttttttggtatgacccccaatctgtatttattaacttccatgaatgcgggttaccgaagacttggctagattgctgcaacattttgtccctgatggaaacatggatagagagacagagagggtgcttccagtttcccctgaacctaccattatcatattaacctgaaggccgagaaaggtgaaaggcgcagcgagagcttccagattttggtcactttttaagaatgtattaaccccatgttgtatagcagtttccagtaactgtgctgaggggagagagagaaagagaggagagcaaggagacaatttacatgagtttttagtggtggtgtggagaggaagtctttccctgcattttcttttggaaccttttctggcgtcttcatctatgttccattttgagttgaggtctcctcttttaagttgtgtgcagaggagttccgattttgtcttcagggaactttgaccgtatctatcgaccttcatatgtaaatcaacatctctatatagtttgtgtgccctctgttgtatgcctgcggccccttgcaccaaacgaattgtctctctaactcgtgagattgctgtcctcgtttggtcgtattacatctgaatctaagcatttgatgttacgcaaatacatgccaatggctgcattgcgacatgtagcagacggccaatgttcaaacaaaaatcttaacttatgaagtatactagtacctccatcctaaaatataacaatttgggactgatgtgtatatcctagtccaatgaatctggcccttgtctaaattcattggactggatatgtctcatccactttcaaatagctatattttgggacgacactttcaaataactatattttggaacggagggagtaaataattataaatactagtactatcaatttggcacatggtgtcaagtccacttggtgcatggtcatctaggcttccctttggtgcctctcttaagaaccttctaagcgtttaacacaaattaaaatcgaagtaagaatctgacacgaattgaattcgaaatttgctctcacaatgagacaaaaacaaaagaatttggcgaatacagcagtaacgccgtggacgaagacaataataatagtctcggactcgggagttgttcagtcagtgtccgtca

SEQ ID NO: 3 - DNA, 2049 bp, Oryza sativa
  OML4 promoter sequence (rice)
cttactgtcagatggactactttgagaaaaaaagggggcaaaataactatatcaataaattaacctctgtcaaaacaggcaacaattaaaattaagagcagcttagaccattctttctaattttctagttataagatgcacattctacttcagttttcgttagcgcgtttttcaaactgctaaacgatatgttccgtgcgaaaactttctatataagtagcttaaagatatcaaataaatccattattcaattttgtaataatcaaaaactcaattaatcatacgctaatgactttatattcccttactcaatcttcatctatcttaaattgggccatgtctctttttttaattaagatgcagattttacttcggttttcattagcacgatttttaaatcgctaaatggtgtgtttcatacggaggatctacttttgaaaatttttaatgatttaaactcaattatttatatattaatagctctctcattctgcgtgcccttacttaatcctcatcctcattacttacaaacactgcataacggagtaatagtattattattaatgttatgttaatcctgatcctaatccctaatccaaagagaaccatctaaatacccggcgcaagcaaccccctctgctctgtcgtaaccaaaaatttccctctcccctgcgaactcccaccacccaaatttaactcccccaacctcccgcccgtcgcgccagctgacccgtcactgacagggtgggccccacgccccggcgcggtgggtcccacgcgtcagcgaccgtgggtagggtgggcgcgggtgcgccccccccccacccggtcccgtgctccgcggtggcggtcaccgggtgcggggggtgggccgcgtatataggcgggccgccgcgccgcgcgctgctggctaggtgtaggagcttcagctttggcccacatcgcccccctctcgccctcttccttcgctttcgtctcaccgcccccaccgcctcgcctgggggaggggaggggaggggagcccttcgccggagcggccaggttccggcgagcatctagaggaggaggagggggagggcggggaatggggagcggcggccggaagagggggcacgtcgtcgctgctgctgctgctgttgttgttgcgtccctctagggttaggtaggggcgttgctggagtagctttctcccacccccaattttttttgttcgttctctttcgctctcgaggtctctctctctctctctctctccccacctccgccccgccgcgtcggggggttggtcctccttgccggcggcgttcgtcgtcgtcgtcgtcgcattgaggggggagaggtgatccggccgtagtccattccagctcggggaagggggggggcatgggggcagctggtccgcgtggtggtgccgccgctctcgaattcgtgcggggattttggttttgaagagggaggtgacccgcacgcgccgatctggtgaggccttgctcgttttgtgctgttttttgtgcctagctttggtcggaggtgtttgaattgttggggaattttgagcttttgctgtgatctgagcttcaaatttcggtgggggttaacttggcctgggcacctcggaatttctgtttaatttttggtggggtttctttgatcacaagatacttgcttgcttggagctttgggagcccgaggcgcattaaattccacatcttctgcgctgttttatcgggaaattaaacatttcgtgctcaagtctgtgggggggtttttccctcggatttgtcaaatctggcggctcttgttcgaaaattttcatcttgggagcttacgaacgcaaaattcttcacatttcttttgcttcctggcttggaagctgtggaatccaaatttttatgtgctgaattgacatggttagccatgttttttttccacagaaccacatgattttagcaaaatttcgccatttctactttgatccggtggaatctagttgccagatgtgtcgactggtaccttgtctaactagctccatggctatgcgcttgcagg

SEQ ID NO: 4 - protein, 402 aa, Oryza sativa
  GSK2 amino acid sequence (rice)
MDQPAPAPEPMLLDAQPPAAVACDKKQQEGEAPYAEGNDAVTGHIISTTIGGKNGEPKRTISYMAERVVGTGSFGIVFQAKCLETGETVAIKKVLQDRRYKNRELQLMRAMDHPNVISLKHCFFSTTSRDELFLNLVMEYVPETLYRVLKHYSNANHRMPLIYVKLYMYQLFRGLAYIHTVPGVCHRDVKPQNVLVDPLTHQVKLCDFGSAKTLVPGEPNISYICSRYYRAPELIFGATEYTTSIDIWSAGCVLAELLLGQPLFPGESAVDQLVEIIKVLGTPTREEIRCMNPNYTEFRFPQIKAHPWHKVFHKRMPPEAIDLASRLLQYSPSLRCTALDACAHPFFDELREPNARLPNGRPFPPLFNFKHELANSSQELISRLIPEHVRRQATHNFFNTGS

SEQ ID NO: 5 - DNA, 4624 bp, Oryza sativa
  GSK2 nucleic acid sequence (rice)
atggaccagccggcgccggcgccggagccgatgctgctcgacgcgcagccgcccgccgccgtcgcctgcgacaaggtatgtgactaaccggatcttggcgtgctgatccgtggttttgcggttctttgctgtgtgctgatttagtgtgctgttcttggtggagcagaagcagcaggagggggaggcgccgtacgcggaggggaatgacgcggtgaccgggcacatcatctccaccaccatcgggggcaagaacggcgagcccaagagggtgagacacgagccttcccccccccccctttgttgttttggtcttggttccatttcttgagttgcagtgaaatgctgccggttcttggtttaggaaggtgttcttgtgtgttctgcagctagtttcttagctccgtgtagtgatttttggtgatgggaaagccattggctctaagagaggcatgtggattagtggtcagattttgcaaaagaagtaaactgttggtagatatcagccaatttatttagtgttagttgttcatgttcttgtattactgcaagatctgttgtaaataactaaatatggcttgtttggtgctcatttttggtggtttgtaggggaaaaagttgggtgtgttggattacattgttgtgaacactagtgctcataattaaattttggtcttaagatggtaattttgtacttgattttcagacaattagctacatggcggagcgcgttgtgggcactggttctttcggtatcgtctttcaggtgattcatctttcagaaagttgttatttgtttctttcttttcgtgctgtcgacttgttggtctgatgtttagcttgctggtttcatgtgtaggctaaatgcttggagacaggagagactgttgccattaagaaggtattgcaggaccgacggtacaagaaccgtgagcttcagcttatgcgcgccatggaccaccccaatgtcatctccctgaagcattgcttcttctcaaccacaagtagggatgagctgttcctcaatcttgtcatggaatatgttccagaaacactctaccgtgtgcttaagcactacagcaatgccaaccaccggatgccacttatctacgtcaagctttacatgtatcaggtgtgtggattgctaatcaatcataaattttgaaatgcctgccttcctgtgtgtctcttctaagtctattctacattggctgcagttatttagggggcttgcgtacattcatactgttccaggggtctgtcatagggatgtgaagccacaaaatgttttggtaggtattcatgatcagattattattttgctatgcgatggcctttgattattggctctgaactcctttcttgcaatacaggtggatcctctaactcatcaagtcaagctctgtgactttgggagcgcaaaaacactggtattggccttttccaccctaaagttttgtaatacgcacacattactttagactttcttttttttaattggactttagacgattcttgctgtagactagtcagttttgaatcttaccatttgttaagttggagctagccctgtgttactgaatcgttcaaagaactcttatatacttggtgaatcttacccctttttttcttcctttttattatgcttgatggaagtttcatggaaattccttagttttacacctttttccaccttattccagatgtttgctacaattgtacttttgataattttgatcttactgtcctaatatccattaatttactattccatcaggtcccaggtgaacccaatatatcatatatatgctcacgctactaccgagcaccggagctcatatttggtgcaactgaatatactacatcaatagatatatggtcagctgggtgtgttcttgcagagctactccttggtcaggttggtttcttttttctatggttgacagatctgcaaacttttggtttagttatttaagcatgatgtcatcactgttgctgtgattttgattatcttgtatttgtttttgctagccattgtttccaggggagagtgcagtcgatcagcttgtagagataattaaggtactgcaagacatgccatgcagttctaattttgctcctactattgagtatgggcatcttctctaaccttgtatgatattcttgcaggttcttggtacaccaacccgtgaggaaatacgttgcatgaacccgaactatacagagtttaggtttccacagataaaagctcacccttggcacaaggtaagcatacaatcttatccatgttgagtcatatatcacgtcatcttttatagtttcctggacaactatgaaaatgtagctgggctcatttccaataatagattctggacaccagatagctttacaatgcaatgtataaataaggaggtgcatacaggtactgatttttctaacttctgcgtaggttttccacaagaggatgcctcctgaagcaatagacctcgcttcacgccttcttcaatattcaccgagtctccgctgcactgctgtgagtatattcttgctgcaattttaagtagcagaacagtagaaaagtgatttttcactactgctcacagcaggggtactgtaaaacgccccttttcttattgctgttatgcaagtttgcctactgtagctggtcatatgagctgttacttttcaccctttaagagttgcacaaatttgagcgtaaccaaggaattttcttaatcactttgccctccaagtgctctttgatttgtgcaactcctgaaatggggtggagtggagaaacactccttgtttctttctcttttttcttttttcctaaagtagattgaagaatgctagtcttcactaactttggttttagtggggcatggccattatggttatgatctttagtggtccattaccaaatcaatgttggggtggatgaatgatagttgtctcatgtttagtcgttattcagtgtaattgcaatagccagatgacaacttaatattgattttttttccgatgtgcttattcatttgaatatctttatgcagcttgatgcatgtgcacatcctttctttgatgagctgcgagagccgaatgcacgcttgccaaacggacgtccatttccaccactattcaacttcaaacacgaagtaagtgaatcagatgaaacataatctgctacacaacttcagatcttggtatccatgagaaaatgtgtactctccttggtgctcattggtgctgccttttggtctctacagctagcaaattcttctcaagagctcatcagcaggctcataccagaacatgttcgacggcaagctacccacaacttcttcaatactgggagctaaaaatgctaaatgcacaccaccagaccttttgttggatcgttttcgcggaaccggtgaagttcacatgaaggctgagtcagatgattcttcgaatccccgcaaaacaagaagaatagaaaatatgattcctcagatgatgatatgcaaatgcttcgttggaagttcaattcaatcatcgaagaagaacaacattgtaaatcgagaagtttttgcatcgcgagtttggtagtgaaaccgggatcagctggtatgacggaggaaaccgaaatgtttagatccatgactgagttttctttcattttttttgcccaattgtaacagaagaatatagttccctaatgtaggcgtagttgtaacctgtaaactgccactgttttgttcacattccatgatgtaaatgccaccatgcctctgatgaataactctccttgtaaccttgttccttccatccttgactgtttaccttaaagccgtggacagtgtacactgtacatgtaccgtgctacacggaaggacatatttgaattttttttctctctctcgaaagactacatcaagcattgctggatttttttttaaaaaaatggcacaactttcgatggtcaaccataagcaattagtgtcgttttaaaaccccttactcccatatgcacaatacttatctttcatttttctagattgtttagcataaaataagatttaaaaagaaagaaaactatattgctatttaattgttgggtgtagaatgggaaaactttttaaatgaaagatgattatttcttaatgtaacaagtagtactgtagtgtggattgaattggggcaaactttaaactctaaaacgagaactatttttgaatagagcgagcatttaaaagatgaattacataccacctatagagatgaaaaaaaaggcatgggacagttagggcttgggccccatagcttgttaggtttgtaagattaaaatttagagtaaatttcacaaaactacacatactatgaccaaactatcacaaaactatatatttaactcgatgtatcataaaactacacatttaagatgaaatgttacaaaactacatgtttagttactacattattacagaactataggtttagaaccaatttagttacaaaacaataatgtttattgctctagcataataatggtgctagggatttaaactctaaattgtgataacttcaatattaaatatgcagttttgtgatacttaactttaaatctatagttttatgatacaac

SEQ ID NO: 6 - DNA, 2247 bp, Oryza sativa
  GSK2 promoter sequence (rice)
ccgtactgatttcggcagcatcaaggactagaggaggaggaggagcaataaacaagtgccgccatgtcgcttgcccggctttcaggggcgcttttggaattctcgtttgaccgcctacaaacccacagcctgtccctgcaaccaatttggccctgcgccacgccacccccaaagctattagtactaaccacaccccctctctctctctctacacttagcagtagtactaagaccctcttttataaaatttttaagtcactatcacattcgttagcagtagtactaagaccctctttgtaaaacttttaagtccctatcacatcgaatgtttggacactaattataaatattaaacgtagactattaataaaacccatccataatcttagactaattcgcgagacgaatctattgagcctaattaagtcatgattagcctatgtgatgatacagtaaacattctctaattatggattaattagacttaaaaaatttgtctcgcaaattagctttcatttatataattagttttgtaaatagtctatatttaatactctaaattagtgtttaaaacagagactaaagttaagtccatgatccaaacaccacctaacatggacaattaggctgtactacaaccttttgccaagctacgtgtacaggtaaatcgcacacatgttgtcatctttggaggctgaaacatggggaaatatcatgtgaaaaccgttaaataagtgaaaactcatgaaaattatatttaaaagttctctaaatttatataaaaattaatagagataaatatagacatgaaatacatttacatcaaatctcaagtcgaaactcaacttttatttgagagaatataaaagacaaattttaggtgaatagtgttctattatttttcatccgaaatttggcatttttgttactcccaaataaagttgagtttaacttgatatttggtgaatatatttcatgcctacacttctctccagtaatttttcatgaatttattaaacttttagttccgattttcacgtgttttcacttatttgatggtttccatcggatatgtccctgaaatatggtattgaagtacaacatagttcatacttgggtcgtgtttgaagtcatgtaagggcgttataagctcataggttttgcttacatacaattggtgggataaaaaggcaccggtaatttcttcaagattgataaaataaatgtctagcgctataaggccatggcacacatcaaatgttgtttagaacaggttatttctagctccataaattgttggatttgaatttgtgattacattgataataattgattgaatcagtttgttcttattttagagaaaataaaaaaaataaaccactataatttaacttacaaactccaaaactagtctggatctgtaatttaggttgtgctaaacaaggcctaaagaaaagaagtaatgtttggagaacatgtttttaaatcaatatggaccatcttctaaaatgggcacactgtgcaaccgaaatggttattagcaacttaatatccaatccttaaaaaaactacattgaaaatatcctaaatcccaaaattaaattttaaaatctaattttggtagcgattgattatttgtaggggcaaatgatgaggccctaaatcaaccatgtttagctacttcctcgctttctttaagtatgtttcatacgctacaaactgatattttttgcaaatactttttattaaaaaaattattttaagtctgcaaaagctaatgtttaattagtcctacactaataatcctccttgtttggcttgccgctgataagcttagtcaaaaccctgatccgaactgcacgtaagaacggtcaagaaaccatttcggttacatcacacaacacagcctcatctctcatgctgtcatgcttgtggtgcacctagcaattcctccctccccatctgtcttcctcctctaatctaatccacctccccactaatccaccagctgtgtacactgcagcagcagcagcagctaaccactctcactaaaaactatagcagctgcagtaacagcagcagcatcacccaccttcttcttggtcaaagccatccatcccaccactcacccatccctcccagtataagccaaaccaatccatagaggaggaagaggaggaccaggtggtggcacacctaagctttgtgcagtgccattcacgcacctgcagcttccagctttgccac

SEQ ID NO: 7 - protein, 925 aa, Triticum aestivum
  OML4 amino acid sequence (wheat)
MEPYKLMDQKTPFGERKLLGHQRHVNLPPTPWRADQDPLQQHDSFSKPLALFPNARKGHLNMTQYENGLFSSSLPDIFDNKLRLTPKNGLVGQPAEKEVNHADDEPFELTQEIEAQVIGNLLPDDDDLLSGVLYNVGHPARANNMDDIDDDIFSTGGGMELEADENNKLLKLNGGANTGQTGFNGLLYGENPSRTLSIRNINTNVEDTELKLLFEQYGDIRTLYTAYKHHGLVMISYYDIRSAERAMKALQSKPFRQWKLEIHYSIPKENPLENDNNQGTLAVINLDQSVTNDDLRHIFGGYGEIKAIHGTSQNGHHKYVDFFDTRAAEAALYALNMRDIAGKKIRLERCCAGDGKRLTTLHRPPELEQEEYGACKLGNANSLPSTYYGSVNMASMTSAGPEHGISRVLRPRVQPPIHQFREGAFLDVPSSTMQSISSPVRIATAVTHNNRSTVGENGHSLGKMGGQINGHLNYGFHGVGAFNPHSLPDFRNGQSNGISCNLGTISPIGVKSNSRTAEGMESRHLYKVGSANLGGHSSGHTEAPGFSRTGSCPLHGHQVAWNNSNNSHHHTSSPMLWPNSGSFINNIPSRPPTQAHGISRTSRMLENVLPVNHHVGSAPAVNPSILDRRTGYAGELMEAPSFHPGSAGSMGFSGSPHLHQLELTSMFPQSGGNQAMSPAHIGARSPQQRGHMFHGRGHIGPPPSSFDSPGERARSRRNESCANQSDNKRQYELDIERIVCGEDSRTTLMIKNIPNKYTSKMLLTAIDENHKGTYDFIYLPIDFFQNKCNVGYAFINMISPEHIVPFYKIFHGKRWEKFNSEKVASLAYARIQGKSSLIAHFQNSSLMNEDKRCRPILFHSDGPNAGDQEPFPMGTHVRSRPGRSRVLSCEESHRDTLSSSANNWTPSNGGGHASGYSKEADPTTA

SEQ ID NO: 8 - DNA, 6947 bp, Triticum aestivum
  OML4 nucleic acid sequence (wheat)
atggacccatacaagttgatggaccagaaaactccctttggtgagcacaagttgttgggccatcaaaggcatgttaacctgccgccaaccccctggagggctgatcaagatcctctacaacaacatgattcgttttcgaagccgttggctttatttcctaatgctagaaaaggacatttaaatatgacccaatatgagaatggacttttctcaagctcccttccagacatttttgacaacaaatgtaagcccttgatccttgtctcttgcagtttttatttcatttattgtagcacttcataacactgaactatgaactgcgtccatccgatatggtactcctcccttcagttcatataaataatactccctccgtcccaaaatgtaagacgctttttgacactatactaatgttaaaaagcgtcttatattatgggacggagggagtagtatgcagataacggaaagggtaaacaaaagaagataaggaaaatatttttatttgcttattaataaaaagcttgtttgcttttattgactgtttcacttcagtgaaatctgagcttttcttgctacatccaagtgagaaacgagacaaactggcctgagcttttcatgctacatccaattgagaaatgagagtctgtcctgtgcttttcatgctaagtccaagtgagaaaagagacaatctgcagtaatattagtgcttaatactaaaccacttttaatttgctgatgtgcagtgagactaacacctaagaatggccttgttggccagccagctgaaaaggaactcaaccatgcagatgacgagccttttgaattaactcaggaaattgaggcacaagtaattggcaatctcctccctgatgatgacgacttattgtcaggtgttctttataatgtgggtcaccctgcccgtgctaataacatggatgacattgatgacgatatattctctactggaggtggaatggaattggaagctgatgaaaataacaaattgctaaaacttaatggaggtgccaacaccggtcagactgggttcaacggcctactgtatggcgaaaacccctcgagaaccctttccattagaaacattaataccaatgttgaggatactgaattgaaactcctatttgaggtaagttccatcttccagcttgactttctcccaactctgaaggcaatatatttcacctgatagcatttattttctttgtagcaatatggagacatccgaacactttacactgcctacaaacatcatggtttagtgatgatatcttactatgatataagatcggcagaacgtgccatgaaagcgcttcaaagcaagccattcaggcagtggaaacttgagatacattactccatcccaacggtatttccttgatataatgccattctgacttgatatgatgtggtgctttgacattacttaatgtgatattactacgatgtttgcttgccattatttgttgcattggtacttaattggcactggaaatgtatttatacttgcaagaatgttcacattctaatgctgactttgttccaataggagaaccctttggagaatgacaataaccagggcacacttgcagtgattaacctagaccagtctgtaactaatgatgatcttcgtcatatatttggtggctatggtgaaatcaaggcggtatggcctgcgcactaaccaactcttatgtcagctagtacactacagatactaacttccttgtttatcagattcacgggacatcacaaaatggccatcacaaatacgttgagttttttgataccagagcagcagaagctgcactttatgctttgaacatgagagatattgcaggaaagaaaatcagattagagcgctgctgcctgggcgacggtaaacggtattactgtgaccataattttgcgcatctgtccatttttagtgcttctagtgccttcgcttttcgaagagtcttattacacaccttgtgtcggctacccttcatgaagttcttttttctccaacacgctttagaatgctgttattttttattagaggaaggtactgaaatggcacaaggtatgaccaatggaagccaaaaatgaacagaaaaactaaaaaaccagcaaacaaaaaaccaaaaaggctaagaagactaacaaaatctaaccaaaactagcataatgacctatatatactataccaattaggaagcaagagacctgaagccccagtaagcagcagaatatggcggtccagtcggtagcagcaaccttcgcagatcaactttgtataaagttatctgggtatctgcgaattgaggaaatatataattcaacggtgtttatggtcttgcatatactgtgaagttggtaaacatatcgtcaatggacatatacagtatacacggggctgatgcaattcctgtctcttcaataaaatatgttttagttattaaacacgcaaacttgtgattgacgtttaatatgattttttagagtcgtcatttgcacttgaattcaaagttggttgtatacttgtatatttcttgttttagggaagtgtgctttggagtttggaggaaattggtaggtgtaaaaaaatcttttcatatgatgtgcaggaagatgtgttttagaacttgatgcagaacgtcccccctatggattattatgcttgtctaaactttatttttggaggaagaaacaagagcatctgactttccttatgcctattcttacaactgtattagtaatgctagtttttgcacaacagtttgacgcggcacaggcctcctgagttggagcacgaagagtatggtgcatgcaagctaggaaatgcaaacagtctgccgtcaacttactacggtatgcagtttgatttcaaatcacgagacatgtttctgctgctaatcgcatttactaacctatgtatggcataatacaaggttctgtcaacatggcttccatgacttccgctggtcctgaacatgggatctctcgggttctgcgtcccagagttcagccaccaatacaccaattcagggagggagctttcctggatgttccctcaaatactatgcaaagtatatcctctcctgttagaattgcaactgcagtaacgcataacaaccggtcgactgtcggtgagaatggtcattcacttggaaagatgggtggacagattaatggacacttgaattatggatttcatggggttggagctttcaatccacattcccttcctgactttcgcaacggccaaagtaatggtatttcttgcaacttaggcacaatatcacccattggagttaagagcaactctagaactgctgaaggaatggagagcagacatctttacaaagttggttctgctaaccttggtggtcattcttctggtcataccgaaggtactaatttgggtgccttatttactgatgtagccatatgtttatggagacgcactgtttccattaggttcatttgccatctctttcccttccagtcattttcttgaaaatgtcaattttgaaagaacatatgctttgatatcaataatacagaagcttttatagcttaatggtaattggtgtagcctaaattatactatttttgaggttgcaactattctgtttagacaatgcaattaggcttacatgggcatgccttgtgttcttgtagcacccgggttttcaagaactggaagctgcccccttcatggccaccaagtagcgtggaataattcaaataactcccatcaccatacctcctctcccatgctatggacgaactcaggatcatttatcaataatataccatctcgacctcccatgcaagcgcatggaatttcaagaacatctcgcatgcttgaaaatgtccttccagtgaatcatcatgttggatctgcaccagctgtcaatccatcaattttggataggagaactggttatgtaggggagctgatggaagcgccaagtttccaccctgggagtgctggaagcatgggtttctctggtagtccgcatctgcatcagttggagctcactagcatgtttcctcagagtggagggaaccaagccatgtcccctgcacacattggtgctcgatctcctcagcagagggggcatatgtttaatggaaggggtcatataggtccccctccatcttcatttgattcaccaggtgaacgtgcaaggagccgaagaaacgagtcatgtgctaatcaatcggataataaaaggcagtatgagctagacattgagcgtatagtctgcggcgaggattcccggactactttaatgataaagaacatcccaaacaagtatacatctgggactttctgattttgttctagtttatgtgcaagtgtcactctatttgaagtcacgccatgttttgatgtttctattgccttaatggtatttcaggtacacctctaagatgcttttgaccgctattgatgaaaatcacaagggaacttatgattttatctatcttccaattgactttaaggtgaatggagcttttgtaaacagctgttgcatgtttatccttggttcgacattacttgcatacaacgaactaatggtgctcatgtgcattttcagaataaatgcaacgtgggctacgcattcatcaatatgataagtcctgaacatattgttccattctataaggtgagagtgagatgttacaagttatgaaatggcggcagtgtattagataaagcttcatgttgacatttttatatgatttttcaccctctgctttccgtcgtcatttctttttccataactacctgtattacactatcatgctacaattgcatggattttggatatcgcatgtcaggtagtcagtagtacctttaccatttctggtttcacgctctaagcattttttacctaatgccagtcgataaatgaacaacatacatgcctgtctctttcagatatttcatgggaaaaggtgggagaaattcaatagcgagaaggtagcatcacttgcatatgctagaatccaaggaaaatcatctctaattgcgcacttccaaaattcaagtttgatgaatgaggataaacgctgccgccctatactttttcactcagatggtccaaatgcaggggatcaagtatgttctctgattgtccatatcctttgctgtattactgtttcgatagggcacctgacttggtgccactaactagatgacctgtatatcttattgtgtgcccatccaatacatgatcggtgaagtccacacacatacctaattttatatcattatatttttattatcttgcatctgaaattaagcagtagaccttacacagtttagtatgtttttttcttatgctacgtcagaacttttcctgagtatttctttcctttagaattgtattgacgcggaaagaaatactgaggaaaaattcttactccctccgttccaaattactcgtcgtggttttagttcaagagtactaattaaaatcctacaaatcatggaatatgatcctcaatttattctaaatcctttgaaacaaggaggtccttgaggattcaagcataggctcatcgttcttgtttcctagtgttgctatgttctttttaatgatacatatagctgtataggctcatcattttcttcacatatggtggtgtttgatgcgcaattgggatacattgtgggttagggagcggaaaataaaaccatcttgttaacattaggcagggctatgagtttggagtagagaatatagtgcatacatgacaagattgcccctcgataatggctttacttaattttgtgtgtatatattttttgtatttttaacattattactcaacctgtctacaaaaaaccatcgttctgattggacttcaaactgtggtatatgaaactacatatcccatgccaaacaccccaatagattgaactcctcccacccactatttccattcttacctcccatgatgagtctgaactgaccatgtttttgttgtaaacttttctcaggaaccttttccaatgggaacacacgtccgttctaggcctgggcgatccagggttttgagctgcgaagaaagtcaccgggacactctgtcatcttctgccaacaattggactccttccaacgggggcggccacgcttcaggctactcaaaggaggctgacccaaccacagcttgaaagctgaagcactaaccacaacatcaacatccaaccttttgacatttgcaatcccagttttcacattaccatcctttcccacctctttttgcttgtggtattttcggagtctgtagctatttagtactttctatgtcgtgggctaccagaggcttcctagaggctgcaaattttgtcgctgagtagaagcaagggaacggacggagggtgctcccagtttctcctgagcctatatgcgtgtattaactgaaggccgtggaaggcaaaactcgtggggagctctctgagattttggactgtaaggtgtaacccagcgttgtacagggtttcctagtaagaatgcatgacggggacagccgacactgtattggtgctgttgtatgaaaggcaggctgtgccatgcagcgtcttttgaaactgttttgatgttaactactccctccgtccgcgaataagtgtacatctagcttttattctaactcaaagttttgaaactttgaccaactttataggtaaaagtagcatcatttatggcactaaattagtatcactagattcgttctgaagtgtatttttataatataccaatttgatgtcatataatcctactactcttttttaacaagttggtcaaaattataaaactttgacttaggaaaaacggtagaagtacacttattcgaggacggagggagtaggaaacatgcccgtgtgttgcaacgggagaaataaaatccttgacataatgataattgtt

SEQ ID NO: 9 - DNA, 3140 bp, Triticum aestivum
  OML4 promoter sequence (wheat)
gaggaggggacaacaagaatgccagatgagaaggggatgatgcacatgccggcaacatgtgatatgtacatgtcttggttagagacttttgtttatgcaacctattaaaaactatgtgcatgtttgcttgatgtgttaaacatttaaatttgaagaaatcaaaatgtttgaatgaaaaagaatatggagaccgagatatgtcgactctgagtctgccgtcgcctgtctagatttcaaatgaaaaacgtcacatatacattctgacaagcacaaatgcagcgacattactcttagaacgcagaaaagttgctatgagtacaaacatgccaacctaacaggaagtgctgtcgagaacgagcccttgccttgatggttggccttggcggtggccaactagcccacctgggtttgatccctaggattaacgcgagtgtttcacctggcgcaaaagaacccataacctagggttcttttgccagttttaaagtatctcacatgtctatggagattcacggagtcgaatgatgcgatttaggtgtctgtcacgaaattgttttggtgtgaatcaaatgatttgcccatattagatgcaaagaagaatcattttaattattttccctatatctgttatctttaatgtattgaaaatgtaaataggaacaacgtaattttcaaggcaatcaataacaatcaatgttgcatttttaggcttgtttgaaaatgcatatagctagtagtagtatattttgtttgaaaacgtgatttcaaatatactcctcactaaactgaattttcccagtgtttttggaagcagagttcttccaacacggaaggtggtaccagtataaacgtgccaacctaacagcaagagccgtcgaaatcctcggtggttcggcggcagcaactccagcggtccagtcccgatcgacccccacacaactcgtactggtgagcgttatcctgtcccacaccagacaatcggagaacgtgacctccgcgtcacctcaccgcgccaacccccacccctccgcgaaataattccgtccccgtccaaacgccgcttcccacccgggccgacgcgcagccacgcgggtcccgacgtctgacaccggccccagctcactgacacgtggggcccctgacccgggtacatgtgccgttcggcacgaacggaatggcggaggagctatacggtgccgcggtggggtgcgccgcgggtgagcctaccgcggtggggaccacaccgggcgcggtatataaaggccccgcaccttctgcatcgcgtttccacttagtccaataaataatatagtaatacagcatttcgccgctcttttaattagatttttttggccttcgtctccgcttcgtctcgtctcgtctcaccaccgccagtccaccaccagctcgccggaattccctcgccggagacgcgctgcggaggcatacctaggaggaggagcgaagaatggggaggagtggaggggtgcccgctgccgcgctcgctcgctagggttaggtgcgcgcgtgcgtgaggatggagctcgctctctgagctcgcctcgaccccccgctgagggcattagctgtgccgtcgctgcgcgctgggtgatctgccctcctgtgagctccgggggagccgttttggcttgcgccatggagccgtctcttccgggcgcggccacgggttgcgtgttgcggtaagctcctggccgcgacaagctgaaggggatctcgactgccgatctggtgagcctacaaattcttccgtttctaacaatttttttgcgggttttcatgcaaattcggggtgcgtttgtgcagaaaaatggtgtttgaattcctggaggtatttgtttgggggtaattttgtgcgtatttttctcgctttgtgtgatctgtgctcgggttcgggggatactttgatgcggttgggcagtattttggtctcttctgcctttttattttgatcacaagtttcttgtgctctttcgagctcgtcgaggacgaggagcattaaatttcccgtcgtcttttgcgctgttttatggctaattagtttgggagcacagagttctgcacggaattttgcataaccttttttaatcgttcaattcgagttgttccggtcctaaattttgcagaatttctccagtgttccgtagccggtctcgtgagttcgattttgggttcacggtcgatcaaatctaggcctcgggactgcattttctcgcgtttattatttgatgatctgcttcagtcgagctacctgaggtgttgaaacttggtatctgtctatctttcaaggtgctagcaggatgccagctagaatcatggagcagaggcaccacatgcccccattccacctccccgtggagtccgaatcgtcttctcccatgtggtaagccaactgcaatagccattattgcccgatatccttaaatgatgtctaatgatggactgcattctttcttactttagggtagggggtactaattggttcagttttggggtgacttggtcagtatcattaacaactagacctaggttaattccttcatcattatcgaattctttttgtagaggcctgttggacacttcaggcaggaatgttttcctgagcatgttagtgactacctgaacattcgtgtaatttgtaggtgcttattagtttattcttcgggtagtttctctcggactaaataaaatgtgacctagcagaacactgttacagtttacggatatgtgggggcatccgaggttccacatatgggacaattgatcgagcaagattggaggatgtgtatcgttgtttccaggtagcttaaggtagctgtcttgctatatatgggtgaagcagctgatttggaaggcacatggtctcacaggggtgattgggtatcgattattgacagatgcgcatggatgttgcctacaatgattcttccattaaataattctgatgttgcttcatcacttctctttgcgctcagtgtttgtgttcgtttttatggctgatttatttcttgttttgaaaaacagaaaaaagattcattgatttcgggaagtaggtctgctgcatcttctccagtcgaaaagccaaagcctattggccaaaggttgtgcatcaattaggactt

SEQ ID NO: 10 - protein, 402 aa, Triticum aestivum
  GSK2 amino acid sequence (wheat)
MEHPAPAPEPMLLDEQPPTAVACEKKQQDGEAPYAEGNDAMTGHIISTTIGGKNGEPKQTISYMAERVVGTGSFGIVFQAKCLETGETVAIKKVLQDRRYKNRELQLMRSMIHSNVVSLKHCFFSTTSRDELFLNLVMEYVPETLYRVLKHYSNAKQGMPLIYVKLYTYQLFRGLAYIHTVPGVCHRDVKPQNVLVDPLTHQVKICDFGSAKVLVAGEPNISYICSRYYRAPELIFGATEYTTSIDIWSAGCVLAELLLGQPLFPGESAVDQLVEIIKVLGTPTREEIRCMNPNYTEFRFPQIKAHPWHKVFHKKMPPEAIDLASRLLQYSPSLRCTALDACAHPFFDELWEPNARLPNGRPFPPLFNFKHELANASQDLINRLVPEHVRRQAGLAFVHAGS

SEQ ID NO: 11 - DNA, 4729 bp, Triticum aestivum
  GSK2 nucleic acid sequence (wheat)
atggagcatccggcgccggcgccggagccgatgctgctcgacgagcagccccccaccgcagtcgcctgcgagaaggtaaccggatctgtgctgggatggtgttggccgtgtgtttcttggcgtggtgttccgttgagctgatgtttagcgtgttgttttcgttgggcgctcttgttgagcagaagcagcaggatggcgaggcgccgtatgcggaggggaacgacgccatgaccggtcacatcatctccaccaccatcggcggcaagaacggcgagcccaagcaggtgagctcagcgtctcttatgtttcgcttgtgtctcttggcctgagtttgcacggccagttcttgccttggtgagatgtgtctgctctcctgcagctattctctttagctatgacaactcattgaaatatagctgtgtggattcttggttagatttttcttcgtttaccaaatacgaaaaaaatgtttcaaagcggctgaatttatcaattatcaaggacgatgtagcttgtcagcctatttttgtagtgctcatttgtttgatcctcatgtaactatggtttgctcaagagatctgttccaaatatgcctgtgtggtgttccatactgtgggttttcgggacaaatttggacggcttcagttagattttggccaacactagtgctcaaatctgttactatgagcaacagctgatacctctttggcgcccagttggtaatgtcctgctttgtttttcagacgattagctacatggcggagcgcgttgtgggcactggttcgtttggcatcgtctttcaggtgattgctctagccattgtttgtttccttgtttgtgttgttgactaccagcctgatgtttagggaaatgttgcatgtgtaggctaaatgcctggagaccggggagacagtggccattaagaaggtactgcaggaccgacggtacaagaatcgtgagctgcagcttatgcgttcgatgatccattccaatgttgtctccctcaagcactgcttcttctcaaccacaagtagagatgagctgtttctgaaccttgtcatggagtatgtcccggagacactctaccgcgtgcttaagcactacagtaatgccaaacaggggatgccacttatctacgtcaagctttacacctatcaggtttgtgaatttccagtgaataaatgtgaaatgtgtgtctgtcattgtgcaactattctaagtcaattttacatttgtggcagctattcagggggctggcgtacattcatactgttccaggagtctgtcacagggatgtgaagccacaaaatgttttggtatgtatcagaggccggggtcttcccctttctgaaaaaaatgtatcagtgaacactgaacagatttgctcagttttcatgtatggcttttcttgcttgattttgaacttgcctccacttgctatattatacaggttgatcctttaacacatcaagttaagatctgcgactttggaagcgcgaaagttctggtatgttggctctttccccaagagtttagtgatacgtacacactgcttcaatccatttgtcctgtcgtgtaggctactcattctattcagtattgaaccagaatcggcatcatggtctgtgctattttgatttagtcttactgttttaggcttatagctggccaggtgttaagattaaaattaagtcacttttatataccttacagtttgacttcttcagatatttttggtttataaactattatctctgtattccgcttattccttcctagattgctgaattcttgctttagccgaatgcaaagtttctgatcttcacttcatttatttacattggatgtccgacactgaatttaaacttttgttcctttactacaatatcaacctgcatagtactttgatgttacttacctgctaatccgatatcgtttttcttgtcctgttctatcaggtggcgggtgagcccaatatatcatacatatgctcacgctactaccgtgctccggagcttatatttggtgcgactgaatatacaacatcgatagatatatggtcagctggttgtgttcttgcagaactgctccttggtcaggttagttccttcgtttcgttcacatatattgcaatctcctaggttccaactaagatgtcaatactgtagtttctgatctttcatttgtttttgctagccattatttccaggcgagagtgcagtcgatcaacttgtagagataatcaaggtctgcaaacattccatatatctttctttcgcttatactattagatgttgttgacctttgtgatgttcttgtaggttcttggaacgccaactcgggaggaaatacgttgtatgaacccgaattatacggagtttaggtttccacagataaaagctcatccttggcacaaggtaggcttgcaatctcattctaatgtccaatcatatatcacatttgctgttattaatatatgtggctcactgttattaatataggtcagccgtatataagatctgctgtaatatacttaaccatgtaatgtgatgcctacgtgtactgattgcactttgctgtgaccaggttttccacaagaaaatgcctcctgaagccatagatcttgcttcacgtcttcttcaatattcaccaagcctccgttgcactgctgtaagttttttcttttcacgttgcttgctcttccaggtgtttgttgcggcaagtaggagaggaacagatgaatgtaaatgtaaatatgaatggtcttttagagacaatcagatatatagttgtccttattgattgttggtaacttatttatgtatatgtgtgtagtgtacgtttgtcaaactagattgatcagtactagtcttcttttttttcttttcgaaaaggggggactccccggcctctgcatcagagcgatgcatacggccacaattataaataaataaagtagttcaacaaggtcttgcaatctgctgcaaaaaagtaggctggctcacaaagagctagaaaaacaaaaaaggcccaaaagccacaaccggctggcataagatagatagataggtaaactaatcgcctatcctattactagtcttctatgagcatcataatcatagttatgaggaccgctaatccagtaccatagatcgatgcttggcagatgacgagttgatatttagtcgtcttgtaacatgttttgctatagcgtgatggctatattcacgcatttgaatatccgcatgcagcttgacgcatgtgcgcatcctttctttgatgagctatgggagcctaacgcgcgcctgccaaatggacgcccgttcccacctctgttcaacttcaagcatgaagtaagtgcatcagagaaaaactaggctgctcatttgcaatttgacaaaaatgtatgcaacctgttcgtgctgttgtgcttatgggatctgcttttttttttttctgcagctggccaatgcttcacaagacctcatcaacaggcttgtgcctgaacatgttcgccgacaagctggtcttgctttcgtgcatgcggggagctaaatatgcgcaccggtgccctcaaccttgcaccttattgttttgccatgggcagaagggtggtggtttaagatggaggcaggtcagatgatccctggagcgatatatgccagattccatcatcaggagtaccggtagagcaccgaggaataacaactgtctagatcatctgccagggaaggagacttgccagggaaacagcatagccttacgccgtggacccgagttttctttcagtttttgccctattgtaagagttattaatagcttcttaatgtactgtagctcgtaagttgtcaactattttgttctccattcactgacgtattgttgcagtaaacttcgctgttcaataagttgtgtcatggcagagcttgcacgcccactgcctgtcatgtagtcaagctgtctattttctgttgggtagttgcgacccgtcgtgagatggcatggctgaactggaattagggttcgtgggatcgagaattggggaagctataggtttagtatggccaaaggctcacaatataatccaatgctgattccagaaaaacgggggaggcttaaattgccccgctagcaacaggtagaaaggaaacaactcggcaagtgaactgatacaataatactccccttgtcttaaaataactgtctcaattttgtaccaactttaatataaagttatactacggttaagacatctattttggaatggcacgagtagtaaataatggttgaatagatggagtcccacgagccgtccgatcctgtgacagacggcgagtcccacgagccgtccgatcctgtgacgagcttcaatcttgagcgtccactaactgaatcttgatgaggagttatataaagcagtttcggcctgacaatacctccccgtgaaagacgaacttgtcctcaaatagttgatgggcgatcgaaccagcctcctattgtttgcttgaacaaggccgggaaggtggtcttgatgaaatcagtgtcttcccaggatgcatcatgatgcggagcatcttacagtcattcccatggacctatcttcgtctctcgagacaccacgtggaccgatgacacgagctcgtgcaagggctaccacgaacgaggttaactctctctttgttgaactctcctttgacccacttgagacatggctactacctcaaatgga

SEQ ID NO: 12 - DNA, 2178 bp, Triticum aestivum
  GSK2 promoter sequence (wheat)
acgttccaaaaggataattcataacctagcaattttagatatctatgaacttcagtatgtgccatcacggtctcaacaggatatggtactctgttttgattttttatcaaaacctaattatcaatttatatatgtgcgtacaatctttaaccattatagctgacgatctttaacgtgtttcatcatctgtaggctgtgcaatgtgagataatcatgtatggtgttaccgaatgggctggaaagtttctaaattatgagctctgcatgattaagtggcgccgggacctgtacttcatttaatgcagagcaagaaaggaacatcaaagaagctgttaaaatggatgggagaatttgctaaaacatgtttacctatttttttaaagattaagtgtattctagaaagaaaataattgtatgtttcatggagtaacaaatcaggaactgtgcaggtatgtgttcatcttgatgggagtttgtccgagatgctgggcgaggggattgtttccgcactgcatgtatccaagtgtttagggatgccttttgcgaacaaatttagtttttttttgagcgaggaaattgatactttttttgtgaatgtattcgagtgggtgaaatactccccatttgtctcgagaggtccaggtgtggaagcttagcactgggtttgtcattattgaggcaagaatagtttgaatatgcacactttcattatgtacttgcagtgcgtaatgcatcatgtgtagaaaaagatagttatatttgtatagagtaaattacaatgtttgaggtatttgaataaaaaatactttgttgtttcatgacatgcaacaatgcgatcttttgtgctcgttattataagtttgagtaaatttgtattagttaatataatgtacgatgactgccacgtttgactaattttatattaatttagacgtgcaaacatttactagtactcaatatggaacacagaaaccgactatcaaagcattgctgatccgatcccgcatactatatataggtcattaactgacatataaaaatgtttggataattttacttctacaaatatactcacaaaaactgagtacatttttagcactggctaaaagggttattattcgaacgaaaacacataattgttgcgtaaaagatgttgtattttcagtaccaaatttacgatttgatccaaaaataaggaggcattaaaatgatgcggatctttgggtctcgggtgccaatgcacttgaatttgatctttttaaaagtattgcgaaattagtcaaaaatttcagaaacttcttgcaaacaagaatgatacggtgttatacccgtgtgacaagtttcacgaatgaatgagttttatggtattttaggttaaacaaaacaaaatcgacactatataaacatattcacaccttttgtttatgtcaaggagtccacggaagtcacttcttcgctaaactttttatacaagtataacactacaagattctcgtctccgaaaattttcaggaatttttgactctttttgttatttataaattattatttttcaaacaggttgcaatgggacccaagattcattaggtatttccgggcattaaaatgacatagtatatagcactaataaggttctcctatatgacatgaatgcgccatcaatttctcccacgaataccctagtatatttgtacagcaattagtgtacaattttcacaaatttctctgacgaataccctcgtatattatcagttcattttccggcagaaattgaaaatatgccgtaaatatattttagcggcattgttatccttttgaccaaaaaatgaatcccattactcggcaataaatgcggcagactatataaaacccaacctgatgcccggggtactcccagcaatttgactcccgaggctcgtctagtctaatccacctccccactaatccaccagctgtttacacagggtcagctaaccgctctctctatagatcaacgtcactccccatcttgttcgtcttggtcacccccacccccactttcccttcactggtcaaaggcaccaccacccacatcacagtacaagccaagccaagccaagccaagccagagaagaggaccaggcgtaggtggatgcaagtgtgagcccaccgtgtccgccccattcacaccctagccac

SEQ ID NO: 13 - protein, 971 aa, Glycine max
  OML4 amino acid sequence (soybean)
MPSEIMEKRGVSASSRFLDDISYVSEKNTGLRKPKFIHDHFLQGKSEMAASPGIIFNTSSPHETNAKTGLLMSQTTLSREITEDLHFGREAGNIEMLKDSTTESLNYHKRSWSNVHRQPASSSYGLVGSKIVTNAASRESSLFSSSLSDMFSQKLRLLGNGVLSGQPITVGSLPEEEPYKSLEEIEAETIGNLLPDEDDLFSGVNDELGCSTRTRMNDDFEDFDLFSSSGGMELEGDEHLISGKRTSCGDEDPDYFGVSKGKIPFGEQSSRTLFVRNINSNVEDSELKALFEQYGDIRTIYTACKHRGFVMISYYDIRAAQNAMKALQNRSLRSRKLDIHYSIPKGNSPEKDIGHGTLMISNLDSSVLDDELKQIFGFYGEIREIYEYPQLNHVKFIEFYDVRAAEASLRALNGICFAGKHIKLEPGLPKIATCMMHQSHKGKDEPDVGHSLSDNISLRHKAGVSSGFIASGSSLENGYNQGFHSATQLPAFIDNSPFHVNSSIHKITRGASAGKVSGVFEASNAFDAMKFASISRFHPHSLPEYRESLATGSPYNFSSTINTASNIGTGSTESSESRHIQGMSSTGNLAEFNAGGNGNHPHHGLYHMWNGSNLHQQPSSNAMLWQKTPSFVNGACSPGLPQIPSFPRTPPHVLRASHIDHQVGSAPVVTASPWDRQHSFLGESPDASGFRLGSVGSPGFNGSWQLHPPASHNMFPHVGGNGTELTSNAGQGSPKQLSHVFPGKLPMTLVSKFDTTNERMRNLYSRRSEPNTNNNADKKQYELDLGRILRGDDNRTTLMIKNIPNKYTSKMLLVAIDEQCRGTYDFLYLPIDFKNKCNVGYAFINMIDPGQIIPFHKAFHGKKWEKFNSEKVAVLAYARIQGKSALIAHFQNSSLMNEDKRCRPILFHTDGPNAGDPEPFPLGNNIRVRPGKIRINGNEENRSQGNPSSLASGEESGNAIESTSSSSKNSD

SEQ ID NO: 14 - DNA, 8150 bp, Glycine max
  OML4 nucleic acid sequence (soybean)
atgccttctgaaataatggagaagaggggtgtttctgcctcatctcgctttttggatgacatttcctatgtttctgaggtaattattaatgtaactgtctaagaatggtttgttctaatttataatgtgaccctcaacaagctaattgttattctaactgtcttataatgtttttttttataatgattatcagttccaagaacattttacagcctaagacttcggttttctttgtcattttgttaatcaatttgacctgtatgcatggcctcaatgctattgccttttcgacaattggttttctaaacatgcgttaaacttttatgggcagaagaatacaggattacggaagccaaaatttattcatgaccattttctacaaggtgagttcaatcaactaattattatttgttcaaaatggtttgtatatcttgtgctgatttacctgtgtatcaattgcatccttaatgccccaaatagattcactaacagatagttaagattcagaccttttgagtgaactgtttacactccagtttagaaattggctagtagctatcattgagtttgaacgtgtgaactttttgaagaatctttccatatatgtttctgtataccttatttttgtatatttcaaagcaatatttctctcaatttttgtttcaattttttatcaatgttttgtttgcttttagaattaatgattgtcaatgttgctaactagtatccttcatacgagtaattatcttaaattctaaaactggtatatttatttcactttatggtgattggtgataatacttgttgatttgtcttttttagcccatacacttctcactttatgctgaaatcaatatgtaatttttattttgcttctggaataatgaatatcactaatcaacgttgcaaattgacatcatctaaaattaatgtatttttctgtttgtggtgacaatgtaattgctgcaaacctatataaattgctgataaaaaaaaaaaaacctatataaattaatgttttatagtgaatgtataaattcaataccttgttctcacaacattttgattgtggtatagctgggataattaatgatgatttcatgaatttagatgctgtgctctgctggactgaagcttatttatgattttggtataaatattattaagaatttgcttttattttaattgtgctaattttgaatgtagtaataatgtaatatctgcatgtatccatatttatgtttgtttacctatgttccattaataagcagttcatctgctgaacatgtaactaatttctggataaagtaatttctatattcaaattttcagggaagagtgaaatggctgcatcacctggcatcatttttaatacttcgtcaccccatgaaaccaatgcaaaaacaggcttgttaatgtctcaaactactctatctcgtgaaattacagaagacctacattttggcagagaagcaggcaatatagagatgctgaaggattctaccacagaatcattgaattatcacaagagatcatggtctaatgtgcatcggcagccagcatctagctcatatggtttagttgggagcaagattgtcaccaatgctgcctcacgggaaagcagtctattttcaagctcattgtctgacatgtttagccaaaagtgtaagaatttgtttcatggatgttaatatagttgcatgcatgtgttatgggtattgtagcataatcaaattctggttgcttttacacttcgtaatattttagatatgagtttctgttgcattcatttgcttgtgtatttgtcattagcaatttagcatagaagaatatatgcttgctatcttttgtaatgtagaaggacaataccctcaccaaacccaccaccaaaaatataaaactaagtaaagttatctatttgttttaggttttgtgattatactttagttctgctcatgtgtcactgtgtgtatatatgtatatcttaatgcagtgaggttattggggaatggagtgctgtctggtcaacccattactgttggttcccttcctgaggaagaaccatataaatctctcgaagaaattgaggctgaaactattggaaatctccttcctgatgaagatgacctgttttctggagtcaatgatgagttaggatgcagtactcgcactagaatgaatgatgattttgaagattttgacttgttcagcagcagtggaggcatggaattggaaggagatgaacatctaatttctggaaaaagaaccagttgcggggatgaagatcctgattactttggagtttctaaaggaaaaattccttttggtgaacaatcttctagaacactttttgttagaaacatcaatagcaatgtagaagattctgagctaaaggctctctttgaggtgaacctttattcttttattctggcggatgctatcttagaattttcatgaaacatttcataccactaataatggcatgtaaatggactattttgtttgttccagcaatatggagatatccgaaccatatatactgcctgcaagcatcgtggatttgttatgatttcttattatgatataagggcagcacaaaatgcaatgaaagcacttcaaaataggtcattgagatctaggaaacttgatatacattattcaattccaaaggtatcattattaataacttctcatgcatgcataattcctttttccttgtcattttgataaagttgttatttttattcttcatcatatcatttattattaccgccatatgttttgcttgttcaattgcttgcatgcctgtttttatggtttgcttatagatatatcttgatttgatgacatgtcagggcaattctccagagaaggatattggccatggtacactgatgatatccaatcttgattcatctgttttggatgatgaactaaaacagatttttgggttttatggagaaattagagaagtaagtcgttcttgttggttttcatccatttttggtgtttgtgttttaaaatgatacaagcattcttaaatattgtctctgtaattgcagatctatgaatatccacaattgaatcatgtcaaatttattgaattttatgatgtccgggctgcagaagcttctcttcgtgcattaaacgggatctgctttgctgggaagcacattaagcttgagcctggtcttcccaagattgcaacatggttgctgttaccgcttcttttttattttcaatttatttttttctctttttatatcaactttttcaactgttttctacttttttaaatgtgcgaatcttaaaacattgttttgtaaatgaagtcttttattttggatcttcatatttatgctcaccacctttaatagtatcctcactttgtagagtttgatagagtgtaaagttgttatcaaccaccttgatggaaaaattacatcttgacaattatgcaaaccttgcttttgtagtatgatgcatcagtcacacaaaggaaaagatgaacctgatgttggtcatagtctgagtgacaacatatccttaagacataaaggtataatttttgtctgctttcacttgtcttttttccctcataaagctaaaatgttcttggtctcactagacataacttacagcaggagtgtcatctggatttattgcatctggtagcagcttggaaaatggatataatcagggatttcattctgcgacacagctacctgcttttattgataactcaccgtttcatgtgaattctagcattcacaagatcacaagaggggcatctgcgggaaaagtatctggtgtttttgaggccagtaatgcttttgatgctatgaaatttgcatccatttcgaggttccatcctcattctttacctgaatatcgtgaaagtttagctactggcagtccttacaacttttcaagtaccattaacacggcttccaatattggaactggatcgacggaatcatctgaaagcaggcacattcagggaatgagttcaactgggaacctagctgagtttaatgcaggaggtaagtttaatgtgctaagaaagcctcatgtatatgcttcctttatttgcagcagttttgaaatgtttccttgtctatagaaaattctgataaggaatcaatttgttgcaaaggttgaacttattgttcactttaaatggcatcctaggagtttgaaaccttataatgaagagcttgattgttaattttaatgatgatgccagcctagggttttcaatattttcattcttctaataacacccaaaaataataattgttgtttaagagccagactattgatctatatgaatctagacttgcctgtccgagtatatgagatttaagcattccaaattgtaaattggtcgaggtcatttttcctacaagcttgtaagtggtagaaggtgctgggaaattttaggctgaagcgatatctaatatggatttaatagttctatatttgaatgctggtatgtaacctttttgtttgattttggaccttcaggaaacggaaaccacccccatcatggactttatcatatgtggaatgggtccaacttgcatcagcaaccttcttcaaatgccatgctttggcaaaaaacaccatcctttgttaatggtgcatgttctccaggtcttccacagatacccagctttcctagaacaccacctcatgttcttagagcatcacatatagaccaccaagtgggatcagcaccagttgttacagcctcaccctgggatagacaacattctttcttgggagagtcacctgatgcttctggttttagattgggttctgttggaagtccaggctttaatggtagctggcagttgcatcctcctgcttctcacaatatgtttcctcatgttggtgggaatggtacagaattgacgtcaaatgctgggcagggctctcctaagcagttgtcacatgttttccctgggaaacttcccatgactttggtttctaaatttgatactaccaatgaacgaatgagaaacctctattctcgtagaagtgaaccaaacactaacaacaatgctgataaaaaacaatatgaacttgacctaggccgcattttacgtggggatgacaaccggacaacactcatgataaaaaatattcccaataagtatgccaattatctccatatcttttttgtgcatttttgctgcttatgctgttatcttctcatccttacttcaccaaagaatgtgattattagttaaataagcaattgcttatttggcttgtccgcttttcatgttggtgcattaaccatacaagtgccctctctcttttgcttgcttacatgcctaaatagcatatactttttacataacagattttacaaaatttgaataacaatttttaaaaacaagcaaattcttttgcactggtctttcagtttgtctctttcatttatatttgtttaaatatttttggcaggtatacttcaaagatgcttcttgttgccatagatgagcaatgtcgaggaacttatgattttctgtatttgccaattgatttcaaggcaagtatttatttggacttgctagttgattgattctttacttaaatgaagtaatcataaatatgtttctaagtcaattctacaaatggttgcagaacaaatgtaatgttggctatgcattcatcaatatgatcgatcctggacaaattattccattccacaaggttatttggaaacccttatgtaatactaatttgatataattatctgtgatttgaattctgagttcatctgattctttttgtttctaagagttgatatatctaatagataaggaatattgtacaggcttttcatgggaaaaaatgggagaagttcaacagtgaaaaggtagcagtactcgcctatgcccgaattcaaggaaaatctgctcttattgctcatttccagaattcaagcctgatgaatgaagataaacggtgccgtcctattctcttccatacagatggcccaaatgctggtgatccggtaaatcagcttgttctttagttgtaactattttccttttgctaactacaatgtattatggaactactatatcagcttgttcttcagttgtaattgttttccttttgctaactacattactacaatgtattatggaactactataggaccagactactaatattcctctcactgatattttattccgtagatctggttttactgatgatacattaatgttttgggctgatgcaagtaaagtgggagctaattataggctttctcacttcaaaacttttttgcctgatgtctaatattaacatccattgggtgtggcaagaaagagtttcttgaaggaatatttccccgatgattatttgacttacacataacaactaaagcattatgtttctgacttgagttggttttaatggctacaaaatgcaatctattagtgtgattttaactgtttctagcattatatgcatttaacaaactgggctttccattaaaagaatatatattggcttgcaacctaattgtactattggtacctggttcttccactgatattatataccgagacaaaattaacctaatctgtctctgcaggagcctttccccttgggtaacaatattagagtgaggcctggaaaaattcgcattaatggtaatgaggagaatcgcagccaagggaatccttcatctttggcaagtggagaagagtccgggaatgcaatagaatctacatcgagctcttcaaaaaattctgactgatttagcatcatgatctaacagttcaatgttgcatgtgatatcaactccaagactgtatatttacattatctttttgttcgatcgagcaagaggagttggagctggtaggaaagggggctcaaaattttttcctatagaggagccttgcaagagtttttggaagttgaggtacataacccgaatgaagtcactgattctattgttttccgttattttcctaaaattttgcatggagtactgctaccatcctacaactttagagaatggcctaactgaagcttaaaattttggctagctgtgaatggacaatgtgacactttgcagtttccttgtgataatgtgcatcattgtgggtttcaagggttctttgctgattttgttttcatggtcatctttgttgttcatatgtaattttgttcccttatattcccgggcattggtatccttatgggttttgttgtctatataatggatttttgaggaaaaacatttaaatgaaacattttcttcggtggtggtagtattcagatatttctgcttcgcttgttatttctgtattcttttatcagtgcttatacacctgttatgcatgtcagggttctagtcattgattagaaaaatgcttaattcagccttagttacatgccactagcaaatgctgtttgataataaaggttcctgagtcatgactatattctttgacagagaaaaaaattgagtatatatagcaattggttagatagatttctctataaacatttaaaagaaaacaagaaggtaaaaatattcagtttcttaataaactaaattcagtttatccatttaacttttggagaagttaaatgaaaagagtttctataaaagttaagtgcatatgcagtaagtttgattactatgaagttatatatattttctttgaagtttgatgatcataaagttttattttatatatgtatgtatatttcaaatgactgtttagacgaagaaaactaatggtacaaattttctgttcacaatcacatacgttggcatttatttgtacgcaagtgactggtgagctttatggcatgactagcagtggtacatttgctgcaaccagacctgatgaaatgagatttgttttctgtccatagatatctggcttttcttatcatgatagtgactcatcattttccgaagtttcaagtctcaacatgtatttttttttcattttaattttttgaccataaagtgaaatctgttgaaaaagtagtgcaatggtatctacaattccaatatatgtgtctgcgcagtgcgcacaaactttacaaaactaaccagtgagacatgattttgcacttttgccttttggtttcaagagtcaaga

SEQ ID NO: 15 - DNA, 3577 bp, Glycine max
  OML4 promoter sequence (soybean)
caccaaaccataaaacatatgaatgtgtgcaattaaattatttcattggttagtagatatgcaaaagaaaaacagttccattgtgaaaaaaacagtttcatcatttgaatataacaaatttgatatatatatatatatatatatatatatatatatatatatatataatgattggaatatatttaaaaatagagtaaatactttcttattgttcacaaaacattatcttagaagaaagcatttatggaaattttttttagagaaaatattgaaagtagccattaatcaatagaattgtataaaattttcctaaatgaaattttacaagaatatagaaacccaacttcccatcttaagctcaatacacatgcctgcatccaatataaagaggggtttcaacttgttgtttttcactgtaaaaaaaaaagttttaagcatattattattataatccacactactccctgcacctatttattatcatttttttaagataagattgtaattaataaatttgttaaatagaatttaatgaattaaaagttagtaattaattatcatttttcttcgagataagaatgtaattaataaatttgttaaatataatttaatgaattaaaattcaaggtattaataaattacaattttaaaaataattaaaattttcacttaaatagtaatttcattaccaaatattttaaggacaccaataaagtaaatgtatctagtaattacttaaaaataaatatttgtttaatttttaatgatgttatttttactaaattcctaatatggtaaaaaaaattcacttaatatttttctatttgttcatggactaatagtttttcttatgtacattggtagtaatgaggatcgaaaccatccatctactataaaacccttgctatcagatgaccctagtgaattagactaataatttaagccatctttagtagtaaattaaattaaattaacttattatatacatgcttatatgctgaaaacaataataaatcacatgtgagtaaatacatgaacaatttattttaaagacaaattttttttcttttgaatacgggtacgttatgagaatccatttaaaacatttatataaccattattttgtcgcatgcatataccattatgtatcctgggattttatgatgcacaataactataactacaaaattaaataactatggaattttaaaaattaaataacaaatgtttttcagttataccttaaaatgctaaatgtgtatttaaaactaggaaaataaaatagcaagtacttagattaattagtgtgggttatttcaatttatccattaatcttgaattaaaatctaaaagtatataattgtattaaatatttttattcaagcaagtgcatctcttgcatgtgttttttgaaatcaatatattttcattgtcggaaactaaaaactttgaaactaaaaagacaactttgacaccgactctcaaagcttaaagattatcaaagtgattttacctctttaaacaaaatatttttcatacaaataacacatttggaaaaccaaaaaattaaaaagtataataaaatcaccagtaattcacaataaaacatagatatgtgtatagatgaattattacattaagctggtattcaagcaacactaaaaaaaaaaagaaacagattcatgttggataaaatcaaaaaacaaaacgaaacggtttcattatttgaatgtaacaaacctgacacctgatattataataataacaacacattaaattatatttttaaaatacaaatattatttttttgtgaatatattaaatttatttgaaaccattatcttagaggaaacatttatggaaaagagttttaaaaaaaatactgaaaaagtaactgtaataaaagtatccattagtgaataaatttgcataaaaaattcccaaacagaattttacaagaatttagaaagccccactttccaagtttccatgttaagaacacacagacttgcatccaataaagagagaagcttatcttgttattttccactgtaaaaaaaaaaaaaaaacaagtttagtttgaagctgcttattattaaaacacactatttctgcgcctatttattctccctcctcatttctagttttcattttttaattattattatttttattttttttgtttttgttttcaaagccaactaacacccttttcctttcactttctctgtcagaagactaaaaaaacacctctgttgttgccttgtccgtttattttctctccaaaccagagagcgaccgccggcgagtctcacctcgccggaaattcgctcttcctcgccggaacctccatttttccctttcctattgcgcttgctttttcttcccaccggctccttcaagaggagccgtttccgtgcaatagttttgttgcttctttttttttttcgttttctgagcgcctgagattgcaatgcagagggaaggaggttcctgtcgcggcaagaagcgtgggatctctcgtttctgaaattcctatgatgtggagcgtttgagagattcgtttttttcttcttcttttttttgtctctttctctttgttctcgagtttctggatgaggagcgcgctgaattttgtggaggagaagtttttgtgctagcgagggcgtgttgaaattcagaaggtgtgaatttgttttgttagcttgagaaaaaaaaagtgctaattatggtttgtgtttatgtgtgtttctttttgctttttttttttttctggttaaatggttttttctctttagtggaagtggtttttatgagtttatggagagatgagaatctctttggtgttcatgtttttgatctgctggtgttttcagattatggcctctctgaaattttgttcttttgtttttattttttctctggatctgttagtgtttgaggctttccctgtgaaattccctctccatatgtgtaattttgtgatagaaccaaggtgtagtgatataaatttaagttaaatgcttgtttttttttttttttttggtttgggaaaaaaagggagagttgtggttacagttgaatgggattttatttttgttttgttttaatttccatggatagtttttgtttttaattttttaagttttttgttaagcaaatggcctaataaccgcattaggttgttagtaggtaagcatcgagcttctttcttctcctaatacatccttccttctggtaatgtatagttgaagatgcaactatttgtggctttgttttccactgctctttttaaaatttgcaattgaatatttgaagtgctttggtagggttttcattagtccatttttttgtcacttttttttgtggtggtgatgttagtaaagttccaattgttatgatagatgatatttttctgggtctgaatttttctttctgcctgtagcagttatgtatgatatgaaatgcgatgttcattgttatcagtcctttcctatgacaagggaatgaccttgaatttctcgcagatgtcgcacatagagcttgacgaaaactaacaaggaaggaggttttcagg

SEQ ID NO: 16 - protein, 420 aa, Glycine max
  GSK2 amino acid sequence (soybean)
MASLPLGHHHHHHKPAAAAIHPSQPPQSQPQPEVPRRSSDMETDKDMSATVIEGNDAVTGHIISTTIGGKNGEPKETISYMAERVVGTGSFGVVFQAKCLETGEAVAIKKVLQDRRYKNRELQLMRLMDHPNVISLKHCFFSTTSRDELFLNLVMEYVPESMYRVIKHYTTMNQRMPLIYVKLYTYQIFRGLAYIHTALGVCHRDVKPQNLLVHPLTHQVKLCDFGSAKVLVKGESNISYICSRYYRAPELIFGATEYTASIDIWSAGCVLAELLLGQPLFPGENQVDQLVEIIKVLGTPTREEIRCMNPNYTEFRFPQIKAHPWHKVFHKRMPPEAIDLASRLLQYSPSLRCTALEACAHPFFDELREPNARLPNGRPLPPLFNFKQELAGASPELINRLIPEHIRRQMGLSFPHSAGT

SEQ ID NO: 17 - DNA, 5793 bp, Glycine max
  GSK2 nucleic acid sequence (soybean)
atggcctccttgcccttggggcaccaccaccaccaccacaaaccggcggcggcggctatacatccgtcgcaaccgccgcagtctcagccgcaacccgaagttcctcgccggagctccgatatggagacagataaggtacttccgctcattgtactcttcacgaaccctcggagtggtttccgactttccggagctccgatctccgtcgattcgcctcgaagctccggcgtcgccggagtttcgaccgatctaccggttttccgtgctcgccagagattttctccggcgacgccgctgatcggaatggttattgttttcttcgagagcgatgttgattctcgttgacgaactccaaaaatagaaaagaaaattaggttttacttttttggagtgtgttttggttgatgcttttttggtagggatcttaacactgaagaaaaaattagaattttctgttttaggtgtcggagaaaaggaaaggaatcaatgtgaaaatgtggaatcctgtgctttgattttttgtttcctttaattcaaggagagagattctgattaggtgtacttagctgacctgagttaacattcttatttcacattctaacatttttatgtttctttcacttatctctaatctactgttaattttctttagctatgttaattctgtgctattatatggtctattatgggggtatagtttttgtttacattttttggggtttgtgtgtgtgatttcctttacttcccttgtggtggattgttgttcaaaaggtcaaacggttataatttgctttgcttcagggaattagtgtccttagattctctctgtattgtgtcttaagttatagcgttgaagtttttctttatgctttcttgtgagctgcggttacctgatttaactttagtttatgtgtgtgctctttgagctctttacactttgcctttcttcaacttcacattctgaactttgtctgatttcttctggtaaccctctggttcatatgtttcattgccatgcaattttcttctcataacacttgtttcaaccagtaaactgtcatgagataccccctttccttatatttgcatcttctcaaattaacttcactgtctatatgcatgtttgttgccatgtctggcacggcatgcgtttgatagttgataggcacatgttgttgccatattttgtggacgttgctaaacaaaattattgatgacaatatctgtaaagctaaatttaaatatgattgatattgtatcaataaaaatctgacattcaagtactgtattaggagatttgctttactgcattaaatatctaattcttgttataagttgcaggatatgtcagctactgtcattgaggggaatgatgctgtcactggccacataatctccaccacaattggaggcaaaaatggggaacctaaagaggtgagaatgtgttctaactcccaacccctttcctcctgaacttacaatttttattaaaaaattcatttcataccctcataaatatatgttattgtatatactgattattgttttgataatggttcacttccttatggggatagagtggaagtagagttagtggttggggaatctaaattaataaattgcctattatattcaaggccttccaaagatataatactggttgtcaatcagagtttggcttatttccccagtgcttacctcatctgataatttttattcgccaacaatatattagctcttacaagatgtataattttgaagaatttaattatgatggttcaataaatttatgcttaaaattggtgatatttatctgagtttccttatgtgggcttggttgaaggggtgggaaagggatttcaatgtcccttttctccagtggtcctcagcaatgtccttagcttttatttaatgcttcttggaagggcagggtttgtggtttgttcttgagatgtttgttatgtttttacagaagtattatattctatgtatcttttagtactactggtacttttcatgcattaatatatattatctttggagtccaaaaaaaaataaaatttattcttcgtaaataacttatttgttatgattacttccatgataccacctgcagaccatcagttacatggcagaacgtgttgttggcactggatcatttggagttgtttttcaggtatggatgaacaatcacctagatgacaaatattcctattaagctttctctgctgtcacatatctcattgttttccacccctggatggcattcctcttttacctaaaatataggcaaagtgcttggagactggagaagcagtggctattaaaaaggtcttgcaagacaggcggtacaaaaatcgtgaattgcagttaatgcgcttaatggatcaccctaatgtaatttccctgaagcactgtttcttctccacaacaagcagagatgaactttttctaaacttggtaatggaatatgttcccgaatcaatgtaccgagttataaagcactacactactatgaaccagagaatgcctctcatctatgtgaaactgtatacatatcaagtatgaacttttctattctgtttggaatttagctcatgtgttgttttataacattgtaacaatcgagtttggatatgatgtttagatctttaggggattagcatatatccataccgcactgggagtttgccatagggatgtgaagcctcaaaatcttttggtatgctttctttcaatgcttttcctctatgagttgtattcatttattctaaatcttaaccttttgcatatatgactacaggttcatcctcttactcaccaagttaagctatgtgattttgggagtgccaaagttctggtatgttggtctgcattgttcttgtacacatcattgcttcatgtacatatgccaccatgataatggaggactactaaaatcaaattcttcctaccggacatagctatgctaaaacttgtataagatctttccataaatgcaatattgatttaacctgtttatgtgatgatttgttattagtaaatagcaattgaagtgaaaatgatgccaagaatcttgactctgacccattttttcctattatatgaaaaataaatagaagaaaagttatcgattggcatcatgtggattttttattcaattatcaatttcatgaagcttctcatgttcaccacttggtaggatatagttatgatattatttttccacaaaaaatttatatcaggtcaagggtgaatcaaacatttcatacatatgttcacgttactatcgggctccagaactaatatttggtgcaacagaatacacagcttctattgatatctggtcagctggttgtgttcttgctgaacttcttctaggacaggttataaatttctggaaatctatgcattaatgttgttgatacttaagatttttttgctttctttctgggatatgttatattgacttcacgtagtttctaatgtttgtatagccattatttcctggagaaaaccaagtggaccaacttgtggaaattatcaaggtgatgtcccttctatatgagtgtctccatggattgcagaatatatctgcagagataattatttagatgtcttcttgtaggttcttggtactccaacacgcgaggaaatccgttgtatgaacccaaattatacagagtttagattccctcagattaaagctcatccttggcacaaggtaatgacatttctcatccatcctccttttgatattcatcacttgccattggactttaaaatggggattaaaaagatgaaaaaatagttgtcaaaatcaaattcaatagcatgcgttacaagttacaactaggtttttgaggttgctttccatattctttgttttgtaattgatgagcatgatagcattgattgatgtaacctactacctcactaataggataaagcattggctagtgaattatgcatttattttggtgctctatgtttcaggttttccacaagcgaatgcctcctgaagcaattgaccttgcatcaaggcttctccaatattcacctagtctccgctgcactgcggtgagtaggatgaactatgatacctcccttcacttttcccctttaaataaaaggaaaacatacacaggaaaaagtttgcttattttaaccttcttgctgtgatattatatctatattccttatggcatgttttttaatttagttaactcaattggcttaattttcactggtggcttttattatttcagctggaagcatgtgcacatcctttctttgatgagcttcgcgaaccaaatgcccggctacctaatggccgtccactgcccccacttttcaacttcaaacaggaggtatatatcttagtcgtattttttttattaaatgtgacgtgtcaaggctgttgctttgtccactgttcattatgtatatctgtatgactacttactttaccatctgttctgcatgatccaaaccaaacacaagggaatccaattaacaatttctcttatatcaaaattgtgaacagtatttaacaccagaatattatcttaatcattctgcaatgaaaacttaactacagttagctggagcatcacctgaactgatcaataggctcatcccagagcatattaggcggcagatgggtctcagcttcccgcattctgccggtacatagatgtaaagggataatgaaacgatgagtcaacctacatagtgatcgatgtgaatcaacagaagggctgtttgaggcctatgtataactgggagtcccaacataatatgcagtttttcctcccccttgtgaagatgtatacatgtgttggttgctcggtaaagcttgaaagttggtgattctgtgtagtatttcattcaagttaaagcatacttatccctgcatctgtatattgttttggtcagatttcagaaagctaggagtataaaatgatagcaatcatgtcttcataggtagaggggcccagctgaattgaggggcccctatagtagtttggctttgctttttatgagattaaattcagcatgtcgtttatattatgtttataacaatctcttgattcaaaacaagaaattttctcgttgtttaatactctagtaaccccgttccttctacccaagaagattttgtttgtcatatgtggacaagaagaaaggattcaatcaaaaagttgattacggaagaaaaaaatatgaattctttatgttgatgacaagggtgtgtgcacttagggtgacttgttaacaacatacgttgagatgagggttaatatacttcgttgctatatattcaattatatttcatttctatttgtgttgaagtctaagtcagaatttgaagtcacatatggttaggacttgggagcaaaatatataagtgaaaaagaatcacaaacctaacgctttaagatcatccactatgcatattgaattgtttagaagctttttcggtggtcctacacttcacctcagatttaaaagtttttttttcctccgatgataacattaaatgaattgtttaaatgaacttaaaagatgttttttttttttataaaaaaatttggttaagcagcaattcttaaatgctatgttatccgctctaatggtaaattctgttaaacaatgttgtttctgaacgtataatataatgtaatcaacgaaataaaattactatcaatcaaagatactagggtattaacataattatgagttgatttagtttgaatttagaaaaaaaactgattaaactggtttgatttg

SEQ ID NO: 18 - DNA, 2233 bp, Glycine max
  GSK2 promoter sequence (soybean)
gaaattattttaagtaagatatctatttataaattaggtccaaattcacattttttaaacattataaatagaattatctacctgaacataaagtgttaaacaatttagaatgtctaattttaaatttgaaaagtaaaaaagaaaatttactcagagtttcacttatcacaaaattgatagtaaaaaattagtttcagtgtataatttttacattactgaagaaaaaaatttgtgactttaagagctcattatacacatttaatatgttttggattctagctagtctgatttaatataatttaaatataaaatatttcataattgtttgttatgcatgttttgaacaccactccttccataagggggagtttacactgttcaattacttttacatgatcacgtcaaggtcaagattatgattcttaatcgcatccatagctagcaagaagcaaaagcagttaccagaggtttctggaattcccagcttctctctctctctctctctctctctatatatatatatatatatatatgattgccaaatgttacattttggggcttatgtgaagtgataataaattcaattgaacgtcccttctcttcctttaggatatttctttttctataacataaaggatagtttagaatacaatatataactacctgttttaggttttaactattgaatcgggtaaaaactgaaaacaactaatgctgaaaaataaaataaaatctaaaattggaaaattggccaggttaaaaataaaagaggttaatttctaatctataaatttaatgtatgtttagattgcagttggaagagtttaaaatattttgtctcaaagtagtagtttttttttcttcattatttcgtacattaaaatttttaaaatttattctcaacctttattcaaatatagtcttataattagtactaattaaatagtagtgtcaaaaatcctacactcaaatatattccaattaaattttaaaaaatattatttttcatgaagttacggagtgctgtgcactaatgagatgaaaccgagcaaattattagaatatactacatagttacaattataataaatgaaaattaaaatattttttacattatttggatatgcatatagaaattatatacttatatatatatatatatatatatatatatatatatatatatatatatatatatatatatcagtaatataatttattaaacccaatggaaagtacttatgagaaggagctgtaattttttattttattttcaaagtatttcccaataaataaatatctaagtaaaagattaattaattgaaaaaaatagtatgcatgagttttattagtgaatattttaaaaattttggttaaaaagtacttagtatattgttatgaaatattttatttttcaactaattaaaatatttaatataactgtataatatacatataatcttatgatcacttgttaaaaagactcattcaattttaaatagatcaaactgtacatttgatttaatgttcattcttattttatttcttaagttgacaattcataacaaagtcataaatgcatatatgtaggacagcgttttcattttgaatgaatcaattttctttaagatgtgtttattttaattacatttctttctttcttttgtaagaggttttcaaagatgttcatactatattaactgcgtgaaccatgcatcggatgtttcgtgttcacaatgatttttaatgaatatttaattaataaataaagaaaatatcaaaatgtcttttaacgtcatcaaacgttaaatatatatatatatatatatatatatatatatttatcataaaaaatcaaaatatttattaagaagattaaatataaaaatgtaaatttatcatcaatttggacttgagttatgaagcactacctttcgttttaaattctctagataaactgttttacaaaatattgcatgcaagtacgtaacattatacgaacatcgaatttgcttcggttctcctgccccttacggcaccagatcactgctccctttcatcacgaccctgattcgcgcgtgctctcaatctccctaaactcgcgtgaactcactctttctctcttcttgaacaaaaacagggcaagagagagagaagaaaaacgaagaaaggtaatagagagagaaagggaagaggagagagaaacgaagaagaagagtgtttctcacatcac

SEQ ID NO: 19 - protein, 996 aa, Zea mays
  OML4 amino acid sequence (maize)
MPFQVMDPRHHLSQFTNTTVAASSFSEEQLRLPTERLVGFWKQESLHHIGSKSVASSPIEKPQPIGTKTMGRVDPQPYKPRGQKSAFSLEHKTFGQERHVNMPPSLWRADQDPYVQSDSSLFPDGRSTNPYEAYNENGLFSSSLSEIFDRKLGLRSNDVLLHQPLEKVEPTHVDDEPFELTEEIEAQIIGNILPDDDDLLSGVDVGYTAHASNGDDVDDDIFYTGGGMELETVENKKSTEPNSGANDGLGSLNGTMNGQHPYGEHPSRTLFVQNINSNVEDSELKVLFEHYGEISNLYTACKHRGFVMISYYDIRSSWNAMRALQNKPLRHRKLDIHYSIPKDNPSGKDINQGMLVVFNVDPSVTNNDIHKIFSDYGEIKEIRDAPQKGHHKVIEFYDVRAAEGAVRALNRSDLAGKKINLGTVGLSGVRRLTQHMSKESGQEEFGVCKLGSLSTNSPPLPSLGSSYMVAMTSSGRENGSIHGLHSGLLTSMSPFREASFPGLSSTIPQSLSSPIGIASATTHSNQAPLGELSHSLSRMNGHMNYGFQGLGALHPHSLPEVHDGANNGTPYNLNTMVPIGVNSNSRTAEAVDCRHLHKVGSSNLNGHSFDRVGEGAMGFSRSGSGPVHGHQLMWNNSNNLQRHPNSPVLWQNPGSFVNNVPSRSPAQMHGVPRAPSHMIENVLPMHHHHVGSAPAINPSLWDRRHGYAGELTEASSFHLGSVGSLGFPGSPQLHGLELNNIFSHTGGNRMDPTVSSAQISAPSPQQRGPMFHGRNPMVPLPSFDSPGERIRSMRNDSGANQSDNKRQYELDVDRIMRGVDSRTTLMIKNIPNKYTSKMLLAAIDESHKGTYDFIYLPIDFKNKCNVGYAFINMTNAQHIIPFYQTFNGKKWEKFNSEKVASLAYARIQGKTALIAHFQNSSLMNEDKRCRPILFHSDGPNAGDQEPFPMGTNIRARSGRSRTSSGEENHHDIQTVLTNGDTSSNGADTSGPTKDTE

SEQ ID NO: 20 - DNA, 19111 bp, Zea mays
  OML4 nucleic acid sequence (maize)
atgccatttcaagtcatggatccgaggcaccacctctcccagttcaccaatacaaccgtagctgcgtcctccttctctgaggagcagcttcgccttcccacagaggtaataatctgcagttgcagaattgttgccctatttattgttttctgtttttgttagtttatgataaggctagtggtgtctttattgttttagttcatgtttgatacctaccatgttgtcactcgattttctggatatctatgacatgcactaatttttttaatctatctttgcagaggctggtgggtttttggaagcaggagtcgttgcatcacattggtgagtacttaatttgattcaataccccttagctttttgctcatttccatgcaaagaatgctctttggctgcaaaaatccacatgttattgcggggaaattttgtgcatttaataacattttatgcgtgactaagggctagtttgaatccactagagctaataattagttgtctaaaaaattgctagtagaattagctagctaacaaataactagctaactattagctaatttactaaaaatagctaatagttcaactattagctatattgtttggatgtctatagagctaattttagcagctaactattatctctagtgcattcaaacagggcctaaataacataaataatttgtttgcttgtgatgaatatgattttagctttttaccctaactttatcaggaatagaaggctttgtttttgttgttgtcgtgttggacatgttttattgcacttttcatttgttgtttatgtatttatagtctcaagccattgttttgtgttcaccttgggttgcaatggatttatgacatatttgatgtccaggttatcctttgatatgcaattggttgcgctagtttctaccttattattcctttttacttatttggcactcctgtcgtactctctctttgttctcacaatggttcatgcattttgttgttcattatcaagatgtcttctcaaaggcaagctgtttctattgttgtcagggagcaagtcagttgcatcttctccaattgaaaagccccaacccattggtacaaaaacaatgggtcgggtagatccacaaccatacaagccgagaggccagaagtctgcatttagccttgaacacaaaacttttggtcaagagaggcatgttaacatgccaccatctctgtggagagctgatcaagacccttatgttcaatctgattcatctttatttcccgatggaaggagtactaatccatatgaggcctacaacgagaatgggcttttctcaagctccctgtcagaaatttttgacagaaaatgtgagacagcttactctggcactttcatcaacttcattagagcgattgattatactgcagtgagcctgcaccatgagaaccattctcttcatcttagaaaatgcattgaactgtatcacacattccatagtatgtattgtgtatgtgtgtgccttgaaatcaacagaaaggaataaaaagtacaataaaggatattagtgagtatgaatgggaagaaaaaataaaaaaaatacttaacatatttttttagcatttttgcatcttattttcgaaggaaccttacctgctttatttttctttggcccaagaatcctttcacttaagtttggtatcgttatccttttattttcagtaacactttgtgcaagatttgggcagtcagacactccgattaaatcattgctattgtagtaagcaatacataattcatatttattgctttctaacaaattatatgcttcaatgtgtagtgggactgagatcaaatgatgtgcttctacatcaaccacttgaaaaggttgaaccaactcatgtagatgatgagccctttgagttaacagaggaaatcgaggctcaaataataggaaacatacttcctgatgatgatgatctactatcaggtgttgatgttgggtacacagcccatgctagcaatggtgatgatgttgatgatgatatattttacactggaggtgggatggaactggagaccgttgaaaataaaaaaagtacagaacctaacagtggagctaatgatggtcttgggtcgctaaatggcacaatgaatggtcaacatccatatggggaacacccttcaagaactcttttcgtccagaacattaatagcaatgttgaggattctgaattaaaggtcctatttgaggtatgttccttttttctgttttctgcttaaacctatcgttcctgtacagaacatttgtttctgaaaatcatttactctttacccacagcattatggagaaatcagcaacctttacactgcctgcaaacatcgcggttttgtaatgatatcttactatgacataaggtcatcatggaatgccatgagggcacttcaaaacaagccactaagacatagaaaacttgacatacattactccattccgaaggtattcacgagtcttactggcttgatgtgtagacatattttgcccaaggatgccagtatgtagctagtttactgttatcagttttgtagttcttgtgctaattttcaccttttttcccttaggataatccttcggggaaggatattaaccaggggatgcttgttgtatttaatgttgacccgtctgtaacaaacaatgatatccataagatatttagtgactatggtgaaataaaagaggtatgctatgctcttacattaactacctactacattataactagaactataatgtcttaaattaattgcagattcgtgatgcaccgcaaaagggccatcacaaagttatagaattttacgatgtcagagcagctgaaggtgcagttcgtgctttaaacaggagtgatcttgctggcaagaaaataaatttggggactgttggtctgagtggtgttagacggtatgcctttgaaatgttatcctgctgttcattcacatatttcagtaacaatacttattacttttggacagtccatatttaactgttgatcatttgatcgtgattcttgcttaggcatctttggtatatagtaccatcacttattctatatgacggtacctgtcgatagaatgcacattagttgatctggatttttattttcttttctcaagtggaaaatctcttcctggagctgtaaacattgcactgtttttattttgtcatgcatagatagttgatctttgtttctttatttctatgtatgggctctgatgtcctacacaaaacagatttttgtttgttctttcatattgtagtcttattctatgtattgcatttaggtgtatggatatatacttagtatgttagttatctaagtcatccagaaaaaagagcaattattatgtgacaacattctaattttgattttaccgtgcaaacttttgaaaacattggttttaatcactgctctaacattgattttaatgttgttttataacagattaacacagcacatgtccaaagagtcggggcaagaagaatttggtgtatgcaaactgggcagtctaagcacaaatagccctccattgccttcattgggtatgctgttggtttttttcatctttaatgtatgtcatgtctatagctacatttcctgacatggaggataattcttcaaggttcatcttatatggtagccatgacatcttctggccgtgaaaatgggagtattcatggtttgcattctggactgctcacatcaatgagcccgttcagagaggcttcttttccgggcctatcatctaccataccacagagcctgtcctctcccattggaattgcatctgctacaactcatagtaatcaggctccccttggtgagctcagccactcacttagtcggatgaatgggcatatgaattatggttttcaaggcttgggtgctcttcatccccattctcttcctgaagttcacgatggagcaaataatggcaccccgtacaatctaaacaccatggtaccaattggtgtgaatagcaactcaagaacagccgaagcagttgactgcagacatcttcataaagtgggttctagcaacctcaatggacattcatttgatcgtgtcggtgaaggaggtaagtttgtaaatttggacattctaatctccatttttatgtttgaacccattgtcatttctattccttaaacatgtgttttgtaataaagctgttaggtttatcaggattgtgaaaactgaactgtgaaaatttgatcaattaatgtatgttatttaactgttccgttcatgattgcatctgtaacaaattttgcagctatgggattttcaagaagtggaagtggtcctgtccatggtcaccagctaatgtggaataattcaaataacttacaacgtcatcccaattcccctgtgctgtggcaaaatccaggatcatttgtaaataatgtaccgtctcgctccccagcacaaatgcatggagttccaagagcaccatcacacatgattgagaatgtccttccaatgcatcatcatcatgtgggctctgcgccagcaatcaatccatcactttgggacaggcggcatggctatgcaggggaattgacagaagcatcaagttttcatcttggcagtgttgggagcttgggatttcctggtagccctcagcttcatggcctggagctaaataacatattttctcacactggtgggaatcgcatggatccaaccgtgtcttcggctcagatcagcgcaccatctcctcaacagagaggtcctatgttccatggaaggaatcctatggttccccttccatcatttgattcacctggtgagcggataagaagcatgagaaatgactcaggtgctaaccagtctgataataaacggcagtacgagcttgatgttgaccgcataatgcgaggggtagactcacgaactacactgatgataaagaatatcccaaataagtatgttttgagatcaccaaattttatgctacatttatgttctgtctcaatatattcttttgttctggttggttctttcgggtttcaggtatacctccaagatgctcttggctgctattgatgaaagtcataagggcacttatgactttatttacttgccaattgattttaaggtagtttgaaactttgaatttaactcataagcgaccggggccttgtattagttgagactacttttgtgttcatgttactaaatgagatcaatctccttttcagaataaatgtaatgttggctatgctttcatcaacatgaccaatgctcagcatatcattccattttatcaggtcagaaaattattccaattgacgaagtgctactgcattgatgtaaagttgtaaactagcctttggtcaacttatatgccttgccaaatttgtactttgataaaatatccggcttgaacatcgacgtgctatcctgagccattttgtcatctttttcagacttttaatggtaaaaagtgggagaagtttaacagtgagaaggtggcatcacttgcttatgctagaatccaagggaaaacagctctgattgctcatttccagaactctagtttgatgaatgaggacaaacgttgccgccccatactcttccactcagatggtcctaatgcaggagatcaggtatgcttatttcttttttattttgtcgttggtactttccctgctatcttgttctccagttacattatgtttcgctgcagtgcactgtgacgagtcttctatataatccatataccttgaatccttgatggggctgatggcagataaaaacataggttttgtgaaaataaaatggggggaggtaaatgtccacctgccatttttgctgcattaactgccctgtgacaagacttctctataccatcgtacaaaggccctgtttgaatgcactaaagctaatagttagttggctaaaaagtttagagaattggctagctaacaaatagttggctaactattagctgatttgctagaagtagctaatagttgaattattagccagactgtttggatgtctgcagctaattttagcagctaactattaactctagtggattcaaacagggccaaagtcatcaatatataccttgaatccttgatgggctgatggcagctaaaaacataggttttgtgtggcgaatccttctaaattatatggcccacatgcacttgtctttatcccaaagacctcagacgactatgcatatgtaccagataacttaaaagaatttgtcccagtatctcgaaggacctcgggaaatccactttacaaccaagatcgcaagattaagtacacacaaatcacataccgaagttttgtagcggaattcatattacaataagtttacaaattacaatatcgaaaagggcgtacccaatgctgtaggcttcccgcactgtgcggggtctgggggagggtatctttaagcgccaagccttacccgcataatatgtagaggctggggctcgaaccagggaccttccggttacagacggtaggctctaccgctgcactaggcctgcccttcacaaattacaatatcgaaatgagtacaaatttgatatgaaagtaatacaactttgaatgacatgaattacaattttaagttcaaaatacattgctatcttaaatgacaaaactcaggtggaagtacagaaaatatacttatataagaagaccgagtccaccgacacttagcttctatctacaacagaacaagaacatcactcgcaacatggtgggataaaaccctgagtacacaagtactccacaaggcttacccgactaaagaaaatgactccaagggcatgcaagaattggggattcaaggtgaggttatagcaagaataaaaaactcctttgcataaaagcttactagaagtggatccttaagccatatttgaatttatcaacttagctctctcctaaatctagattagcctaatctagatcaaacacttgccaaaccattgtcttcattttaccagatctcatttctcttcttaactacgatgcacttaacccttgcatatgtcaacccaatcttcgagtggtccaagaccaaaacgggtttgggccacctgatagcacagtactccaccctccaacccatgctagttgggcacacactactctccttaatcgactcggacggaaacactgcaccgagacgaaaacactgcacaaatctcatttttctccttaatcgactcagatggaaacactgcaccgagactcctttctcgatgcaagttacccacccggtctcatattaattcacctttttcacatttctttaacatatctcaatattcagcggaattggaaacattttctgaaaacccctaattggaaacattacactctatttggtgcatgcaaggagaaaaatcttgtttccccatctactcgactgggacaaataatcatgcgtcaccttgtttccagccatataaagaaacgtgcatgctctggtaggaaaatggagaagggcacatgcttctacagtaggatagtagtagtacgccttattttttagacaaaatctaaaactttatacgccttgttttttaggatggacgaagtatataagtatatatgtccagaaacatatggatgactaaatggacgaccagctcgactagggtcgattagtcgacctagtcgacgactaatcacaactaacaaggttttaaagtcgtttgactaatcgcgattagtcggccttattgctggagtaagcacgattaggggcttcgactcgactagggcgactaggaagcgattagtcatcctagtcactgactaatcgtgattagtcgcccgattaggggttatgtccgactagctagtcctgttttgggccagtaattcgtcctttgttctatgggccggcaggcggcccaccatctctgcagaaagtagaaaacttgtgttgccctacctgtaccgcagtagcagcacagtagccgtcgtcccttctctagcgcgcagttgcgcaccctctgcagcccttcttcagcgtgcggctgcgtcctctctgctccggctgcgatccctctgctcctgccagcgcgtggttgttgcagaggcctctgttaagccgatgccctctagtatggcgcacgcctctgctccaggactccaccgaagtccaccctccagcgcaagagcgtcctccactgatccactctgactcctccatcatacttcttcagagtgaattagtttagagtttgttctgaacttcagaaatcagaaatcagaaattcagacttcagacttcggagtccagagaacatcagagttcagacttctgagttcacagttcaggctttcagagtctgttgttttgctatagctataatatattgttgtcctgctacagctctagtgtactgctatattgcagtactgctacagctatatatattgatatatatatttatacatatagtcctgttataggtggacgactataggacgactaggagtctactagactcgactaatcgagcaaatcgatgactaatcgtgattagtcgccttatcggtgctcaggcgactagaatcgactagccgactttaaaaccttgatgactaatcgactggtcggtagctatacgactaggttcgattagacgacttgaaaatagttatctcgagcagactccctatcccacttcactccctatttcaaactacactatgcaaacaatataatctatagtgcaaaacagtactttgcacgctcgtttacatggtatgctggagatgaccttagtgcttgttagacgatattcacttggcgattatctcccaacctagcacttgatctgtccatccatcttcaggttggtctgccgtcatcgtcttgtggttggctttgatccacgttctttactccgcgtaatcaactaacgtacctgaatgagatgcacgatgcatatgtatgagcataaaatgaaccaatgctacagtgaagaaaatcaaacacttaatggcaaggcattgccacaatcctacgcaagtactagatacatattgtcactaaccttgattaggcgagataataaccccctcgggtactgtagcatatatatgtaggcagacaagaatatatgggctttatgggccttaacaccccctatcgaactcaaggcggaagtggaggatttgaagcattgagtttgattagatgaaactgatgttgtgccctagtttgtgcttttgtgaagaaatctgcaagctataattctcagggcacatattgaagatcaatggtcttctgatgacaatgagatctagtgaacgatacatcaacaccaatgtgttttgtgagttcacgcttcactggatcatgacaaatttgtatagttccaatgttgtcacagtgaagaggcgtaggcgagtcacaagaaacgcctagatcagccaagagccaacgaatccagataatcttagcagtagtagtagccagggctcgaagttctgcttcagtactagatctagatacaacagcttgcttcttggatttccaagcaacaggggatgatccaagaagaatacagtaaccagtgatggagcgacgatctgtaggatcactggcccaggtagcatcagagtaagcacgaagctgaagtggggaatttgagtcataaaataaacattgtgtttttgtccctcgtaaatatctaagcacacgaagtaagtgcccataatgaactgatgtaggagcagatacaaactcataatcatagacggtttgacgctgttcaccatagcagccatcactttaccatcattaagctgccatgttttgatatcagcagcattgcgacgatcatccgcaagaacgggtgccgcatcagtcaaatgaaagagtaatccatgtcccctgagtgcagtctcaacacagaaagcccactccggataatttcggccatcaagagtgatattgaccacaatagcatttgtcgccatattgaattcaatgaaaatcagggagaacaggagacctgaaaccaaacaaaccagaggacgagttgacggaggtcctgggcgcggaaaccgagttggacagtctgctcgcaatggcagccaccggcgcagaaacagggacgacgcagatggcgacgaggcagggcgcagcagacggcagcgcagatccggatccgcagactgtttgcggcgtgattatcggatcgatggcagttgcacaaatcttctctgcagcgactgtttgcggcgtgcagcaggcggacggcgacagggcgcagtaggcggacgacggcagccgagcgcagcaggcaggtgcagacggcggacgacggcggccgggaagcccagatccgcccgcggggatggaagaaaccgcggccgggcgcagcaggcaggtgcggacggcggcgaccgggtagcccagatccgcccgcggcagaggcggggaggggaaaagccgcggcggccggatctgcctcgaggacggccgcggatctggacgggatccgcgacgacggacgggcggtggccggatctgcgcgacggcggacgggatccacgatggcggccgcggatctggacgagggcggcgcagatgagcccgcaacgacggaatccgcgacggcggacgggcggcggccggatctgcgcgaccgcggacgggatccgcgatggcggccgcggatgtggacgggggcgacacagatgagcctgcagccgcgacggcgggtgggaggaaggtggagagagggtcgcaacggcggccgggaggagacgatggtggctaaaaaaatctaagaaaccctaatcgtgacctgctctgttaataggtcactaaccttgattaggcgagataataaccccttcgggtactgtagcatatatataggcgacaagaatatatgggctttatgggccttaacacatataactcactaaacacaacaatcacgttcttccagtttaaccagatctaactcaaacatcaagaaataataaactatgtgtaagtcctatatcttctttaggtagtgcccaacatcagaagactagcaaaacctagactcatcattcttagacacctaaattcagaatgagaatagaagcaatctaactagcactctaaaccaccttttggtgaaagagtaattgtgggaatgacttgattctattccacgacaatgtgtgcgtatacataggagaggccggggttgctcacaaggcaaccgcacaggcgtacaagccaatcaagggcagcctacaatcaagggctgactaccataattaggctttctataattacaatagtctaacatttgggactaactcgcatagcacaacatctaaataaaacatcacactattagatctagcaggcagaacatcattaaagatcacagtctttcacaaaaccacaacttaaaaccaaaagacctaaaacactaatgtgcaatgcccactatgcagtattaagatttcaactaaagcagacctagcgatgttatttgcttcgagatacttggagaagcaatcaacatccatctatgacatttaaccggtcactaaggccctgtttggacagctccagctccagaaaattcggtagagttggtggagcaggtcattaggtgctccataaaatcgtggagttggagctgtaagccttcagaagacattttgtctttgataagtcatgcccccgcagtctaatcgggagcatcgctaacggtcaggctggaccgaaactcctggaacaacgaggtgggtggtcccttggtgaagacatctgcgtactgagatgtcgtaggaacatgaagaacccgagcgtgtccgagggcgaccttctctcggacaaagtggagatcgatctcaacatgcttcgtccgttgatgctgcactggattgctggagagatatacaacactgacgttgtcacaatagactagggtggcacggcgaggcgggtgccgaagctcaatgagtaactaacgtaaccaagtagcttcagcaacgccatttgccacaacacggtattcggcctcggcactggaccgggaaaccgtgtgctggcgcttggaggaccacgacactaggttgtctccaaggaacactgcgtagccagaggtcgaccggcgagtgtcgggacatccagcccagtcggcatctgtgtagacaacgagcttcgtaggggaagatcggcgcatagtcagtccaagagatatagtgccttgcaggtagcgcaagatgcgcttgagagccgcgaggtggggctctcgtggatcatgcatatagaggcaaatctgctgaacaacgaaggcaatgtccggacgggtgaaagtcaaatactgtagagcacctgccaggctgcggtactgagtagcgtcatcaacgggaggtccatctgcagataatttggagtggagatcaacaggtgtgctacacggcttgcacacgctcatcccgacgcgctccaaaatatcctgagtgtactgtcgttgagagagaaacagaccattggcagaacgtgtcacagaaatgcccaaaaaatggtgaagctgacccatgtccgtcatagcaaactcacgctggagagccccaatcacatactgaagaaactttgcagaggaggcagtgagaacaatatcatcaacatacagcagcaaataggcagtgtctggcccttggtgatagatgaacagtgaactatctgacttggtttcaataaatccaagggaaaaaagatgggatgcgaacctgtgatgccaagcacgaggagcctgcttcaagccatatagggatttgttgagccgacagacaagatccggatgagaggaatccacaaaaccagagggttgtacgcagtacactgtctcggtgagggtgccatgtaagaatgcattcttcacatctagctgatggatggaccagttctgagagagagccaacgagagaacaactcgaactgttgcaggcttgacaaccggactgaaagtctcatcataatccacaccggggcgctgggtaaacccacggaggacccaacgagccttgtagtgatcaagagacccatctgcgagcagtttgtgtcgaaaaatccacttgccagttaccacattgactccaggaggccgtgctactaaactccaggtgtcattggcgagtagagcatcatactcagcttgcatagcggagcgccaattggggtctgacaatgcatcacgaacggagcgaggcagtggcgacatagacacaacgtggaggttgaggcgatccacgggctgtgccatgccggttttgccgcgagtgtgcatgggatgcgcattggcgataggggtgatggagaccggacgagtgtctgccgtgcgaccggtggccgcggtgctgctggcaacggcctgtgcaggatgcacaggggcagcatcacccgtcgatgcggtcggcgaggcggcgggggcactgctggcagcagcctgtgcagggtgcacaggggcagcagcgccggtcgacgtggcggagggactgggcgtccccaatccagtgggaggagacgtgggtgcctctacacgcccatgggcgacgtgaggtgtacctgcatgcacaagtcttgctccaggaataggagcggttagatcatgttcatcaagaagaaaatccaaggcggaggatgccatgggagtggtagacatggctgcgaaagggaagaaggactcgtcaaaaacgacatgtctagaaataagaatgcggttcgactcaagctggagacaccaatagcctttgtgttccgaggaatagccgagaaaaacgcataaggaggagcggggggcaagtttgtgaggtgctgtggaggacatgttaggataacaggcactcccaaaaactttaagatgatcataggagggttgggaggaaaagagggcactatatggtgtggagaaagcaagggttttagtggggagccgattcacaagatatgtcgcagtgtgaagggcttcaacccaataagccggaggtatactggcctgaaacaaaagagaacgcagaatgtcatttatggtgcgaagagaacgttctgctttcccattttgctgagaagtttaggggcacgacatgcgtaagacaatgccgtgggagagaaaaaatgtgcgggcctgggaattatcaaattcacggccattgtcgcactggatgctcttgatgacggtgccgaattgggtgcgaatataggtgaaaaagttggcaagggcggaaaaagtctcggactttagacggagtggaaacgtccaaatgtagtgggagcagtcatcaagaattaccagataatatttatagcccgacacactaacaattggggaggtccataaatcacagtgtattaagtcaaaattgtgagaagctcgagagctagatgaactgaatggcaaacgaacatgacgaccaagttgacacgcatgacagatgtggttgacatcatctttattacaggaaataacactggaggtaataagtttggacaaagcttgatgcccaagatgaccgagacgacgatgccacagggaggtgggtgcagcgaggaatgcaggggtgctggtggagggtgcatagaacgggtagaggtcaccggagctattgcacctggcgatcacgttcctggtttgcaaatccttcacagaaaggccaaagggatcaaactcaatggagcaattattgtcggtggtaaaacgacggatagaaattagattcttaataatgttaggagacacgaggacattattgagaactaaattgtgatgcgggaaagaaaaaatatgtgatccagtggctgtgacaggaagcaagacaccatttcccacaatgatagatggagtgaatgaagtgggcaaggaaatggtggaaagtttaccagcgtccgaggtcatgtgcgatcctgcaccggagtcggcgtaccactctgaagtagcgttcggcgggttgagggtcatggtgttgaaagagtgcaagggcgtcctgatgccatgctcctccgtgagtggggttccagggcgccgcttggtacgccggtgcgggcgcctggaaaccaggggctccagttcccccgtagtgcataccgtacggaggggatggagcgccgtagaaactgccataggcgttgtattgtggtactgcgttgaatgctggcggcggtggtgggcgcccggactgatcgtatggccacaaccgtacagtgccaacccaaggatgcgcgaaggacggatgcatgccggggggcacctccctgtctggcaccaggaggcgttggttggccctggtgatggccgttgcgtccaccgcggccgcgacgacggccgttacgttggccgtttggcaccgaggggcatgctggaggatgtgcccctggacgtggaggtgctggagccccgggagccgcagctcgcagcgttgcagcgacgagggcggatggcggggacggaggtcgtgcgtcgatttccagctcctccaacagcaggtgcgctcgtgcctctgcgaacgtggggaacggcctgtgcatcttgaggatggacaccatctggcggaacttaccgccgaggccgcgaaggagcgtgagcaccatctgccgatcgtcgatgggatcgccgaactcggcaagggaagccgccatcgattcgagctggcggcagtagtcggtgattctcagggactcttggcggaagttgcggaattttgtttcgagcagaagcgcccgagactccctctggccgaggaactcgtcctcgaggtagcaccacgccccgcgagcggggccctgtcgcatcatcagagattgctgcaggtcaccggagacggtgctgtagatccatgtcaggacgcagcaattggcttgaacccatgccgggcgcgacgggaacgcttcatcttcaaggacgtgacgagtcagggcatatttgccaaggacagtgaggaacatgccacgccacttggtgtaggtatttgtcgcctgatcgagaaggacagggatgagcgccttcacgttgacgacggcagtggcctgcgcccagagggcctcatgggcatgctcatacgcgtccagagcggcagcacggaggcggccttcctcggcgcggcgggcgtctttggcacggtgtgcagcagcctcagccgtagggcgctggtcctcggcggcagcgtcgtggtcgtccgccatcgggagggagacgcgacggctgggcagccgagctggagccgctgcagactggacgggagggaggcgcgacggggattagcgtggtctggaggctcgaccgcgcccgaccagagggaacgaccacgcgatctggacgggaatcagccgagggagacgcgacggggatccgccgatctggtgccgcgtcggatcagcgagcgtggcgggcgagcggatcagcggccgcgctcgcggtctggagctgcgaccgcgcgacgaggcgggtgagcggatcagcggccgcacccggcagcaacaacgacggggcgggtgatcgaacggacggcgcaggcgatgggatcagcgacgctccaggcgacgaggtctgcaggggcggcgatcggatcggcgacggcgcggtcttgggttgcggaagtgtggtggatcggaaccttgataccatgaaagagtaattgtgggaatgacttgattctattccacggcaatgtgtgcgtatacataggagaggccggggttgctcacaaggcaaccgcacaggcgtacaagccaatcaagggcagcctacaatcaagggctgactaccataattaggctttctataattacaatagtctaacatttgggactaactcgcatcgcacaacatctaaataaaacatcacactattagatctagcaggcagaacatcaccaaagatcacagtctttcacaaaaccacaacttaaaaccaaaagacctaaaacactaatgtgcaatgcccactatgcagtattaagatttcaactgaagcagacctagcgatgttatttgcttcgagatacttggagaagcaatcaacatccatctatgacatttaaccggtcactaaggccctgtttggacagctccagctccagaaaattcggtagagttggtggagcaggtcattaggtgctccataaaatcgtggagttggagctgtaagccttcagaagacattttgtctttgataagtcattttgattattatttaggttaaaaatattttttaaaactatttaaattaatattataaactatagctccgcgctggagctggaatttagagtcatcccaaacaccaactaaatatagagtataatgaccactagagcaaggcatcgactttatcaaataaataaaatcgacacaaacaacactgagaacatgttggctagccgattgaaatactaaacctatctttcacgtcatcaattgacaatacattgcatacttgtctaccaaaacactcttctaggagatggtatcattctcactgtttccagagcaagtttggtacatagtttgcaaatcgcaccatacttaaatggtcccagtgtctgcttaacaatttcagaacttgctgtatttttgtgtttgcagttcttctaagcacatggttgtaattttgacattttgttgtgatctttctcaggaacctttccctatgggtacaaacatccgagccaggtctgggagatcccggacttcctctggtgaagaaaatcaccatgatatccagacagtcttgaccaacggtgacacttcttccaatggagctgacacttcaggtcccaccaaggacactgagtagctgaactgcagcttgctgcgttgctgaccacaaaggcccaaactataactttttgcaaacccattttcagttctttcccccctttcccattttggttctgttttgtaaagtctcccgatctgtatttattgacttccacgatgcgggtcaccgaagacttaggttgctgcaaaattttgtccctgacgggaagctatatgcaagagggtggtactggctatgtgcttgttaacctgaaggccgagaaaggtgaaaagcgcagggagagcctccagattttggtcgctgtaagaattaaccccatgttgtacagcaggtcccagtaacttgtagtgatgggagagtggagtcattttcatcagtttttagtggtggttgtgtggagaggaagagtcttgcctgcgttttcttttggaaccttctcttgtgcctttacatttttttagtcgagggttcctcttaaattgtgtgcagagggggctcaattttgttaaccggaacaaggcgcatgtgcgtcttggatcaaccccggtcttgtcttcaggcactgttaccttatttatcaaacatatgtacacctccatctatatatagtatgagttttgatgcctatctattttgtggctgtcgtctcacaaggttatttatctatatatagtgtgagtaattcttgttcaaatcctttctccttactataaatatttgtcacaatacgcgatcgctcccaataactgctataaatatttgtctccgccgtggcctccatccctaaacggagcacagagccagccccactccctttctccttactccgacaggagatgcggatgccgccgagggccgttccacatggcccctaaaaacagtggggtcctaagctgctggacactagcattttccctatagtttatctgctttatagtttatctactttagacacaaatacgcaaagagcatcgcactgtcatcctgtcttatttagattgttatcctaatatctcaattgcttatcaaacattatttactatacccacgatggttatattggttgagagactttttaaaattgaaattattggggaactatttaaggcctgcaatgattgaaggaagattaaatagtttggcaattctatgcatggagaaaaagttggatgctattgatctcaatggtataatctttgactttgtatcacaatgttagaagacatttttagcgtgatatgaatgagacgtgcgacagcgcagccacacaatagcacacacttttatacgg

SEQ ID NO: 21 - DNA, 2944 bp, Zea mays
  OML4 promoter sequence (maize)
catacttgttggcaagagcgccaatcacggtgcctcaaaacaggttattgacaacgtcgaacattctctcctcttcaggagtgaactgttcgggtttcccctgtgcggcgtgataacagttcattgcagccaaccacaatatcatcttactacgtcatcttttgtaaaatgtcctatcaaaaggttcacttggttttaaagtagcaacaaaaccactaacagaaaaatgcctaatatcaggtttttggattgttagagaaatatgcattttcagttttaatttaatccagaaaatcacagtgatgtatgtgatgacatgtatgtgcatatgtgtatcactactcacataagttgtaaacaacagtaaattatacacaaatactaagaacagagtgtaccctgtggagggaccgatgttgcaaggcatcagtggctctattcacacgagacatctcatgtgtatgttcgatgtagtcatacgcagtcgatgtagacagatgtacgtagtgcagtccctcgaacgacgccggcgacgaggaacttgatcagcgttgattcagcggacgaagcgagcagtcgtgagtacgctccccaaaaacctaatcgtccgcacacctgtgcaagtaacagacagcgatttcggaggcctgctctcccaaactctctgtgctcgcagaaggtgggacgagaatggctgtgtgcaacgcgtctgagactctacgtgcgtactgtgaatagaagcagcctccactcctccatataagtacacgcgcagagggaggtgaacagacagtaacagtcaccatcagagctaccgttatagacagccagaaattgataccattagtgacgtccgttactagccgacaaccattacagcccgtccgttatagccataacacaggaaacaaccagtaacagacgataataatggactgtcattactctaggcaaaatatgcaacccttaggacggaatattcggatcaaagtccgatccaccacggccccgccggcggcggcgcgcgcgcatgatagtccttcatcattttctcagctttatcaatagatgcaccaatgatacttctatttaagttgattgaattgtcacttgaacttccggtatggtactaaagtactagtacactgtagcattaaaatgagcctttaacattaactattattgaatattaatttgtgccagacccacattaattcaacagtcgttgcaactagccatttttggatccaaaaaatttaaaaaaattgcaaaaaccacaaatttcaccccaatctctttagaaataccctacgcggatggagctcgttacacaaaccattccattatgttgtgcgatttctgagcgttcaaataaacgtgcgtgaattacttaattctgaaataaaaaagctatagaggctgtagtctgctacaatctatgtactagagcattagagatgaagtgaagtcgagagctgatatgatatggacgagaggaggatgctgcactagaacgaggctaatccaagcagtgagtgagaggagaacaatctggcgcaagcaagcaagcagcaaggcttgccgcccgtcctaaccaactcagcccaaagccgtcgcctcccccaactcccaccacccaaatttgaacccaccgcacaccaatgcaccgctctcttccgtcgatcccactgcagtactggtcccacccctgtatcaagtcactgacaagacagcccgcctagagtgggccacatctcgtcagtttcaggtggtatgaacaagccccaggacagccgcgcgcgccgcggtcccgtgctccgcggtggcagtcaccgggcgtaaccgcagggtacggtataaagggcgtgccgcccgtctcttgccgcccgcatttgggtaggtagttgctgttcccctgcgaggcccgcgtctccccttcgtctcaacacccaccgcctcctcgccggagccagtaagcttccggcgaagaaatccggcgggcacatcacaagggggccgagcagggggacctaggcgagcgccggaatgggggccagcgcccgggggctcgccgtcgtcgtggctctagggttagataggtgtccgtagcttttttcttcgctcgccctcccccacgcggccagggtgtgcagccccggcgtcgcattgggtcccgggacgaccgtaaggccgcgtgatccacccgtgctcgagctcggacagggtcccggttgcgtggcatgggggcatctcttccgcctgctcccgctgcctgcgagtttggcaccgtttttgagctctgaagaggaggaggtggtggcagcaggcaccgatctggtgagcccccactcgctttccgttctctacatggatggtttctgtttgggatgtttcaattttgggaaattttgaaagctctcgtataagtcgttttgtttcgtgggtgtccttgcttgctgtatgtaacctgagcttgaattcggggtctgacaattattttgggttgtgttctgccgggaattcctcgttttattttgatggtttctttgatcactagggacttgctggtttggagctcgtagagcccgaggcgcattaaattttacatcttctgtgctgtcgtattgggggaaattaaacatttctctcaaatttgtgggattcgcactctggtttgtcaaacctactggttctgattcagaagtattgactttggaagctcacacgagctaaaatccgcctttttctctgctgccctgtggctcggttgtcatggattgacagatttctgcccgtaaaattgctcctattcgtcatgttaacccctcgacacttcatcttttccgcaagttttattaattttgcgttgatcctgggcaattgagatacggtgctgttgtctaggtttgtgcctaacacgttatatggtctggacgcctgcagg

SEQ ID NO: 22 - protein, 406 aa, Zea mays
  GSK2 amino acid sequence (maize)
MEAPPVPELMDLDAPPPAAADAAAAAPVPPAVSDKKKEGEGGDTVTGHIISTTIGGKNGEPKRTISYMAERVVGTGSFGIVFQAKCLETGETFAIKKVLQDRRYKNRELQLMRAMEHPNVICLKHCFFSTTSRDELFLNLVMEFVPETLYRVLKHYSNANQRMPLIYVKLYMYQLFRGLAYIHNVPGVCHRDVKPQNVLVDPLTHQVKLCDFGSAKVLIPGEPNISYICSRYYRAPELIFGATEYTTSIDIWSAGCVLAELLLGQPLFPGESAVDQLVEIIKVLGTPTREEIRCMNPNYTEFRFPQIKAHPWHKIFHKRMPPEAIDLASRLLQYSPSLRCSALDACAHPFFDELRAPNARLPNGRPFPPLFNFKHELANASPDLINRLVPEQIRRQNGVNFGHTGS

SEQ ID NO: 23 - DNA, 5830 bp, Zea mays
  GSK2 nucleic acid sequence (maize)
atggaggcgccgccggtaccggagctcatggatctggacgcgccccctcccgccgcagccgacgccgcagccgcggcgccggttccccccgccgtcagcgacaaggtgagcgagtgccccagatccggagctgggctcggatctgcggccgtggtcgcggctgggcgcctcccgatctgctgcctccgcgagcgacgttgctaatggtggtggcctgtctattttttcctctctcactttccgtttgtgttgcagaagaaggaaggggaagggggagacactgttacgggtcacatcatctccaccaccatcggtgggaagaacggcgagccgaagcgggtaaagctacgcttctctcgctgtctgtttgtctatctgtcgtgccgatgtgcgcgtgaatgctgctgcggttagtgcggctgaagtgcccccgcttgtttcgtagcggccttgcggtcggaatccgttttgatctgacggtttgcgcatggggtcgtgttctgcgcctcttgtttagcggctacacagctacagctagcatgctggtgaaatttggtgggtttgttctggttttgttgatgtattatgctctccccgctactctgggcctctggggattctggctgggttgcgcttccttggcttagtgtttgcagctgaattatgtgtctgaccgcttcatttcgtgcttcgttacttggttttttaaggctaacatgcatttaggaagcacggtctaccattcttgtgattagttctgccgtgtgcagaacagaaatggtctaactgttagtttaggtccaggtatgagtgaggattcgaattccttcctgctcagttgctctgacgcctgcctagtttgttaccctcttcgtgtcctcagttgctcatttgttcttcttctggccttaattgcagaccatcagttacatggcagaacgtgtcgtgggtacgggctcatttgggatcgtcttccaggtatggtgcttggtcatgggagctcttctttgtacgtgcctaacatttgttgatgtaacatgcactgaattaactttgacatgtaggctaagtgtttggagactggagagaccttcgccattaagaaggtgctgcaggatcggcgttacaagaaccgggagctgcaacttatgcgtgccatggagcaccccaacgtcatctgcctgaagcactgcttcttctcaacaacgagcagggacgagttgtttctaaaccttgtcatggaatttgtccccgagaccctgtaccgtgtcctgaagcactacagcaacgcgaaccagaggatgcctcttatctacgtcaagctctacatgtatcaggtttgtgaaccagcatcttaacttatatgaagctgctaatgtgtgctttcattgttttgctaactgtctctttttttgtaatgttcgcagcttttcagaggcctagcctatattcataatgtaccaggagtctgccatagggatgtaaagccacaaaacgttttggtacgtgtcatgtggacaaggtttcgttctttctttgatttggtaactagttctgagttggtctgatccttctttgatatacaggttgatcctctcacccaccaggtcaagctctgtgactttggtagcgcaaaagtcctggtatgttgtttttctttccttgaggatttgtagtcacatccagttgttgtatgctttctcttttgaaatattcttatcaaaggcttgtttttctttccttgaggatatgtagtcacatccagttgttgtatgctttcttttttgaaatattcttatcaaaggctatccatactattggcatggcattagtggtttgtgtcactatgtaaaatgtatcatcagtatgctgctattgcctgttatgattaattgtgattgtagttggttggtcctatggaacaaaacacatcttgaaggtagcttaagtatagatgcaaggctcgtggatatatttctcagtgaactattgatacaaaaactgtcctgttacatagttttggtctagatatcttgcagatcaatgttggctacattttagtcaagcttatcaaatttgtcttcatcatgtgcagttatatttatcctatttgagctatgacttattcaattgtttctggggctgtctttgtatgtaatagcattgattcttttgcttctatccgaagttccaatcttggaattgactgtaattcatgtgttataacaattagattctttacttgtatgctgtatttttttatccacatactaatcagttccatatgttgttttgtcagattcctggtgaaccgaacatatcttacatatgctctcgttattatcgtgctccagagctcatatttggagcgacggagtatacaacttcaatagacatatggtcagctggctgtgttctagctgagttgcttcttggtcaggttggttgcattcatataatgattaacctaatatttttgtacctcgatttgaccaaatcatgtatggtgtgattctaacctcttggggtcttggatcttatgtatcagccactgtttccgggagagagtgctgttgatcagttggtagagattatcaaggtactgcaaaatgttccaaagtagacattctattcttctaccggggtgtttcttatggttatgtgatgtgcctgtaggttcttggtactccaacccgtgaggagatacgatgcatgaatcccaactatactgagttcaggtttcctcagataaaggctcatccgtggcacaaggtattcttatgttaaaatcatgtttctgtccacatctatgattccatttcaccagcagctactgtagttatatacttgtagcccacggtccaaaatgttattgaagggcgcttaaagaaattgtttgcagatcttagcgaaaatttgagctcagaatgcatcagttactgactgattgttccactttccgttttatcctccagattttccacaagagaatgcctccggaagccattgaccttgcttcccgtctccttcagtattcgccaagtcttcgctgctctgctgtgagtatttttttttactttgtttatttagattagagtcagctttgatgcttatagtttgaggggaatagacaatggaacgcgacagactaaggtctttgaggtctttgtgttgcatatggtctattttacttggctttggttattcgaaagcttccactgttgtcaattatccgtagtcctgtagaccatgaccaattaaaagtggctaaaatccatgtggaattatgtcctctcaaaccatagcgtatggtcctgcatgtatatggtaattatgctgcccagtggtccagaaggctagtagaaccatcagttttgatggatgttagctgatgaagagtgggtgcaactttatagtcacatgtgcttgttaagtgtactagctagtggcccacctaaaaagagcagtcctagttctcacatgctgtagggtggcacacaccataatctttaatgcatcagtttgttggttagaaatgttctaatgtgcttcatgtattctatttctattcagcttgatgcatgcgctcatcccttctttgatgagctccgggcgccgaatgcacgtctaccaaacggccgtccattccctccgctctttaacttcaaacatgaggtaagcaaactaaacacagtgcaaagttcgtttagccagacgcttcagttcggtcattaaagacctgaaaggatccagtttgcacttgctctactgttttgctcatcagttacccccccccctttttttttgctatgcagctagcaaatgcctccccggacctcatcaacaggcttgtaccggagcagattagacggcagaacggtgtcaactttgggcataccgggagctaggagggcaggcggctgccatggtcaagtttttggtcttggtaccccatgtgcagggccgattgcaggtgacggtgatattgctgcaccatctggagaggaggggctcagcagtacctgagagagctgaaactatgtaaattatctgaccgcgaaggagtacggccagtgttaagccagtaaactggcgcatgttggtccagagtagttaagaatgtagcaggtggagactggtaaatgcctagggtcgtttttagttgttgttactagtattttgtaatgtaatgttcgtcggtacttcccagcagtagtgtagctgctcatgttttgttcgcccgtcatgatgtaaatgatcatcacccaactggaacccctgttatctcgttacatgcttaggcctctgatcgttccgtagttgctgtgatagagctctgacaagtctgagcaggaaagtgggtaggaattgcttctggtgaaatctggacaagttttgtcgaatacagatgcatctgctgattgatcgtctggttgcaagtagtctgcacattcccaaggccacagatcattactttcagattgttgataacgaccaaatggcaagtaacagaaacgaccgaaattcgcaagcaggcaattacagacgcggccgcgccagcacatcgccgccgtagtccttgaccttgctccacaaaaccggccatgcgtccttcctgatggcggctaggttctctccatcccagtctctggctgcgagctcctcagccatggcgaccgccgcggccacgacgtcctccacgccgccgtccacggccgcatccacgatgcctcggcgcgcggcctgcgcggccgtcatcttctcccccttcatcaccaggtccctcctagcggccgcgtcggggaccttctgccgaaccagctcgccgacaaaatcgacgatcttgatcccggcgtcgacctcgctcatgtagaggaacccgcgggaggcgcgcatggcgacggcgtcgtgcgccagcgcgagcgcgcagccggcgcccgcggcgtggcccgtgacggccgcgaccgttggcacggggagcgcgagcaggtcggcgacgaggccgcggaacgcggcgcgcatctcggagaggcggtgccccggcgccggggccggccccgcgcgcgcccacgcgaggtcgtagccgttgctgaagaacttgccctcgccggccaggacgagcgcaccgggggaggcgcggcgggcggctgcgaccgcggagcggagggcggagagcagggccgggctgaggcggtgctcctccgcgccggtgagggtgatgacgtgcacccgcccgcgcttctccacggcgcacaggctttcctccatcgttggagtggatttggggctcctcactgctatgccactgatagtatgtttactttttcccctcttgcatctgggaaggtccaaaatgtccctggtccagctctagtacagaagtgttcaactaaaccttttctgttctggcgtcaacaccaaggccctagagcacaaaccaaatttaggagtgaaactaaattatatcaggaacataataattggaggtgaatttaattgtatagactaccttattcaaattatgagcctattttaattggatgtgacttcaacattattagatatgtgaaagacaagaacactatgaatggtgttcataaacacaaaaccatctcaattctttgattcacaattttatgaactttgagagttggtaatgtgtggtgggctttttcacttggtcaaacaatcaagagttccccattttgggaaaaacttgatcgaattcttgtctcaaaaggtgagaagtcattttttccacaagctatggataaatgaatccctagagagatttatgaacacaatcccctgcttctttcaactggaaact

SEQ ID NO: 24 - DNA, 2711 bp, Zea mays
  GSK2 promoter sequence (maize)
aagtgaaggatatcttctttgcgaatgggatattccgagttaacaatggacaggacacaaggttttgggaggacaaatggctgggggatttctcgctccagcatagattcccgagcctatataacctagtgcagcggaagaatgctactgtggccaatgtgctagggtctgtacctctcaatgtatcctacaagagaggcttacatggtgctaatttggagagatggcataccttagtcagcctagtagtggatacgacgttgaaccaggcaagagatagttttcgttggagccttcatcaaaacgggttgttctccactcagtctatgtatgcggcattgattgggaacggacaagtacggcaggatggcctcatctggaaactaaaactccccttaaagatcaagatcttcttctggttcttaagacaaggggtaaccttaactaaagataaccttgccaagagaaattggtcaggatcaaaaaaatgtgttttctgtccacaagatgaaaccattcaacatcttttcctccagtgtcattatgcgagatttctatggcgtacggtatattttacatttggcattagagaaccaactagtatagaagatatgtgttcttcttggcttcaggggtttcaccctaatgttaaagctaagatatatgtgagcgctatagctatttgttgggcgttgtggctaagtagaaacgatgtggtttttaataaatctcctacccaaacttatttacaggtactcttccgaggaacttactggtgtcgtttctagggatgcttcaaaggcataaagaggacactagaagcatgagggaggcctgcagacttttggagacatcgatgatgcaagtcttctcgacgtatggttggaccttcagtaatagattaactatgtgatgtttgtctattctcccaactgcgtttgggttttgtggccaaatgtggcgttgtttcggtactttatgttgtggtgtgtggacggccgtcatcagctgatgtaggtcgggatttggtttttttttcccgttatctaaaaaatatatgtggctagatttatcatcatccaggtaaatatagacataaaaattaagatctcaaatgaataatatcttcgaccggatggagtatgacataattttacatcacgatttctaaacaattgctaagttctttccgctcattcggtctattgtacatatgtatcaacatcttatactcatccgtctcaaattaagattcgttttacttaattaatgggttcatacaacacttgatttatatgttatgtatgtgtctaggttcatcttcatttatttgaatattgatataaaaatcaagagttaaaacaactattattttgggacgcggtgagtattttttctccatttcctcgcacctagggatttcacgcgatggatacacattctatgtaaaaaaagattgggcgttaacagtcagtcattaaaaatattctttttctaaaaaattaaaaaaagaggatctccattggaaatatgttttttcgaaactactggagatgctctaggtattgtgaacagtttttttctcattaaaaagatgctgcaaaatccgttgatgctcctagatcactcgacaactacagttaccatcgttcatgccttcggttttagcaacaaaaaacagtgcaatcctaaacaaaagcatctattaatcacaattggttgctgccattggtactgcactcagcaactctgttagcaaaggtaatgcactcttgtagtctttgaccggatcttttggctagggaaaactaaggatgcgtttggttacgggacaggcaggatagagatgtccccaggcgtactctctcgtcactctaatttcgaggggcaactagagacaacattggaataatcctgtctcaacccctgattctgaactaaacaaccttatttaaggtacgtcctatctcatcccgttctgtcattataaccaaacgcaccctaaaaaaatgttcatgaaggagagaattaaaaggttccagttttcagtatgctagtttagcaacgagtgtattgcaattaattatcactattgttcggaccctccattttggtagtacaggtaaatccctactaagcaagaataatatgtttttttatgctacacataggtagcgtttgagtagacttgtattttaaataaaatgctactgctgataagactataacggtacgggaaaaagaagacaatttagagcttgccaaatttctttagcagccaattaattcctaccacggtcctgtcctcagaattttttttagtaacaaatcagtgcactactgattcctaaaccaggctgaaaccggaaacggctcgctgcgctgccgctgcgtcactgtcgctggcaaagaaaacaactcccggccaggggtccgagcaggagcagcagtatattttcccgccgctaataaaaacagtcagcggcacacttcgccaagcgaggcaggcagcggctgtcccgagctgtcgaaagcgaggcgcggcggcagtcctcgcagcagggccgaccggtcaaaagcactgctgctccacaccacccccaccatccctttccccaacccccgaagccgagccagcgaaccaccccgcccgcagccgcaagcaagcagccaagcagtgtgaactgaccgtccgttccgtccagcccacc

SEQ ID NO: 25 - protein, 917 aa, Brassica napus
  OML4 amino acid sequence (B. napus)
MMPSDIMEQRGVSTPSHFREDTRISSERQFGFLKTDLIPENQGGRDRFSNLPKSSWTPESHQLKPQSSLSGVHPSVSPNARNTTNGSQWESSLFSSSLSDTFSRKLRLQRSDMLSPMSANTVVTHREEEPSESLEEIEAQTIGNLLPDEDDLFAEVMGDVGRKSRAGGDDLDDFDLFSSVGGMELDGDVFPPMGPRNGERGRNNSVGEHHRAEIPSRTILAGNISSNVEDYELKVLFEQFGDIQALHTACKNRGFIMVSYYDIRAAQNAARALHNKLLRGTKLDIRYSIPKEIPSGKDASKGALLITNIDSSISNEELNRMVKSYGEIKEIRRTMHDNPQIYIEFFDIRASEAALGGLNGLEVAGKQLKLALTYPESQRYMSQFVAHDAEGFLPKMPFTNTSSGHMGRHFPGIIPSTSIDGGPMGISHSSVGSPVNSFIERHRSLSIPIGFPPLANVISASKPGIQEHVHPFDNSNMGIQSMPNLHPHSFSEYLDNFTNGSPYKSSTAFSEVVSDGSKANDAFMLHNVRGVDGFNGGGIGSPMNQNSRRPNLNLWSNSNTQQQNPSGGMMWPSSPSHLNSITSQRPPVTVFSRAPPVMVNMASSPVHHHIGSAPVLNSPFWDRRQAYVAESLESPGFHIGSHGSMGFPGSSPSHPMEIGSHKSFSHVAGNRMDINSQNAVLRSPQQLSHLFPGRNPMVSMPGSFDSPNERYRNLSHRRSESSSSHADKKLFELDVDRILRGDDVRTTLMLKNIPNKYTSKMLLSAIDEHCKGTYDFLYLPIDFKNKCNVGYAFINLIEPEKIVPFYKAFNGKKWEKFNSEKVATLTYARIQGKVALIAHFQNSSLMNEDKRCRPILFHTDGPNAGDQEPFPMGTNIRSRPGKPRSSSIDNHNGFSIASVSENREEPPNGTDPFLKEN

SEQ ID NO: 26 - DNA, 5843 bp, Brassica napus
  OML4 nucleic acid sequence (B. napus)
atgatgccgtctgatataatggaacagagaggtgtatcaacaccttcccactttcgtgaagatactcgtattagttcagaggtaactttttcttttactgtgtagcaccatctttgtcacattatctgccactattttctatgatgtttaaaactgttttctttttgtttctcaagtatacttgttcttttgtctggcagaggcaatttgggtttctgaaaacagacctgattcctgaaaaccaaggtggtcgtgatagattttcaaatctgccaaagagttcctggacacctgaaagtcaccagctgaagccacaatctagcttgtctggggtgcacccctctgttagccctaacgcaagaaacaccacaaatggtagccagtgggaaagtagtttattttccagctcactgtctgatacatttagtagaaaacgtaagcttctggttcacttttatgaattgttacttattatgttgattttgttttatcctctacggtaaagaaacgccgtttgttaatctagtacatcatagacgatcgtgaaagtttgtttctttctcctttaacttactgtactttaactacttgactgcgtctccaaattcttggtttttgcagtacggttacagagaagtgatatgctatctcctatgtctgcgaacacagttgttacccaccgtgaggaagaaccctctgaatctttagaagaaattgaggcgcaaactattggaaatcttctgccagatgaagatgacctctttgcagaagtgatgggtgacgttgggcgtaaatctcgtgccggtggagatgatctagatgattttgaccttttcagcagtgttggtggcatggagctagatggagatgtttttcctcctatgggccccagaaacggagagagaggccgcaataattctgttggcgaacatcatcgagcggaaattccatccagaacaattttggccggaaatatcagtagcaatgtcgaagactatgagctgaaggtcctttttgaggtaccttattccagcagcgtttccccccacagatttgtttatataatctggaattgattacttcgtactgagaatacttttacttgttcagcaatttggagacatccaggctcttcatacagcttgcaagaatcgtggttttatcatggtatcctactatgatataagggctgctcaaaatgcggcgagagcactccacaataagctgttaagaggaacgaaacttgatattcgttattctatccctaaggtatgattccttgtttttatgaaatatattgtctttgctctgtggacagtatttgtgacttatgttgatttgtatctatcttacaattttcttggctccaggaaattccttcaggaaaagacgccagtaaaggagccctgttgattactaatattgattcgtctatttcaaatgaagaactcaatcgaatggtcaaatcgtatggagaaatcaaagaggttgatatattgagatgctccgtttagttacttttctgaggtagattctaatgatgtttctgtggtttgcagattcgtagaaccatgcacgataacccacagatatacatagaattctttgacatccgagcgtcagaggctgctcttggtggcctgaatggactcgaggttgctgggaagcagcttaaacttgcgttaacctatccagagagtcaaaggtgggtgactggttgttttttttttctccctggtttatattcctttgtgggctgtgaatgaatacaaaatcctaaatcaaaatgatttgaacatgtgctttgctgttaagtatttacgaggatgccagttgtgttgatgtatggggttcacccattcttttttctttatttcaggtacatgtcacagtttgttgcacatgatgctgaagggtttctacctaaaatgccttttactaatacatcatctgggcacatgggtatgcttttgcattcagcatttgtaattcttttttttattgaatgatttgtcatcttgatactcaaaccactgccgttaaatatctctgtgtcagggagacatttcccaggaataattccttcaacctccattgatggtggacctatggggattagtcatagttctgttggatcgcctgtgaactccttcattgaacgtcataggagtctcagcattcctattggatttccacctttggcaaacgtcatctcagccagcaagcccggaattcaggagcatgtccacccttttgacaattcaaatatggggatccaaagcatgccaaaccttcatcctcattctttttcagagtacctcgacaactttacaaatggtagtccatataagtcctcgacagcattttctgaagtcgtcagtgatggctcgaaagcaaatgatgcctttatgttacataatgttcgtggagtggatggctttaacggagggggtaagctctttatctctaaattgctactgttttgataaatttgtcgaagaataatgatgatatgtagttgacaattgtgagtttaagaagaatgtctgccgtagcacactgttaggatggtccttacaattttagtggaatctgaaatgtgctacagcgatgaaaattctaggtactgtttctgtagacaactttttttaaaagcattcttggtgtaaaacttgtcatcctgggaaaatattattagtattatgttcttaattgcagtcatatagacagataactgtgctgggtttgaaattgaatttgaaagtggctgaaacattcgttgtgtatgtcaacagaattgcacaattactgagtgctagtatttcttctactgtcatacataatattgtttttttctttctcacttttagttgttgtggtcttttgactgtaggcatagggtctcccatgaaccaaaactcccgccgccctaaccttaatttatggagcaattctaacactcagcaacaaaatccttcaggtggcatgatgtggcctagctcgccgtctcacctcaacggcaaacgtcatctcagccagcaagcccggaattcaggagcatgtccacccttttgacaattcaaatatggggatccaaagcatgccaaaccttcatcctcattctttttcagagtacctcgacaactttacaaatggtagtccatataagtcctcgacagcattttctgaagtcgttagtgatggctcgaaagcaaatgatgcctttatgttacataatgttcgtggagtggatggctttaacggagggggtaagctctttatctctaaattgctactgttttgataaatttgtcgaagaataatgatgatatgtagttgacaattgtgagtttaagaagaatgtctgccgtagcacactattaggatggtccttacaattttagtggaatctgaaatgtgctacagcgatgaaaattctaggtactgtttctgtagacaactttttttaaaagcattcttggtgtaaaacttgtcatcctgggaaaatattattagtattatgttcttaattgcagtcatatagacagataactgtgctgggtttgaaattgaatttgaaagtggctgaaacattcgttgtgtatgtcaacagaattgcacaattactgagtgctagtatttcttctactgtcatacataatattgtttttttctttctcacttttagttgttgtggtcttttgactgtaggcatagggtctcccatgaaccaaaactcccgccgccctaaccttaatttatggagcaattctaacactcagcaacaaaatccttcaggtggcatgatgtggcctagctcgccgtctcacctcaacagcattactagtcagcgcccacctgttactgtattctctagagcacctcctgttatggtgaatatggcatcttcccctgtgcaccaccacattggatctgcgcccgtattaaactcgcctttctgggatagaagacaagcctatgttgctgaatctctagaatcgcctggcttccacataggttctcatggtagcatggggtttcctggctcttcaccctcacatccaatggaaattggttctcacaagtccttttcccatgttgctgggaatcgcatggatataaattcccaaaatgctgtactgcgatctccccaacagttgtctcatctcttccccgggaggaacccaatggtttcaatgccgggttcgtttgactcgcctaatgaacgatacaggaatctctcacaccgtagaagcgagtctagctctagtcatgctgacaagaaactgtttgagcttgatgttgaccgtatattacgtggggatgatgtcaggacaacactgatgcttaaaaacattcctaataagtaagtggattcagtgtctttcctttattccttgttatatatcttttgttagcttcgtaggttgtttgatgttttccttttcaattctgaactctataaaatgctgctatggtttaggtatacttctaagatgcttctctccgccattgacgagcattgtaaaggaacgtatgatttcctttatttgccaattgatttcaaggcaagcaggcgtccgtcctacctttttatataatagtcttatgtagaaaatgggcttttggtatttgcaatatcagtatttttttgctaacctaattttaccttctcgtttcagaacaaatgcaatgtgggatacgctttcatcaaccttattgaacctgaaaagattgtaccattttataaggtacagccagccttttctgttgctgctttttatatattttttggctttttctcttgaagagcattggttaaaagtttaaaaaaaacttgcaggcttttaatggaaaaaagtgggaaaagtttaacagcgagaaggtggcaactcttacatatgctcgaattcaaggaaaagtagcacttattgcccatttccagaactcaagcttaatgaacgaagacaaacgttgccggcctattcttttccacaccgatggtccaaatgctggtgatcaggtgaatgttactaacacatcagataacatcatcttgttagggttctcatttcgtagtagttgctcaatttcgctctccctttggttgcacatattgaaatgggttcttagtgagatctcataagttcaaagatgtggtgatgctcagttactcaataagagattgatttgtttcatatttgtcacctttgttgttattatttgcaggaaccatttccaatgggaaccaacatacgatcaagaccaggaaagccacgaagcagtagcattgataaccacaacggctttagcatcgcttccgtttcagaaaacagagaagaacctcctaatggaaccgatcctttcttgaaggagaactaaccaatgagcaaaaaaaccaagcagaggtaaaagaaagttaaggaaaaatgaagagctaaagatataacacaagttttatattattataatcatatcatcagcacaccctagagttctgtaaatcgggggtgttaaatttaccctgacaaaactgtttttgcggtgaagatatatttttggagagatcattaaactttgttgacctcaaaccttcacaggttgcttcaccagttttgttgtattatcaaatatcccctgagaaatatcttcgagagtttctctttactttttgtttttttttttgtcttgtttggggttattcaagtatttttgtcttcttgctatcgatgtagtatgtaacaagccttggatttacattcaacgtctttgctggctatttgtggccatttcatgttgtaacttttttggagattttaatgaatgcttcctttttggataaa

SEQ ID NO: 27 - DNA, 2619 bp, Brassica napus
  OML4 promoter sequence (B. napus)
agtaattaatattcttttcgttccacaaatataatttttttagtattttcacacatattaagaaaacacgctaaactaccataataaatgtattgttttatgtaattttcaattttcaataacttttaaccaatagtaattcaataaagtcaattaatttctttgaaatttacaaatttttcatagaaaacacaaaaatacatatttgtgaaacaaactttttcaaaaaagtctatcttgatgaaacggatggagtattatgtataatatttttattatatattttattgctaaataaaaattttatgacttttgtttactttttcaccaataaaagactataatgcaaaatgtaaaatatttaaagtttaatttgaagttgttatttcggaaataatcaccttcgaagtttaaatttgtaatattgcaaactttatttggagatgttttcacggtcgacttgctacatgactcttttttttttgtagcatgctacatgaccctctattcttttttttcccctatttattgttactttacaattgaaataataaggcaaaatacaatagtggatgacttttttccccataccacctttttcggttttctctatttggttgttcgaacctgcacatgctcatttgatagcgtggaaggattggccatcaaacaaataaaaattcacaatcaaggatatttattatcagtttttgttgttgtgcacttcattgtaaaataaaaaaaatccaaacacggatgataacaaccgtggatcacgagtaaactaattcactcagtcataaaaagaaagagatatagtgagcaaaaaatcattttaagatagtattgatccaaccaaccaaacattatcttcaaaaattacaatgtttttacgacagttgataaaaaaaaagcttatttagtaaacataaaaactatggagtagttttttttgtaaacacaaactatagtttcagactttgttttgtattctttcaacaaagagtgtaactataaaaacattcttatcaacttttcgctcaagttgttacagaaaaaaacttaatcaagaattaaaatgacacttataaaattatcaatataataaaattattaatttatagaggttatatcaattaaaaactaacaacttttattcgtgttttcattcatatataatagtaaagtgtaatttcctaacttcatttgaacatattctaataaatagtttgtagattaaaaacaaatcacactttgaaaagaaaaaaaaatcaaatagtccacatgttcaataaataggctgctccttggttacaaaaccgcgctcatcgactgctcgctgccgtcgagactctcgtgtgagaccgtaatttttgtcagttttagttataatctacggtccagatttaatatcgtacgaaaccactagatccacgatacatccaacacagaagagtgctctcctctcctcaactctattttgtttttttcctctcattctttttttagtcgaaactctaaaccaactaaccgaaaaaaacaaaaaactctttctctcctctccatttctctctctaggagagacaaccggaatcgcacgtcgacgggaagagtatcgccggaactattataattaccgccggtcgcatagattattcgttggaaacaacgcgtcgtgagaggagaggaaattcgaaaaaaagaagaaaaaaattagaaacaccgattcactttttttttgggggttattttaattgatttgtgtgaattaaatattctgcgatggatgtgattggatagaaggaaacaaaaaggaaaggaggaagataaaagagaaggcgaattattctgctcctctctctctctctctctctttcttctctgtcgaacatcgctgttgctgctgtgtgttttcttcgtgcatccttttatttttcaaggtaatgaatttcacgagatccattcttcacaagtttctttctttttttaaatttaatttaatttagtggaaaaaatgtttgggaggaagcgtaattgtgtttgtttgtaaattaggtaagctctttgtatttgtttttttatttgctggtgagtaatttaggtttattttcttaaattaagttaaactgggtgcccaagtttgtgaattaggtaggagttggttccctgtttgcatataatgagctgaacaaggatcatgaattaggcgaaattgtagtctcttatggctttttgaaatacctaatctttgtcttccaggtgtttctactccgctttaaaggagagaggtttaagatgatttttttcgtattgaacttcttcttagagtacgtaaagttgctgactttgtttggatttagggtttgattttgcttagttctaattgaattcttgtgttgtttttttttgtgtcctttgagttattttgcttaatcttttttgtctggcaagatccttctttgcaatgaatagtggattttgtttcttttggagacttactggctttgaatctaaaactggttgttcatctttcaggggaagtgatatggtccgttgaaaaagactaaaaagctacaaaagagattttgttttattattccaaattttgctgtcatctgc

SEQ ID NO: 28 - protein, 410 aa, Brassica napus
  GSK2 amino acid sequence (B. napus)
MTSLSLGPQPPATAQPPQLRDGDASRRRSDMDTDKDMSAAVIEGNDAVTGHIISTTIGGKNGEPKQTISYMAERVVGQGSFGIVFQAKCLETGESVAIKKVLQDRRYKNRELQLMRLMDHPNVVSLKHCFFSTTSRDELFLNLVMEYVPETLYRVLKHYTNSSQRMPIFYVKLYTYQIFRGLAYIHTVPGVCHRDVKPQNLLVDPLTHQCKLCDFGSAKVLVKGEANISYICSRYYRAPELIFGATEYTSSIDIWSAGCVLAELLLGQPLFPGENSVDQLVEIIKVLGTPTREEIRCMNPNYTDFRFPQIKAHPWHKVFHKRMPPEAIDLASRLLQYSPSLRYTALEACAHPFFNELREPNARLPNGRPLPALFNFKQELAGASPELINRLIPEHIRRQMSGGFPSQPGH

SEQ ID NO: 29 - DNA, 4412 bp, Brassica napus
  GSK2 nucleic acid sequence (B. napus)
atgacatcactatcattgggccctcagcctccggctactgctcagccgccgcagcttcgcgacggagatgcttccaggcgtcgttccgatatggatacagacaaggttgctctctccctctctctctctctctctctctctactttaacgtttggtgaacaaattgcatttcgattgcgtttggtggctattgtagatctcggctagatctagcttcgatttcactttttttttgcggtttctcagcgaatcgatctgtgttttctcttgctatcgtcgtagttcgtagttcgtagtagctagctagtcttactattcagctgaatgtttcaaccaatcatattgaagatcttgagctatgttttgattactagtattagggtgaagaacattggttctctctgggtttgaaattcgatttcacagacgatgtagatcttaattactagattgtttaactaatcacacacttgttccatgactgtaagtgatttgatgtattggatttacatttgtttgttatctacgtgattggactctgagctaggccttgactgttcttggatttgaagatttcatatgtttaaagaatggttttgtctattgattgtttcgtaatctcatgtttgttgttttcaggagaagagcactatttttttttttaatcagttttctttgttctttcttgacgagaatagtttgatgatatgttgaggtttggttgcaggatatgtctgctgctgtgatagagggaaacgatgctgttacaggccacatcatttctactacaattggaggcaaaaacggtgaacctaaacaggtttgagttcctttctttgtttgaaatcttcaaatgtcataattagtaacattgttaatgattacatttaatcatatgttcacttgcttttccacttacagcttaaaacaataactaaacagagactctttgtggttcatttattacaactttaagtaggctactcacttatgttttactctttctgtttttttgcagaccatcagttacatggccgaacgggttgttggacaaggatcattcggaatcgtgttccaggtacctttgtgcttctcaatcactgttaccctttgtaggcggtagctttcttctttcctttctgatcgaagtatgaacttaccattgtaggccaagtgcttggaaactggagaatcagtcgccattaagaaggttttgcaagaccggcgctacaagaatcgtgagctgcagttgatgcgactaatggaccacccaaatgtggtttccttgaagcattgtttcttctctacaacgagtagagatgagctcttcctcaatctcgttatggagtatgtacccgagactttgtaccgggttttgaagcactatactaattcaagccagagaatgcctattttctatgtcaaactctacacataccaagtatgcattgttattatgtgtttccctttcaggcagtatctctctttgttgattctaaaacgggtaagaatactttttttctgcagatcttcagaggcttggcttatatccatactgttcctggtgtctgtcacagagatgtgaaaccacaaaatcttttggtacgttgattctattttgggtttgtctttgataatcttgatagattgttaactaattctcttgtacgttctgcaggttgatcctctcactcatcagtgtaagctgtgtgattttggaagtgcaaaagtattggtaaggagctttacctttaatatcctgctttgcttatttcaactgtgtatgtgttctgtctcatgaaatctttgcgacacatgattattcggattaggtgaaaggtgaagcaaacatatcatacatttgctctcggtattaccgagctccagagctcatctttggggccacagagtatacatcctccatagacatatggtctgctggttgtgttttggcagagctccttcttggccaggttagtgtaaactattttatctgtttaactctagaatgttccgctatcatttttgatatttataattttttatctgtcagccgttgttcccgggagaaaattctgtggaccagctggtagagatcatcaaggtgaagtttcattttgatcatatgttatcttgctgtcgtattctgttttgtatataaaattcatataatcttatagatttgtaatgatatatgtgctgcgtttgtttaggttcttggtactccaactcgagaagaaatccgatgcatgaatccaaactacacagacttcagattccctcaaatcaaagctcacccgtggcataaggtatttatatgcatgtccgatcatacagtggctaaatagttgaatcgcttctcattatattcgtataaatgaaaaactaaacaaattcacatacttctctctgaccttcaggttttccataagaggatgcctccagaagccattgacctcgcatctcggcttcttcaatactcaccgagcctgcgttacactgcggtcagtatctctaaaccaccaagtactcttaattgttaagagtgttctctctggattcattggacctgcactgcactgtccaatgttgctgatgttttcttttaactgacatttttttgttgtttctgtgtaaaagcttgaagcatgtgcacatccatttttcaatgaactccgtgagccgaatgctcgtcttcctaacggccgacctctaccagccttgttcaacttcaaacaagaggtacgtcaatcacagcaaaaaaaaaaaaagtaatatagctccaaaccattactagaatgttcagttttaaacagttgcctaatctgtaatctctctctctattcgaatgttcataacagttagctggggcttcaccagagctgataaacaggctcataccggagcacataaggcgacagatgagtggaggcttcccatcacagcctggtcattagaaaaggaatatggaaactgggatgcttttgcggagcaaatgccttatggaaaagaggagagaagatctctgatttttcagagggtttaactaaaatatcagcttatgagtagagagatgattggccaattaagctttttgagaaatcaggaggtggtgatgattgtgtctaatatacaattctctcttttctctttttatgttataattcgcttttgacttgtagagataccttttctcgttgtattatttgtatatgtttttgtccgtaagacagcaaaccgcgatgatggaagaatggaatgaatgaatgatgtctaaaacttaagcctaataacaaggtcggagctcatacatatatataaagttagaatgtgagagctccatgttaaaataaccttaacattggcacgtgaatacaattgcatgattgaatttctggtacgtcgagaggaagtaagtttatagaaagttgtttgtgaacaaacaaatggagaaacatttgttttgttgcaaagaaacgtatggttccataatgtagaagaggcatttgaatgtgagctttaaaacctttcatgaaagaaaaggaaagttatgggtcactaaccggaaaatatatcatttgaaatgtgtataaaacttaatgggctgaaaactgtagataaggaattccggattctgggaaccctattaactgagccacaagcaaagatacgaggaccaaaccctaaatcttctctctttttttccccctcattcaggtgtttttcattagtcacattcgttctttatacttttattatctttgattgttaatagattgtctgaaaacgcatgtccacttgtttctgttttatttgtttttttcttttgctgcaggctttggaagtccacactaaggtgaaacaactctccctaatctatacgcctttcacctctttccccgcctttgatcctttgagagtttttttttcttttttttttttgaaaattcaaattttattcaacctgagaatcgggaaatcatattcggttacaaatccgctttgaacaaaagttccaaaatcaaactattactatctttgcccactcactaaacctgacacattctgctagcctgtttttcgaaattcttcagaatcggttgccgttctaaacttttgacgaaacccagaggactccttgttgctgcagatgccgggacttcctatgtcgtctccggagtagctagttcccgatgaactccacaaaactcataaccgtcaatgtctatgaagcgcttgacggttcaataggggatggtttcaggtacttacgattagaggctttgcaaccaccaccaaaaccgggcttgtacacaaacgccacacagcgctcgaaaccgaatcccaaaccgggcaagcgcccgctactgccgccactaaaccaaccttcaagaaagcgggccgattttgct

SEQ ID NO: 30 - DNA, 2777 bp, Brassica napus
  GSK2 promoter sequence (B. napus)
atgcgttctaagtatcaagatcctattactactactactacaccttgtaatgagaatcataaggtgaagataaatggatcttctactccagaagggaaagagagactagagaacttgagctcagcttcacgcactaaaaccagcaaaaactttggtgagctcttggctagtgatgacaatacatgggaaccttattctgaggctcctgttgctgagaaaactctgtatgtagacactgtgcattcagtacacaagaaggtacaagaagagtctttattaaaagattacccttcactagaagttgttcctgttaaagaagatgttcagaacttgattggagccagtgaagaagctatctcaggtctaaaagttgaagaatgtgctgatcaagctatttctgaagtagtagagattacaaaggattttgaatgttcaaggcttcatcatcatcacattgttgcaccaccatcattgccaaaagctccttcagattcttggttaaagcgtacgttgccaacaatcccatcaaagaacaactcattcacatggttgcagtctcttggcattgatgataataataatcaaatcaccaagagtattcaagaaaatctcaagtgggaaactatggtcaaaacctccaatacacaacaagggtttgtgtgcatctccaaggtaagctaatgtgtatttttcaaagtcaatggttggccaaatgtttttgtttttttttttgtttttgacaagttgattagcttactttgttgaccattatttttgtctttcaggacacactcaaccctataccagaggcatagcaataccaaattacaagtaaatttcaacaataaaaaaaggatgagccaataaagttttgtttttgttcatcttccaaattttctcctctttaattatatgtaaatctgaaataaaaggttcctaaaaagagaaaagctatggagatgaaataaaaggtctcaaatattgtctgtcacttgtggggtttgggggggggtcttattgaagtgatgtacagctcatgttaacagagattttgttgcaataatactccataattccatgtgacattgtttcttttgaccttctttatatattctctgctagtaatagactttttgttttgttcttttgtaattatgtttctgtaatgtagagcactaaagagacctgaaaactgcagaactcaattgaatgcattggctaaatggttatgagaggaattattgaaacaatttatggtgtgagaagttcaaatattattctctttatagtgtcatggatagatcagatatagttcaggagaaagtaaagaaagaaaaaaaaactttataaaggtatcttcattagttaagatatacatgaaagaaactgctgctttaggagatgttttgttgatcttcatgattcttctatctttatcacttgtatgattgtatccatggcggtttttgcttgcttcaaaaacaagaaagagaagaatggttcctgtagctgtggcagttgttggtggctgcggttgtggtggtggaggctgcgtttagtattacacaaatgagatatatcttggtccttggcgagtttcctggtaatgattttggtttagagcatctttatccgggtataccaaagggtttcttagcctgtgggtcccgtgtaggacccattttttttaagaaatcggttacaaaaactactaaatagtagtcggttattaagggtttcttacactgttcgcggaccccgctaacacgtgacggctaacgattggttcattttttttttttaaattcgaaaaagtaaaaaaaataaaataaaaaaaattaggaaactctatttggagtttcagggataatgatgctattaggtcttcaccgattgtgactgatttacttaagcagccattctatatatagtttacattacgtacatatagaacaaaaatatatacataaaatatcagataaattcagaatcaaatatatatgcgatatgtttttgtaaattatttgttcaaattttcaagtctacaaataatgagtcacaaaacaaaatataccaagaaatggattgcgatcgtccatgtgatacatccagggccctctaagacttttaaacgtatctcgtattgaaccaaatgttaaaaccccgttgaaaaggtagccatcttgctcgtataaacgaaaattttcatagatggtagggggtgattggttgaactgtagcaagtgactttaactttaatttttatctacagttttaaaaaccatcaatcgtgctttatattagtttttaaagctaccaccaaaaaataaaaagtacagccaaaaaaacaaaaaaaaaaataactgtaaaaaatttaatttctaaagctccatttttttggatgtaggaaattttaaagctctgttcacgcgtgggccatccttttcaaacatactatactagttgttatttgttacccaaaatgtaaatacatgctatgtccttactaggcagtatatagaaattagtttgttttaatgaatctggaacaatactaacttcaataattaattgcaaggttatccacccttgactgatgaggaggttagtcgcgttctcattggtgcgttactcttacgcgctctatcgacgcgtggacgatatccgaagctcttttaataatacaaagagagagagagagagaagggaaagatagtctttactcttcagtggtgggtagagagcgaaagttagagaaagagagagaagaatagcac

SEQ ID NO: 31 - DNA, 410 bp, Artificial Sequence
  GSK2 RNAi sequence
tcccaggtgaacccaatatatcatatatatgctcacgctactaccgagcaccggagctcatatttggtgcaactgaatatactacatcaatagatatatggtcagctgggtgtgttcttgcagagctactccttggtcagccattgtttccaggggagagtgcagtcgatcagcttgtagagataattaaggttcttggtacaccaacccgtgaggaaatacgttgcatgaacccgaactatacagagtttaggtttccacagataaaagctcacccttggcacaaggttttccacaagaggatgcctcctgaagcaatagacctcgcttcacgccttcttcaatattcaccgagtctccgctgcactgctcttgatgcatgtgcacatcctttctttgatgagctgcga

SEQ ID NO: 32 - DNA, 4206 bp, Bacteria <prokaryote>
  CAS9 nucleic acid sequence
atggctcctaagaagaagcggaaggttggtattcacggggtgcctgcggctatggataagaagtacagcattggtctggacatcgggacgaattccgttggctgggccgtgatcaccgatgagtacaaggtcccttccaagaagtttaaggttctggggaacaccgatcggcacagcatcaagaagaatctcattggagccctcctgttcgactcaggcgagaccgccgaagcaacaaggctcaagagaaccgcaaggagacggtatacaagaaggaagaataggatctgctacctgcaggagattttcagcaacgaaatggcgaaggtggacgattcgttctttcatagattggaagaaagtttcctcgtcgaggaagataagaagcacgagaggcatcctatctttggcaacattgtcgacgaggttgcctatcacgaaaagtaccccacaatctatcatctgcggaagaagcttgtggactcgactgataaggcggaccttagattgatctacctcgctctggcacacatgattaagttcaggggccattttctgatcgagggggatcttaacccggacaatagcgatgtggacaagttgttcatccagctcgtccaaacctacaatcagctctttgaggaaaacccaattaatgcttcaggcgtcgacgccaaggcgatcctgtctgcacgcctttcaaagtctcgccggcttgagaacttgatcgctcaactcccgggcgaaaagaagaacggcttgttcgggaatctcattgcactttcgttggggctcacaccaaacttcaagagtaattttgatctcgctgaggacgcaaagctgcagctttccaaggacacttatgacgatgacctggataaccttttggcccaaatcggcgatcagtacgcggacttgttcctcgccgcgaagaatttgtcggacgcgatcctcctgagtgatattctccgcgtgaacaccgagattacaaaggccccgctctcggcgagtatgatcaagcgctatgacgagcaccatcaggatctgacccttttgaaggctttggtccggcagcaactcccagagaagtacaaggaaatcttctttgatcaatccaagaacggctacgctggttatattgacggcggggcatcgcaggaggaattctacaagtttatcaagccaattctggagaagatggatggcacagaggaactcctggtgaagctcaatagggaggaccttttgcggaagcaaagaactttcgataacggcagcatccctcaccagattcatctcggggagctgcacgccatcctgagaaggcaggaagacttctacccctttcttaaggataaccgggagaagatcgaaaagattctgacgttcagaattccgtactatgtcggaccactcgcccggggtaattccagatttgcgtggatgaccagaaagagcgaggaaaccatcacaccttggaacttcgaggaagtggtcgataagggcgcttccgcacagagcttcattgagcgcatgacaaattttgacaagaacctgcctaatgagaaggtccttcccaagcattccctcctgtacgagtatttcactgtttataacgaactcacgaaggtgaagtatgtgaccgagggaatgcgcaagcccgccttcctgagcggcgagcaaaagaaggcgatcgtggaccttttgtttaagaccaatcggaaggtcacagttaagcagctcaaggaggactacttcaagaagattgaatgcttcgattccgttgagatcagcggcgtggaagacaggtttaacgcctcactggggacttaccacgatctcctgaagatcattaaggataaggacttcttggacaacgaggaaaatgaggatatcctcgaagacattgtcctgactcttacgttgtttgaggatagggaaatgatcgaggaacgcttgaagacgtatgcccatctcttcgatgacaaggttatgaagcagctcaagagaagaagatacaccggatggggaaggctgtcccgcaagcttatcaatggcattagagacaagcaatcagggaagacaatccttgactttttgaagtctgatggcttcgcgaacaggaattttatgcagctgattcacgatgactcacttactttcaaggaggatatccagaaggctcaagtgtcgggacaaggtgacagtctgcacgagcatatcgccaaccttgcgggatctcctgcaatcaagaagggtattctgcagacagtcaaggttgtggatgagcttgtgaaggtcatgggacggcataagcccgagaacatcgttattgagatggccagagaaaatcagaccacacaaaagggtcagaagaactcgagggagcgcatgaagcgcatcgaggaaggcattaaggagctggggagtcagatccttaaggagcacccggtggaaaacacgcagttgcaaaatgagaagctctatctgtactatctgcaaaatggcagggatatgtatgtggaccaggagttggatattaaccgcctctcggattacgacgtcgatcatatcgttcctcagtccttccttaaggatgacagcattgacaataaggttctcaccaggtccgacaagaaccgcgggaagtccgataatgtgcccagcgaggaagtcgttaagaagatgaagaactactggaggcaacttttgaatgccaagttgatcacacagaggaagtttgataacctcactaaggccgagcgcggaggtctcagcgaactggacaaggcgggcttcattaagcggcaactggttgagactagacagatcacgaagcacgtggcgcagattctcgattcacgcatgaacacgaagtacgatgagaatgacaagctgatccgggaagtgaaggtcatcaccttgaagtcaaagctcgtttctgacttcaggaaggatttccaattttataaggtgcgcgagatcaacaattatcaccatgctcatgacgcatacctcaacgctgtggtcggaacagcattgattaagaagtacccgaagctcgagtccgaattcgtgtacggtgactataaggtttacgatgtgcgcaagatgatcgccaagtcagagcaggaaattggcaaggccactgcgaagtatttcttttactctaacattatgaatttctttaagactgagatcacgctggctaatggcgaaatccggaagagaccacttattgagaccaacggcgagacaggggaaatcgtgtgggacaaggggagggatttcgccacagtccgcaaggttctctctatgcctcaagtgaatattgtcaagaagactgaagtccagacgggcgggttctcaaaggaatctattctgcccaagcggaactcggataagcttatcgccagaaagaaggactgggatccgaagaagtatggaggtttcgactcaccaacggtggcttactctgtcctggttgtggcaaaggtggagaagggaaagtcaaagaagctcaagtctgtcaaggagctcctgggtatcaccattatggagaggtccagcttcgaaaagaatccgatcgattttctcgaggcgaagggatataaggaagtgaagaaggacctgatcattaagcttccaaagtacagtcttttcgagttggaaaacggcaggaagcgcatgttggcttccgcaggagagctccagaagggtaacgagcttgctttgccgtccaagtatgtgaacttcctctatctggcatcccactacgagaagctcaagggcagcccagaggataacgaacagaagcaactgtttgtggagcaacacaagcattatcttgacgagatcattgaacagatttcggagttcagtaagcgcgtcatcctcgccgacgcgaatttggataaggttctctcagcctacaacaagcaccgggacaagcctatcagagagcaggcggaaaatatcattcatctcttcaccctgacaaaccttggggctcccgctgcattcaagtattttgacactacgattgatcggaagagatacacttctacgaaggaggtgctggatgcaacccttatccaccaatcgattactggcctctacgagacgcggatcgacttgagtcagctcggtggcgataagagacccgcagcaaccaagaaggcagggcaagcaaagaagaagaagtga

SEQ ID NO: 33 - DNA, 23 bp, Artificial Sequence
  CRISPR target sequence for OML4
gtgggttccggcaacctcaatgg

SEQ ID NO: 34 - DNA, 23 bp, Artificial Sequence
  CRISPR target sequence for GSK2
aggggaatgacgcggtgaccggg

SEQ ID NO: 35 - DNA, 20 bp, Artificial Sequence
  CRISPR protospacer sequence for OML4
gtgggttccggcaacctcaa

SEQ ID NO: 36 - DNA, 20 bp, Artificial Sequence
  CRISPR protospacer sequence for GSK2
aggggaatgacgcggtgacc

SEQ ID NO: 37 - protein, 77 aa, Oryza sativa
  OML4 RNA recognition motif 1
SRTLFVRNINSNVEDSELKLLFEHFGDIRALYTACKHRGFVMISYYDIRSALNAKMELQNKALRRRKLDIHYSIPKD

SEQ ID NO: 38 - protein, 71 aa, Oryza sativa
  OML4 RNA recognition motif 2
QGTIVLFNVDLSLTNDDLHKIFGDYGEIKEIRDTPQKGHHKIIEFYDVRAAEAALRALNRNDIAGKKIKLE

SEQ ID NO: 39 - protein, 86 aa, Oryza sativa
  OML4 RNA recognition motif 3
LMIKNIPNKYTSKMLLAAIDENHKGTYDFIYLPIDFKNKCNVGYAFINMTNPQHIIPFYQTFNGKKWEKFNSEKVASLAYARIQGK

SEQ ID NO: 40 - protein, 71 aa, Oryza sativa
  large1-1 amino acid sequence
MPSQVMDQRHHMSQYSHPTLAASSFSEELRLPTERQVGFWKQESLPHHMGSKSVASSPIEKPQPIGTRMAD